(19) United States
(12) Patent Application Publication
(10) Pub. No.: US 2026/0308186 A1
(43) Pub. Date: Oct. 8, 2026

(54) STAT DEGRADERS AND USES THEREOF

(71) Applicant: Recludix Pharma, Inc., San Diego, CA (US)

(72) Inventors: Neil Bifulco, San Diego, CA (US); Howard Bregman, San Diego, CA (US); Giovanni Cianchetta, San Diego, CA (US); Brian Hodous, Arlington, MA (US); Samuel K. Reznik, San Diego, CA (US); Yong Tang, San Diego, CA (US); Andrew Tasker, San Diego, CA (US); Rishi G. Vaswani, San Diego, CA (US); Ernest Allen Sickmier, San Diego, CA (US); John Yeoman, San Diego, CA (US); Xia Tian, San Diego, CA (US)

(21) Appl. No.: 19/479,610

(22) PCT Filed: May 15, 2024
(86) PCT No.: PCT/US2024/029364

§ 371 Date: Oct. 28, 2025

Related U.S. Application Data
(60) Provisional application No. 63/466,563, filed on May 15, 2023.

(51) Int. Cl. C07F 9/6561; A61K 31/675

(52) U.S. Cl. CPC C07F 9/6561; A61K 31/675

(57) ABSTRACT

Provided are compounds of Formula (I): and pharmaceutically acceptable satis and compositions thereof, which are useful for treating a variety of conditions associated with STAT3 and/or STAT6.

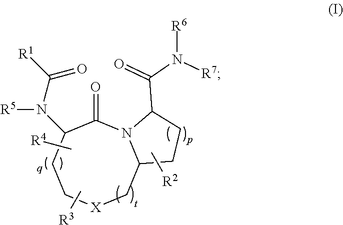

### BACKGROUND

The Signal Transducer and Activator of Transcription (STAT) family of proteins consists of transcription factors that play an essential role in the regulation of cell processes, such as proliferation, differentiation, apoptosis and angiogenesis. Seven STAT genes have been identified in the human genome: STAT1, STAT2, STAT3, STAT4, STAT5a, STAT5b, and STAT6.

STAT3 has received particular attention because it is strongly associated with the promotion of tumor growth and immune evasion, and the only STAT family member whose genetic deletion results in embryonic lethality. Indeed, aberrantly elevated STAT3 activity has been estimated to occur in more than 70% of human cancers. Activated STAT3 mediates critical gene expression changes and molecular events that dysregulate cell growth and apoptosis, promote angiogenesis, invasion, metastasis, and the development of resistance to apoptosis, and suppress the host's immune surveillance of the tumor, thereby making constitutively-active STAT3 a critical mediator of carcinogenesis and tumor progression.

Another STAT protein that has gained recent interest is STAT6. Recent studies have shown that STAT6 signaling is essential for IL-4- and IL-13-induced epithelial mesenchymal transition (EMT) and aggressiveness of colorectal cancer cells (CRC) cells. STAT6 is involved in several aspects of inflammatory disease and other related conditions.

### SUMMARY

Given their role in the regulation of cell processes, modulating the activity of one or more STAT proteins, particularly STAT3 and/or STAT6, represent a pivotal area of investigation for the treatment of cancer, inflammatory conditions, and other therapeutic needs.

Targeted protein degradation has become an advancing area as seen with rapid progression of PROTACs (proteolysis targeting chimeras) to clinic. PROTACs are protein degraders, which utilize the cell's own waste disposal machinery to eliminate, instead of inhibit, a target protein PROTACs are bifunctional in that they simultaneously bind a target protein and an E3 ligase protein. This event subsequently ubiquitylates the target, marking it for proteasomal degradation. This, and related processes, represent a new therapeutic modality for the treatment of diseases.

The present invention provides novel bifunctional compounds for degradation of STAT3 and/or STAT6 by recruiting them to E3 uniquitin ligase for degradation. The advantages of using the methods of the invention are that a broad range of targets that may be modulated by recruiting E3 ubiquitin ligase for degradation.

In certain aspects, the invention provides effective degraders of the STAT3 and/or STAT6. The degraders of STAT3 and/or STAT6 have general Formula A:

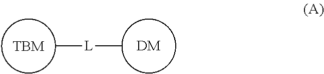

wherein, TBM is target binding moiety; DM is degradation moiety; and L is a linker between TBM and DM. A TBM is a moiety that binds to a target protein that should be degraded. In certain embodiments, the TBM is the moiety that binds to STAT3 and/or STAT6. The degradation moiety is responsible for including degradation of the target. In certain embodiments, the degradation moiety acts by recruitment of ubiquiting E3 ligase to the target attached to TBM, and thus results in degradation of the target.

In certain aspects, the compounds of the invention provide that the TBM moiety comprises a STAT3 and/or STAT6 binding moiety. The STAT3 and/or STAT6 binding moeity are inhibitors and/or modulators of STAT3 and/or STAT6. In certain embodiments, the STAT3 and/or STAT6 binding are compounds provided in WO 2023/164680, which is incorporated by reference in its entirey.

In certain embodiments, the STAT3 and/or STAT6 binding moiety is a compound of Formula (I′):

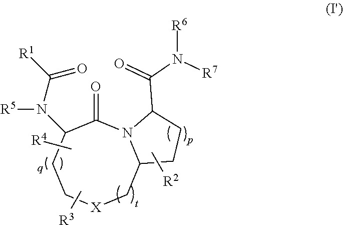

wherein:
q is 0 or 1 and t is 0, 1, or 2, provided that at least one of q or t is 1;
p is 1 or 2;
X is selected from S, SO₂, —S(═O)═NH, and NR⁸;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}]. —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1b}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}], wherein said aryl may be further optionally substituted with 1 or 2 groups independently selected from cyano, (C₁-C₄)alkoxy, and halo; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2b}P(O)[OR^{1b}][NH(AA)C(O)OR^{T}], —P(O)OR^{1b}OR^{2b}, —[P(O)[NHR^{Ty}][NH(AA)C(O)OR^{T}], —[P(O)[NH(AA)C(O)ORT][NH(AA)C(O)ORT], or —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₄)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
R⁵ and R⁶ are each independently selected from hydrogen, phenyl, and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 10-membered heteroaryl, —(C₁-C₄)[5- to 10-membered heteroaryl], phenyl, —(C₁-C₄)alkylphenyl, —C(O)R^{Ha}, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 10-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R^{Ha} is selected from (C₁-C₁₀)alkyl, (C₂-C₁₀)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R^{O} and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, cyano, and 4- to 6-membered heterocyclyl optionally substituted with C₁-C₄alkyl;
R^{O} is selected from halo, (C₁-C₄)alkoxy, OH, phenyl, NH₂, —NH(C₁-C₁₀)alkyl, —N[(C₁-C₁₀)alkyl], (C₃₋₆cycloalkyl), 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano;
R^{Hb} is hydrogen or (C₁-C₄)alkyl;
AA is the residue of an alpha or beta natural or non-natural amino acid;
R^{T} and R^{Ty} are each independently selected from (C₁-C₄)alkyl, benzyl, and phenyl, wherein said phenyl is optionally substituted with 1 or 2 groups selected from halo, (C₁-C₄)alkyl, and halo(C₁-C₄)alkyl; R^{Q} and R^{U} are each independently selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, R^{X}, and R^{Z} are each independently selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{e}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

Provided herein are compounds having the structural Formula I:

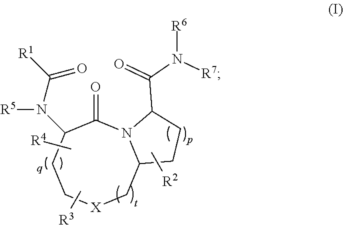

and pharmaceutically acceptable salts and compositions thereof, wherein:
q is 0 or 1 and t is 0, 1, or 2, provided that at least one of q or t is 1;
p is 1 or 2;
X is selected from S, SO₂, —S(═O)═NH, —NR⁸, and —NR⁹;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an aryl substituted with —CR^{1a}R^{2b}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}], wherein said aryl may be further optionally substituted with 1 or 2 groups independently selected from cyano, (C₁-C₄)alkoxy, and halo; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], —[P(O)[NH(AA)C(O)OR^{T}][NH(AA)C(O)OR^{T}], or —P(O)[OR^{1b}][NH(AA)C(O)OR^{T}], and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2b}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl, [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O-+(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)-[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₁-C₄)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S}.
R⁵ and R⁶ are each independently selected from hydrogen, phenyl, and (C₁-C₄)alkyl;
R⁷ is selected from E, —R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q};
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 10-membered heteroaryl, —(C₁-C₄)[5- to 10-membered heteroaryl], phenyl, —(C₁-C₄)alkylphenyl, —C(O)R^{Ha}, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 10-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R⁹ is selected from —R¹⁰E and —C(O)R¹⁰E;
R¹⁰, R^{10A}, R^{10B}, R^{10C}, and R^{10D} are each independently a chemical spacer unit;
R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with-OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with-OR^{10C}E, —NHR^{10C}E, —[N(C₁-C₄)alkyl]R^{10C}E, or R^{10C}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with-OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
R^{Ha} is selected from (C₁-C₁₀)alkyl, (C₂-C₁₁)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R^{O} and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, cyano, and 4- to 6-membered heterocyclyl optionally substituted with C₁-C₄alkyl;
R^{O} is selected from halo, (C₁-C₄)alkoxy, OH, phenyl, NH₂, —NH(C₁-C₁₀)alkyl, —N[(C₁-C₁₀)alkyl], (C₃₋₆cycloalkyl), 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano;
R^{Hb} is hydrogen or (C₁-C₄)alkyl;
AA is the residue of an alpha or beta natural or non-natural amino acid;
R^{T} and R^{Ty} are each independently selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} and R^{U} are each independently selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocylcyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, and R^{X} are each independently selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{e}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)—NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

In one aspect, the disclosed compounds of Formula I and pharmaceutically acceptable salts thereof act as protein degraders of STAT3 and/or STAT6, and are useful in a variety of therapeutic applications such as, for example, in treating cancer and inflammatory conditions.

Pharmaceutical compositions comprising the compounds and pharmaceutically acceptable salts of Formula I, as well as methods for their preparation are also included.

Methods of treating conditions responsive to the degradation of STAT3 and/or STAT6 using the described compounds, pharmaceutically acceptable salts, and compositions thereof are also included.

### DETAILED DESCRIPTION

### 1. General Description of Compounds

The present invention provides novel bifunctional compounds for degradation of STAT3 and/or STAT6 by recruiting them to E3 uniquitin ligase for degradation. The advantage of using the methods of the invention are that a broad range of targets that may be modulated by recruiting E3 ubiquitin ligase for degradation.

In certain aspects, the invention provides effective degraders of the STAT3 and/or STAT6. The degraders of STAT3 and/or STAT6 have general Formula A:

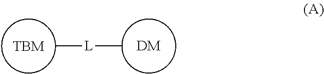

wherein, TBM is target binding moiety; DM is degradation moiety; and L is a linker between TBM and DM. A TBM is a moiety that binds to a target protein that should be degraded. In certain embodiments, the TBM is the moiety that binds to STAT3 and/or STAT6. The degradation moiety is responsible for including degradation of the target. In certain embodiments, the degradation moiety acts by recruitment of ubiquiting E3 ligase to the target attached to TBM, and thus results in degradation of the target.

In certain aspects, the compounds of the invention provide that the TBM moiety comprises a STAT3 and/or STAT6 binding moiety. The STAT3 and/or STAT6 binding moeity are inhibitors and/or modulators of STAT3 and/or STAT6. In certain embodiments, the STAT3 and/or STAT6 binding are compounds provided in WO 2023/164680, which is incorporated by reference in its entirey.

In a first embodiment, the invention provides compounds wherein the TBM is a compound of Formula (I′):

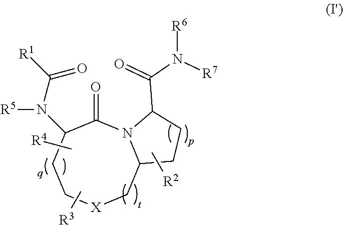

wherein R¹, R², R³, R⁴, R⁵, R⁶, R⁷, X, q, t, and q are described above.

In a second embodiment, provided herein is a compound of structural Formula I:

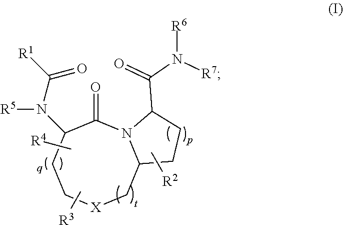

or a pharmaceutically acceptable salt thereof, wherein:
q is 0 or 1 and t is 0, 1, or 2, provided that at least one of q or t is 1;
p is 1 or 2;
X is selected from S, SO₂, —S(═O)═NH, —NR⁸, and —NR⁹;
R¹ is selected from an 8- to 10-membered fused bicyclic heteroaryl substituted with-CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}[NH(AA)C(O)OR^{T}], or —P(O)[OR^{1b}][NH(AA)C(O)OR^{T}], wherein said aryl may be further optionally substituted with 1 or 2 groups independently selected from cyano, (C₁-C₄)alkoxy, and halo, a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], —[P(O)[NH(AA)C(O)ORT][NH(AA)C(O)ORT], or —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocycyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
R⁵ and R⁶ are each independently selected from hydrogen, phenyl, and (C₁-C₄)alkyl;
R⁷ is selected from E, —R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q};
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 10-membered heteroaryl, —(C₁-C₄)[5- to 10-membered heteroaryl], phenyl, —(C₁-C₄)alkylphenyl, —C(O)R^{Ha}, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 10-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R⁹ is selected from-R¹⁰E and —C(O)R¹⁰E;
R¹⁰, R^{10A}, R^{10B}, R^{10C}, and R^{10D} are each independently a chemical spacer unit;
R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with-OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10C}E, —NHR^{10C}-E, —[N(C₁-C₄)alkyl]R^{10C}E, or R^{10B}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
R^{Ha} is selected from (C₁-C₁₀)alkyl, (C₂-C₁₀)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R^{O} and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, cyano, and 4- to 6-membered heterocyclyl optionally substituted with C₁-C₄alkyl,
R^{O} is selected from halo, (C₁-C₄)alkoxy, OH, phenyl, NH₂, —NH(C₁-C₁₀)alkyl, —N[(C₁-C₁₀)alkyl], (C₃₋₆cycloalkyl), 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano;
R^{Hb} is hydrogen or (C₁-C₄)alkyl;
AA is the residue of an alpha or beta natural or non-natural amino acid;
R^{T} and R^{Ty} are each independently selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} and R^{U} are each independently selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{e}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, and R^{X} are each independently selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{c}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₁-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

### 2. Definitions

When used in connection to describe a chemical group that may have multiple points of attachment, a hyphen (-) designates the point of attachment of that group to the variable to which it is defined. For example, —NR^{c}C(O)R^{e} means that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

Unless otherwise specified, the term “alkyl” when used alone or as part of a larger moiety, such as “haloalkyl”, and the like, means saturated straight-chain or branched monovalent hydrocarbon radical.

The term “haloalkyl” includes mono, poly, and perhaloalkyl groups where the halogens are independently selected from fluorine, chlorine, bromine, and iodine.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

The term “oxo” means the group ═O.

The term “imino” means the group ═NH.

Unless otherwise specified, the term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, benzothiopheneyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

Unless otherwise specified, the term “heterocyclyl” means a 4- to 12-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”. “heterocyclic moiety”, and “heterocyclic radical”, are used interchangeably herein. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, terahydropyranyl, pyrrolidinyl, pyrrolidonyl, piperidinyl, oxazolidinyl, piperadinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3,4]oct-6-enyl, 6-thia-2,7-diazaspiro[3,4]octanyl, 2,6-diazaspiro[3,3]heptanyl, spiro[indoline-3,3′-pyrrolidine]-yl, thiochromanyl, and the like. It will be understood that when specified, optional substituents on a heterocyclyl group may be present on any substitutable position and, include, e.g., the position at which the heterocyclyl is attached (where valency permits).

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “aryl” refers to an aromatic carbocyclic single ring or two fused ring system containing 6 to 10 carbon atoms. Examples include phenyl, indanyl, tetrahydronaphthalene, and naphthyl. In one aspect, the aryl is phenyl or naphthyl.

The terms “cycloalkyl”, used alone or as part of a larger moiety, refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The term alkylene refers to a refers to a bivalent radical of a saturated, straight or branched chain hydrocarbon (e.g., CH₂) of the specified length.

The term alkynylene refers to an alkylene group that has at least one triple bond within the chain e.g., —(CH₂)₅CCH—.

The “residue of an amino acid” is the moiety remaining after formation of a bond between a reactive group in another compound (e.g., an amino group) and the carboxylic acid in the amino acid, after formation of a bond between a reactive group in another compound (e.g., a carboxylic acid) and the amino group in the amino acid, or both. As a consequence of the bond(s) formation, the carboxylic acid in the amino acid no longer has the OH group and instead has a bond between the carbonyl group and the reactive group in the compound; the amino group has only one hydrogen atom and instead has a bond between the reactive group in the other compound and the nitrogen of the amino group; or both. For example, the “residue of an alpha amino acid” can be depicted structurally as NH₂CR′R—C(O)—, —NHCR′R—C(O)OH or —NHCR′R—C(O)—; and the “residue of an beta amino acid” can be depicted structurally as or NH₂CR′RCH₂—C(O)—, —NHCR′RCH₂—C(O)OH or —NHCR′RCH₂—C(O)—, where R′ is H or C₁-C₆ alkyl and R is H or C₁-C₆ alkyl optionally substituted with 1 to 3 groups selected from halo, (C₁-C₃)alkoxy, OH, NH—, —NH(C₁-C₄ alkyl), —N[(C₁-C₄ alkyl)]₂, SH, S(C₁-C₄ alkyl), imino, COOH, —COO(C₁-C₄ alkyl), —CO(C₁-C₄ alkyl), —CONH(C₁-C₄ alkyl)phenyl, phenyl, and 5- to 10-membered heteroaryl, wherein said C₁-C₆ alkyl may also be optionally interrupted by a sulfur or nitrogen heteroatom and wherein said phenyl is optionally substituted with 1 to 3 groups selected from OH, cyano, (C₁-C₄ alkyl), and halo(C₁-C₄ alkyl); or R is taken together with the nitrogen atoms from the alpha or beta amino acid residue to form a 4- to 6-membered heterocyclyl. For naturally occurring alpha amino acid (i.e., amino acids that occur in nature), R′ is H and R is selected from hydrogen, methyl, isopropyl, —CH₂CH(CH)₂, —(CH₂)₂SCH₃, —CH(CH₃)(CH₂CH₃), CH₂OH, —CH(OH)(CH₃), CH₂SH, —CH₂C(O)NH₂, —(CH₂)₂C(O)NH₂, benzyl, p-hydroxybenzyl, —CH₂(indolyl), —(CH₂)₄NH₂, —(CH₂)₃NHC(═NH₂)NH₂, —CH₂(imidazolyl), —(CH₂)COOH, and —(CH₂)₂COOH; or R taken together with the nitrogen atom of the alpha or beta amino acid residue forms a pyrrolidinyl ring.

Non-natural amino acids are known in the art and include e.g., alpha-alkyl amino acids (e.g., alpha methyl), alpha-alkylalkoxy amino acids (e.g., alpha —CH₂OCH₃), N-methyl amino acids, homo-amino acids, etc.

A chemical spacer unit refers to refers to a cleavable or non-cleavable that joins the E3 ligase binder to the remainder of the molecule described herein. In one aspect, the chemical spacer is uncleavable in vivo. In one aspect, the chemical spacer comprises one or more cyclic ring systems. In another aspect, the chemical spacer comprises an alkyl chain optionally substituted by and/or interrupted with one or more chemical groups. In one aspect, the chemical spacer comprises optimal spatial and chemical properties to effectuate optimal therapeutic activity.

A chemical moiety that binds to E3 ligase refers to a chemical structure that binds to the pocket or surface of E3 ligase. E3 ligase binders are known in the art and include, but are not limited to CRBN, VHL, IAP, or MDM2 based E3 binders. See e.g., Angewandte Chemie International Edition 59(36). May 2020. DOI:10.1002/anie.202004310; Medicinal Chemistry Communication 10(10), August 2019, DOI:10.1039/C9MD00272C; and Molecules 2022, 27(19), 6515; https://doi.org/10.3390/molecules27196515.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to isomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of the ring, whereas “trans” refers to substituents oriented on opposite sides of the ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

The terms “subject” and “patient” may be used interchangeably, and means a mammal in need of treatment. e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the terms “treatment.” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin. buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that is sufficient to achieve the desired therapeutic effect (such as treatment of a condition recited herein) under the conditions of administration.

### 3. General Description of the Compounds of the Invention

In certain aspects, the invention provides effective degraders of the STAT3 and/or STAT6. The degraders of STAT3 and/or STAT6 have general Formula A:

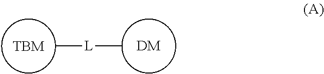

wherein, TBM is target binding moiety; DM is degradation moiety; and L is a linker between TBM and DM. A TBM is a moiety that binds to a target protein that should be degraded. In certain embodiments, the TBM is the moiety that binds to STAT3 and/or STAT6. The degradation moiety is responsible for including degradation of the target. In certain embodiments, the degradation moiety acts by recruitment of ubiquiting E3 ligase to the target attached to TBM, and thus results in degradation of the target.

In certain aspects, the compounds of the invention provide that the TBM moiety comprises a STAT3 and/or STAT6 binding moiety. The STAT3 and/or STAT6 binding moeity are inhibitors and/or modulators of STAT3 and/or STAT6. In certain embodiments, the STAT3 and/or STAT6 binding are compounds provided in WO 2023/164680, which is incorporated by reference in its entirey.

In certain embodiments, the STAT3 and/or STAT6 binding moiety is a compound of Formula (I′):

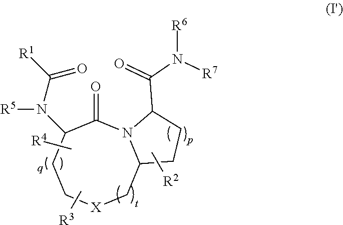

wherein:
q is 0 or 1 and t is 0, 1, or 2, provided that at least one of q or t is 1;
p is 1 or 2;
X is selected from S, SO₂, —S(═O)═NH, and NR⁸;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}], an aryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}], wherein said aryl may be further optionally substituted with 1 or 2 groups independently selected from cyano, (C₁-C₄)alkoxy, and halo; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], —[P(O)[NH(AA)C(O)ORT][NH(AA)C(O)ORT], or —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
R⁵ and R⁶ are each independently selected from hydrogen, phenyl, and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 10-membered heteroaryl, —(C₁-C₄)[5- to 10-membered heteroaryl], phenyl, —(C₁-C₄)alkylphenyl, —C(O)R^{Ha}, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 10-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R^{Ha} is selected from (C₁-C₁₀)alkyl, (C₂-C₁₀)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R^{O} and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, cyano, and 4- to 6-membered heterocyclyl optionally substituted with C₁-C₄alkyl;
R^{O} is selected from halo, (C₁-C₄)alkoxy, OH, phenyl, NH₂, —NH(C₁-C₁₀)alkyl, —N[(C₁-C₁₀)alkyl], (C₃₋₆cycloalkyl), 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano;
R^{Hb} is hydrogen or (C₁-C₄)alkyl;
AA is the residue of an alpha or beta natural or non-natural amino acid;
R^{T} and R^{Ty} are each independently selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} and R^{U} are each independently selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)—NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{c}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{e}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, R^{X}, and R^{Z} are each independently selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{g}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alknyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₁-C₁₀)alkynl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

In certain embodiments from the present invention, the L and DM moieties are described herein. In certain embodiments, the L and DM moieties are provided in WO 2024/064080, which is incorporated by reference in its entirety.

In some embodiments, DM is an E3 ligase ligand well known to one of ordinary skill in the art including those described in M. Toure, C M. Crews. Angew. Chem. Int. Ed., 2016, 55, 1966, T. Uehara et al. Nature Chemical Biology, 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, US 2019/0076540, WO 2017/197046, US 2019/0076542, WO 2017/197051, US 2019/0076539, WO 2017/197055, US 2019/0076541, and WO 2017/197056, the entirety of each of which is herein incorporated by reference.

In certain embodiments. L is attached to a modifiable carbon, oxygen, or nitro substitution or replacement group on one or both of DM and TBM.

In certain embodiments, the DM is a cereblon E3 ubiquitin ligase binding moiety (hereinafter also referred to as LBM).

In some embodiments, LBM is selected from the group consisting of:

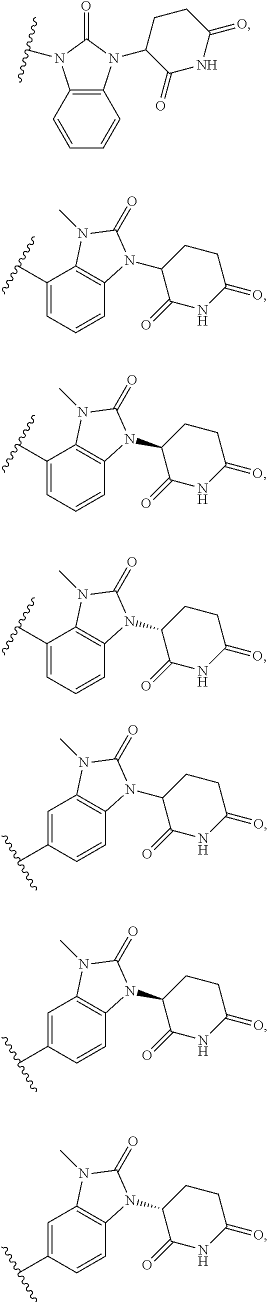

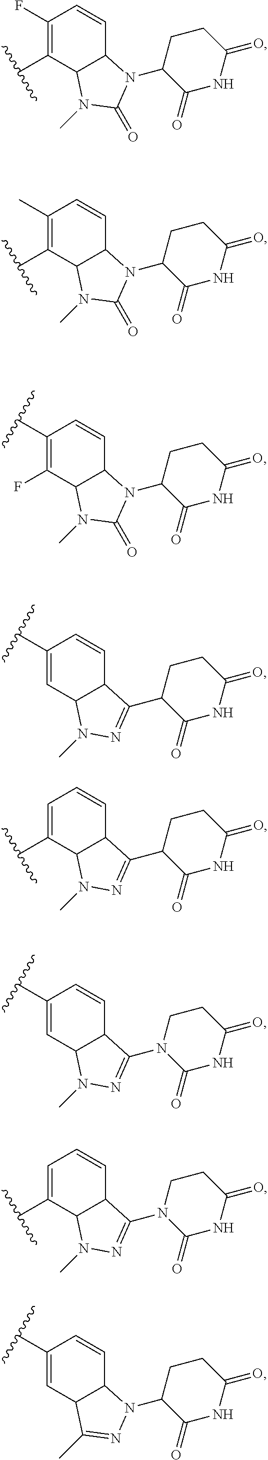

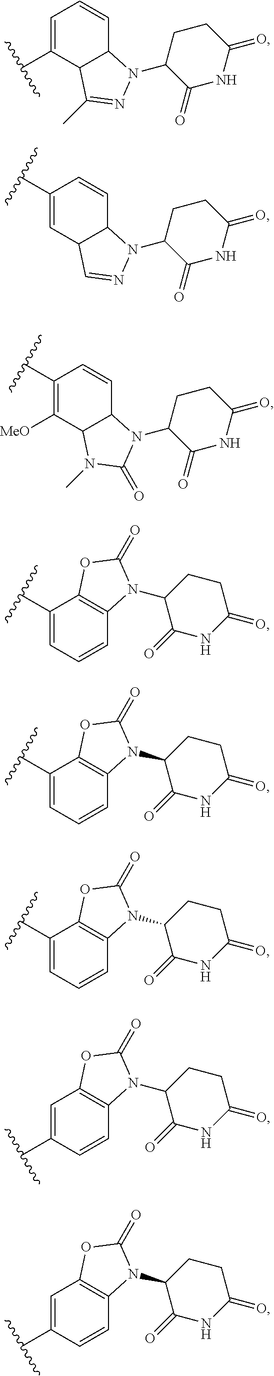

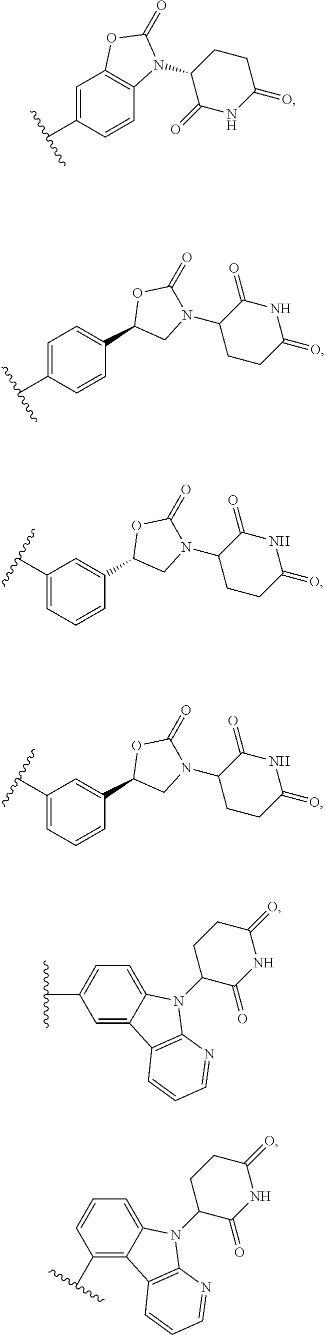 [SMILES: *c1ccc2c(c1)oc(=O)n2[C@@H]1CCC(=O)NC1=O ; *c1ccc([C@@H]2CN(C3CCC(=O)NC3=O)C(=O)O2)cc1 ; *c1cccc([C@H]2CN(C3CCC(=O)NC3=O)C(=O)O2)c1 ; *c1cccc([C@@H]2CN(C3CCC(=O)NC3=O)C(=O)O2)c1 ; *c1ccc2c(c1)c1cccnc1n2C1CCC(=O)NC1=O ; *c1cccc2c1c1cccnc1n2C1CCC(=O)NC1=O]

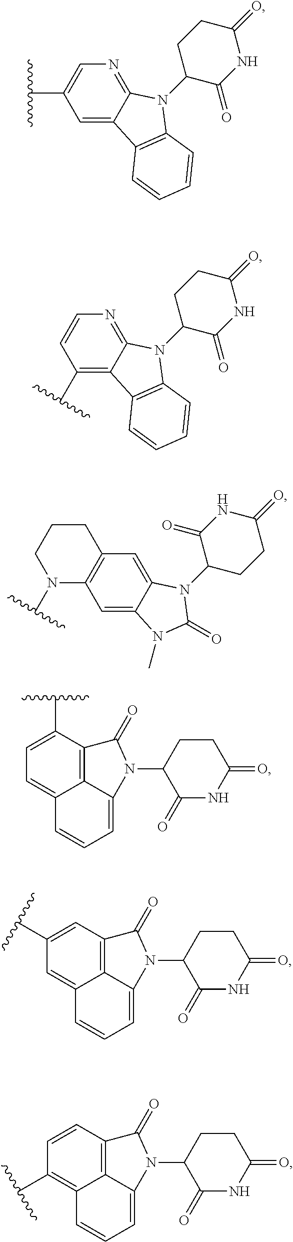

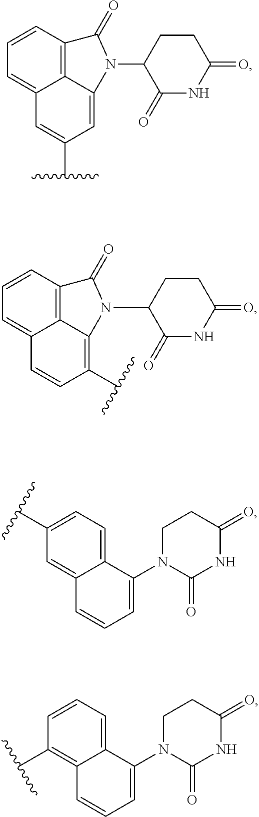 [SMILES: *c1cc2c3c(cccc3c1)C(=O)N2C1CCC(=O)NC1=O ; *c1ccc2cccc3c2c1N(C1CCC(=O)NC1=O)C3=O ; *c1ccc2c(N3CCC(=O)NC3=O)cccc2c1 ; *c1cccc2c(N3CCC(=O)NC3=O)cccc12]

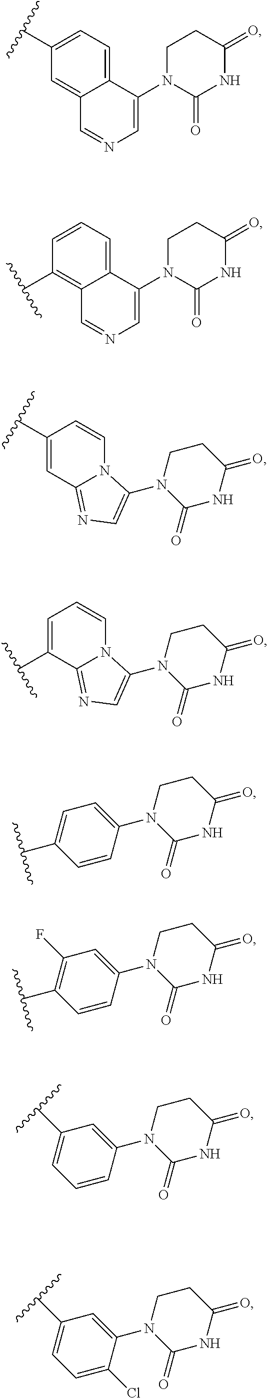

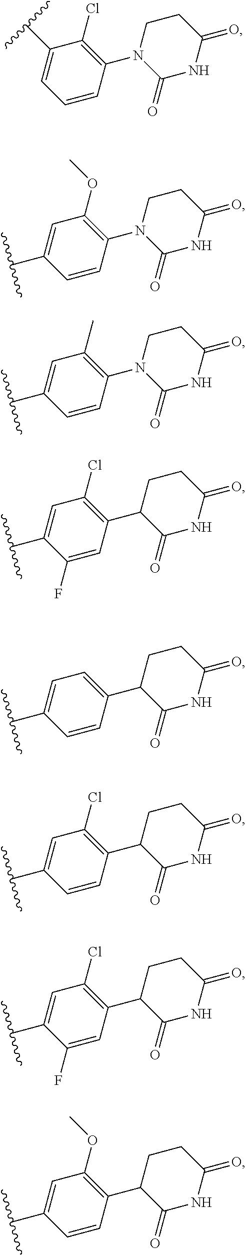

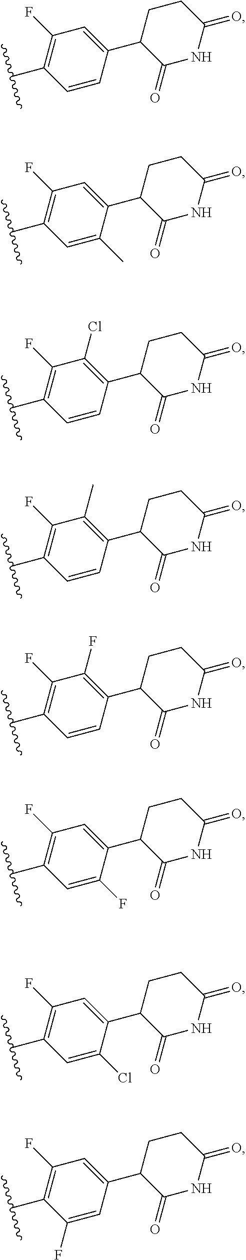

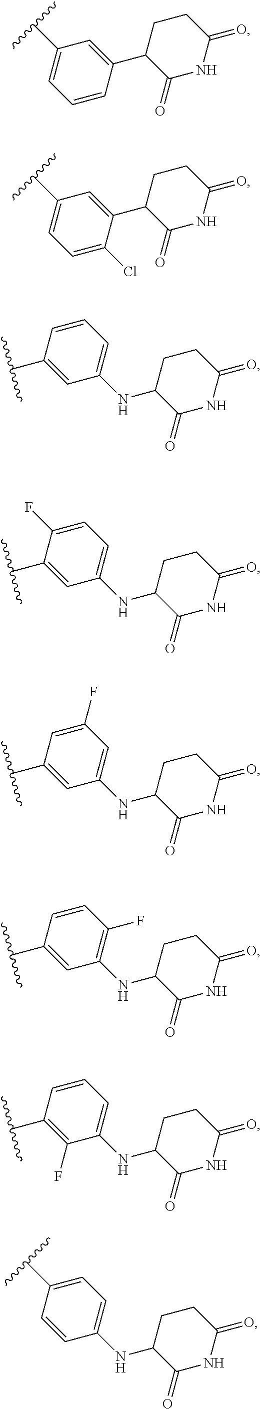

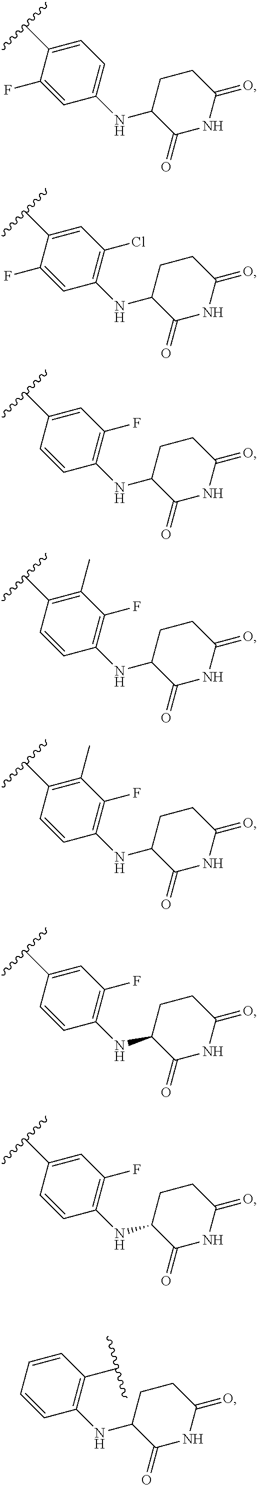

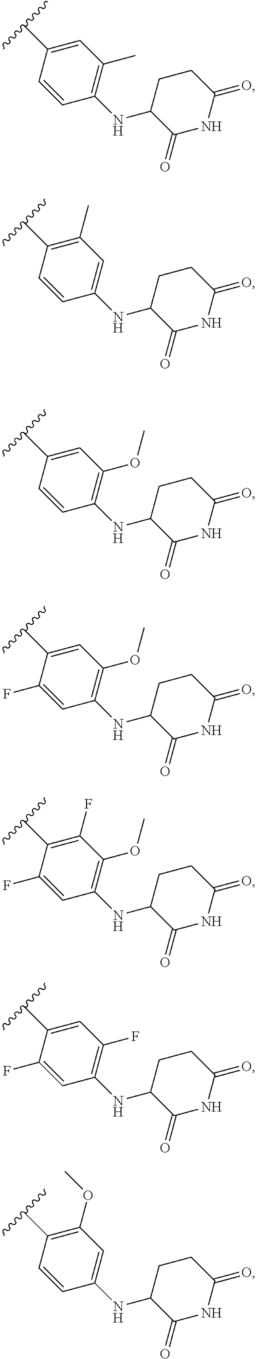

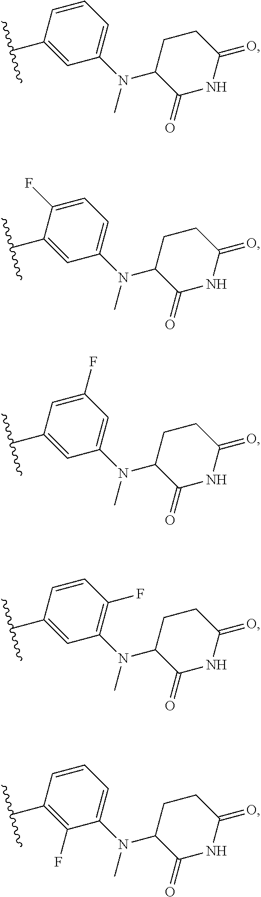 [SMILES: *c1cccc(N(C)C2CCC(=O)NC2=O)c1 ; *c1cc(N(C)C2CCC(=O)NC2=O)ccc1F ; *c1cc(F)cc(N(C)C2CCC(=O)NC2=O)c1 ; *c1ccc(F)c(N(C)C2CCC(=O)NC2=O)c1 ; *c1cccc(N(C)C2CCC(=O)NC2=O)c1F]

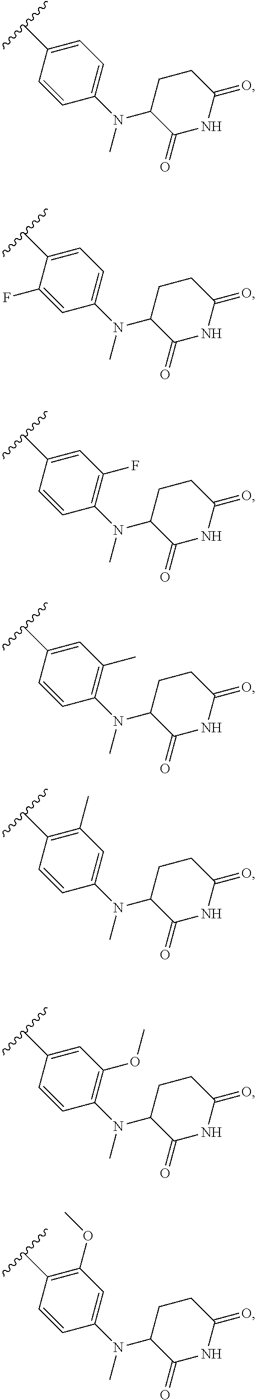

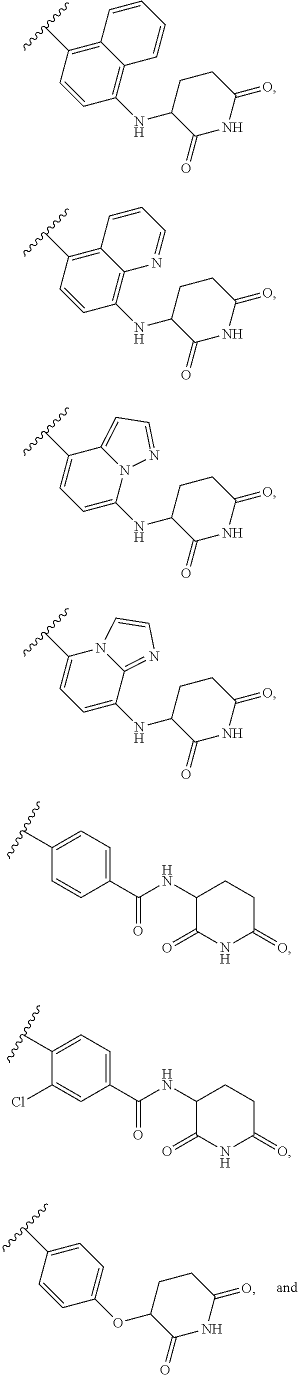

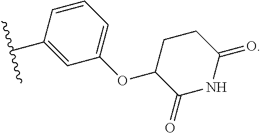 [SMILES: *c1cccc(OC2CCC(=O)NC2=O)c1]

In some embodiments, LBM is E3 Ubiquitin ligase binding moiety recited in Varfolomeev, E. et al., IAP Antagonists Induce Autoubiquitination of c-IAPs. NF-κB activation, and TNFα-Dependent Apoptosis, Cell, 2007, 131(4): 669-81, such as, for example:

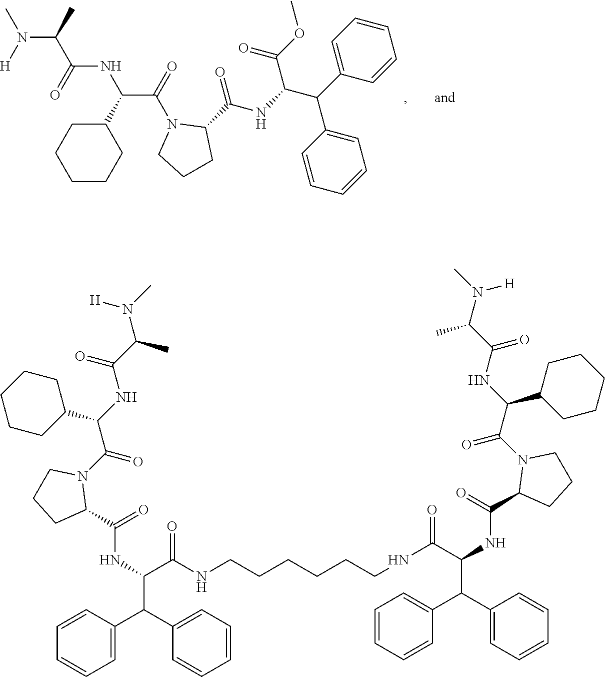

wherein L is attached to a modifiable carbon, oxygen, nitrogen or sulfur atom.

In certain embodiments, LBM is selected from the group consisting of:

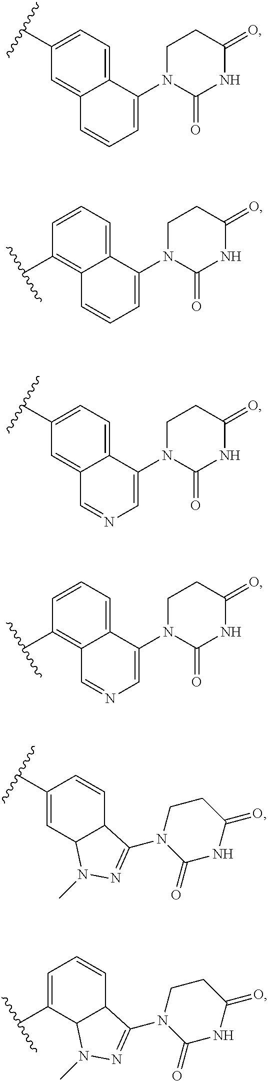 [SMILES: *c1ccc2c(N3CCC(=O)NC3=O)cccc2c1 ; *c1cccc2c(N3CCC(=O)NC3=O)cccc12 ; *c1ccc2c(N3CCC(=O)NC3=O)cncc2c1 ; *c1cccc2c(N3CCC(=O)NC3=O)cncc12 ; *C1=CC2C(C=C1)C(N1CCC(=O)NC1=O)=NN2C ; *C1=CC=CC2C(N3CCC(=O)NC3=O)=NN(C)C12]

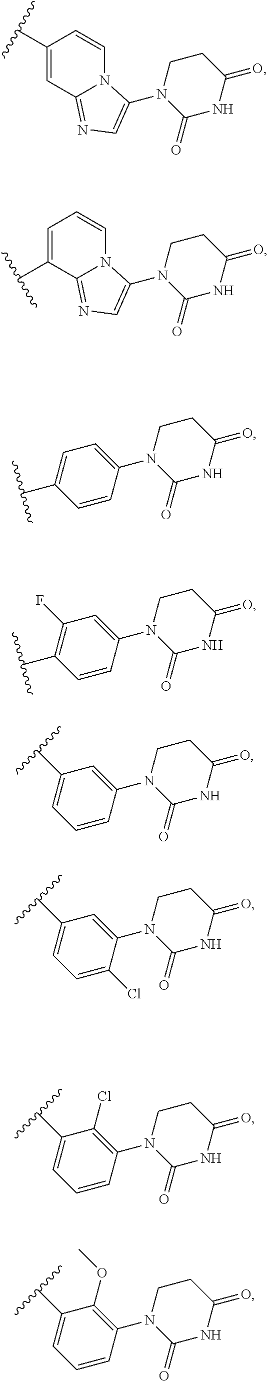

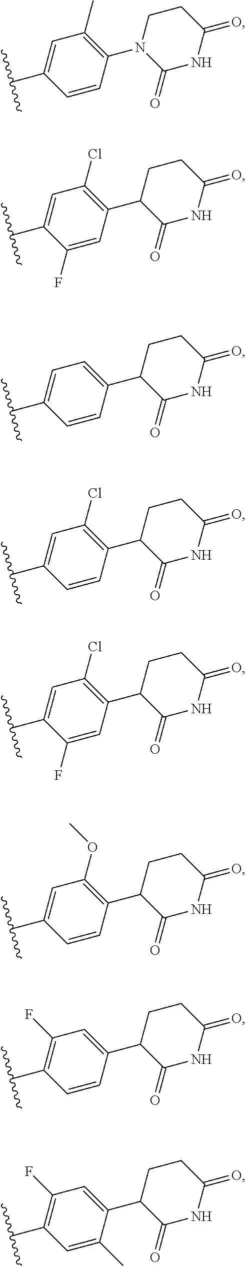

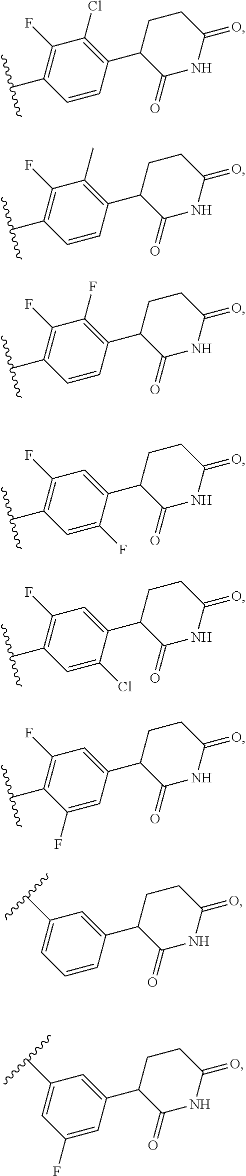

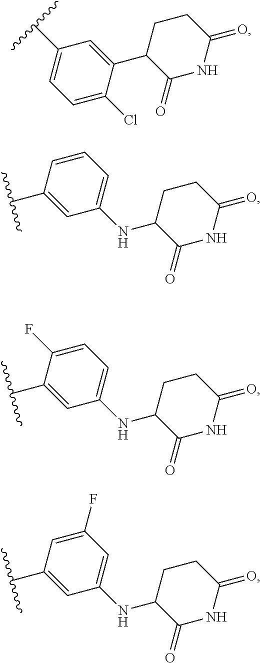 [SMILES: *c1ccc(Cl)c(C2CCC(=O)NC2=O)c1 ; *c1cccc(NC2CCC(=O)NC2=O)c1 ; *c1cc(NC2CCC(=O)NC2=O)ccc1F ; *c1cc(F)cc(NC2CCC(=O)NC2=O)c1]

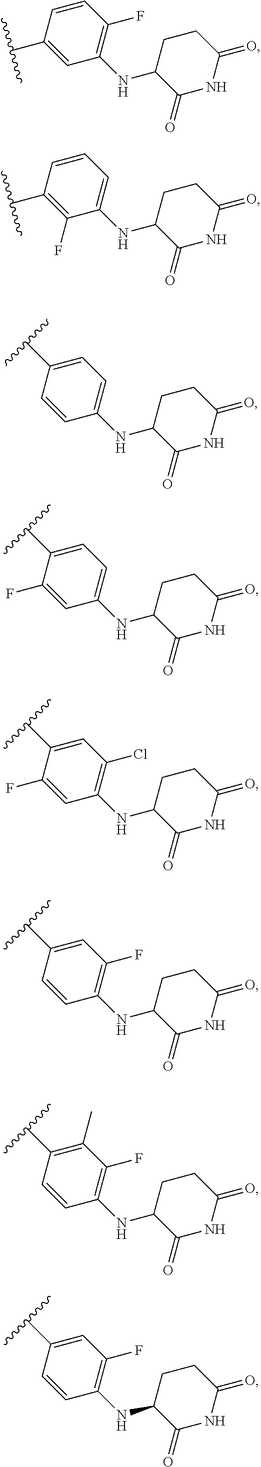

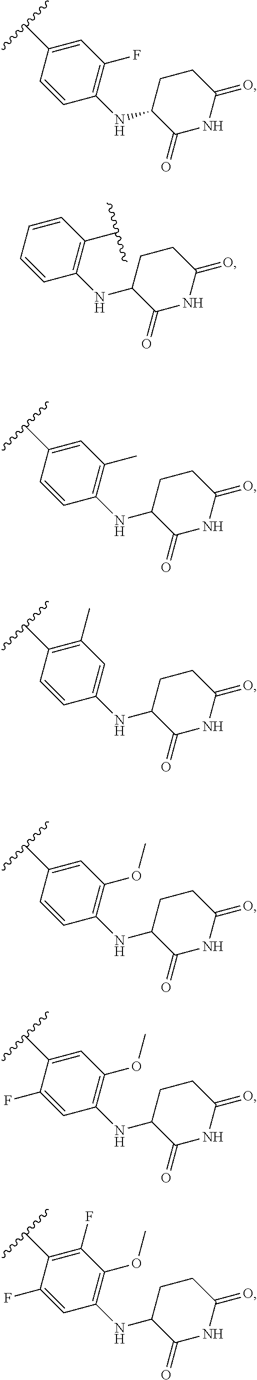

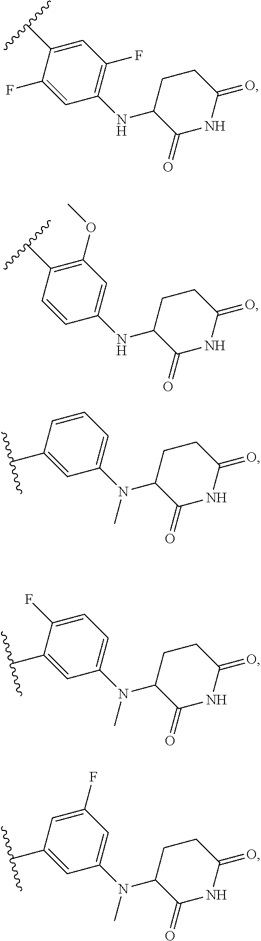 [SMILES: *c1cc(F)c(NC2CCC(=O)NC2=O)cc1F ; *c1ccc(NC2CCC(=O)NC2=O)cc1OC ; *c1cccc(N(C)C2CCC(=O)NC2=O)c1 ; *c1cc(N(C)C2CCC(=O)NC2=O)ccc1F ; *c1cc(F)cc(N(C)C2CCC(=O)NC2=O)c1]

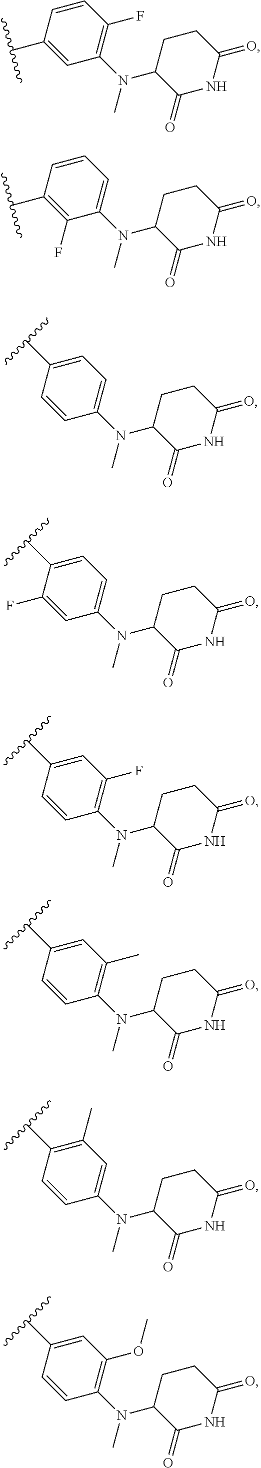

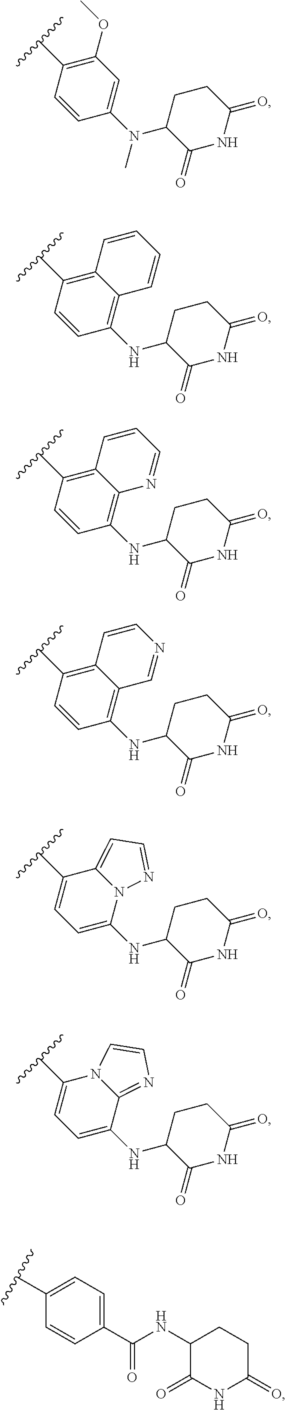

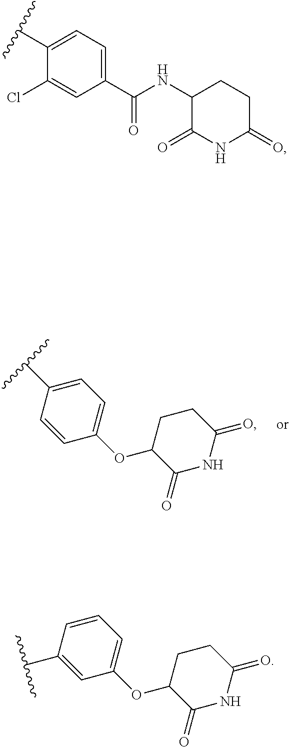 [SMILES: *c1ccc(C(=O)NC2CCC(=O)NC2=O)cc1Cl ; *c1ccc(OC2CCC(=O)NC2=O)cc1 ; *c1cccc(OC2CCC(=O)NC2=O)c1]

In certain embodiments, the LBM is selected from the group consisting of:

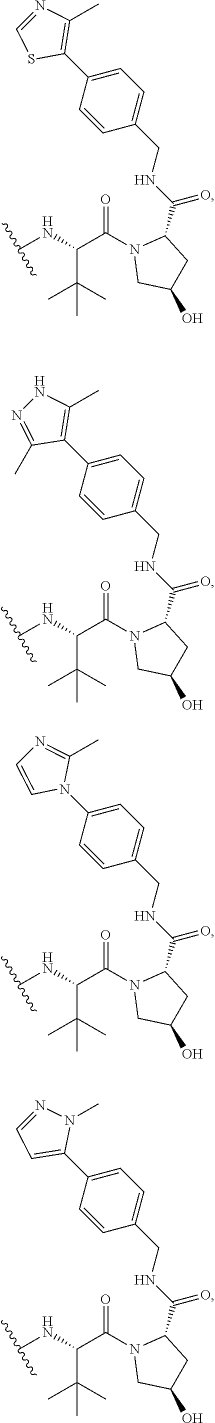

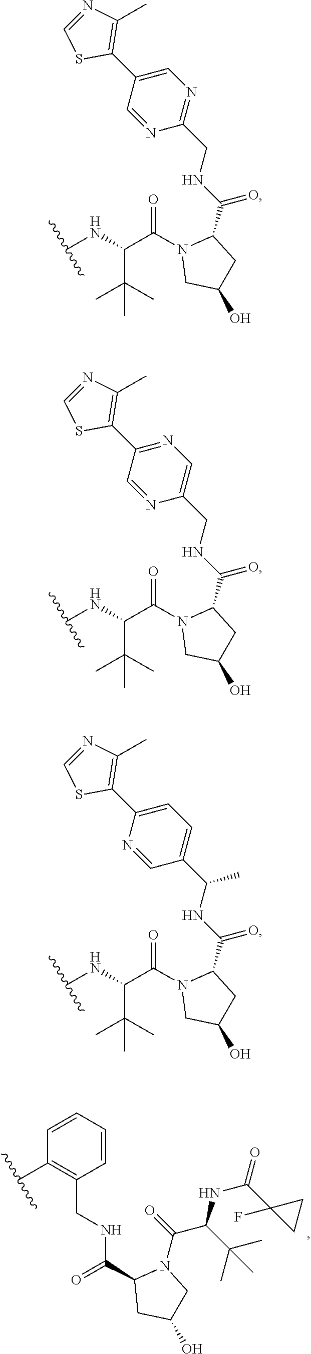 [SMILES: *N[C@H](C(=O)N1C[C@H](O)C[C@H]1C(=O)NCc1ncc(-c2scnc2C)cn1)C(C)(C)C ; *N[C@H](C(=O)N1C[C@H](O)C[C@H]1C(=O)NCc1cnc(-c2scnc2C)cn1)C(C)(C)C ; *N[C@H](C(=O)N1C[C@H](O)C[C@H]1C(=O)N[C@@H](C)c1ccc(-c2scnc2C)nc1)C(C)(C)C ; *c1ccccc1CNC(=O)[C@@H]1C[C@@H](O)CN1C(=O)[C@@H](NC(=O)C1(F)CC1)C(C)(C)C]

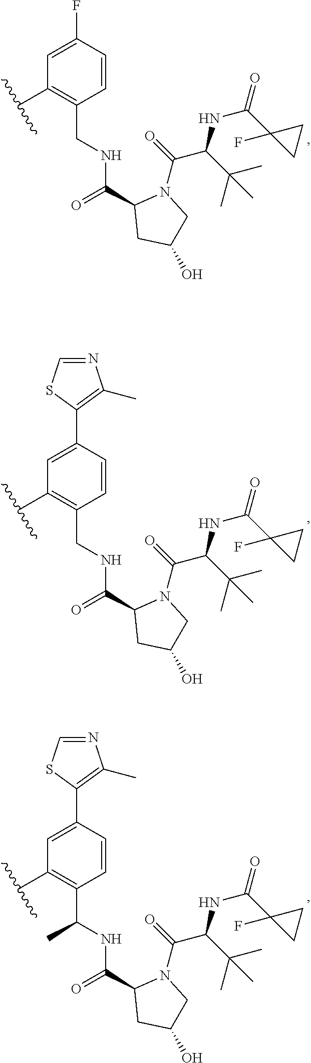 [SMILES: *c1cc(F)ccc1CNC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(F)CC1)C(C)(C)C ; *c1cc(-c2scnc2C)ccc1[C@H](C)NC(=O)[C@@H]1C[C@@H](O)CN1C(=O)[C@@H](NC(=O)C1(F)CC1)C(C)(C)C]

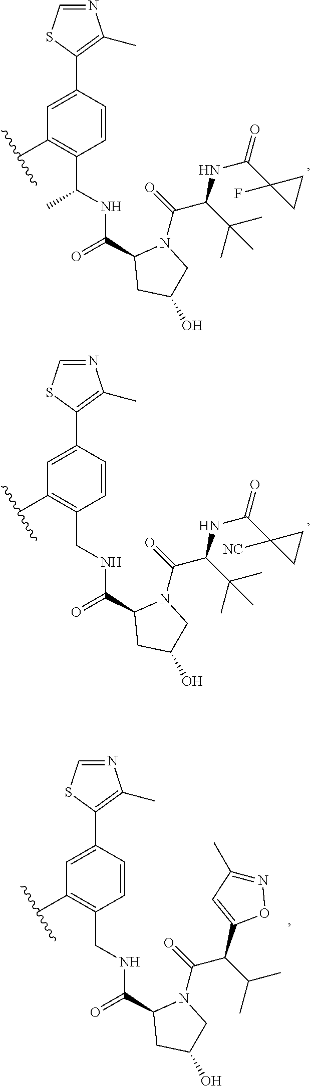 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(C#N)CC1)C(C)(C)C ; *c1cc(-c2scnc2C)ccc1CNC(=O)[C@@H]1C[C@@H](O)CN1C(=O)[C@@H](c1cc(C)no1)C(C)C]

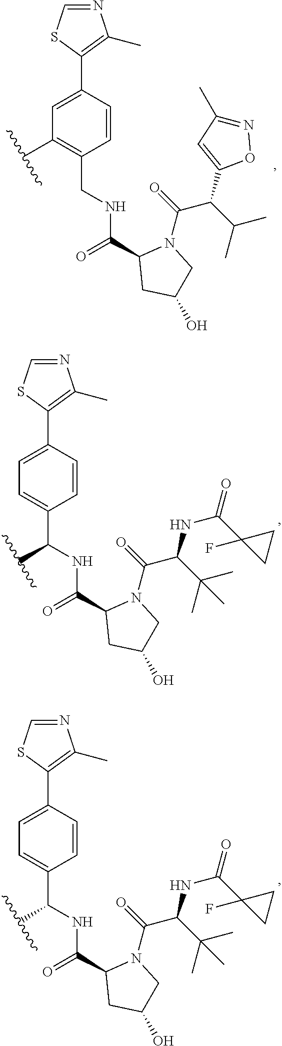 [SMILES: *c1cc(-c2scnc2C)ccc1CNC(=O)[C@@H]1C[C@@H](O)CN1C(=O)[C@H](c1cc(C)no1)C(C)C ; *[C@H](NC(=O)[C@@H]1C[C@@H](O)CN1C(=O)[C@@H](NC(=O)C1(F)CC1)C(C)(C)C)c1ccc(-c2scnc2C)cc1 ; *[C@@H](NC(=O)[C@@H]1C[C@@H](O)CN1C(=O)[C@@H](NC(=O)C1(F)CC1)C(C)(C)C)c1ccc(-c2scnc2C)cc1]

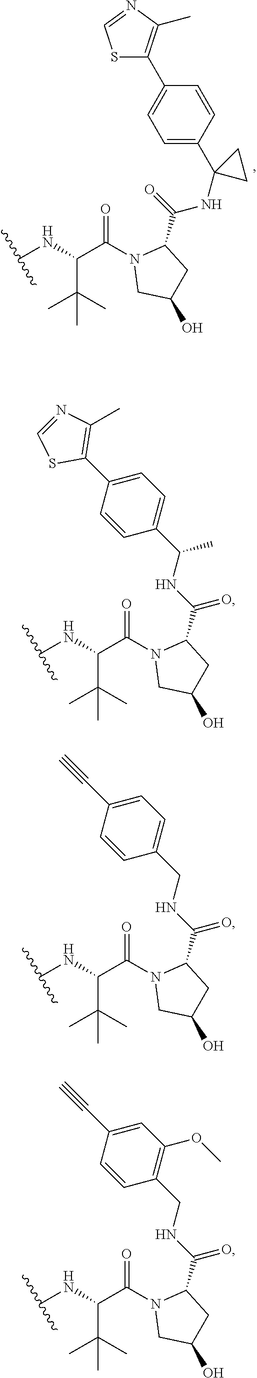 [SMILES: *N[C@H](C(=O)N1C[C@H](O)C[C@H]1C(=O)NC1(c2ccc(-c3scnc3C)cc2)CC1)C(C)(C)C ; *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)NCc1ccc(C#C)cc1)C(C)(C)C ; *N[C@H](C(=O)N1C[C@H](O)C[C@H]1C(=O)NCc1ccc(C#C)cc1OC)C(C)(C)C]

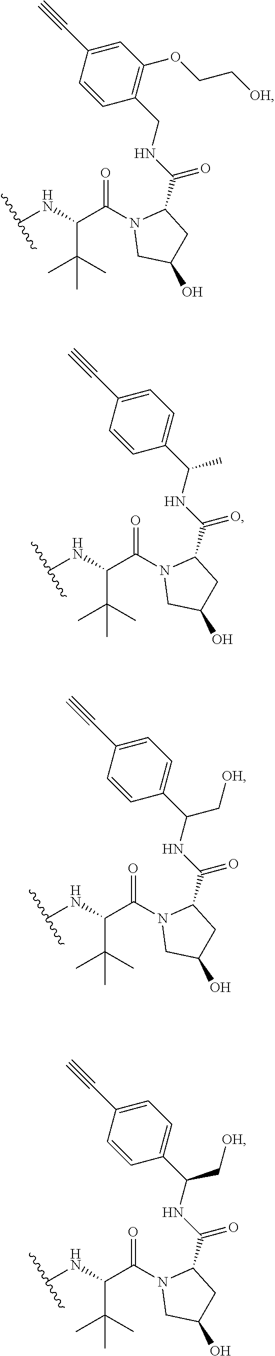 [SMILES: *N[C@H](C(=O)N1C[C@H](O)C[C@H]1C(=O)NCc1ccc(C#C)cc1OCCO)C(C)(C)C ; *N[C@H](C(=O)N1C[C@H](O)C[C@H]1C(=O)N[C@@H](C)c1ccc(C#C)cc1)C(C)(C)C ; *N[C@H](C(=O)N1C[C@H](O)C[C@H]1C(=O)NC(CO)c1ccc(C#C)cc1)C(C)(C)C ; *N[C@H](C(=O)N1C[C@H](O)C[C@H]1C(=O)N[C@H](CO)c1ccc(C#C)cc1)C(C)(C)C]

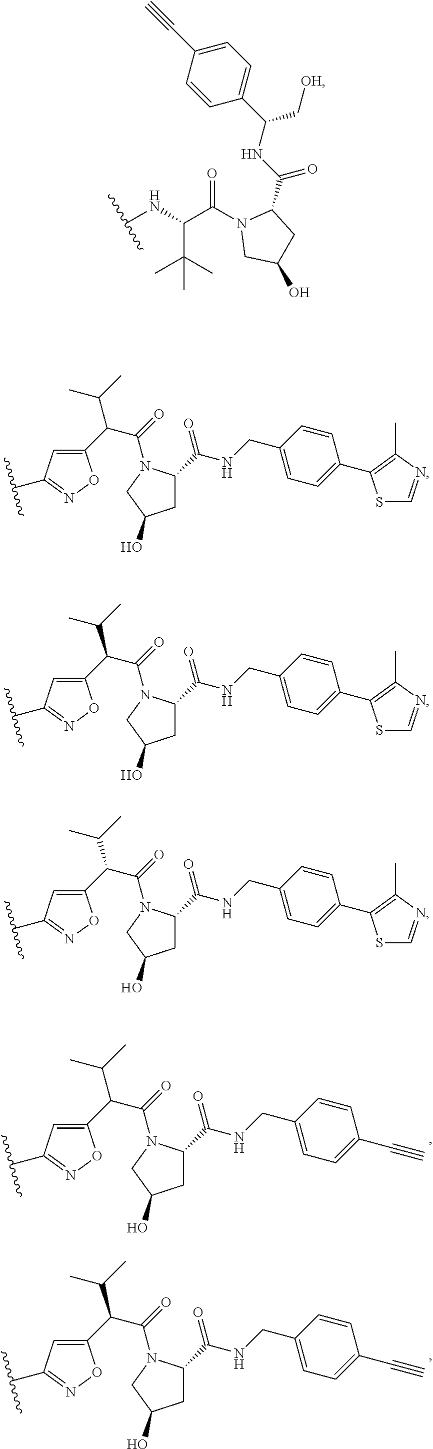

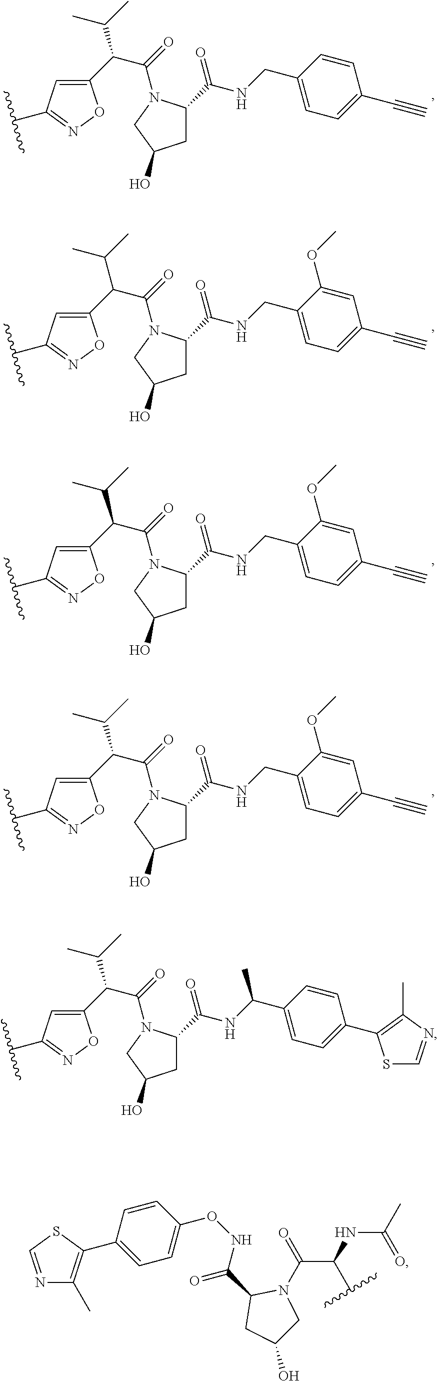 [SMILES: *c1cc([C@@H](C(=O)N2C[C@H](O)C[C@H]2C(=O)NCc2ccc(C#C)cc2)C(C)C)on1 ; *c1cc(C(C(=O)N2C[C@H](O)C[C@H]2C(=O)NCc2ccc(C#C)cc2OC)C(C)C)on1 ; *c1cc([C@H](C(=O)N2C[C@H](O)C[C@H]2C(=O)NCc2ccc(C#C)cc2OC)C(C)C)on1 ; *c1cc([C@@H](C(=O)N2C[C@H](O)C[C@H]2C(=O)NCc2ccc(C#C)cc2OC)C(C)C)on1 ; *c1cc([C@@H](C(=O)N2C[C@H](O)C[C@H]2C(=O)N[C@@H](C)c2ccc(-c3scnc3C)cc2)C(C)C)on1 ; *[C@H](NC(C)=O)C(=O)N1C[C@H](O)C[C@H]1C(=O)NOc1ccc(-c2scnc2C)cc1]

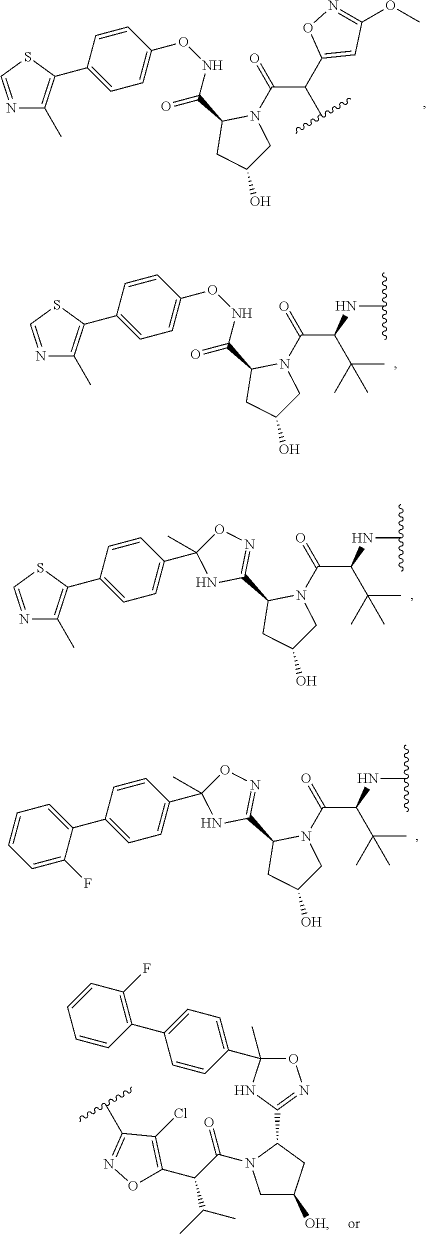 [SMILES: *C(C(=O)N1C[C@H](O)C[C@H]1C(=O)NOc1ccc(-c2scnc2C)cc1)c1cc(OC)no1 ; *N[C@H](C(=O)N1C[C@H](O)C[C@H]1C(=O)NOc1ccc(-c2scnc2C)cc1)C(C)(C)C ; *N[C@H](C(=O)N1C[C@H](O)C[C@H]1C1=NOC(C)(c2ccc(-c3scnc3C)cc2)N1)C(C)(C)C ; *N[C@H](C(=O)N1C[C@H](O)C[C@H]1C1=NOC(C)(c2ccc(-c3ccccc3F)cc2)N1)C(C)(C)C ; *c1noc([C@H](C(=O)N2C[C@H](O)C[C@H]2C2=NOC(C)(c3ccc(-c4ccccc4F)cc3)N2)C(C)C)c1Cl]

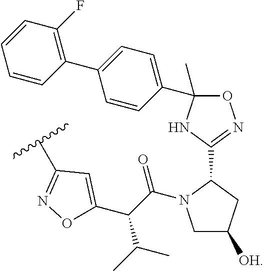 [SMILES: *c1cc([C@H](C(=O)N2C[C@H](O)C[C@H]2C2=NOC(C)(c3ccc(-c4ccccc4F)cc3)N2)C(C)C)on1]

In certain embodiments, LBM is selected from the group consisting of:

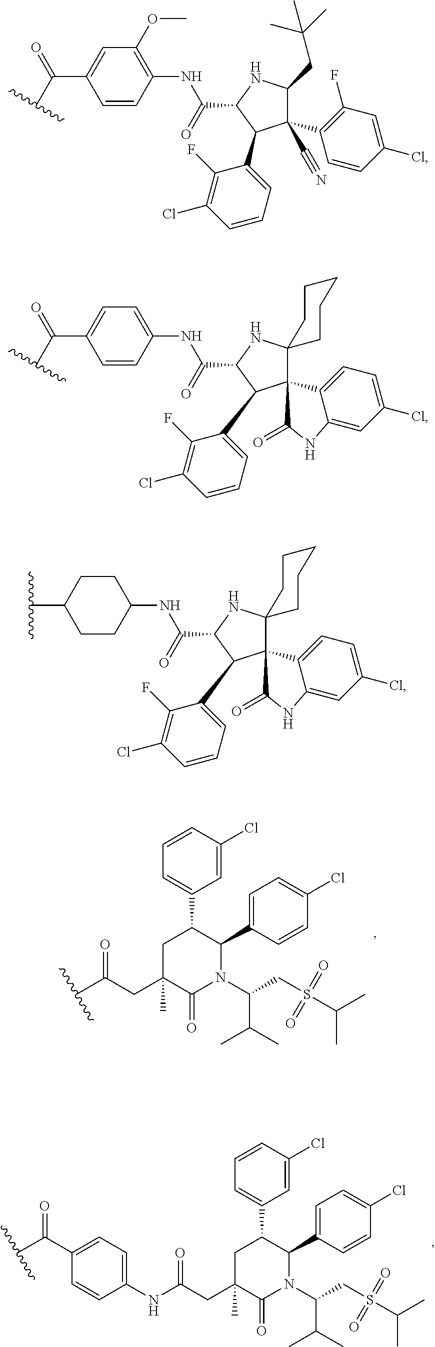 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; *C(=O)c1ccc(NC(=O)[C@@H]2NC3(CCCCC3)[C@@]3(C(=O)Nc4cc(Cl)ccc43)[C@H]2c2cccc(Cl)c2F)cc1 ; *C1CCC(NC(=O)[C@@H]2NC3(CCCCC3)[C@@]3(C(=O)Nc4cc(Cl)ccc43)[C@H]2c2cccc(Cl)c2F)CC1 ; *C(=O)C[C@@]1(C)C[C@H](c2cccc(Cl)c2)[C@@H](c2ccc(Cl)cc2)N([C@H](CS(=O)(=O)C(C)C)C(C)C)C1=O ; *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

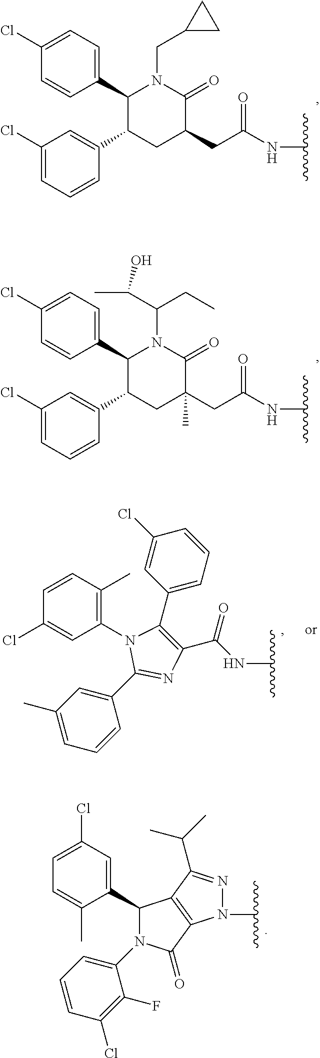 [SMILES: *NC(=O)C[C@H]1C[C@H](c2cccc(Cl)c2)[C@@H](c2ccc(Cl)cc2)N(CC2CC2)C1=O ; *NC(=O)C[C@@]1(C)C[C@H](c2cccc(Cl)c2)[C@@H](c2ccc(Cl)cc2)N(C(CC)[C@H](C)O)C1=O ; *NC(=O)c1nc(-c2cccc(C)c2)n(-c2cc(Cl)ccc2C)c1-c1cccc(Cl)c1 ; *n1nc(C(C)C)c2c1C(=O)N(c1cccc(Cl)c1F)[C@H]2c1cc(Cl)ccc1C]

As described herein, L is a bivalent moiety that connects TBM and DM.

In certain embodiments, L is a bivalent moiety that connects TBM to a lysine mimetic.

In certain embodiments, L is a covalent bond or a bivalent, saturated or partially unsaturated, straight or branched C₁₋₅₀ hydrocarbon chain, wherein 0-6 methylene units of L may optionally be replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, NRC(O)O—,

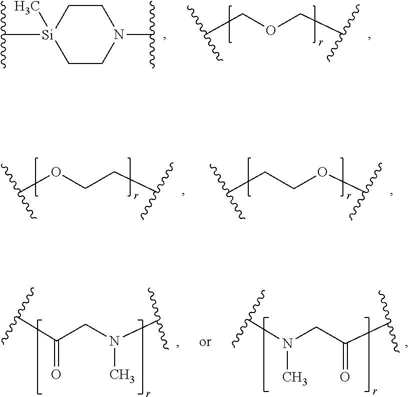

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
r is 0, 1, 2, 3, 4, 5, 6, 7, 8, 9, or 10.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro-carbocyclylenyl. In some embodiments, each -Cy- is independently an optionally substituted 8-10 membered bicyclic saturated or partially unsaturated carbocyclylenyl. In some embodiments, each -Cy is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro-heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is substituted with C₁₋₆ alkyl (e.g., methyl, ethyl, isopropyl). In some embodiments, -Cy- is substituted with oxo. In some embodiments, -Cy- is substituted with halogen. In some embodiments, -Cy- is substituted with fluoro. In some embodiments, -Cy- is substituted with geminal difluoro. In some embodiments, -Cy- is substituted with —OH. In some embodiments, -Cy- is substituted with —NR₂.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, L is —NR—(C₁₋₁₀ aliphatic)-. In some embodiments, L is —(C₁₋₁₀ aliphatic)-NR—(C₁₋₁₀aliphatic)-. In some embodiments, L is —(C₁₋₁₀ aliphatic)-NR—(CH₂CH₂O)₁₋₁₀CH₂CH₂. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy- (C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH2CH2O)₁₋₁₀CH₂CH₂—. In some embodiments, L is -Cy-CONR—(C₁₋₁₀ aliphatic). In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments. L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments. L is -Cy-(C₁₋₁₀ aliphatic)-Cy-CONR—. In some embodiments. L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In certain embodiments, L is any bivalent linker provided in WO 2024/064080, incorporated herein by reference in its entirey.

### 4. Compounds

In a first embodiment, provided is a compound of structural Formula I:

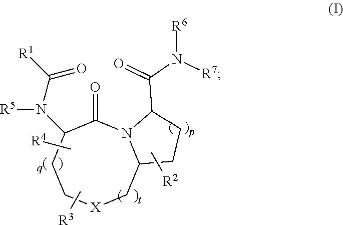

or a pharmaceutically acceptable salt thereof, wherein the variables are as described above.

In a second embodiment, X in the compound of Formula I, or pharmaceutically acceptable salt thereof, is NR⁹:
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}; and
R^{Z} is selected from halo, cyano, (C₁-C₄)alkyl, (C₃-C₆cycloalklyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocycyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo, wherein the remaining variables are as described above for Formula I. Alternatively, as part of a second embodiment, X in the compound of Formula I, or pharmaceutically acceptable salt thereof, is X is selected from S, SO₂, —S(═O)═NH, —NR⁸—;
R⁷ is selected from E, —R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo, wherein the remaining variables are as described above for Formula I. In another alternative, as part of a second embodiment, in Formula I or pharmaceutically acceptable salt thereof, X is selected from S, SO₂, —S(═O)═NH, —NR⁸;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo, wherein the remaining variables are as described above for Formula I. In another alternative, as part of a second embodiment, in Formula I or pharmaceutically acceptable salt thereof, X is selected from S, SO₂, —S(═O)═NH, —NR⁸;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo, wherein the remaining variables are as described above for Formula I. In another alternative, as part of a second embodiment, in Formula I or pharmaceutically acceptable salt thereof, X is selected from S, SO₂, —S(═O)═NH, —NR⁸;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alklphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo, wherein the remaining variables are as described above for Formula I.

In a third embodiment the compound of Formula I is of the structural Formula II:

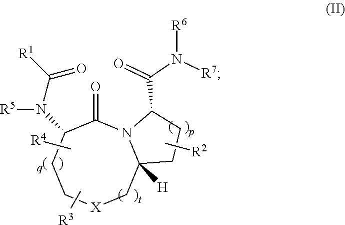

or pharmaceutically acceptable salt thereof, wherein the variables are as described above for Formula I or the second embodiment.

In a fourth embodiment, p in the compound of Formula I or II, or pharmaceutically acceptable salt thereof, is 1, wherein the remaining variables are as described above for Formula I or the second embodiment.

In a fifth embodiment, R² in the compound of Formula I or II, or pharmaceutically acceptable salt thereof, is hydrogen, wherein the remaining variables are as described above for Formula I or any one of the second or fourth embodiments.

In a sixth embodiment, R³ in the compound of Formula I or II, or pharmaceutically acceptable salt thereof, is hydrogen, wherein the remaining variables are as described above for Formula I or any one of the second, fourth or fifth embodiments.

In a seventh embodiment, R⁴ in the compound of Formula I or II, or pharmaceutically acceptable salt thereof, is hydrogen, wherein the remaining variables are as described above for Formula I or any one of the second and fourth to sixth embodiments.

In an eighth embodiment, q in the compound of Formula I or II, or pharmaceutically acceptable salt thereof, is 0 and t is 2, wherein the remaining variables are as described above for Formula I or any one of the second and fourth to seventh embodiments. Alternatively, as part of an eighth embodiment, q in the compound of Formula I or II, or pharmaceutically acceptable salt thereof, is 1 and t is 1, wherein the remaining variables are as described above for Formula I or any one of the second and fourth to seventh embodiments. In another alternative, as part of an eighth embodiment, q in the compound of Formula I or II, or pharmaceutically acceptable salt thereof, is 0 and t is 1, wherein the remaining variables are as described above for Formula I or any one of the second and fourth to seventh embodiments.

In a ninth embodiment the compound of Formula I is of the structural Formula III, IV, V, VI, VII, or VIII:

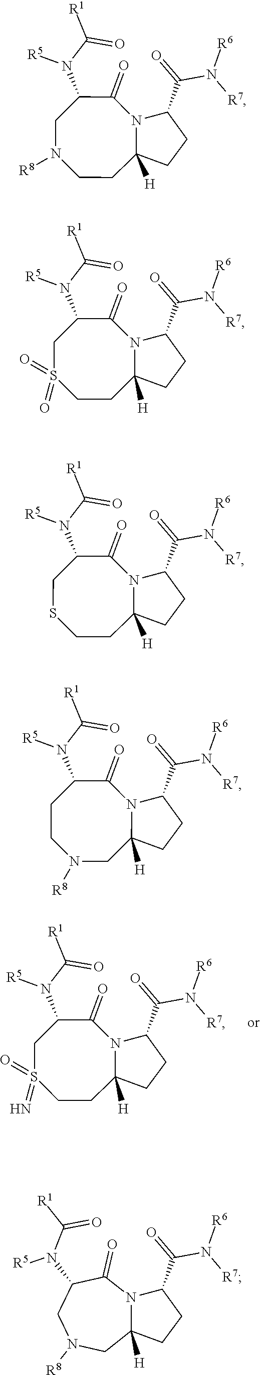

or pharmaceutically acceptable salt thereof, wherein the variables are as described above for Formula I or the second embodiment.

In a tenth embodiment, R⁵ in the compound of structural Formula I, II, III, IV, V, VI, VII, or VIII, or pharmaceutically acceptable salt thereof, is hydrogen, wherein the remaining variables are as described above for Formula I or any one of the second and fourth to eighth embodiments.

In an eleventh embodiment, R¹ in the compound of structural Formula I, II, III, IV, V, VI, VII, or VIII, or pharmaceutically acceptable salt thereof, is selected from 8- to 10-membered fused bicyclic heteroaryl and aryl, each of which are substituted with —CR^{1a}R^{2a}P(O)OR^{1b}OR^{2a}, wherein the remaining variables are as described above for Formula I or any one of the second, fourth to eighth, and tenth embodiments. Alternatively, as part of an eleventh embodiment, R¹ in the compound of structural Formula I, II, III, IV. V, VI, VII, or VIII, or pharmaceutically acceptable salt thereof, is selected from benzothiophenyl, indolyl, naphthalenyl, thienopyridinyl, benzothiazoyl, quinolinyl, isoquinolinyl, 4,5,6,7-tetrahydropyrrolopyridinyl, 5,6,7,8-tetrahydroimidazopyrazinyl, each of which are substituted with —CR^{1a}R^{2a}P(O)OR^{1b}OR^{2b}, wherein the remaining variables are as described above for Formula I or any one of the second, fourth to eighth, and tenth embodiments. In another alternative, as part of an eleventh embodiment, R¹ in the compound of structural Formula I, II, III, IV, V, VI, VII, or VIII, or pharmaceutically acceptable salt thereof, is selected from

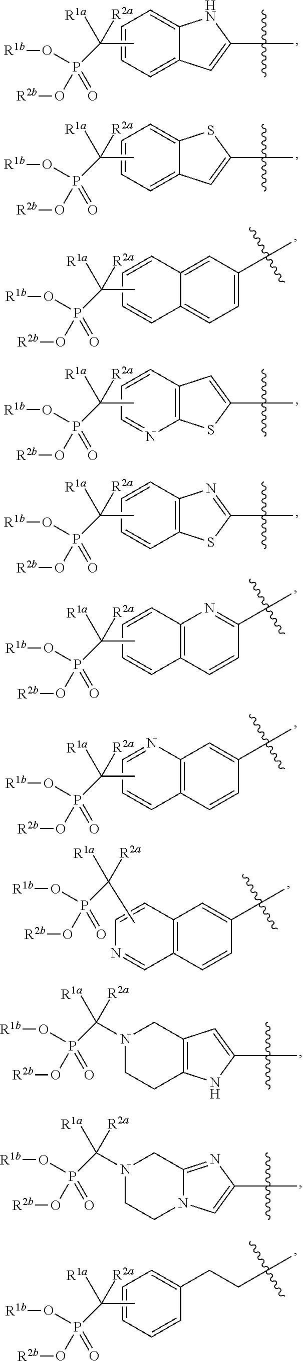

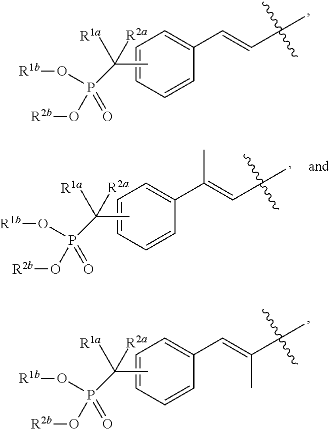

wherein the remaining variables are as described above for Formula I or any one of the second, fourth to eighth, and tenth embodiments. In another alternative, as part of an eleventh embodiment, R¹ in the compound of structural Formula I, II, III, IV, V, VI, VII, or VIII, or pharmaceutically acceptable salt thereof, is

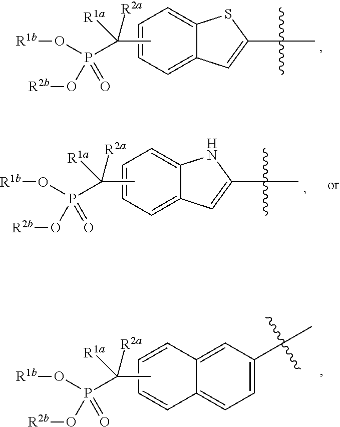

wherein the remaining variables are as described above for Formula I or any one of the second, fourth to eighth, and tenth embodiments.

In a twelfth embodiment, R^{1a} in the compound of structural Formula I, II, III, IV, V, VI, VII, or VII, or pharmaceutically acceptable salt thereof, is hydrogen and R^{2a} is fluoro or R^{1a} is fluoro and R^{2a} is fluoro, wherein the remaining variables are as described above for Formula I or any one of the second, fourth to eighth, tenth, and eleventh embodiments. Alternatively, as part of an twelfth embodiment, R^{1a} in the compound of structural Formula I, II, III, IV, V, VI, VII, or VIII, or pharmaceutically acceptable salt thereof, is fluoro and R^{2a} is fluoro, wherein the remaining variables are as described above for Formula I or any one of the second, fourth to eighth, tenth, and eleventh embodiments.

In a thirteenth embodiment, R^{1b} and R^{2b} in the compound of structural Formula I, II, III, IV, V, VI, VII, or VIII, or pharmaceutically acceptable salt thereof, are each independently selected from hydrogen, (C₁-C₄)alkyl, —[(C₁-C₄)alkyl]-OC(O)—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-OC(O)NR^{2c}R^{2d}], [(C₁-C₄)alkyl]-OC(O)O-[5- to 7-membered heterocyclyl], and phenyl, wherein the 5- to 7-membered heterocyclyl of [(C₁-C₄)alkyl]-OC(O)O-[5- to 7-membered heterocyclyl] is optionally substituted with C(O)OR^{2c}, wherein any of the (C₁—C)₄alkyl groups are optionally substituted with 1 or 2 (C₁-C₄)alkyl, wherein the remaining variables are as described above for Formula I or any one of the second, fourth to eighth, and tenth to twelfth embodiments. Alternatively, as part of a thirteenth embodiment, R^{1b} and R^{2b} in the compound of structural Formula I, II, III, IV, V, VI, VII, or VIII, or pharmaceutically acceptable salt thereof, are each —[(C₁-C₄)alkyl]-OC(O)—[(C₁-C₄)alkyl], wherein the remaining variables are as described above for Formula I or any one of the second, fourth to eighth, and tenth to twelfth embodiments. In another alternative, as part as part of a thirteenth embodiment, R^{1b} and R^{2b} in the compound of structural Formula I, II, III, IV, V, VI, VII, or VIII, or pharmaceutically acceptable salt thereof, are each hydrogen, wherein the remaining variables are as described above for Formula I or any one of the second, fourth to eighth, and tenth to twelfth embodiments.

In a fourteenth embodiment, —CR^{1a}R^{2a}P(O)OR^{1b}OR^{2b} in the compound of structural Formula I, II, III, IV, V, VI, VII, or VIII, or pharmaceutically acceptable salt thereof, is selected from

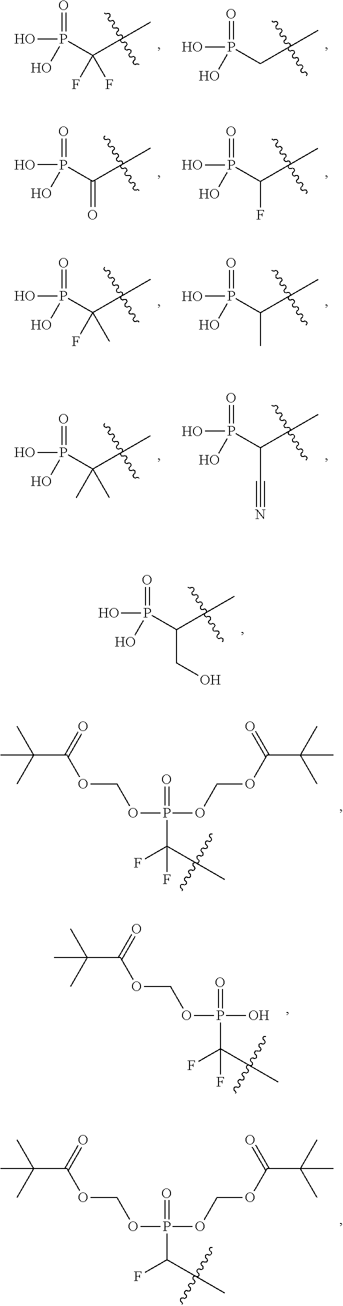

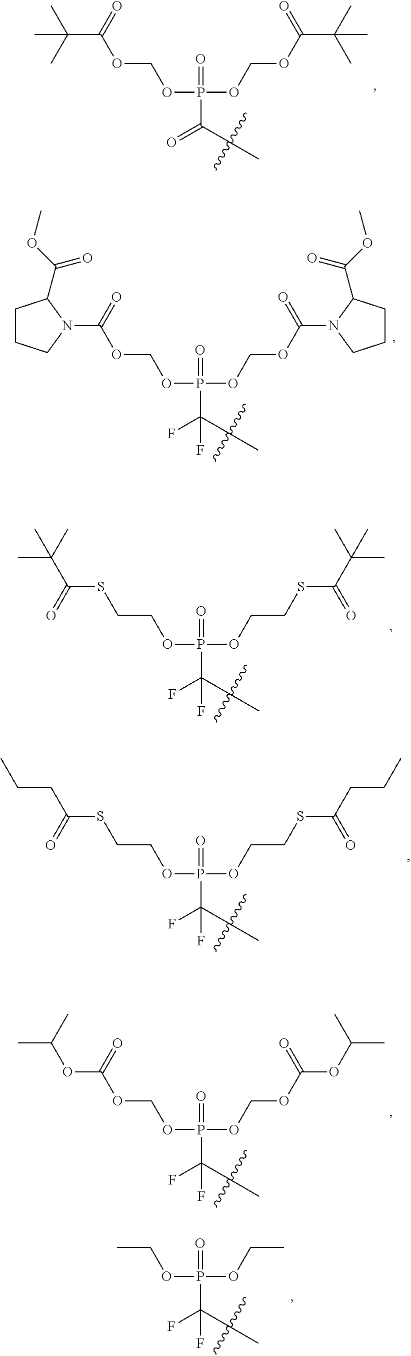

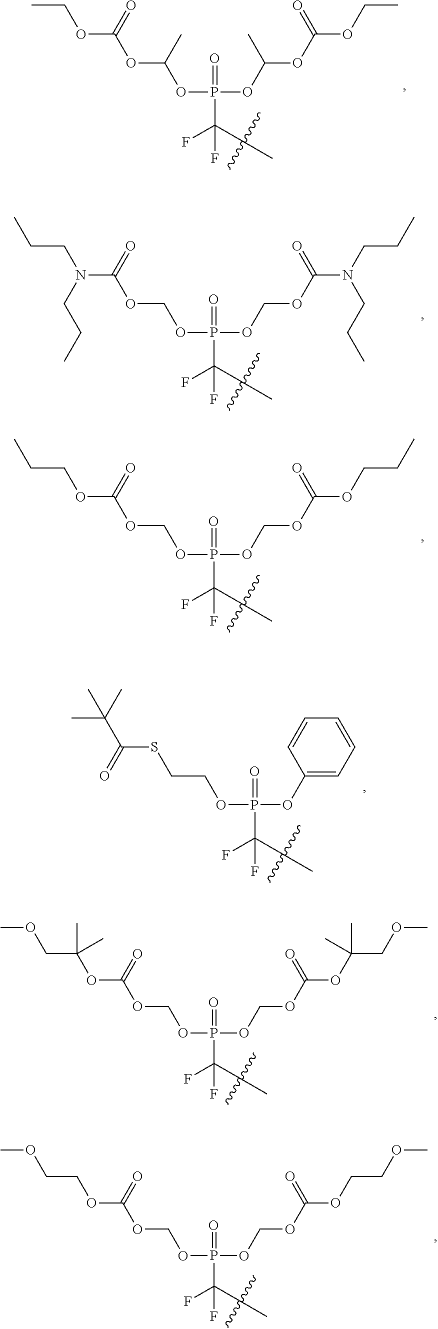

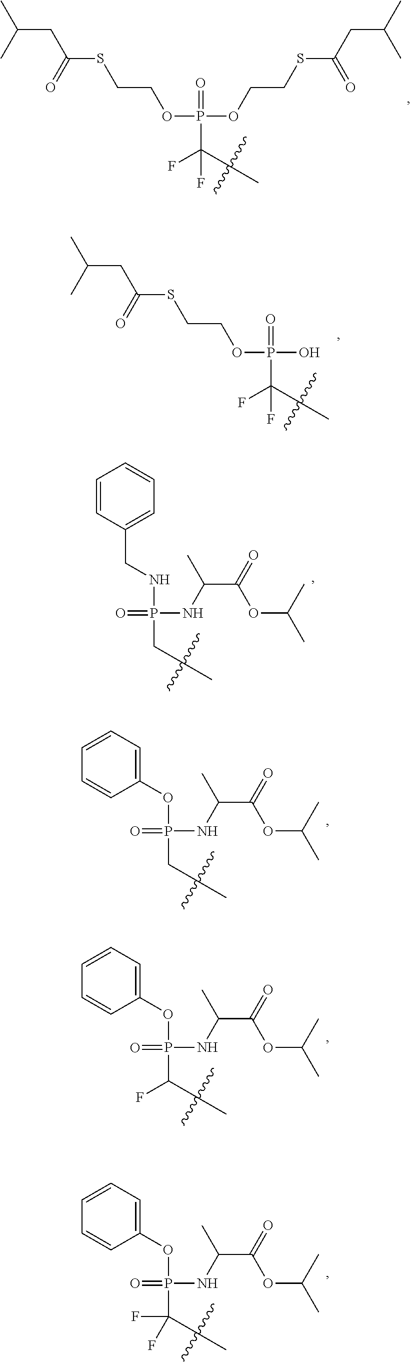

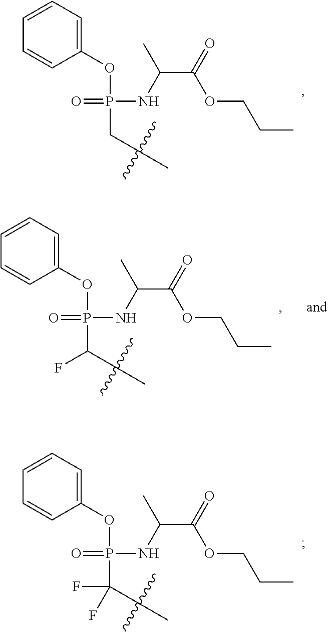 [SMILES: *CP(=O)(NC(C)C(=O)OCCC)Oc1ccccc1 ; *C(F)P(=O)(NC(C)C(=O)OCCC)Oc1ccccc1 ; *C(F)(F)P(=O)(NC(C)C(=O)OCCC)Oc1ccccc1]

wherein the remaining variables are as described above for Formula I or any one of the second, fourth to eighth, and tenth to thirteenth embodiments. Alternatively, as part of a fourteenth embodiment, —CR^{1a}R^{2a}P(O)OR^{1b}OR^{2b} in the compound of structural Formula I, II, III, IV, V, VI, VII, or VIII, or pharmaceutically acceptable salt thereof, is selected from

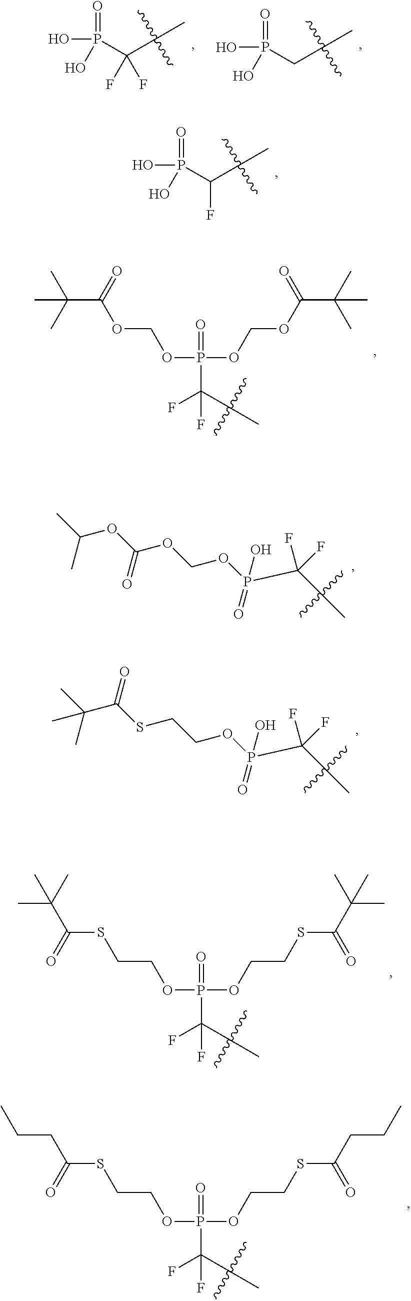

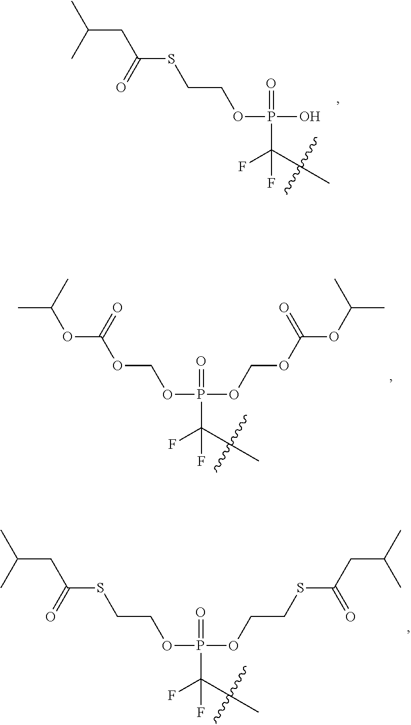

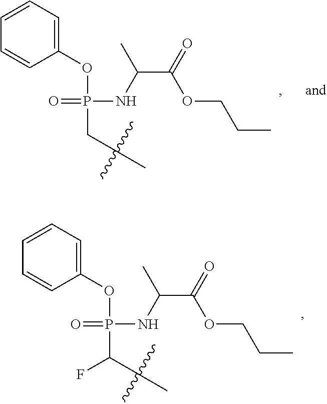 [SMILES: *CP(=O)(NC(C)C(=O)OCCC)Oc1ccccc1 ; *C(F)P(=O)(NC(C)C(=O)OCCC)Oc1ccccc1]

wherein the remaining variables are as described above for Formula I or any one of the second, fourth to eighth, and tenth to thirteenth embodiments.

In a fifteenth embodiment, R⁶ in the compound of structural Formula I, II, III, IV, V, VI, VII, or VIII, or pharmaceutically acceptable salt thereof, is selected from hydrogen and (C₁-C₄)alkyl, wherein the remaining variables are as described above for Formula I or any one of the second, fourth to eighth, and tenth to fourteenth embodiments.

In a sixteenth embodiment, R⁷ in the compound of structural Formula I, II, III, IV, V, VI, VII, or VIII, or pharmaceutically acceptable salt thereof, is selected from E, —R^{10A}E, and phenyl, wherein the remaining variables are as described above for Formula I or any one of the second, fourth to eighth, and tenth to fifteenth embodiments. Alternatively, as part of a sixteenth embodiment, R⁷ in the compound of structural Formula I, II, III, IV, V, VI, VII, or VIII, or pharmaceutically acceptable salt thereof, is phenyl substituted with R¹¹, wherein the remaining variables are as described above for Formula I or any one of the second, fourth to eighth, and tenth to fifteenth embodiments.

In a seventeenth embodiment, R¹¹ in the compound of structural Formula I, II, III, IV, V, VI, VII, or VIII, or pharmaceutically acceptable salt thereof, is —NHC(O)R^{10B}E, wherein the remaining variables are as described above for Formula I or any one of the second, fourth to eighth, and tenth to sixteenth embodiments.

In an eighteenth embodiment, R⁶ and R⁷ in the compound of structural Formula I, II, III, IV, V, VI, VII, or VIII, or pharmaceutically acceptable salt thereof, together with the nitrogen atom to which they are attached form a 4- to 6-membered monocyclic heterocyclyl optionally substituted with, as valency permits, R¹² and/or 1 to 3 groups selected from R^{Q}, wherein the remaining variables are as described above for Formula I or any one of the second, fourth to eighth, and tenth to seventeenth embodiments. Alternatively, as part of a eighteenth embodiment, R⁶ and R⁷ in the compound of structural Formula I, II, III, IV, V, VI, VII, or VIII, or pharmaceutically acceptable salt thereof, together with the nitrogen atom to which they are attached form azetidinyl, pyrrolidyl, or piperidinyl, each optionally substituted with, as valency permits, R¹² and/or 1 to 3 groups selected from R^{Q}, wherein the remaining variables are as described above for Formula I or any one of the second, fourth to eighth, and tenth to seventeenth embodiments.

In a nineteenth embodiment, R^{Q} in the compound of structural Formula I, II, III, IV, V, VI, VII, or VIII, or pharmaceutically acceptable salt thereof, is cyano, phenyl, or 5- to 7-membered monocyclic heteroaryl optionally substituted with, as valency permits, 1 to 3 groups selected from R^{F}, wherein the remaining variables are as described above for Formula I or any one of the second, fourth to eighth, and tenth to eighteenth embodiments. Alternatively, as part of a nineteenth embodiment, R^{Q} in the compound of structural Formula I, II, III, IV, V, VI, VII, or VIII, or pharmaceutically acceptable salt thereof, is cyano, phenyl, or pyridinyl optionally substituted with, as valency permits, 1 to 3 groups selected from R^{F}, wherein the remaining variables are as described above for Formula I or any one of the second, fourth to eighth, and tenth to eighteenth embodiments.

In a twentieth embodiment, R^{F} in the compound of structural Formula I, II, III, IV, V, VI, VII, or VIII, or pharmaceutically acceptable salt thereof, is —NR^{a}R^{b}, wherein the remaining variables are as described above for Formula I or any one of the second, fourth to eighth, and tenth to nineteenth embodiments.

In a twenty-first embodiment, R¹² in the compound of structural Formula I, II, III, IV, V, VI, VII, or VIII, or pharmaceutically acceptable salt thereof, is E, wherein the remaining variables are as described above for Formula I or any one of the second, fourth to eighth, and tenth to twentieth embodiments.

In a twenty-second embodiment, R⁸ in the compound of structural Formula I, II, III, IV, V, VI, VII, or VIII, or pharmaceutically acceptable salt thereof, is —C(O)R^{Ha}, wherein the remaining variables are as described above for Formula I or any one of the second, fourth to eighth, and tenth to twenty-first embodiments.

In a twenty-third embodiment, R^{Ha} in the compound of structural Formula I, III, III, IV, V, VI, VII, or VIII, or pharmaceutically acceptable salt thereof, is 8- to 10-membered bicyclic heteroaryl optionally substituted with C₁-C₄alkyl, wherein the remaining variables are as described above for Formula I or any one of the second, fourth to eighth, and tenth to twenty-second embodiments. Alternatively, as part of a twenty-third embodiment, R^{Ha} in the compound of structural Formula I, II, III, IV, V, VI, VII, or VIII, or pharmaceutically acceptable salt thereof, is indazolyl or benzoisoxazolyl, each optionally substituted with C₁-C₄alkyl, wherein the remaining variables are as described above for Formula I or any one of the second, fourth to eighth, and tenth to twenty-second embodiments.

In a twenty-fourth embodiment, R⁹ in the compound of structural Formula I, II, III, IV, V, VI, VII, or VIII, or pharmaceutically acceptable salt thereof, is selected from-R¹⁰E and —C(O)R¹⁰E, wherein the remaining variables are as described above for Formula I or any one of the second, fourth to eighth, and tenth to twenty-third embodiments.

In a twenty-fifth embodiment, R¹⁰, R^{10A}, R^{10B}, R^{10C}, and R^{10D} in the compound of structural Formula I, II, III, IV, V, VI, VII, or VIII, or pharmaceutically acceptable salt thereof, are each independently selected from (C₁-C₁₀)alkylene and (C₂-C₁₀)alkynelene each optionally substituted with one or more groups selected from halo and oxo, and wherein said (C₁-C₆)alkylene and (C₂-C₁₀)alkynelene may also be optionally interrupted by one or more heteroatoms selected from S, N, and O, and/or optionally interrupted by one or more rings selected from 5- to 7-membered heteroaryl, (C₃-C₆)cycloakyl, phenyl, and 4- to 7-membered heterocyclyl. Alternatively, as part of a twenty-fifth embodiment, R¹⁰, R^{10A}, R^{10B}, R^{10C}, and R^{10D} in the compound of structural Formula I, II, III, IV, V, VI, VII, or VIII, or pharmaceutically acceptable salt thereof, are each independently selected from (C₁-C₁₀)alkylene and (C₂-C₁₀)alkynelene, wherein the remaining variables are as described above for Formula I or any one of the second, fourth to eighth, and tenth to twenty-fourth embodiments. In another alternative, as part of as part of a twenty-fifth embodiment, R¹⁰, R^{10A}, R^{10B}, R^{10C}, and R^{10D} in the compound of structural Formula I, II, III, IV, V, VI, VII, or VIII, or pharmaceutically acceptable salt thereof, are each independently selected from (C₁-C₁₀)alkylene and (C₂-C₁₀)alkynelene,

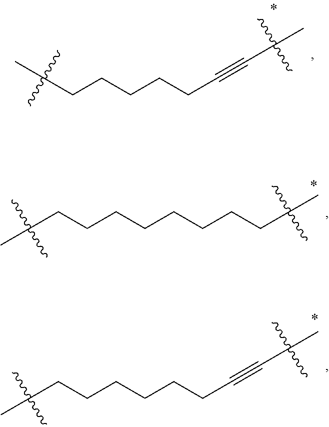 [SMILES: *C#CCCCCC* ; *CCCCCCCC* ; *C#CCCCCCC*]

CH₂, and (CH₂)₂, (CH₂)₃, (CH₂)₄, wherein the remaining variables are as described above for Formula I or any one of the second, fourth to eighth, and tenth to twenty-fourth embodiments.

In a twenty-sixth embodiment, E in the compound of structural Formula I, II, III, IV, V, VI, VII, or VIII, or pharmaceutically acceptable salt thereof is selected from a CRBN, VHL, IAP, or MDM2 based E3 binder, wherein the remaining variables are as described above for Formula I or any one of the second, fourth to eighth, and tenth to twenty-fifth embodiments. Alternatively, as part of a twenty-sixth embodiment, E in the compound of structural Formula I, II, III, IV, V, VI, VII, or VIII, or pharmaceutically acceptable salt thereof is selected from

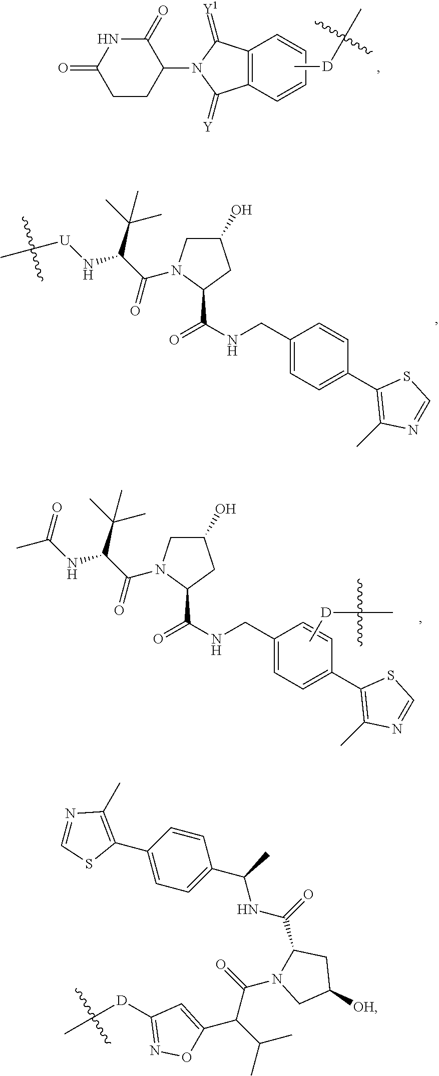

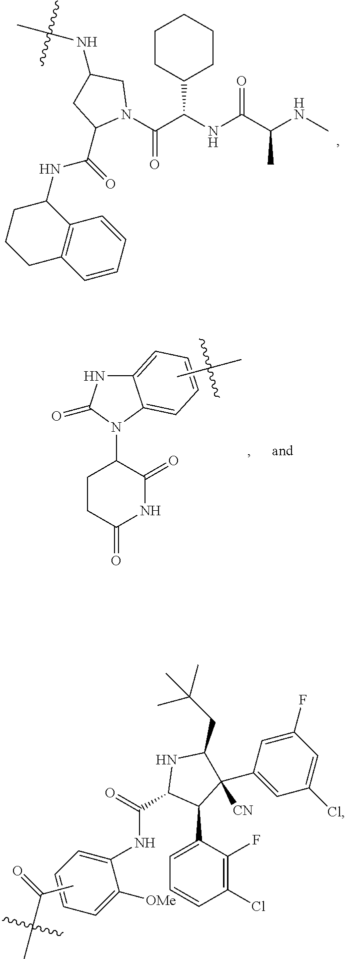

wherein:
Y and Y¹ are each independently is O or CH₂;
D is O, NH, or a bond;
U is absent or C(O); and
R is halo or cyano, wherein the remaining variables are as described above for Formula I or any one of the second, fourth to eighth, and tenth to twenty-fifth embodiments. In another alternative, as part of a as part of a twenty-sixth embodiment, E in the compound of structural Formula I, II, III, IV, V, VI, VII, or VIII, or pharmaceutically acceptable salt thereof is

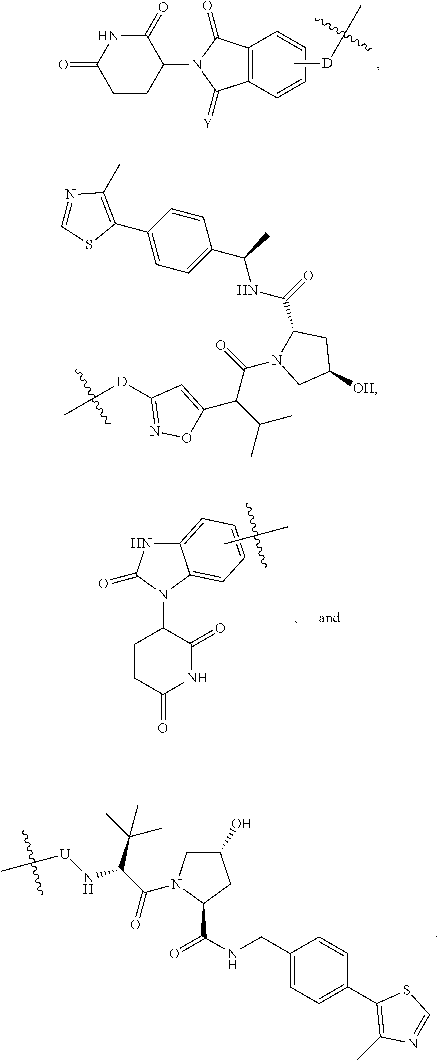

Compounds having the Formula I are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of STAT proteins, in particular STAT3 and/or STAT6. In some aspects, the compounds, pharmaceutical acceptable salts, and pharmaceutical compositions described herein degrade STAT3 and/or STAT6.

In some aspects, the compounds and pharmaceutical compositions described herein are useful in a condition responsive to the modulation (or degradation) of STAT3 and/or STAT6. Thus, provided herein are methods of treating a condition responsive to the modulation (e.g., degradation) of STAT3 and/or STAT6 in a subject, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a condition responsive to the modulation (e.g., degradation) of STAT3 and/or STAT6. Also provided is a compound described herein, or a pharmaceutically acceptable salt thereof, or a pharmaceutical composition comprising a disclosed compound or pharmaceutically acceptable salt thereof, for use in treating a condition responsive to the modulation (e.g., degradation) of STAT3 and/or STAT6.

In one aspect, the condition responsive to the modulation (e.g., degradation) of STAT3 and/or STAT6 include, but are not limited to, cancer, a neurodegenative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

In another aspect, the condition responsive to the modulation (e.g., degradation) of STAT3 and/or STAT6 include, but are not limited to, cancer (see, e.g., Turkson & Jove. Oncogene 2000, 19:6613-6626), diabetes (see. e.g., Gurzov et al., FEBS 2016, 283:3002), cardiovascular disease (see, e.g., Grote et al., Vasc. Pharmacol. 2005, 43:2005), viral disease (see, e.g., Gao et al., J Hepatol. 2012, 57(2):430), autoimmune diseases such as lupus (see, e.g., Goropevsek et al., Clin. Rev. Alleg. & Immun. 2017, 52(2):164), and rheumatoid arthritis (see, e.g., Walker & Smith, J. Rheumat. 2005, 32(9): 1650), autoinflammatory syndromes (see. e.g., Rauch et al., Jak-Stat 2013, 2(1):e23820), atherosclerosis (see, e.g., Ortiz-Munoz et al., Arterio., Thromho., Vase. Bio. 2009, 29:525), psoriasis (see. e.g., Andres et al., Exp. Derm. 2013, 22(5):323), allergic disorders (see. e.g., Oh et al., Eur. Respir. Rev. 2019, 19(115):46), inflammatory bowel disease (see. e.g., Sugimoto, World J Gastroenterol. 2008, 14(33):5110), inflammation (see, e.g., Tamiya et al., Arierio. Thrombo., Vasc. Bio. 2011, 31:980), acute and chronic gout and gouty arthritis, neurological disorders (see, e.g., Campbell. Brain Res. Rev. 2005, 48(2): 166), metabolic syndrome, immunodeficiency disorders such as AIDS and HIV (see, e.g., O'Shea et al., N. Engl J. Med. 2013, 368:161), destructive bone disorders (see. e.g., Jatiani et al, Genes & Can. 2011, 1(10):979), osteoarthritis, proliferative disorders, Waldenstrom's Macroglobulinemia (see, e.g., Hodge et al., Blood 2014, 123(7):1055) infectious diseases, conditions associated with cell death, pathologic immune conditions involving T cell activation, and CNS disorders.

Proliferative disorders, include, but are not limited to a benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-I driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments, the cancer to be treated is selected from glioma, breast cancer, prostate cancer, head and neck squamous cell carcinoma, skin melanomas, ovarian cancer, malignant peripheral nerve sheath tumors (MPNST), and pancreatic cancer. In other embodiments, the cancer to be treated is cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes. In other embodiments, the cancer is selected from solid tumors (e.g., prostate cancer, renal cancer, hepatic cancer, pancreatic cancer, gastric cancer, breast cancer, lung cancer, cancers of the head and neck, thyroid cancer, glioblastoma. Kaposi's sarcoma, Castleman's disease, uterine leiomyosarcoma, melanoma etc.), hematological cancers (e.g., lymphoma, leukemia such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

Compounds, salts, and compositions described herein are also useful in the treatment of inflammatory or obstructive airways diseases, resulting, for example, in reduction of tissue damage, airways inflammation, bronchial hyperreactivity, remodeling or disease progression. Inflammatory or obstructive airways diseases include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds, salts, and compositions described herein are also useful in the treatment of heteroimmune diseases including, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Compounds, salts, and compositions described herein are also useful in the treatment of other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. Compounds, salts, and compositions described herein are also useful in the treatment of bronchitis including, but not limited to, acute, arachidic, catarrhal, croupus, chronic or phthinoid bronchitis. Compounds, salts, and compositions described herein are also useful in the treatment of pneumoconiosis (an inflammatory, commonly occupational, disease of the lungs, frequently accompanied b % airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Compounds, salts, and compositions described herein are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds, salts, and compositions described herein are also useful in the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis. Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy. Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ectodermal dysplasia, Behcet's disease, incontinentia pigmenti. Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases. COPD (reduction of damage, airways inflammation, bronchial hyperreactivity, remodeling or disease progression), pulmonary disease, cystic fibrosis, acid induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, cardiovascular diseases which can be treated according to the present methods include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the present methods include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Preparation of Compounds

The compounds claimed herein were prepared following the procedures outlined in the following schemes. Compound names were generated using the software built into ChemDraw. To the extent that there are discrepancies between the name of a compound and its depicted structure, the depicted chemical structure is to be taken as the appropriate compound.

The following intermediates can be prepared according to the protocols described in PCT/US2023/063318:
5-((diethoxyphosphoryl)difluoromethyl)-1H-indole-2-carboxylic acid;
(difluoro(2-((4-nitrophenoxy)carbonyl)benzo[b]thiophen-5-yl)methyl)phosphonic acid;
(difluoro(7-((perfluorophenoxy)carbonyl)naphthalen-2-yl)methyl)phosphonic acid;
7-((diethoxyphosphoryl)difluoromethyl)-2-naphthoic acid;
perfluorophenyl 7-((bis(2-(butyrylthio)ethoxy)phosphoryl)difluoromethyl)-2-naphthoate;
perfluorophenyl 7-((bis(2-((3-methylbutanoyl)thio)ethoxy)phosphoryl) difluoromethyl)-2-naphthoate;
(difluoro(7-((perfluorophenoxy)carbonyl)naphthalen-2-yl)methyl)phosphonic acid;
(fluoro(7-((perfluorophenoxy)carbonyl)naphthalen-2-yl)methyl)phosphonic acid;
((7-((perfluorophenoxy)carbonyl)naphthalen-2-yl)methyl)phosphonic acid;
7-(fluoro((((S)-1-oxo-1-propoxypropan-2-yl)amino)(phenoxy)phosphoryl)methyl)-2-naphthoic acid; and
7-(((((S)-1-oxo-1-propoxypropan-2-yl)amino)(phenoxy)phosphoryl)methyl)-2-naphthoic acid.

Tert-butyl 3-(4-fluoropyridin-3-yl)azetidine-1-carboxylate can be prepared according to the protocols described in PCT/US2023/010447.

### Preparation of 8-(2-(2,6-dioxopiperidin-3-yl)-1-oxoisoindolin4-yl)oct-7-ynoic acid

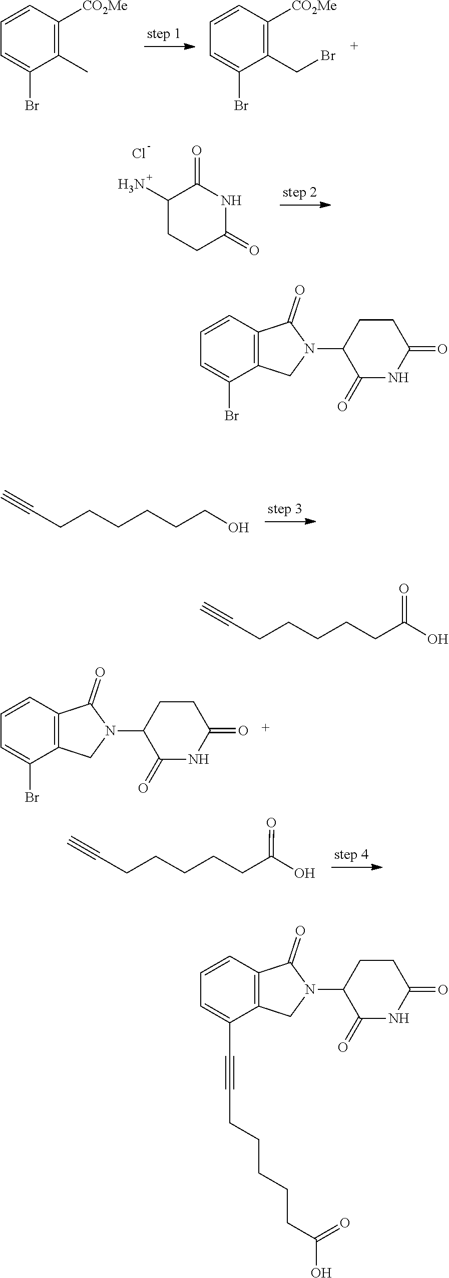

### Step 1: Methyl 3-bromo-2-(bromomethyl)benzoate

To the solution of methyl 3-bromo-2-methylbenzoate (5 g, 21.39 mmol) in carbon tetrachloride (anhydrous) (65 mL, 671.9 mmol) under N₂ atmosphere was added N-bromosuccinimide (4.30 g, 23.92 mmol) and benzoyl peroxide (500.0 mg, 2.023 mmol). The reaction mixture was heated at reflux (90° C.) and stirred at this temperature for 4 h. The precipitate was filtered off and the solvent was removed on a rotary evaporator. The crude material was purified by flash-chromatography using a 80 g silica gel cartridge eluting with a gradient of 0 to 20% EtOAc in heptanes in 15 CV to afford methyl 3-bromo-2-(bromomethyl)benzoate (6.6 g, 89%) as a colorless oil. LCMS (ESI): m/z=308.9 (M+H)⁺. ¹H NMR (400 MHz, CDCl₃) δ 7.92 (d, J=7.8 Hz, 1H), 7.80 (d, J=7.8 Hz, 1H), 7.32-7.23 (m, 1H), 5.17 (s, 2H), 4.00) (s, 3H).

### Step 2: 3-(4-Bromo-1-oxoisoidolin-2-yl)piperidine-2,6-dione

A mixture of methyl 3-bromo-2-(bromomethyl)benzoate (6.6 g, 20.36 mmol), 3-aminopiperidine-2,6-dione HCl salt (4.1 g, 24.41 mmol) and N,N-diisopropylethylamine (18 mL, 102.3 mmol) acetonitrile (70 mL) was heated to 90° C. for 18 h. The reaction mixture was filtered and the filter cake was washed with ether to give 3-(4-bromo-1-oxoisoindolin-2-yl)piperidine-2,6-dione (5.3 g, 80%) as a purple solid. LCMS (ESI): m/z=323.0 (M+H)⁺. ¹H NMR (400 MHz, DMSO-d₆) δ 11.01 (s, 1H), 7.87 (d, J=7.8 Hz, 1H), 7.77 (d, J=7.1 Hz, 1H), 7.51 (t, J=7.7 Hz, 1H), 5.15 (did, J=13.2, 5.1 Hz, 1H), 4.46-4.22 (m, 2H), 2.97-2.86 (m, 1H), 2.59 (d, J=17.4 Hz, 3H), 2.54-2.41 (m, 1H), 2.07-1.98 (m, 1H).

### Step 3: Oct-7-ynoic acid

Preparation of Jones' reagent: chromium trioxide (2.05 g, 20.6 mmol, 1.6 eq) was dissolved in water (4 mL) (dark red solution) and sulfuric acid (1.8 mL) was added (precipitate). Minimal amount of water (15 drops) was added until a dark red solution was observed.

7-Octyn-1-ol (2 g, 12.9 mmol, 1 eq) w as dissolved in acetone (40 mL) then Jones' reagent was added dropwise (careful: highly exothermic). Upon addition of Jones' reagent, the solution turned green and by the end of the addition, a red mixture with green precipitate was observed. The reaction was stirred for 2 h and then quenched with dropwise addition of i-PrOH (reaction turned green). The reaction was diluted with MTBE and filtered over Celite. Silica gel was added to the filtrate and the latter was evaporated to dryness. The crude residue was purified by flash-chromatography on a 40 g silica gel cartridge eluting with 0-75% EtOAc in heptanes to give dec-9-ynoic acid (1.99 g, 92%) as a yellow oil. ¹H NMR (400 MHz, CDCl₃) δ 11.42 (br. s., 1H), 2.37 (t, J=7.6 Hz, 2H), 2.20 (td, J=7.0, 2.7 Hz, 2H), 1.97-1.92 (m, 1H), 1.71-1.61 (m, 2H), 1.60-1.51 (m, 2H), 1.50-1.42 (m, 2H).

### Step 4: 8-(2-(2,6-Dioxopiperidin-3-yl)-1-oxoisoindolin-4-yl)oct-7-ynoic acid

Triethylamine (1.60 mL, 11.44 mmol) was added to a mixture of 3-(4-bromo-1-oxoisoindolin-2-yl)piperidine-2,6-dione (200 mg, 0.618 mmol), oct-7-ynoic acid (95.0 mg, 0.677 mmol), copper(I) iodide (30.0 mg, 0.156 mmol) and trans-dichlorobis(triphenylphosphine) palladium(II) (55.0 mg, 0.078 mmol) in N,N-dimethylformamide (3 mL). The resulting mixture was purged and refilled with nitrogen three times and then stirred at 80° C. for 18 h under nitrogen. The reaction mixture was directly purified by reverse phase chromatography using a 50 g C₁₈ cartridge eluting with a gradient of acetonitrile in water (5% for 4 CV then 5 to 100% in 18 CV) to give 8-(2-(2,6-dioxopiperidin-3-yl)-1-oxoisoindolin-4-yl)oct-7-ynoic acid (145 mg, 56%) as a brown solid. LCMS (ESI): m/z=383.2 (M+H)⁺. ¹H NMR (400 MHz, DMSO-d₆) δ 10.99 (br. s., 1H), 7.70 (d, J=7.8 Hz, 1H), 7.63 (d, J=7.3 Hz, 1H), 7.54-7.49 (m, 1H), 5.13 (dd, J=13.0, 4.9 Hz, 1H), 4.48-4.26 (m, 2H), 2.96-2.85 (m, 1H), 2.59 (d, J=18.6 Hz, 1H), 2.49-2.40 (m, 3H), 2.33-2.12 (m, 3H), 2.02 (d, J=5.1 Hz, 1H), 1.62-151 (m, 4H), 1.48-1.40 (m, 2H).

### Preparation of [(2-{[(5S,8S,10aR)-3-{8-[2-(2,6-dioxopiperidin-3-yl)-1-oxo-2,3-dihydro-1H-isoindol-4-yl]oct-7-ynoyl}-8-[methyl(phenyl)carbamoyl]-6-oxo-decahydropyrrolo[1,2-a][1,5]diazocin-5-yl]carbamoyl}-1H-indol-5-yl)difluoromethyl]phosphonic acid (Compound 1)

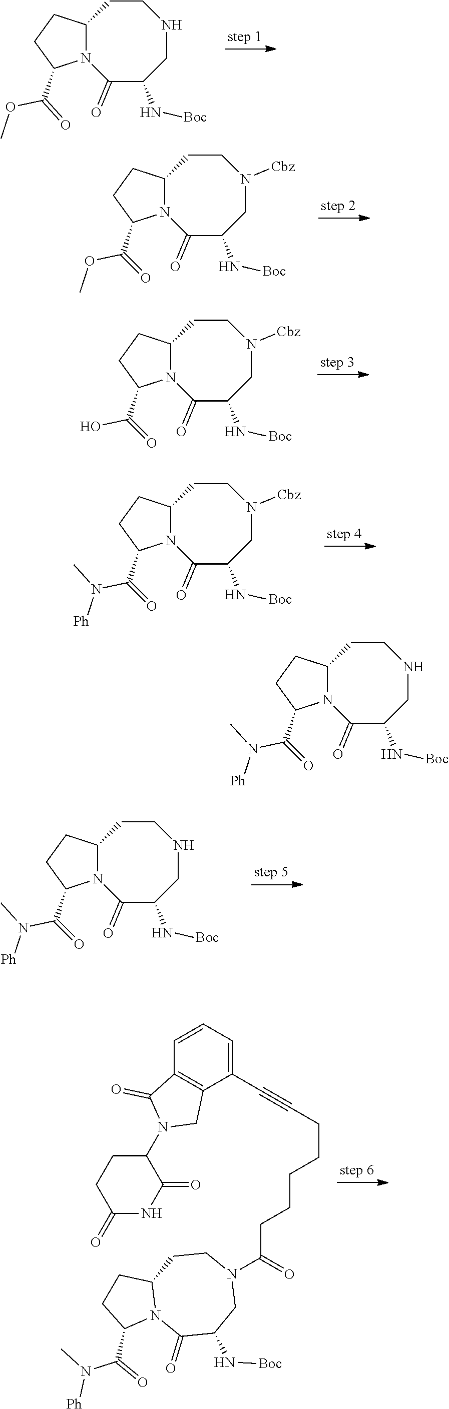

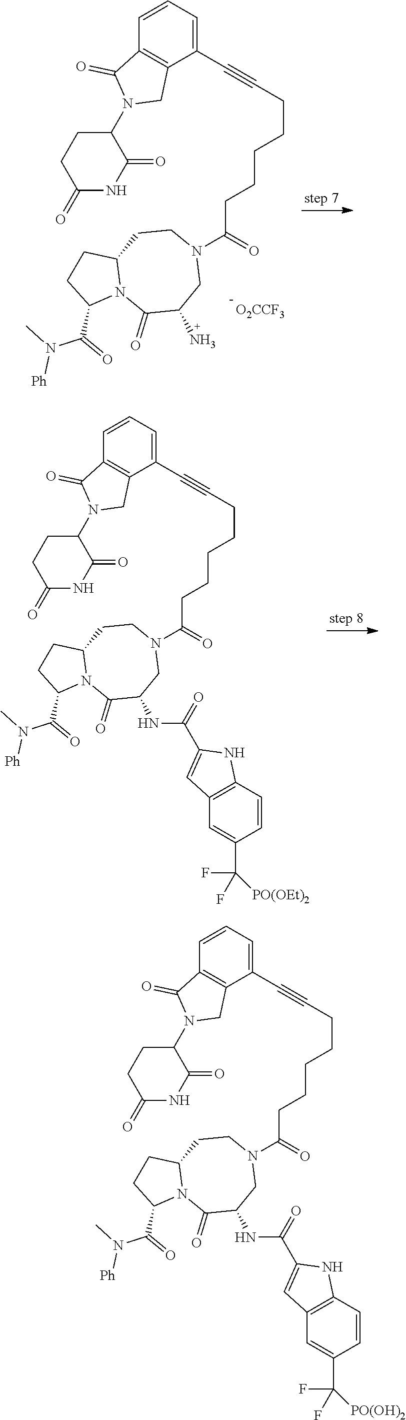

### Step 1: 3-Benzyl 8-methyl (5S,8S,10aR)-5-{[(tert-butoxy)carbonyl]amino}-6-oxo-decahydropyrrolo[1,2-a][1,5]diazocine-3,8-dicarboxylate

Benzyl chloroformate (582 μL, 4.08 mmol, 1.4 eq) and sodium bicarbonate (735 mg, 8.76 mmol, 3 eq) were added to a solution of methyl (5S,8S,10aR)-5-{[(tert-butoxy)carbonyl]amino}-6-oxo-decahydropyrrolo[1,2-a][1,5]diazocine-8-carboxylate (1 g, 2.92 mmol, 1 eq) in anhydrous dioxane (30 mL). The mixture was stirred at room temperature for 18 h. The reaction mixture was filtered through Celite and then the filtrate was concentrated to give crude 3-benzyl 8-methyl (5S,8S,10aR)-5-{[(tert-butoxy)carbonyl]amino}-6-oxo-decahydropyrrolo[1,2-a][1,5]diazocine-3,8-dicarboxylate as a clear oil. The crude product was used for the next step without further purification. LCMS (ESI): m/z=376.2 (M-Boc+2H)⁺, 420.2 (M-t-Bu+2H)⁺.

### Step 2: (5S,8S,10aR)-3-[(Benzyloxy)carbonyl]-5-{[(tert-butoxy)carbonyl]amino}-6-oxo-decahydropyrrolo[1,2-a][1,5]diazocine-8-carboxylic acid

To a solution of crude 3-benzyl 8-methyl (5S,8S,10aR)-5-{[(tert-butoxy)carbonyl]amino}-6-oxo-decahydropyrrolo[1,2-a][1,5]diazocine-3,8-dicarboxylate (1.4 g, 2.94 mmol, 1 eq) in tetrahydrofuran (8 mL) and water (2 mL) was added lithium hydroxide monohydrate (370 mg, 8.82 mmol, 3 eq). The reaction mixture was stirred for 4 h at room temperature. The reaction mixture was quenched with the addition of 1 N aqueous HCl solution (20 mL) and then the product was extracted with DCM (3×30 mL). The combined organic extracts were dried with sodium sulfate, filtered and concentrated under reduced pressure. The crude residue was directly purified by reverse phase chromatography using a 50 g C₁₈ cartridge eluting with a gradient of acetonitrile in water (5% for 3 CV then 5 to 100% in 18 CV) to give (5S,8S,10aR)-3-[(benzyloxy)carbonyl]-5-{[(tert-butoxy)carbonyl]amino}-6-oxo-decahydropyrrolo[1,2-a][1,5]diazocine-8-carboxylic acid (1.07 g, 80% over 2 steps) as a white solid. LCMS (ESI): m/z=362.2 (M-Boc+2H)⁺, 406.2 (M-t-Bu+2H)⁺. ¹H NMR (400 MHz, DMSO-d₆) δ 12.39 (br. s., 1H), 7.41-7.26 (m, 5H), 7.04-6.85 (m, 1H), 5.12-4.98 (m, 2H), 4.54-4.40 (m, 1H), 4.27-4.15 (m, 2H), 3.64-3.37 (m, 4H), 2.25 (dt, J=12.0, 7.7 Hz, 1H), 2.11-1.97 (m, 1H), 1.92-1.78 (m, 2H), 1.76-1.57 (m, 2H), 1.40-1.27 (m, 9H).

### Step 3: Benzyl (5S,8S,10aR)-5-({[(tert-butoxy)carbonyl]amino}-8-[methyl(phenyl)carbamoyl]-6-oxo-decahydropyrrolo[1,2-a][1,5]diazocine-3-carboxylate

To a solution of (5S,8S,10aR)-3-[(benzyloxy)carbonyl]-5-{[(tert-butoxy)carbonyl]amino}-6-oxo-decahydropyrrolo[1,2-a][1,5]diazocine-8-carboxylic acid (500 mg, 1.08 mmol, 1 eq) in 1,2-dichloroethane (10 mL) were added N-methylaniline (173 μL, 1.61 mmol, 1.5 eq), a 50% w/w solution of T3P in EtOAc (1.02 mL, 1.61 mmol, 1.5 eq) and N,N-diisopropylethylamine (1.12 mL, 6.47 mmol, 6 eq). The reaction mixture was stirred for 18 h. The solvent was evaporated and the crude residue was directly purified by reverse phase chromatography on a 50 g C₁₈ cartridge eluting with 5-100% acetonitrile in water. The combined fractions were concentrated under reduced pressure. The isolated product was re-purified by normal phase chromatography using a 24 g silica gel cartridge eluting with a gradient of EtOAc in heptanes (0 to 100% in 18 CV) then with MeOH in DCM (0 to 10% in 6 CV) to give benzyl (5S,8S,10aR)-5-{[(tert-butoxy)carbonyl]amino}-8-[methyl(phenyl)carbamoyl]-6-oxo-decahydropyrrolo[1,2-a][1,5]diazocine-3-carboxylate (311 mg, 52%) as a yellow solid. LCMS (ESI): m/z=551.2 (M+H)⁺.

### Step 4: tert-Butyl N-[(5S,8S,10aR)-8-[methyl(phenyl)carbamoyl]-6-oxo-decahydropyrrolo[1,2-a][1,5]diazocin-5-yl]carbamate

10% Palladium on carbon (50% wet) (238 mg, 113 μmol, 0.2 eq) was added to a mixture of benzyl (5S,8S,10aR)-5-{1[(tert-butoxy)carbonyl]amino}-8-[methyl(phenyl)carbamoyl]-6-oxo-decahydropyrrolo[1,2-a][1,5]diazocine-3-carboxylate (311 mg, 564 μmol, 1 eq) in methanol (15 mL). Hydrogen was bubbled into the suspension for 2 min, and then the reaction mixture was stirred under 1 atm. of hydrogen for 18 h. The reaction mixture was filtered on Celite and the pad was rinsed with MeOH. The filtrate was concentrated in vacuo. The crude residue was purified by reverse phase chromatography on a 50 g C₁₈ cartridge eluting with 5-80% acetonitrile in water. The combined fractions were concentrated under reduced pressure to give tert-butyl N-[(5S,8S,10aR)-8-[methyl(phenyl)carbamoyl]-6-oxo-decahydropyrrolo[1,2-a][1,5]diazocin-5-yl]carbamate (125 mg, 53%) as a white solid. LCMS (ESI): m/z=417.2 (M+H)⁺.

### Step 5: tert-Butyl N-[(5S,8S,10aR)-3-{8-[2-(2,6-dioxopiperidin-3-yl)-1-oxo-2,3-dihydro-1H-isoindol-4-yl]oct-7-ynoyl}-8-[methyl(phenyl)carbamoyl]-6-oxo-decahydropyrrolo[1,2-a][1,5]diazocin-5-yl]carbamate

To a solution of tert-butyl N-[(5S,8S,10aR)-8-[methyl(phenyl)carbamoyl]-6-oxo-decahydropyrrolo[1,2-a][1,5]diazocin-5-yl]carbamate (125 mg, 300 μmol, 1 eq) and 8-[2-(2,6-dioxopiperidin-3-yl)-1-oxo-2,3-dihydro-1H-isoindol-4-yl]oct-7-ynoic acid (126 mg, 330 μmol, 1.1 eq) in N,N-dimethylformamide (5 mL) and N,N-diisopropylethylamine (311 μL, 1.79 mmol) was added HATU (148 mg, 390 μmol, 1.3 eq). The resulting brown solution was stirred for 30 mm, at room temperature. The reaction mixture was directly purified by reverse phase chromatography on a 50 g C₁₈ cartridge eluting with 5-100% acetonitrile in water. The combined fractions were concentrated under reduced pressure to give tert-butyl N-[(5S,8S,10aR)-3-{8-[2-(2,6-dioxopiperidin-3-yl)-1-oxo-2,3-dihydro-1H-isoindol-4-yl]oct-7-ynoyl}-8-[methyl(phenyl)carbamoyl]-6-oxo-decahydropyrrolo[1,2-a][1,5]diazocin-5-yl]carbamate (123 mg, 52%) as a beige solid. LCMS (ESI): m/z=781.4 (M+H)⁺.

### Step 6: (5S,8S,10aR)-5-amino-3-{8-[2-(2,6-dioxopiperidin-3-yl)-1-oxo-2,3-dihydro-1H-isoindol-4-yl]oct-7-ynoyl}-N-methyl-6-oxo-N-phenyl-decahydropyrrolo[1,2-a][1,5]diazocine-8-carboxamide

To a solution of tert-butyl N-[(5S,8S,10aR)-3-{8-[2-(2,6-dioxopiperidin-3-yl)-1-oxo-2,3-dihydro-1H-isoindol-4-yl]oct-7-ynoyl}-8-[methyl(phenyl)carbamoyl]-6-oxo-decahydropyrrolo[1,2-a][1,5]diazocin-5-yl]carbamate (25 mg, 32.0 μmol, 1 eq) in methylene chloride (1 mL) was added trifluoroacetic acid (0.5 mL). The resulting yellow solution was stirred for 1 h at room temperature. The reaction mixture was concentrated under reduced pressure. The crude TFA salt was used directly for the next step without further purification. LCMS (ESI): m/z=681.3 (M+H)⁺.

### Step 7: Diethyl [(2-{[(5S,8S,10aR)-3-(8-[2-(2,6-dioxopiperidin-3-yl)-1-oxo-2,3-dihydro-1H-isoindol-4-yl]oct-7-ynoyl}-8-methyl(phenyl)carbamoyl]-6-oxo-decahydropyrrolo[1,2-a][1,5]diazocin-5-yl]carbamoyl}-1H-indol-5-yl)difluoromethyl]phosphonate

To a solution of crude (5S,8S,10aR)-5-amino-3-{8-[2-(2,6-dioxopiperidin-3-yl)-1-oxo-2,3-dihydro-1H-isoindol-4-yl]oct-7-ynoyl}-N-methyl-6-oxo-N-phenyl-decahydropyrrolo[1,2-a][1,5]diazocine-8-carboxamide TFA salt (25.4 mg, 32.0 μmol, 1 eq) and 5-[(diethoxyphosphoryl)difluoromethyl]-1H-indole-2-carboxylic acid (12.3 mg, 35.5 μmol, 1.1 eq) in N,N-dimethylformamide (2 mL) and N,N-diisopropylethylamine (33.5 μL, 193 μmol, 6 eq) was added HATU (15.9 mg, 41.9 μmol, 1.3 eq). The resulting yellow solution was stirred for 30 min. at room temperature. The reaction mixture was directly purified by reverse phase chromatography on a 50 g C₁₈ cartridge eluting with 5-100% acetonitrile in water. The combined fractions were concentrated under reduced pressure and then dried under high vacuum to give diethyl [(2-{[(5S,8S,10aR)-3-{8-[2-(2,6-dioxopiperidin-3-yl)-1-oxo-2,3-dihydro-1H-isoindol-4-yl]oct-7-ynoyl}-8-[methyl(phenyl)carbamoyl]-6-oxo-decahydropyrrolo[1,2-a][1,5]diazocin-5-ylcarbamoyl}-1H-indol-5-yl)difluoromethyl]phosphonate (22.0 mg, 67%) as a yellow solid. LCMS (ESI): m/z=1011.4 (M+H)⁺.

### Step 8: [(2-{[(5S,8S,10aR)-3-{8-[2-(2,6-dioxopiperidin-3-yl)-1-oxo-2,3-dihydro-1H-isoindol-4-yl]oct-7-ynoyl}-8-[methyl(phenyl)carbamoyl]-6-oxo-decahydropyrrolo[1,2-a][1,5]diazocin-5-yl]carbamoyl}-1H-indol-5-yl)difluoromethyl]phosphonic acid

To a solution of diethyl [(2-{[(5S,8S,10aR)-3-{8-[2-(2,6-dioxopiperidin-3-yl)-1-oxo-2,3-dihydro-1H-isoindol-4-yl]oct-7-ynoyl}-8-[methyl(phenyl)carbamoyl]-6-oxo-decahydropyrrolo[1,2-a][1,5]diazocin-5-yl]carbamoyl}-1H-indol-5-yl)difluoromethyl]phosphonate (22 mg, 21.7 μmol, 1 eq) in methylene chloride (3 mL) at 0° C. was added N,O-bis(trimethylsilyl)trifluoroacetamide (17.2 μL, 65.1 μmol, 3 eq) and trimethylsilyl iodide (26.0 μL, 130 μmol, 6 eq). The reaction mixture was stirred for 15 min. at 0° C. The solvent was removed under reduced pressure at 0° C. DMSO (1 mL) was added.

The crude residue was purified by reverse phase chromatography on a 50 g C₁₈ cartridge eluting with 5-40% acetonitrile in water. The combined fractions were directly freeze-dried to give [(2-{[(5S,8S,10aR)-3-{8-[2-(2,6-dioxopiperidin-3-yl)-1-oxo-2,3-dihydro-1H-isoindol-4-yl}oct-7-ynoyl}-8-[methyl(phenyl)carbamoyl]-6-oxo-decahydropyrrolo[1,2-a][1,5]diazocin-5-yl]carbamoyl}-1H-indol-5-yl)difluoromethyl]phosphonic acid (Compound 1 6.7 mg, 32%) as a white solid. LCMS (ESI): m/z=954.3 (M+H)⁺. ¹H NMR (400 MHz, CD₃OD) δ 7.96-7.88 (m, 1H), 7.75-7.34 (m, 10H), 7.27-7.16 (m, 1H), 5.20-4.96 (m, 2H), 4.53-4.47 (m, 1H), 4.37-4.35 (m, 1H), 4.26-4.20 (m, 1H), 4.05-4.00 (m, 1H), 3.85-3.81 (m, 1H), 3.75-3.45 (m, 2H), 3.28-3.18 (m, 3H), 2.92-2.79 (m, 1H), 2.77-2.62 (m, 2H), 2.54-2.50 (m, 3H), 2.21-2.09 (m, 1H), 2.04-1.85 (m, 4H), 1.79-1.54 (m, 7H), 1.39-1.31 (m, 3H).

### Preparation of 5-((bis((pivaloyloxy)methoxy)phosphoryl)difluoromethyl)-1H-indole-2-carboxylic acid

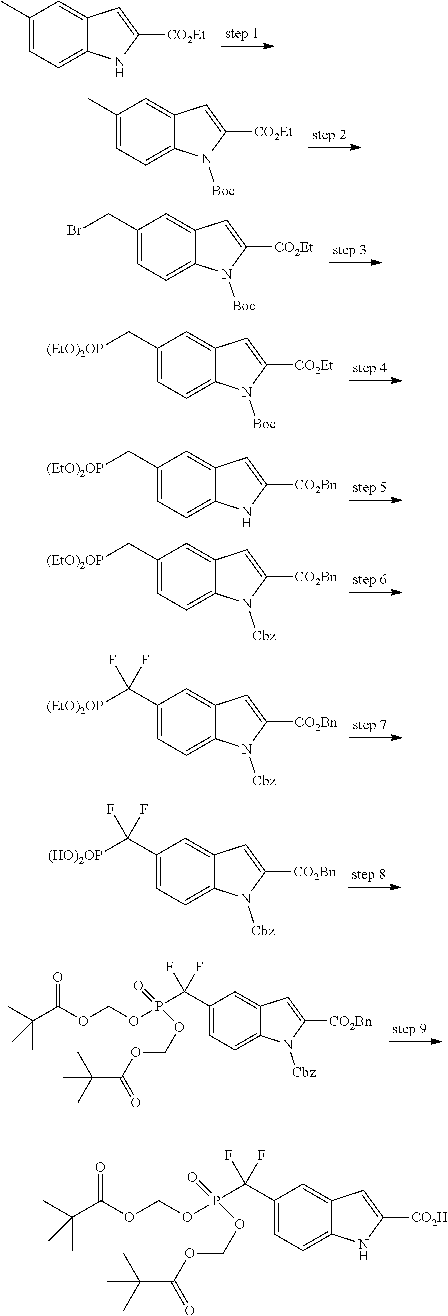

### Step 1: 1-(tert-Butyl) 2-ethyl 5-methyl-1H-indole-1,2-dicarboxylate

To a suspension of sodium hydride (60% dispersion in mineral oil) (1.60 g, 40 mmol) in tetrahydrofuran (anhydrous) (400 mL) under N₂ atmosphere at 0° C. was added ethyl 5-methylindole-2-carboxylate (4 g, 19.09 mmol) over 15 min. The reaction mixture was allowed to stir at WTC for 30 min. Then di-tert-butyl dicarbonate (6 g, 27.22 mmol) was added in one portion. The reaction mixture was stirred at room temperature for 18 h. The reaction mixture was treated with ice-water (200 mL). The product was extracted with ethyl acetate (3×150 mL). The combined extracts were washed with brine (2×100 mL), dried with anhydrous sodium sulfate, filtered, and concentrated under reduced pressure to give crude 1-tert-butyl) 2-ethyl 5-methyl-1H-indole-1,2-dicarboxylate (7.6 g, 131%) as an orange oil. The crude product was used for the next step without any further purification. ¹H NMR (400 MHz, CDCl₃) δ 7 96 (d, J=8.6 Hz, 1H), 7.38 (s, 1H), 7.24 (d, J=8.6 Hz, 1H), 7.06-7.00 (m, 1H), 4.38 (q, J=7.2 Hz, 2H), 2.44 (s, 3H), 1.63 (s, 9H), 1.40 (t, J=7.2 Hz, 3H).

### Step 2: 1-(tert-Butyl) 2-ethyl 5-(bromomethyl)-1H-indole-1,2-dicarboxylate

To a solution of 1-(tert-butyl) 2-ethyl 5-methyl-1H-indole-1,2-dicarboxylate (7.6 g, 25.0 mmol) in anhydrous carbon tetrachloride (125 mL) under N₂ atmosphere was added N-bromosuccinimide (4.5 g, 25.0 mmol) and benzoyl peroxide (250 mg, 1.01 mmol). The reaction mixture was heated at reflux (78° C.) and stirred at this temperature for 18 h. The precipitate was filtered off and the solvent was removed on a rotary evaporator. The crude material was purified by flash-chromatography on a 40 g silica gel cartridge (with a pre-column of 50 g of silica gel) using a gradient of 0 to 40% EtOAc in heptanes in 18 CV to afford 1-tert-butyl) 2-ethyl 5-(bromomethyl)-1H-indole-1,2-dicarboxylate (7.1 g, 54%) as a yellow oil. The isolated product contained a mixture of brominated and di-brominated product in a ratio of ≈75:25. LCMS (ESI): m/z=326.0 [M-C₄H₉+2H]⁺. ¹H NMR (400 MHz, CDCl₃) δ 8.06 (d, J=8.8 Hz, 1H), 7.66-7.60 (m, 1H), 7.46 (dd, J=8.8, 1.7 Hz, 1H), 7.07 (s, 1H), 4.65-4.60 (m, 2H), 4.43-4.37 (m, 2H), 1.64 (s, 9H), 1.41 (t, J=7.2 Hz, 3H).

### Step 3: 1-(tert-Butyl) 2-ethyl 5-((diethoxyphosphoryl)methyl)-1H-indole-1,2-dicarboxylate

1-(tert-Butyl) 2-ethyl 5-(bromomethyl)-1H-indole-1,2-dicarboxylate (7.1 g, 13.54 mmol) and triethyl phosphite (3.1 mL, 17.74 mmol) were placed in a round bottomed flask equipped with a magnetic stirring bar. The reaction mixture was heated at 100° C., and stirred at this temperature for 6 h. The reaction mixture was directly purified through reverse phase chromatography on a 275 g C₁₈ cartridge using a gradient of 5-100% acetonitrile in water. The fractions containing the product were concentrated under reduced pressure to afford 1-(tert-butyl) 2-ethyl 5-((diethoxyphosphoryl)methyl)-1H-indole-1,2-dicarboxylate (4.8 g, 81%) as a brown oil. LCMS (ESI): m/z=440.1 (M+H)⁺. ¹H NMR (400 MHz, CDCl₃) δ 8.02 (d, J=8.6 Hz, 1H), 7.55-7.52 (m, 1H), 7.35 (dt, J=8.6, 2.0 Hz, 1H), 7.05 (s, 1H), 4.38 (q, J=7.3 Hz, 2H), 4.07-3.92 (m, 4H), 3.29-3.19 (m, 2H), 1.63 (s, 9H), 1.40 (t, J=7.1 Hz, 3H), 1.24 (t, J=7.1 Hz, 6H).

### Step 4: Benzyl 5-((diethoxyphosphoryl)methyl)-1H-indole-2-carboxylate

A mixture of 1-(tert-butyl) 2-ethyl 5-((diethoxyphosphoryl)methyl)-1H-indole-1,2-dicarboxylate (4.3 g, 9.77 mmol), benzyl alcohol (22 mL, 212.0 mmol) and titanium (IV) isopropoxide (300 μL, 2.59 mmol) were placed in a round bottomed flask equipped with a magnetic stirring bar. The reaction mixture was heated at 100° C. for 18 h. The reaction mixture was cooled down to 35° C., and quenched with 1 N aqueous HCl solution (20 mL). The product was extracted with EtOAc (2×100 mL) and the combined organic extracts were washed with brine (2×100 mL), dried with anhydrous sodium sulfate, filtered and concentrated under reduced pressure. The crude product was directly purified by reverse phase chromatography using a 150 g C₁₈ cartridge eluting with a gradient of acetonitrile in water (5% for 3 CV then 5 to 100% in 18 CV) to give benzyl 5-((diethoxyphosphoryl)methyl)-1H-indole-2-carboxylate (3.1 g, 74%) as a yellow oil. LCMS (ESI): m/z=402.1 [M+H]⁺. ¹H NMR (400 MHz, CDCl₃) δ 9.11 (br. s., 1H), 7.64 (br. s., 1H), 7.53-7.48 (m, 2H), 7.48-7.36 (m, 5H), 7.32 (d, J=6.6 Hz, 1H), 5.44 (s, 2H), 4.11-3.97 (m, 4H), 3.33-3.24 (m, 2H), 1.27 (t. J=7.0 Hz, 6H).

### Step 5: Dibenzyl 5-((diethoxyphosphoryl)methy)-1H-indole-1,2-dicarboxylate

To a suspension of sodium hydride (60% dispersion in mineral oil) (950 mg, 23.75 mmol) in tetrahydrofuran (anhydrous)(110 mL) under N₂ atmosphere at 0° C. was added benzyl 5-((diethoxyphosphoryl)methyl)-1H-indole-2-carboxylate (3.1 g, 7.19 mmol) in tetrahydrofuran (anhydrous) (10 mL) over 5 mm. The reaction mixture was allowed to stir at 0° C. for 10 min. Then benzyl chloroformate (1.6 mL, 10.87 mmol) was added dropwise. The reaction mixture was stirred at room temperature for 18 h. The reaction mixture was treated with ice-water (100 mL). The product was extracted with EtOAc (3×100 mL). The combined extracts were washed with brine (2×100 mL), dried with anhydrous sodium sulfate, filtered and concentrated under reduced pressure. The crude material was directly purified by reverse phase chromatography using a 150 g C₁₈ cartridge eluting with a gradient of acetonitrile in water (5% for 3 CV then 5 to 100% in 18 CV) to give dibenzyl 5-((diethoxyphosphoryl)methyl)-1H-indole-1,2-dicarboxylate (3.67 g, 88%) as a yellow oil. LCMS (ESI): m/z=536.2 [M+H]⁺. ¹H NMR (400 MHz, CDCl₃) δ 8.01 (d, J=8.6 Hz, 1H), 7.54 (br. s., 1H), 7.43-7.33 (m, 1H), 7.13 (s, 1H), 5.35 (s, 2H), 5.22 (s, 2H), 4.07-3.91 (m, 4H), 3.27-3.17 (m, 2H), 1.23 (t, J=7.0 Hz, 6H).

### Step 6: Dibenzyl 5-((diethoxyphosphoryl)difluoromethyl)-1H-indole-1,2-dicarboxylate

A mixture of dibenzyl 5-((diethoxyphosphoryl)methyl)-1H-indole-1,2-dicarboxylate (360 mg, 0.6400 mmol) and Accufluor NFSI (620 mg, 1.927 mmol) in tetrahydrofuran (anhydrous) (10 mL) were placed in a round bottomed flask equipped with a magnetic stirring bar. The reaction mixture was cooled down to −78° C. (dry ice+acetone). To this solution was added sodium bis(trimethylsilylamide) (1 M in THF)(2 mL, 2 mmol) over 10 min. The reaction mixture was stirred at −78° C. for 2 h before warming up to room temperature over 3 h. The reaction mixture was quenched with saturated NH₄Cl aqueous solution (20 mL). The product was extracted with EtOAc (3×40 mL). The combined extracts were washed with brine (2×50 mL), dried with anhydrous sodium sulfate, filtered and concentrated under reduced pressure. The crude residue was directly purified by reverse phase chromatography using a 50 g C₁₈ cartridge eluting with a gradient of acetonitrile in water (5% for 3 CV then 5 to 100% in 18 CV) to give to give dibenzyl 5-((diethoxyphosphoryl)difluoromethyl)-1H-indole-1,2-dicarboxylate (300 mg, 82%) as a yellow oil. LCMS (ESI): m/z=572.1 [M+H]⁺. ¹H NMR (400 MHz, CDCl₃) δ 8.15 (d, J=8.8 Hz, 1H), 7.89 (s, 1H), 7.67 (d, J=9.0 Hz, 1H), 7.46-7.32 (m, 10H), 7.23-7.18 (m, 1H), 5.37 (s, 2H), 5.23 (s, 2H), 4.26-4.06 (m, 4H), 1.30 (t. J=7.1 Hz, 6H).

### Step 7: ({1,2-bis[(Benzyloxy)carbonyl]-1H-indol-5-yl}difluoromethyl)phosphonic acid

1,2-Dibenzyl 5-[(diethoxyphosphoryl)difluoromethyl]-1H-indole-1,2-dicarboxylate (1.9 g, 3.32 mmol) was dissolved in methylene chloride (15 mL) and cooled to 0° C. N,O-Bis(trimethylsilyl)trifluoroacetamide (5.28 mL, 19.9 mmol) was added followed by a solution of iodotrimethylsilane (1 M in DCM, freshly prepared) (13.2 mL, 13.2 mmol) which was added dropwise at 0° C. The reaction mixture was stirred at 0° C. for 20 min, and the solvent was then removed under vacuum at 0° C. The crude product was dissolved in a mixture of water:CH₃CN:TFA (2:1:0.1) and directly purified by reverse phase chromatography using a 50 g C₁₈ cartridge eluting with a gradient of acetonitrile in water (5% for 4 CV then 5 to 100% in 18 CV) to give ({1,2-bis[(benzyloxy)carbonyl]-1H-indol-5-yl}difluoromethyl)phosphonic acid (1.16 g, 68%) as an orange solid. ¹H NMR (400 MHz, DMSO-d₆) δ 7.99-7.85 (m, 2H), 7.65 (d, J=9.0 Hz, 1H), 7.53-7.27 (m, 10H), 6.69 (br. s, 3H), 5.41 (s, 2H), 5.22 (s, 2H).

### Step 8: 1,2-Dibenzyl 5-{[bis({[(2,2-dimethylpropanoyl)oxy]methoxy})phosphoryl]difluoromethyl}-1H-indole-1,2-dicarboxylate

Sodium hydroxide (185 mg, 4.64 mmol) in H₂O (2 mL) was added dropwise to a stirred suspension of ({1,2-bis[(benzyloxy)carbonyl]-1H-indol-5-yl}difluoromethyl)phosphonic acid (1.20 g, 2.32 mmol) in H₂O (6 mL). When the mixture became clear (pH ~9), silver nitrate (985 mg, 5.80 mmol) was added. After 2 h at 0° C., the gray precipitate was collected by filtration and dried under vacuum. The powder was suspended in dry toluene (14 mL) and iodomethyl 2,2-dimethylpropanoate (1.68 g, 6.96 mmol) was added. The mixture was stirred for 24 h at room temperature. After filtration, the solvent was removed in vacuo. The crude residue was directly purified by normal phase chromatography using a 40 g silica gel cartridge eluting with a gradient of EtOAc in heptanes (0 to 60% in 18 CV) to give 1,2-dibenzyl 5-{[bis({[(2,2-dimethylpropanoyl)oxy]methoxy})phosphoryl]difluoromethyl}-1H-indole-1,2-dicarboxylate (820 mg, 48%) as a brown oil. ¹H NMR (400 MHz, DMSO-d₆) δ 8.17-8.08 (m, 1H), 7.99-7.92 (m, 1H), 7.60 (d, J=8.8 Hz, 1H), 7.52-7.30 (m, 1H), 5.74-5.59 (m, 4H), 5.46-5.41 (m, 2H), 5.28-5.20 (m, 2H), 1.13-1.06 (m, 18H).

### Step 9: 5-{[bis({[(2,2-Dimethylpropanoyl)oxy]methoxy})phosphoryl]difluoromethyl}-1H-indole-2-carboxylic acid

10% Palladium on carbon (50% wet) (350 mg, 164 μmol) was added to a mixture of 1,2-dibenzyl 5-{[bis({[(2,2-dimethylpropanoyl)oxy]methoxy})phosphoryl]difluoromethyl}-1H-indole-1,2-dicarboxylate (820 mg, 1.10 mmol) in anhydrous tetrahydrofuran (20 mL). Hydrogen was bubbled into the suspension for 2 min, and then the reaction mixture was stirred under 1 atm. of hydrogen for 18 h. The reaction mixture was filtered on a Celite pad and the pad was rinsed with Me-THF. The filtrate was concentrated in vacuo. The crude residue was purified by reverse phase chromatography using a 50 g C₁₈ cartridge eluting with a gradient of acetonitrile in water (5% for 3 CV then 5 to 100% in 18 CV) to give 5-{[bis({[(2,2-dimethylpropanoyl)oxy]methoxy})phosphoryl]difluoromethyl}-1H-indole-2-carboxylic acid (444 mg, 78%) as a light green solid. ¹H NMR (400 MHz, DMSO-d₆) δ 13.18 (br. s., 1H), 12.12 (s, 1H), 7.88 (s, 1H), 7.55 (d, J 8.6 Hz, 1H), 7.37 (d, J=9.0 Hz, 1H), 7.21 (s, 1H), 5.72-5.61 (m, 4H), 1.15-1.09 (m, 18H).

### Preparation of ((((2-(((5S,8S,10aR)-3-(8-(2-(2,6-Dioxopiperidin-3-yl)-1-oxoisoindolin-4-yl)oct-7-ynoyl)-8-(methyl(phenyl)carbamoyl)-6-oxodecahydropyrrolo[1,2-a][1,5]diazocin-5-yl)carbamoyl)-1H-indol-5-yl)difluoromethyl)phosphoryl)bis(oxy))bis(methylene) bis(2,2-dimethylpropanoate) (Compound 2)

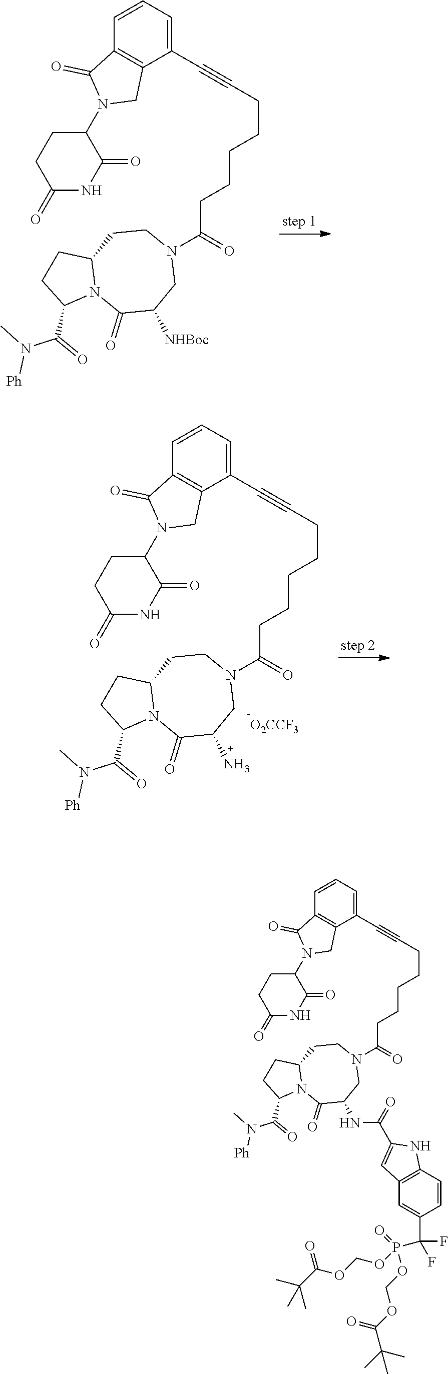

### Step 1: (5S,8S,10aR)-5-Amino-3-{8-[2-(2,6-dioxopiperidin-3-yl)-1-oxo-2,3-dihydro-1H-isoindol-4-yl]oct-7-ynoyl}-N-methyl-6-oxo-N-phenyl-decahydropyrrolo[1,2-a][1,5]diazocine-8-carboxamide

To a solution of tert-butyl N-[(5S,8S,10aR)-3-{8-[2-(2,6-dioxopiperidin-3-yl)-1-oxo-2,3-dihydro-1H-isoindol-4-yl]oct-7-ynoyl}-8-[methyl(phenyl)carbamoyl]-6-oxo-decahydropyrrolo[1,2-a][1,5]diazocin-5-yl]carbamate (60 mg, 76.8 μmol, 1 eq) in methylene chloride (2 mL) was added trifluoroacetic acid (1 mL). The resulting yellow solution was stirred for 1 h at room temperature. The reaction mixture was concentrated under reduced pressure. The crude TFA salt was used directly for the next step without further purification. LCMS (ESI): m/z=681.3 (M+H)⁺.

### Step 2: ((((2-(((5S,8S,10aR)-3-(8-(2-(2,6-Dioxopiperidin-3-yl)-1-oxoisoindolin-4-yl)oct-7-ynoyl)-8-(methyl(phenyl)carbamoyl)-6-oxodecahydropyrrolo[1,2-a][1,5]diazocin-5-yl)carbamoyl)-1H-indol-5-yl)difluoromethyl)phosphoryl)bis(oxy))bis(methylene) bis(2,2-dimethylpropanoate)

To a solution of (5S,8S,10aR)-5-amino-3-{8-[2-(2,6-dioxopiperidin-3-yl)-1-oxo-2,3-dihydro-1H-isoindol-4-yl]oct-7-ynoyl}-N-methyl-6-oxo-N-phenyl-decahydropyrrolo[1,2-a][1,5]diazocine-8-carboxamide TFA salt (61 mg, 76.8 μmol, 1 eq) in N,N-dimethylformamide (3 mL) was added N,N-diisopropylethylamine (79.5 μL, 457 μmol, 6 eq). The reaction mixture was stirred for 5 min, and then 5-{[bis({[(2,2-dimethylpropanoyl)oxy]methoxy))phosphoryl]difluoromethyl}-1H-indole-2-carboxylic acid (47.5 mg, 91.5 μmol, 1.2 eq) and HATU (37.6 mg, 99.1 μmol, 1.3 eq) were added. The resulting yellow solution was stirred for 30 min. at room temperature. The reaction mixture was directly purified by reverse phase chromatography on a 30 g C₁₈ cartridge eluting with 5-100% acetonitrile in water (5% acetonitrile in water 3 CV, 5-100% acetonitrile in water 2 CV then 100% acetonotrile in 7 CV). The combined fractions were concentrated. The isolated product was re-purified by flash-chromatography on a 12 g silica gel cartridge eluting with 0-20% DCM in methanol to give ((((2-(((5S,8S,10aR)-3-(8-(2-(2,6-dioxopiperidin-3-yl)-1-oxoisoindolin-4-yl)oct-7-ynoyl)-8-(methyl(phenyl)carbamoyl)-6-oxodecahydropyrrolo[1,2-a][1,5]diazocin-5-yl)carbamoyl)-1H-indol-5-yl)difluoromethyl)phosphoryl)bis(oxy))bis(methylene) bis(2,2-dimethylpropanoate) (Compound 2, 45.0 mg, 50%) as a beige solid LCMS (ESI): m/z=1182.4 (M+H)⁺. ¹H NMR (400 MHz, CD₃OD) δ 7.92-7.84 (m, 1H), 7.74-7.32 (m, 10H), 7.28-7.20 (m, 1H), 5.76-5.66 (m, 4H), 5.27-4.90 (m, 2H), 4.51-4.47 (m, 1H), 4.41-4.36 (m, 1H), 4.27-4.15 (m, 1H), 4.12-3.92 (m, 1H), 3.90-3.75 (m, 1H), 3.67-3.57 (m, 1H), 3.56-3.37 (m, 1H), 3.28-3.18 (m, 3H), 2.91-2.80 (m, 1H), 2.75-2.67 (m, 2H), 2.58-2.44 (m, 3H), 2.20-2.09 (m, 1H), 2.06-1.81 (m, 6H), 1.80-1.58 (m, 6H), 1.36-1.27 (m, 1H), 1.21-1.17 (m, 19H).

### Preparation of ((2-(((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphonic acid (Compound 3)

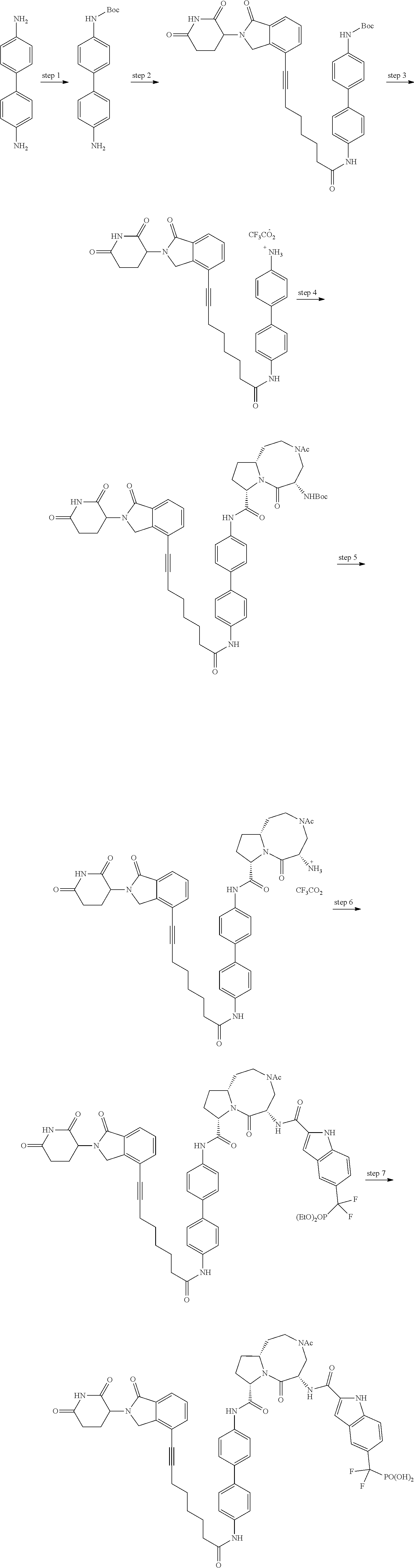

### Step 1: tert-Butyl (4′-amino-[1,1′-biphenyl]-4-yl)carbamate

To a stirred solution of benzidine (500 mg, 2.71 mmol, 1 eq) in a mixture of DMF (3 mL). THF (10 mL) and H₂O (1.5 mL) were added potassium carbonate (317 mg, 2.30 mmol, 0.85 eq) and di-tert-butyl dicarbonate (471 mg, 2.16 mmol, 0.8 eq) at room temperature. The resulting mixture was stirred for 18 h at room temperature. The reaction mixture was then diluted with water (50 mL) and the product was extracted with EtOAc (3×20 mL). The organic layers were combined, washed with brine (2×10 mL), dried with anhydrous sodium sulfate and concentrated under reduced pressure. The crude residue was purified by reverse phase chromatography on a 50 g C₁₈ cartridge eluting with 5-100% acetonitrile in water. The combined fractions were concentrated under reduced pressure to give tert-butyl (4′-amino-[1,1′-biphenyl]-4-yl)carbamate (420 mg, 55%) as a yellow solid. LCMS (ESI): m/z=285.2 (M+H)⁺.

### Step 2: tert-Butyl (4′-(8-(2-(2,6-dioxopiperidin-3-yl)-1-oxoisoindolin-4-yl)oct-7-ynamido)-[1,1′-biphenyl]-4-yl)carbamate

To a solution of 8-[2-(2,6-dioxopiperidin-3-yl)-1-oxo-2,3-dihydro-1H-isoindol-4-yl]oct-7-ynoic acid (50 mg, 130 μmol, 1 eq) and tert-butyl (4′-amino-[1,1′-biphenyl]-4-yl)carbamate (44.0 mg, 155 μmol, 1.2 eq) in N,N-dimethylformamide (2 mL) and N,N-diisopropylethylamine (134 μL, 779 μmol, 6 eq) was added HATU (64.2 mg, 169 μmol, 1.3 eq). The resulting brown solution was stirred for 30 min. at room temperature. The reaction mixture was directly purified by reverse phase chromatography on a 50 g C₁₈ cartridge eluting with 5-100% acetonitrile in water. The combined fractions were concentrated under reduced pressure to give tert-butyl (4′-(8-(2-(2,6-dioxopiperidin-3-yl)-1-oxoisoindolin-4-yl)oct-7-ynamido)-[1,1′-biphenyl]-4-yl)carbamate (40.0 mg, 47%) as a beige solid. LCMS (ESI): m/z=649.3 (M+H)⁺.

### Step 3: N-(4′-Amino-[1,1′-biphenyl]-4-yl)-8-(2-(2,6-dioxopiperidin-3-yl)-1-oxoisoindolin-4-yl)oct-7-ynamide

To a solution of tert-butyl (4′-(8(2-(2,6-dioxopiperidin-3-yl)-1-oxoisoindolin-4-yl)oct-7-ynamido)-[1,1′-biphenyl]-4-yl)carbamate (40 mg, 61.6 μmol, 1 eq) in methylene chloride (2 mL) was added trifluoroacetic acid (1 mL). The resulting yellow solution was stirred for 1 h at room temperature. The reaction mixture was concentrated under reduced pressure. The crude TFA salt was used directly for the next step without further purification. LCMS (ESI): m/z=549.2 (M+H)⁺.

### Step 4: tert-Butyl ((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamate

To a solution of (5S,8S,10aR)-3-acetyl-5-{[(tert-butoxy)carbonyl]amino}-6-oxo-decahydropyrrolo[1,2-a][1,5]diazocine-8-carboxylic acid (25 mg, 67.6 μmol, 1 eq) and N-(4′-amino-[1,1′-biphenyl]-4-yl)-8-(2-(2,6-dioxopiperidin-3-yl)-1-oxoisoindolin-4-yl)oct-7-ynamide TFA salt (44.8 mg, 67.6 μmol, 1 eq) in N,N-dimethylformamide (2 mL) and N,N-diisopropylethylamine (70.4 μL, 405 μmol, 6 eq) was added HATI (33.3 mg, 87.8 μmol, 1.3 eq). The resulting brown solution was stirred for 2 h at room temperature. The reaction mixture was directly purified by reverse phase chromatography on a 50 g C₁₈ cartridge eluting with 5-80% acetonitrile in water. The combined fractions were concentrated under reduced pressure to give tert-butyl ((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamate (41.0 mg, 67%) as a beige solid. LCMS (ESI): m/z=901.6 (M+H)⁺.

### Step 5: (5S,8S,10aR)-3-Acetyl-5-amino-N-(4′-(8-(2-(2,6-dioxopiperidin-3-yl)-1-oxoisoindolin-4-yl)oct-7-ynamido)-[1,1′-biphenyl]-4-yl)-6-oxodecahydropyrrolo[1,2-a][1,5]diazocine-8-carboxamide

To a solution of tert-butyl ((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamate (40 mg, 44.4 μmol, 1 eq) in methylene chloride (2 mL) was added trifluoroacetic acid (1 mL). The resulting yellow solution was stirred for 1 h at room temperature. The reaction mixture was concentrated under reduced pressure. The crude TFA salt was used directly for the next step without further purification. LCMS (ESI): m/z=800.4 (M+H)⁺.

### Step 6: Diethyl ((2-(((5S,8S 10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphonate

To a solution of 5-[(diethoxyphosphoryl)difluoromethyl]-1H-indole-2-carboxylic acid (10.9 mg, 31.6 μmol, 1.1 eq) and (5S,8S,10aR)-3-acetyl-5-amino-N-(4′-(8-(2-(2,6-dioxopiperidin-3-yl)-1-oxoisoindolin-4-yl)oct-7-ynamido)-[1,1′-biphenyl]-4-yl)-6-oxodecahydropyrrolo[1,2-a][1,5]diazocine-8-carboxamide (26.3 mg, 28.8 μmol, 1 eq) in N,N-dimethylformamide (2 mL) and N,N-diisopropylethylamine (29.9 μL, 172 μmol, 6 eq) was added HATU (14.2 mg, 37.4 μmol, 1.3 eq). The resulting brown solution was stirred for 2 h at room temperature. The reaction mixture was directly purified by reverse phase chromatography on a 50 g C₁₈ cartridge eluting with 5-80% acetonitrile in water (with 0.1% formic acid). The product was repurified purified by reverse phase chromatography on a 50 g C₁₈ cartridge eluting with 5-80% acetonitrile in water (with 0.1% formic acid). The combined fractions were concentrated under reduced pressure to give diethyl ((2-(((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphonate (15.0 mg, 46%) as a white solid. LCMS (ESI): m/z=1130.4 (M+H)⁺.

### Step 7: ((2-(((5S,8S,10aR)-3-Acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphonic acid

To a solution of diethyl ((2-(((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphonate (15 mg, 12.3 μmol, 1 eq) in methylene chloride (5 mL) at 0° C. was added N,O-bis(trimethylsilyl)trifluoroacetamide (19.5 mL, 73.8 μmol, 6 eq) followed by a 1 M solution of trimethylsilyl iodide in DCM (49.2 μL, 49.2 μmol, 4 eq). The reaction mixture was stirred for 15 mm, at 0° C. The reaction mixture was quenched with the addition of a 2:1 mixture of H₂O and acetonitrile (with 0.1% TFA) (0.5 mL). The solvent was removed under reduced pressure at 0° C. The crude residue was purified by reverse phase chromatography on a 30 g C₁₈ cartridge eluting with 5-40% acetonitrile in water. The combined fractions were directly freeze-dried to give ((2-(((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphonic acid (Compound 3, 5.9 mg, 45%) as a white solid. LCMS (ESI): m/z=1073.4 (M+H)⁺. ¹H NMR (400 MHz, CD₃OD:D₂O 1:0.1) δ 7.94 (s, 1H), 7.68-7.59 (m, 3H), 7.57-7.38 (m, 10H), 7.31-7.26 (m, 1H), 5.27-5.22 (m, 1H), 5.12-5.08 (m, 1H), 4.67-4.60 (m, 1H), 4.50-4.38 (m, 3H), 3.97-3.62 (m, 4H), 2.95-2.77 (m, 2H), 2.57-2.50 (m, 2H), 2.50-2.36 (m, 4H), 2.26-2.24 (m, 3H), 2.21-2.06 (m, 3H), 2.04-1.87 (m, 2H), 1.85-1.75 (m, 2H), 1.72-1.67 (m, 2H), 1.62-1.55 (m, 2H), 1.38-1.22 (m, 1H).

### Preparation of ((((2-(((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphoryl)bis(oxy))bis(methylene) bis(2,2-dimethylpropanoate) (Compound 4)

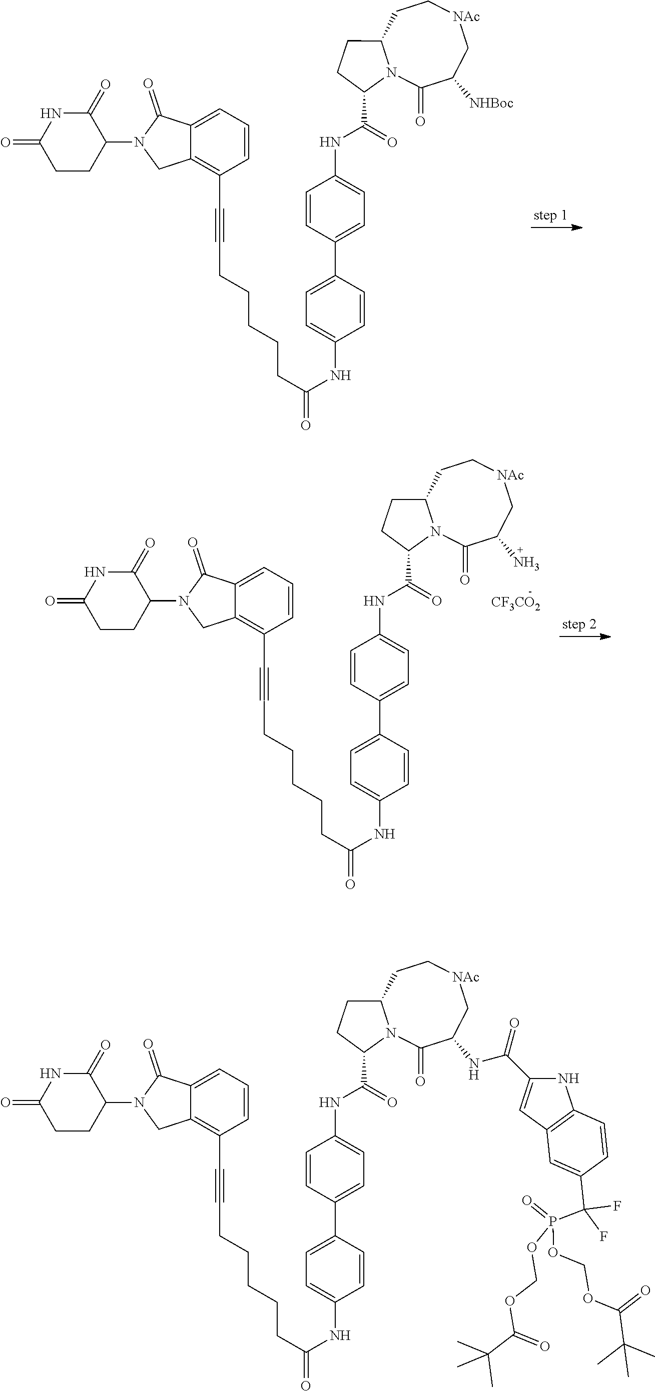

### Step 1: (5S,8S,10aR)-3-Acetyl-5-amino-N-(4′-(8-(2-(2,6-dioxopiperidin-3-yl)-1-oxoisoindolin-4-yl)oct-7-ynamido)-[1,1′-biphenyl]-4-yl)-6-oxodecahydropyrrolo[1,2-a][1,5]diazocine-8-carboxamide

To a solution of tert-butyl ((5S,8S,10aR)-3-acetyl-8-((4′-(8-(2-(2,6-dioxopiperidin-3-yl)-1-oxoisoindolin-4-yl)oct-7-ynamido)-[1,1′-biphenyl]-4-yl)carbamoyl)-6-oxodecahydropyrrolo[1,2-a][1,5]diazocin-5-yl)carbamate (200 mg, 222 μmol, 1 eq) in methylene chloride (5 mL) was added trifluoroacetic acid (2 mL). The resulting yellowish solution was stirred for 1 h at room temperature. The reaction was concentrated under reduced pressure. The crude residue was purified by reverse phase chromatography on a 50 g C₁₈ cartridge eluting with 5-60% acetonitrile in water. The combined fractions were concentrated under reduced pressure to give (5S,8S,10aR)-3-acetyl-5-amino-N-(4′-(8-(2-(2,6-dioxopiperidin-3-yl)-1-oxoisoindolin-4-yl)oct-7-ynamido)-[1,1′-biphenyl]-4-yl)-6-oxodecahydropyrrolo[1,2-a][1,5]diazocine-8-carboxamide (80 mg, 45%) as a white solid. LCMS (ESI): m/z=800.4 (M+H)⁺.

### Step 2: ((((2-(((5S,8S,10aR)-3-Acetyl-8-((4′-(8-(2-(2,6-dioxopiperidin-3-yl)-1-oxoisoindolin4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphoryl)bis(oxy))bis(methylene) bis(2,2-dimethylpropanoate)

To a solution of (S,8S,10aR)-3-acetyl-5-amino-N-(4′-(8-(2-(2,6-dioxopiperidin-3-yl)-1-oxoisoindolin-4-yl)oct-7-ynamido)-[1,1′-biphenyl]-4-yl)-6-oxodecahydropyrrolo[1,2-a][1,5]diazocine-8-carboxamide TFA salt (40 mg, 43.8 μmol, 1 eq) in N,N-dimethylforamide (2 mL) was added N,N-diisopropylethylamine (52.1 μL, 300 μmol, 6.8 eq). The reaction mixture was stirred for 5 min. then 5-{[bis({[(2,2-dimethylpropanoyl)oxy]methoxy})phosphoryl]difluoromethyl}-1H-indole-2-carboxylic acid (28.5 mg, 55.0 μmol, 1.25 eq) and HATU (24.7 mg, 65.0 μmol, 1.5 eq) were added. The resulting yellow solution was stirred for 30 min. at room temperature. The reaction mixture was directly purified by reverse phase chromatography on a 30 g C₁₈ cartridge eluting with 5-100% acetonitrile in water (5% acetonitrile in water 3 CV, 5-100% acetonitrile in water 2 CV then 100% acetonitrile in 7 CV). The combined fractions were concentrated. The isolated product was purified by flash-chromatography on a 12 g silicagel cartridge eluting with 0-20% DCM in methanol to give ((((2-(((5S,8S,10aR)-3-acetyl-8-((4′-(8-(2-(2,6-dioxopiperidin-3-yl)-1-oxoisoindolin4-yl)oct-7-ynamido)-[1,1′-biphenyl]-4-yl)carbamoyl)-6-oxodecahydropyrrolo[1,2-a][1,5]diazocin-5-yl)carbamoyl)-1H-indol-5-yl)difluoromethyl)phosphoryl)bis(oxy))bis(methylene) bis(2,2-dimethylpropanoate) (Compound 4, 22.9 mg, 40%) as a beige solid. LCMS (ESI): m/z=1301.5 (M+H)⁺. ¹H NMR (400 MHz, CD₃OD) δ 7.88 (br. s., 1H), 7.71-7.62 (m, 3H), 7.59-7.36 (m, 10H), 7.29-7.23 (m, 1H), 5.76-5.62 (m, 4H), 5.25-5.10 (m, 2H), 4.68-4.57 (m, 1H), 4.52-4.38 (m, 3H), 4.03-3.58 (m, 4H), 2.94-2.82 (m, 1H), 2.81-2.71 (m, 1H), 2.59-2.47 (m, 3H), 2.46-2.34 (m, 3H), 2.28-2.06 (m, 7H), 2.02-1.84 (m, 2H), 1.84-1.74 (m, 2H), 1.74-1.66 (m, 2H), 1.67-1.62 (m, 2H), 1.18 (s, 18H).

### Preparation of ((2-(((5S,8S,10aR)-3-(8-(2-(2,6-dioxopiperidin-3-yl)-1-oxoisoindolin-4-yl)oct-7-ynoyl)-6-oxo-8-(3-(pyridin-3-yl)azetidine-1-carbonyl)decahydropyrrolo[1,2-a][1,5]diazocin-5-yl)carbamoyl)benzo[b]thiophen-5-yl)difluoromethyl)phosphonic acid (Compound 5)

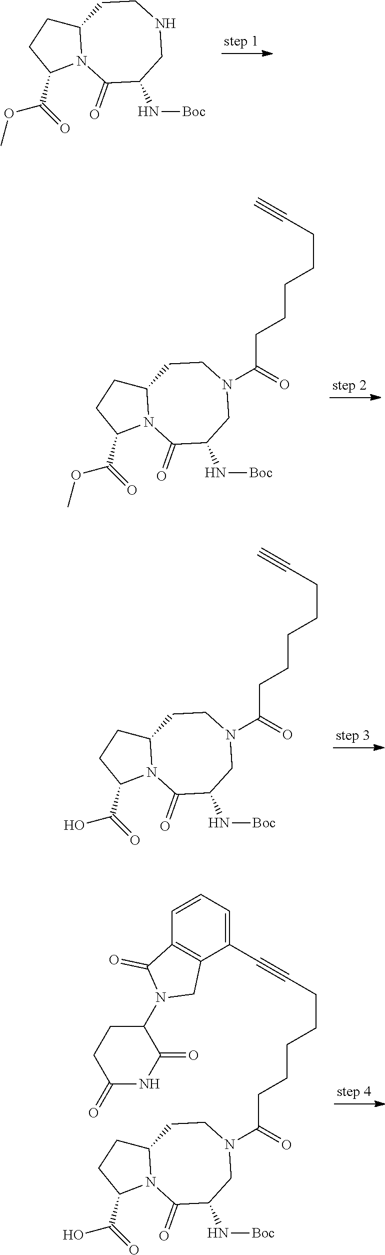 [SMILES: COC(=O)[C@@H]1CC[C@@H]2CCNC[C@H](NC(=O)OC(C)(C)C)C(=O)N21 ; C#CCCCCCC(=O)N1CC[C@H]2CC[C@@H](C(=O)OC)N2C(=O)[C@@H](NC(=O)OC(C)(C)C)C1 ; C#CCCCCCC(=O)N1CC[C@H]2CC[C@@H](C(=O)O)N2C(=O)[C@@H](NC(=O)OC(C)(C)C)C1 ; CC(C)(C)OC(=O)N[C@H]1CN(C(=O)CCCCCC#Cc2cccc3c2CN(C2CCC(=O)NC2=O)C3=O)CC[C@H]2CC[C@@H](C(=O)O)N2C1=O]

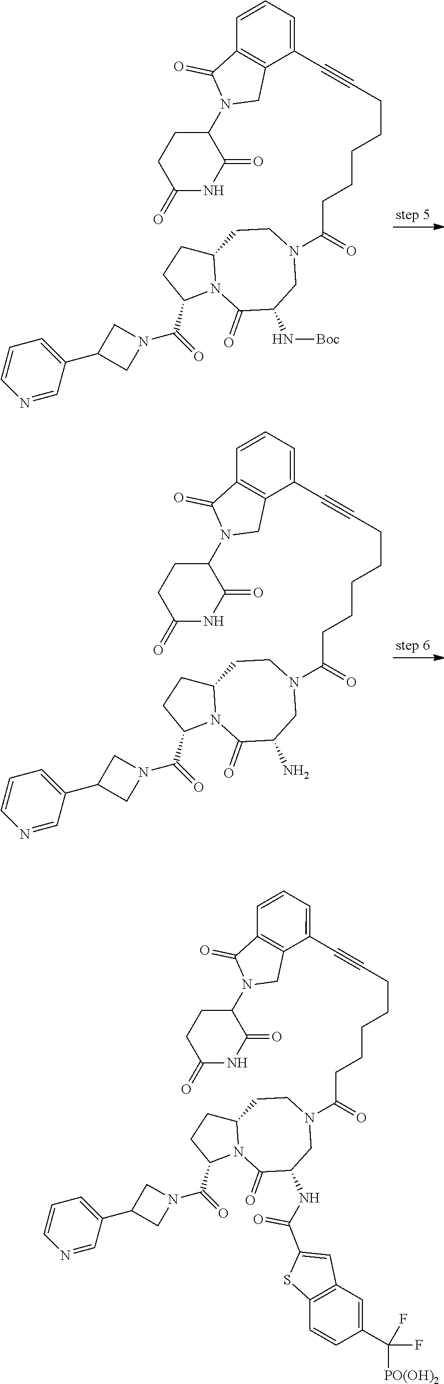

### Step 1: Preparation of (5S,8S,10R)-methyl 5-((tert-butoxycarbonyl)amino)-3-(oct-7-ynoyl)-6-oxodecahydropyrrolo[1,2-a][1,5]diazocine-8-carboxylate

To a solution of oct-7-ynoic acid (0.30 g, 2.20 mmol, 1 eq) in DCM (5 mL) was added HATU (2.50 g, 6.60 mmol, 3 eq) and ethylbis(propan-2-yl)amine (0.33 g, 2.60 mmol, 1.2 eq). The mixture was stirred at 25° C. for 10 minutes. Then, a solution of methyl (5S,8S,10aR)-5-((tert-butoxycarbonyl)amino)-6-oxodecahydropyrrolo[1,2-a][1,5]diazocine-8-carboxylate (0.80 g, 2.40 mmol, 1.1 eq) in DCM (2 mL) was added to the mixture. The mixture was stirred at 25° C. for 2 hour to give a yellow mixture. The reaction mixture was filtered and concentrated under reduced pressure to give a residue. The residue was purified by flash silica gel chromatography to give (5S,8S,10aR)-methyl 5-((tert-butoxycarbonyl)amino)-3-(oct-7-ynoyl)-6-oxodecahydro pyrrolo[1,2-a][1,5]diazocine-8-carboxylate (0.6 g, 58.6% yield, 1.30 mmol) as a white solid. LCMS (ESI) m/z=464.3. ¹H NMR (400 MHz, CHLOROFORM-d) δ 4.55-4.45 (m, 1H), 3.98-3.82 (m, 1H), 3.79-3.73 (m, 2H), 3.73-3.65 (m, 2H), 3.47-3.25 (m, 1H), 3.20-3.10 (m, 3H), 2.64 (s, 1H), 2.43-2.32 (m, 1H), 2.24-2.18 (m, 1H), 2.17-2.06 (m, 1H), 2.03-1.91 (m, 1H), 1.93 (t, J=2.4 Hz, 1H), 1.88-1.77 (m, 1H), 1.75-1.64 (m, 1H), 1.59 (d, J=7.0 Hz, 1H), 1.48-1.46 (m, 4H), 1.46-1.44 (m, 9H), 1.42 (s, 4H).

### Step 2: Preparation of (5S,8S,10aR)-5-((tert-butoxycarbonyl)amino)-3-(oct-7-ynoyl)-6-oxodecahydropyrrolo[1,2-a][1,5]diazocine-8-carboxylic acid

To a solution of (5S,8S,10aR)-methyl 5-((tert-butoxycarbonyl)amino)-3-(oct-7-ynoyl)-6-oxodecahydropyrrolo[1,2-a][1,5]diazocine-8-carboxylate (0.60 g, 1.30 mmol, 1 eq) in methanol (1 mL), THF (1 mL) and H₂O (1 mL) was added lithium hydroxide (0.11 g, 2.64) mmol, 2 eq). The mixture was stirred at 25° C. for 1 hour to give a yellow mixture. The reaction mixture was filtered and concentrated under reduced pressure to give a residue which was purified by prep-HPLC (column: Waters bridge 150×25 mm×10 um; mobile phase: [water (NH₄HCO₃)−ACN]; B %: 0%-30%, 11 min) then lyophilized to give (5S,8S,10aR)-5-((tert-butoxycarbonyl)amino)-3-(oct-7-ynoyl)-6-oxodecahydropyrrolo[1,2-a][1,5]diazocine-8-carboxylic acid (0.2 g, 41% yield, 0.45 mmol) as a white solid. LCMS (ESI) m/z=450.1

### Step 3: Preparation of (5S,8S,10aR)-5-((tert-butoxycarbonyl)amino)-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carboxylic acid

To a solution of (5S,8S,10aR)-5-((tert-butoxycarbonyl)amino)-3-(oct-7-ynoyl)-6-oxodecahydropyrrolo[1,2-a][1,5]diazocine-8-carboxylic acid (0.2 g, 0.45 mmol, 1 eq) in DMF (1 mL) was added 3-(4-bromo-1-oxoisoindolin-2-yl)piperidine-2,6-dione (0.14 g, 0.45 mmol, 1 eq) and copper iodide (17 mg, 89 μmol, 0.2 eq), then triethylamine (0.14 g, 1.4 mmol, 3 eq) and bis((cyclopenta-1,3-dien-1-yl)diphenylphosphane) dihydrochloride iron palladium (33 mg, 45 μmol, 0.1 eq) was added in the mixture at 25° C. under N₂ atmosphere. Then the mixture was stirred at 80° C. for 3 hours to give a brown solution. The reaction mixture was quenched by water (20 mL) and adjusted to pH=2-3 with the addition of IM HCl aqueous solution, then extracted with ethyl acetate 60 mL (20 mL×3). The combined organic layer was washed with brine 20 mL, dried over Na₂SO₄, filtered and concentrated under reduced pressure to give a residue. The residue was purified by column chromatography to give (5S,8S,10aR)-5-((tert-butoxycarbonyl)amino)-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carboxylic acid (0.17 g, 55% yield, 0.25 mmol) as a white solid. LCMS (ESI) m/z=692.6

### Step 4: Preparation of tert-butyl ((5S,8S,10aR)-3-(8-(2-(2,6-dioxopiperidin-3-yl)-1-oxoisoindolin-4-yl)oct-7-ynoyl)-6-oxo-8-(3-(pyridin-3-yl)azetidine-1-carbonyl)decahydropyrrolo[1,2-a][1,5]diazocin-5-yl)carbamate

To a solution of (5S,8S,10aR)-5-((tert-butoxycarbonyl)amino)-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carboxylic acid (60 mg, 87 μmol, 1 eq) in DCM (1 mL) was added Chloro-N,N,N′,N′-tetramethylformamidinium hexafluorophosphate (29 mg, 0.10 mmol, 1.2 eq) and 1-methyl-1H-imidazole (22 mg, 0.26 mmol, 3 eq). The mixture was stirred at 25° C. for 10 minutes and then 3-(azetidin-3-yl)pyridine (12 mg, 87 μmol, 1 eq) was added in the mixture, the mixture was stirred at 25° C. for 1 hour to give a yellow solution. The reaction mixture was filtered and concentrated under reduced pressure to give a residue. The residue was purified by flash silica gel chromatography to give tert-butyl ((5S,8S,10aR)-3(8-(2-(2,6-dioxopiperidin-3-yl)-1-oxoisoindolin-4-yl)oct-7-ynoyl)-6-oxo-8-(3-(pyridin-3-yl)azetidine-1-carbonyl)decahydropyrrolo[1,2-a][1,5]diazocin-5-yl)carbamate (40 mg, 57.10% yield, 49 μmol) as a white solid. LCMS (ESI) m/z=808.3

### Step 5: Preparation of 3-(4-(8-((5S,8S,10aR)-5-amino-6-oxo-8-(3-(pyridin-3-yl)azetidine-1-carbonyl)octahydropyrrolo[1,2-a][1,5]diazocin-3(4H)-yl)-8-oxooct-1-yn-1-yl)-1-oxoisoindolin-2-yl)piperidine-2,6-dione

To a solution of tert-butyl ((5S,8S,10aR)-3-(8-(2-(2,6-dioxopiperidin-3-yl)-1-oxoisoindolin-4-yl)oct-7-ynoyl)-6-oxo-8-(3-(pyridin-3-yl)azetidine-1-carbonyl)decahydropyrrolo[1,2-a][1,5]diazocin-5-yl)carbamate (80 mg, 99 μmol, 1 eq) in DCM (0.5 mL) and TFA (0.3 mL) was stirred at 25° C. for 0.5 hour to give a yellow solution. The reaction mixture was filtered and concentrated under reduced pressure to give 3-(4-(8-((5S,8S,10aR)-5-amino-6-oxo-8-(3-(pyridin-3-yl)azetidine-1-carbonyl)octahydropyrrolo[1,2-a][1,5]diazocin-3(4H)-yl)-8-oxooct-t-yn-1-yl)-1-oxoisoindolin-2-yl)piperidine-2,6-dione (80 mg, crude) as a yellow oil.

### Step 6: Preparation of ((2-(((5S,8S,10aR)-3-(8-(2-(2,6-dioxopiperidin-3-yl)-1-oxoisoindolin-4-yl)oct-7-ynoyl)-6-oxo-8-(3-(pyridin-3-yl)azetidine-1-carbonyl)decahydropyrrolo[1,2-a][1,5]diazocin-5-yl)carbamoyl)benzo[b]thiophen-5-yl)difluoromethyl)phosphonic acid

To a solution of 3-(4-(8-((5S,8S,10aR)-5-amino-6-oxo-8-(3-(pyridin-3-yl)azetidine-1-carbonyl)octahydropyrrolo[1,2-a][1,5]diazocin-3(4H)-yl)-8-oxooct-1-yn-1-yl)-1-oxoisoindolin-2-yl)piperidine-2,6-dione (70 mg, 99 μmol, 1.0 eq) in DCM (1 mL) was added (difluoro(2-((4-nitrophenoxy)carbonyl)benzo[b]thiophen-5-yl)methyl)phosphonic acid (51 mg, 0.12 mmol, 1.2 eq), ethylbis(propan-2-yl)amine (16 mg, 0.12 mmol, 1.2 eq) and 1H-1,2,3-benzotriazol-1-ol (40 mg, 0.30 mmol, 3 eq). The mixture was stirred at 25° C. for 10 minutes to give a yellow solution. The reaction mixture filtered and concentrated under reduced pressure to give a residue. The residue was purified by prep-HPLC (column: Phenomenex Luna C18 150×25 mm×10 um; mobile phase: [water (0.1% TFA)−ACN]; B %: 32%-62%, 10 min). The solution was lyophilized to give ((2-(((5S,8S,10aR)-3-(8-(2-(2,6-dioxopiperidin-3-yl)-1-oxoisoindolin-4-yl)oct-7-ynoyl)-6-oxo-8-(3-(pyridin-3-yl)azetidine-1-carbonyl)decahydropyrrolo[1,2-a][1,5]diazocin-5-yl)carbamoyl)benzo[b]thiophen-5-yl)difluoromethyl)phosphonic acid (Compound 5, 13 mg, 12.80% yield, 13 μmol) as a white solid. LCMS (ESI) m/z=998.2. ¹H NMR (400 MHz, DMSO-d₆) δ 10.98 (s, 1H), 8.83-8.65 (m, 1H), 8.63-8.43 (m, 2H), 8.31 (d, J=10.0 Hz, 1H), 8.19-8.06 (m, 2H), 7.95-7.84 (m, 1H), 7.64 (s, 3H), 7.53-7.40 (m, 2H), 7.24-6.96 (m, 1H), 5.16-5.09 (m, 1H), 5.06-4.95 (m, 1H), 4.89-4.80 (m, 1H), 4.64-4.56 (m, 1H), 4.38 (s, 8H), 3.97-3.85 (m, 5H), 3.77 (s, 1H), 3.68 (s, 1H), 3.49-3.42 (m, 2H), 2.95-2.86 (m, 1H), 2.28-2.16 (m, 1H), 2.11-1.45 (m, 12H).

### Preparation of ((2-(((5S,8S,10aR)-8-((rel-3S,4R)-3-cyano-4-phenylpyrrolidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphonic acid (Compound 6)

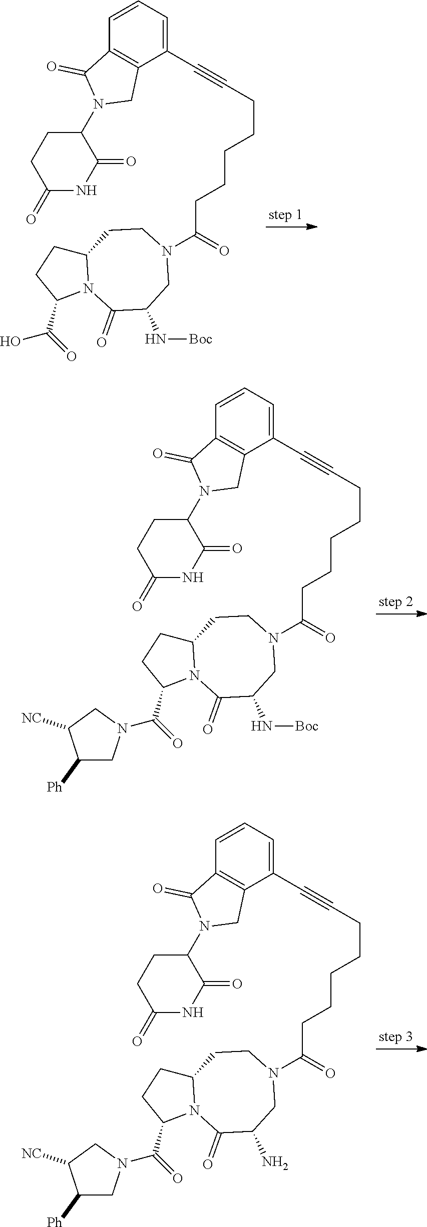 [SMILES: CC(C)(C)OC(=O)N[C@H]1CN(C(=O)CCCCCC#Cc2cccc3c2CN(C2CCC(=O)NC2=O)C3=O)CC[C@H]2CC[C@@H](C(=O)O)N2C1=O ; CC(C)(C)OC(=O)N[C@H]1CN(C(=O)CCCCCC#Cc2cccc3c2CN(C2CCC(=O)NC2=O)C3=O)CC[C@H]2CC[C@@H](C(=O)N3C[C@@H](C#N)[C@H](c4ccccc4)C3)N2C1=O ; N#C[C@@H]1CN(C(=O)[C@@H]2CC[C@@H]3CCN(C(=O)CCCCCC#Cc4cccc5c4CN(C4CCC(=O)NC4=O)C5=O)C[C@H](N)C(=O)N32)C[C@H]1c1ccccc1]

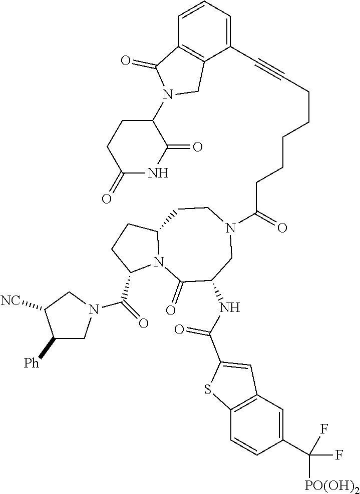

### Step 1: Preparation of tert-butyl ((5S,8S,10aR)-8-((rel-3S,41R)-3-cyano-4-phenylpyrrolidine-1-carbonyl)-3-(8-(2-(2,6-dioxopiperidin-3-yl)-1-oxoisoinidolin-4-yl)oct-7-ynoyl)-6-oxodecahydropyrrolo[1,2-a][1,5]diazocin-5-yl)carbamate

To a solution of (5S,8S,10aR)-5-{[(tert-butoxyl)carbonyl]amino}-3-{8-[2-(2,6-dioxopiperidin-3-yl)-1-oxo-2,3-dihydro-1H-isoindol-4-yl]oct-7-ynoyl}-6-oxodecahydropyrrolo[1,2-a][1,5]diazocine-8-carboxylic acid (0.10 g, 0.14 mmol, 1 eq) in DCM (0.5 mL) was added DIEA (56 mg, 0.43 mmol, 3 eq) and HATU (60 mg, 0.16 mmol, 1.1 eq). The mixture was stirred at 25° C. for 10 minutes and then rac-(3S,4R)-4-phenylpyrrolidine-3-carbonitrile (33 mg, 0.19 mmol, 1.3 eq) was added in the mixture, the mixture was stirred at 25° C. for 1 hour to give a yellow solution. The reaction mixture was filtered and concentrated under reduced pressure to give a residue. The residue w % as purified by flash silica gel chromatography to give tert-butyl ((5S,8S,10aR)-8-((rel-3S,4R)-3-cyano-4-phenylpyrrolidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamate (54 mg, 45% yield) as a white solid. LCMS (ESI) m/z=846.3

### Step 2: Preparation of (3S,4R)-1-((5S,8S,10aR)-5-amino-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carbonyl)-4-phenylpyrrolidine-3-carbonitrile

To a solution of tert-butyl ((5S,8S,10aR)-8-((rel-3S,4R)-3-cyano-4-phenylpyrrolidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamate (70 mg, 83 μmol, 1 eq) in DCM (0.6 mL) and TFA (0.3 mL) as stirred at 25° C. for 30 minutes to give a yellow solution. The reaction mixture was filtered and concentrated under reduced pressure to give (rel-3S,4R)-1-((5S,8S,10aR)-5-amino-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carbonyl)-4-phenylpyrrolidine-3-carbonitrile (70 mg, crude) as a yellow oil. LCMS (ESI) m/z=746.4

### Step 3: Preparation of ((2-(((5S,8S,10aR)-8-((rel-3S,4R)-3-cyano-4-phenylpyrrolidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphonic acid

To a solution of (rel-3S,4R)-1-((5S,8S,10aR)-5-amino-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carbonyl)-4-phenylpyrrolidine-3-carbonitrile (50 mg, 67 μmol, 1 eq) in DCM (1 mL) was added (difluoro(2-((4-nitrophenoxy)carbonyl)benzo[b]thiophen-5-yl)methyl)phosphonic acid (35 mg, 81 μmol, 1.2 eq), 1H-1,2,3-benzotriazol-1-ol (9.0 mg, 67 μmol, 1.0 eq) and ethylbis(propan-2-yl)amine (30 mg, 0.20 mmol, 3 eq). The mixture was stirred at 25° C. for 10 minutes to give a yellow solution. The reaction mixture was filtered and concentrated under reduced pressure to give a residue. The residue was purified by prep-HPLC (column: Phenomenex Luna C18 150×25 mm×10 um; mobile phase: [water (0.1% TFA)−ACN]; B %: 32%-62%, 10 min) to lyophilized to give ((2-(((5S,8S,10aR)-8-((rel-3S,4R)-3-cyano-4-phenylpyrrolidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphonic acid (Compound 6, 12 mg, 17% yield) as a white solid. LCMS (ESI) m/z=1036.5 ¹H NMR (400 MHz, DMSO-d₆) δ 10.98 (s, 2H), 9.06-8.59 (m, 1H), 8.33-8.27 (m, 1H), 8.13-8.06 (m, 2H), 7.72-7.67 (m, 1H), 7.64-7.58 (m, 2H), 7.51-7.46 (m, 1H), 7.44-7.37 (m, 4H), 7.36-7.31 (m, 1H), 7.26-6.94 (m, 1H), 5.17-5.09 (m, 1H), 4.98 (d, J=4.0 Hz, 1H), 4.91-4.69 (m, 1H), 4.68-4.49 (m, 2H), 4.48-4.41 (m, 1H), 4.36-4.32 (m, 1H), 4.31-4.16 (m, 3H), 4.12-4.05 (m, 1H), 4.02-3.97 (m, 1H), 3.90 (d, J=3.2 Hz, 1H), 3.80 (s, 1H), 3.71-3.65 (m, 3H), 2.95-2.81 (m, 2H), 2.06-1.89 (m, 4H), 1.86-1.68 (m, 4H), 1.67-1.57 (m, 5H), 1.54-1.39 (m, 3H), 1.29-1.11 (m, 1H).

### Preparation of (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylic acid

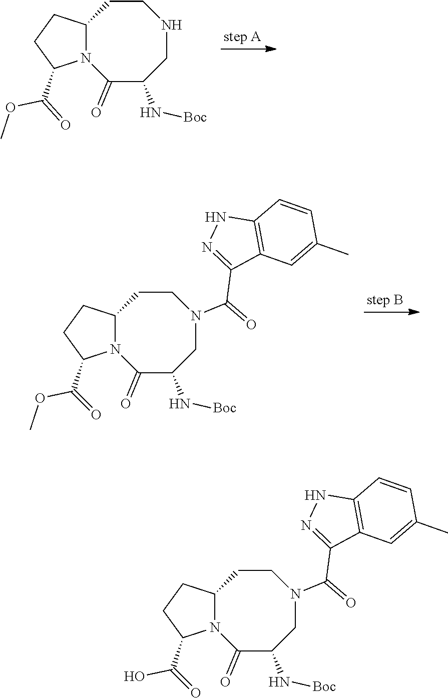

### STEP A: methyl (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylate

To a solution of methyl (5S,8S,10aR)-5-((tert-butoxycarbonyl)amino)-6-oxodecahydropyrrolo[1,2-a][1,5]diazocine-8-carboxylate (1 g, 2.93 mmol, 1.0 eq.) and 7-((bis(2-(butyrylthio)ethoxy))phosphoryl)difluoromethyl)-2-naphthoic acid (516 mg, 2.93 mmol, 1.0 eq.) in DMF (20 mL) were added HATU (1.22 g, 3.22 mmol, 1.1 eq.) and DIEA (1.13 g, 8.79 mmol, 3.0 eq.) at room temperature. The resulting mixture was stirred at room temperature for 2 hrs. After completion, the reaction mixture was concentrated under reduced pressure. The residue was purified by flash column chromatography on silica gel to give methyl (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylate (1.1 g, 2.20 mmol, 75%) as a white solid. LCMS (ESI): m/z=500 [M+H]⁺.

### STEP B: (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylic acid

To a solution of methyl(5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylate (1.1 g, 2.20 mmol, 1.0 eq.) in THF (20 mL) and H₂O (7 mL) was added LiOH (277 mg, 6.60 mmol, 3.0 eq.) at room temperature. The resulting mixture was stirred at room temperature for 2 hrs. After completion, the reaction mixture was cooled down in an ice bath, then neutralized carefully with HCl (1N, aq.) until the pH was adjusted to pH=2-3. The resulting mixture was extracted with EtOAc (20 mL×3), and the combined organic layers were washed with brine (10 mL×2), dried over with anhydrous Na₂SO₄, then concentrated under reduced pressure to give crude (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylic acid (930 mg, quant.) as a white solid, which was directly used without further purification LCMS (ESI): m/z=486 [M+H]⁺.

The following intermediates in Table 1 were prepared according to the procedure described above for (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylic acid utilizing the appropriate starting materials and modifications.

TABLE 1

| Name | Structure | LCMS |
|---|---|---|
| (5S,8S,10aR)-5-((tert-butoxycarbonyl)amino)-3- (5-methylbenzo[d]isoxazole-3-carbonyl)-6- oxodecahydropyrrolo[1,2-a][1,5]diazocine-8- carboxylic acid | 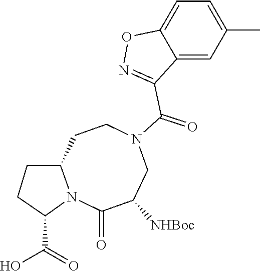 [SMILES: Cc1ccc2onc(C(=O)N3CC[C@H]4CC[C@@H](C(=O)O)N4C(=O)[C@@H](NC(=O)OC(C)(C)C)C3)c2c1] | 487 [M + H]⁺ |

### Preparation of ((difluoro(7-(((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)phosphonic acid (Compound 7) and S,S′-((((difluoro(7-(((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)phosphoryl)bis(oxy))bis(ethane-2,1-diyl)) dibutanethioate (Compound 8)

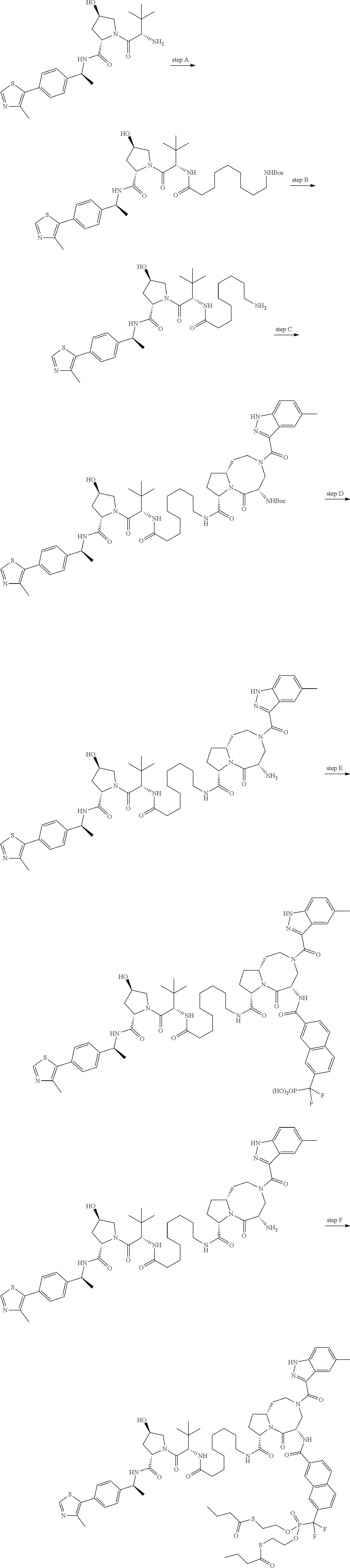

### STEP A: tert-butyl (9-(((S)-1-((2S,4R)-4-hydroxy-2-(((S)-1-(4-(4-methylthiazol-5-yl)phenyl)ethyl)carbamoyl)pyrrolidin-1 yl)-3,3-dimethyl-1-oxobutan-2-yl)amino)-9-oxononyl)carbamate

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (150 mg, 0.34 mmol, 1.0 eq.) and 9-((tert-butoxycarbonyl)amino)nonanoic acid (92 mg, 0.34 mmol, 1.0 eq.) in DMF (2 mL) were added DIEA (132 mg, 1.02 mmol, 3.0 eq.) and HATU (155 mg, 0.41 mmol, 1.2 eq.), and the resulting mixture was stirred at room temperature for 12 hrs. After completion, the reaction mixture was purified by C18 column to afford tert-butyl (9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamate (143 mg, 0.21 mmol, 60%) as a yellow solid. LCMS (ESI): m/z=700 [M+H]⁺.

### STEP B: (2S,4R)-1-((S)-2-(9-aminononanamido)-3,3-dimethylbutanoyl)-4-hydroxy-N—((S)-1-(4-(4-methylthiazol-5-yl)phenyl)ethyl)pyrrolidine-2-carboxamide

To a solution of tert-butyl (9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamate (250 mg, 0.36 mmol, 1.0 eq.) in DCM (3 mL) was added TFA (1 mL), and the resulting mixture was stirred at room temperature for 1 hr. After completion, the reaction mixture was concentrated under reduced pressure to give crude (2S,4R)-1-((S)-2-(9-aminononanamido)-3,3-dimethylbutanoyl)-4-hydroxy-N—((S)-1-(4-(4-methylthiazol-5-yl)phenyl)ethyl)pyrrolidine-2-carboxamide (250 mg, quant.) as a yellow solid, which was used in next step directly without further purification. LCMS (ESI): m/z=600 [M+H]⁺.

### STEP C: tert-butyl ((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-5-yl)carbamate

To a solution of (2S,4R)-1-((S)-2-(9-aminononanamido)-3,3-dimethylbutanoyl)-4-hydroxy-N—((S)-1-(4-(4-methylthiazol-5-yl)phenyl)ethyl)pyrrolidine-2-carboxamide (250 mg, 0.42 mmol, 1.0 eq.) and (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylic acid (204 mg, 0.42 mmol, 1.0 eq.) in DMF (2 mL) were added DIEA (163 mg, 1.26 mmol, 3.0 eq.), EDCl (161 mg, 0.84 mmol, 2.0 eq.), and HOBt (113 mg, 0.84 mmol, 2.0 eq.). The resulting mixture was stirred at room temperature for 12 hrs. After completion, the reaction mixture was purified by C18 column to afford tert-butyl ((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-5-yl)carbamate (270 mg, 0.25 mmol, 60%) as a yellow solid. LCMS (ESI): m/z=1067 [M+H]⁺.

### STEP D: (5S,8S,10aR)-5-amino-N-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)-3-(5-methyl-1H-indazole-3-carbonyl)-6-oxodecahydropyrrolo[1,2-a][1,5]diazocine-8-carboxamide

To a solution of tert-butyl ((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-5-yl)carbamate (150 mg, 0.14 mmol, 1.0 eq.) in DCM (1 mL) was added TFA (0.3 mL), and the resulting mixture was stirred at room temperature for 1 hr. After completion, the reaction mixture was concentrated under reduced pressure to give crude (5S,8S,10aR)-5-amino-N-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)-3-(5-methyl-1H-indazole-3-carbonyl)-6-oxodecahydropyrrolo[1,2-a][1,5]diazocine-8-carboxamide (140 mg, quant.) as a colorless oil, which was used in next step directly without further purification. LCMS (ESI): m/z=967 [M+H]⁺.

### STEP E: (difluoro(7-(((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-1-yl)carbamoyl)naphthalen-2-yl)methyl)phosphonic acid

To a solution of (5S,8S,10aR)-5-amino-N-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)-3-(5-methyl-1H-indazole-3-carbonyl)-6-oxodecahydropyrrolo[1,2-a][1,5]diazocine-8-carboxamide (60 mg, 62.0 μmol, 1.0 eq) and (difluoro(7-((perfluorophenoxy)carbonyl)naphthalen-2-yl)methyl)phosphonic acid (29.0 mg, 62.0 μmol, 1.0 eq.) in DMF 1 mL) was added DIEA (23.9 mg, 186 μmol, 3.0 eq.) at room temperature. The resulting mixture was stirred at room temperature for 2 hrs. After completion, the reaction mixture was purified by prep-HPLC to give ((difluoro(7-(((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)phosphonic acid (Compound 7, 21.4 mg, 17.1 μmol, 28%) as a white solid.

¹H NMR (400 MHz, CDCl₃) δ 8.68 (s, 1H), 8.48-8.32 (m, 1H), 8.23-8.05 (m, 1H), 8.03-7.94 (m, 1H), 7.93-7.33 (m, 6H), 7.31-7.29 (m, 2H), 7.19-7.09 (m, 1H), 6.70-6.55 (m, 1H), 5.72-5.54 (m, 1H), 5.07-4.95 (m, 1H), 4.76-4.57 (m, 3H), 4.50-4.38 (m, 2H), 4.09-4.00 (m, 1H), 3.63-3.41 (m, 5H), 3.00-2.85 (m, 4H), 2.52-2.40 (m, 6H), 2.31-1.96 (m, 8H), 1.85-1.74 (m, 1H), 1.47-1.26 (m, 20H), 1.11-1.05 (m, 2H), 0.98 (s, 9H). LCMS (ESI): m/z=626.5 [M/2+H]⁺.

### STEP F: S,S″-((((difluoro(7-(((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)phosphoryl)bis(oxy))bis(ethane-2,1-diyl)) dibutanethioate

To a solution of (5S,8S,10aR)-5-amino-N-(8-(2-(2,6-dioxopiperidin-3-yl)-1-oxoisoindolin-4-yl)oct-7-yn-1-yl)-3-(5-methyl-1H-indazole-3-carbonyl)-6-oxodecahydropyrrolo[1,2-a][1,5]diazocine-8-carboxamide (60 mg, 62.0 μmol, 1.0 eq.) and perfluorophenyl 7-((bis(2-(butyrylthio)ethoxy)phosphoryl)difluoromethyl)-2-naphthoate (45.1 mg, 62.0 μmol, 1.0 eq.) in DMF (2 mL) was added DIEA (23.9 mg, 186 μmol, 3.0 eq.) at room temperature. The resulting mixture was stirred at room temperature for 1 hr. After completion, the reaction mixture was purified by prep-HPLC to give S,S′-((((difluoro(7-(((5S,8S,10aR)-8-((9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)phosphoryl)bis(oxy))bis(ethane-2,1-diyl)) dibutanethioate (Compound 8, 13.5 mg, 8.94 μmol, 14%) as a white solid. ¹H NMR (400 MHz, CDCl₃) δ 8.87-8.75 (m, 1H), 8.52 (s, 1H), 8.30-8.17 (m, 2H), 8.09-7.86 (m, 5H), 7.80-7.65 (m, 2H), 7.37-7.31 (m, 3H), 7.22-7.15 (m, 1H), 6.53-6.28 (m, 2H), 5.80-5.67 (m, 1H), 4.97-4.86 (m, 2H), 4.74-4.64 (m, 2H), 4.53-4.45 (m, 2H), 4.32-4.08 (m, 8H), 3.75-3.60 (m, 2H), 3.21-3.08 (m, 5H), 3.00-2.94 (m, 1H), 2.58-2.51 (m, 8H), 2.47-2.16 (m, 12H), 2.11-1.93 (m, 5H), 1.89-1.75 (m, 2H), 1.73-1.63 (m, 5H), 1.46-1.26 (m, 8H), 1.05 (s, 9H), 0.97-0.93 (m, 8H). LCMS (ESI): m/z=1511.8 [M+H]⁺.

### Preparation of ((7-(((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphonic acid (Compound 9) and S,S′-(((((7-(((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) dibutanethioate (Compound 10)

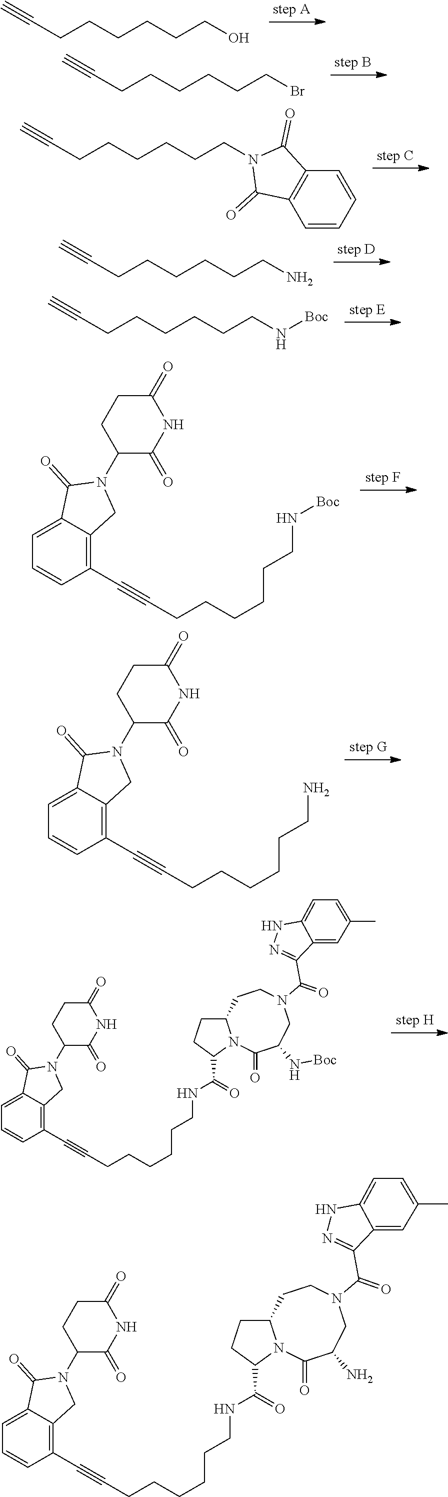

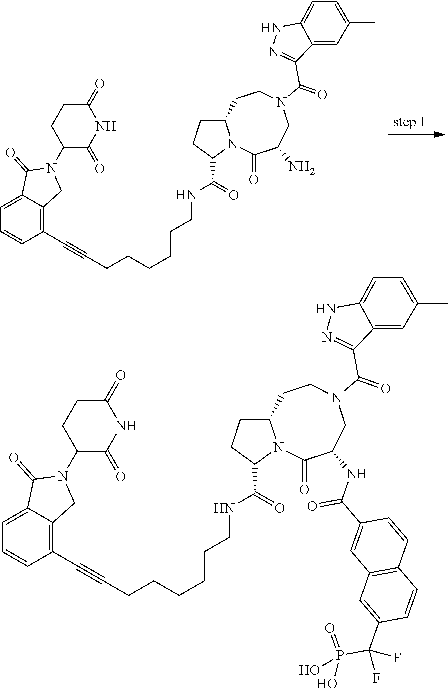

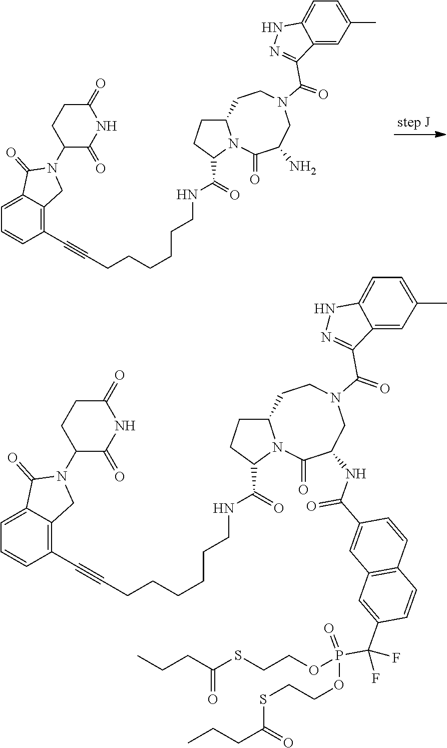

### STEP A: 8-bromooct-1-yne

To a solution of oct-7-yn-1-ol (1 g, 7.94 mmol, 1.0 eq.) and PPh₃ (4.16 g, 15.9 mmol, 2.0 eq.) in dry DCM (50 mL) was added CBr₄ (2.86 mg, 8.73 mmol, 1.1 eq) slowly at room temperature under nitrogen. The mixture was stirred at 40° C. for 1 hr. After completion, the reaction mixture w as concentrated under reduced pressure. The residue was purified by flash column chromatography on silica gel to afford 8-bromooct-1-yne (750 mg, 3.97 mmol, 50%) as a yellow oil. LC-MS (ESI) m/z=189 [M+H]⁺.

### STEP B: 2-(oct-7-yn-1-yl)isoindoline-1,3-dione

To a solution of isoindoline-1,3-dione (531 mg, 3.61 mmol, 1.0 eq.) and K₂CO₃ (1.50 g, 10.8 mmol, 3.0 eq.) in DMF (mL) was added 8-bromooct-1-yne (750 mg, 3.97 mmol, 1.1 eq) slowly at room temperature under nitrogen. The mixture was stirred at 60° C. for 2 hrs. After completion, the reaction mixture was concentrated under reduced pressure. The residue was purified by flash column chromatography on silica gel to afford 2-(oct-7-yn-1-yl)isoindoline-1,3-dione (531 mg, 2.08 mmol, 58%) as a yellow oil. LC-MS (ESI) m/z=256 [M+H]⁺.

### STEP C: oct-7-yn-1-amine

To a solution of 2-(oct-7-yn-1-yl)isoindoline-1,3-dione (1.6 g, 6.26 mmol, 1.0 eq.) in EtOH (50 mL) was added hydrazine hydrate (1.09 g, 21.9 mmol, 3.5 eq.). The mixture was stirred at 80° C. for 4 hrs. After completion, the reaction mixture was concentrated under reduced pressure. KOH (40 mL, 1N, aq.) was added, then extracted with DCM (50 mL×2). The organic layers were combined and washed with brine (150 mL), dried over anhydrous Na₂SO₄, and concentrated under reduced pressure. The residue was purified by flash column chromatography on silica gel to afford oct-7-yn-1-amine (600 mg, 4.79 mmol, 77%) as a green oil. LC-MS (ESI) m/z=126 [M+H]⁺.

### STEP D: tert-butyl oct-7-yn-1-ylcarbamate

To a solution of oct-7-yn-1-amine (250 mg, 1.99 mmol, 1.0 eq.) and Na₂CO₃ (421 mg, 3.98 mmol, 2.0 eq.) in THF (4.5 mL) was added Boc₂O (650 mg, 2.98 mmol, 1.5 eq.). The mixture was stirred at 25° C. for 12 hrs. After completion, the reaction mixture was concentrated under reduced pressure. The residue was purified by flash column chromatography on silica gel to afford tert-butyl oct-7-yn-1-ylcarbamate (320 mg, 1.42 mmol, 71%) as a yellow oil. LC-MS (ESI) m/z=226 [M+H]⁺.

### STEP F: tert-butyl (8-(2-(2,6-dioxopiperidin-3-yl)-1-oxoisoindolin-4-yl)oct-7-yn-1-yl)carbamate

To a solution of 3-(4-bromo-1-oxoisoindolin-2-yl)piperidine-2,6-dione (250 mg, 773 μmol, 1.0 eq.), tert-butyl oct-7-yn-1-ylcarbamate (208 mg, 927 μmol, 1.2 eq.) and Cs₂CO₃ (750 mg, 2.31 mmol, 3.0 eq.) in DMF (15 mL) were added Pd(PPh₃)₂Cl₂ (108 mg, 154 μmol, 0.2 eq.) and CuI (29.2 mg, 154 μmol, 0.2 eq.). The resulting mixture was stirred at 80° C. for 12 hrs. After completion, the reaction mixture was filtered and the filtrate was purified by prep-HPLC to give tert-butyl (8-(2-(2,6-dioxopiperidin-3-yl)-1-oxoisoindolin-4-yl)oct-7-yn-1-yl)carbamate (136 mg, 290 μmol, 38%) as a white solid. LC-MS (ESI) m/z=466.2 [M−H]⁻.

### STEP F: 3-(4-(8-aminooct-1-yn-1-yl)-1-oxoisoindolin-2-yl)piperidine-2,6-dione

To a solution of tert-butyl (8-(2-(2,6-dioxopiperidin-3-yl)-1-oxoisoindolin-4-yl)oct-7-yn-1-yl)carbamate (160 mg, 342 μmol, 1.0 eq.) in DCM (1.5 mL) was added TFA (0.5 mL), and the resulting mixture was stirred at room temperature for 1 hr. After completion, the reaction mixture was concentrated under reduced pressure to give crude 3-(4-(8-aminooct-1-yn-1-yl)-1-oxoisoindolin-2-yl)piperidine-2,6-dione (120 mg, quant.) as a yellow solid, which was used in next step directly without further purification. LCMS (ESI): m/z=368.2 [M+H]⁺.

### STEP G: tert-butyl ((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diazocin-5-yl)carbamate

To a solution of 3-(4-(8-aminooct-1-yn-1-yl)-1-oxoisoindolin-2-yl)piperidine-2,6-dione (120 mg, 326 μmol, 1.0 eq.) and (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylic acid (173 mg, 358 μmol, 1.1 eq.) in DMF (2 mL) were added DIEA (3126 mg, 978 μmol, 3.0 eq), EDCI (125 mg, 652 μmol, 2.0 eq.), and HOBt (88.0 mg, 652 μmol, 2.0 eq.). The resulting mixture was stirred at room temperature for 12 hrs. After completion, the reaction mixture was purified by prep-HPLC to afford tert-butyl ((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diazocin-5-ylcarbamate (160 mg, 191 μmol, 59%) as a yellow solid. LCMS (ESI): m/z=835.1 [M+H]⁺.

### STEP H: (5S,8S,10aR)-5-amino-N-(8-(2-(2,6-dioxopiperidin-3-yl)-1-oxoisoindolin-4-yl)oct-7-yn-1-yl)-3-(5-methyl-1H-indazole-3-carbonyl)-6-oxodecahydropyrrolo[1,2-a][1,5]diazocine-8-carboxamide

To a solution of tert-butyl ((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diazocin-5-yl)carbamate (60 mg, 71.8 μmol, 1.0 eq.) in DCM (1 mL) was added TFA (0.4 mL), and the resulting mixture was stirred at room temperature for 1 hr. After completion, the reaction mixture was concentrated under reduced pressure to give crude (5S,8S,10aR)-5-amino-N-(8-(2-(2,6-dioxopiperidin-3-yl)-1-oxoisoindolin-4-yl)oct-7-yn-1-yl)-3-(5-methyl-1H-indazole-3-carbonyl)-6-oxodecahydropyrrolo[1,2-a][1,5]diazocine-8-carboxamide (45.0 mg, 61.2 μmol, 85%.) as a red solid, which was used directly without further purification LCMS (ESI): m/z=735.0 [M+H]⁺.

### STEP I: ((7-(((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diozocin-5-yl)carbamoyl)naphthalen-2-yl)difluoromethyl)phosphonic acid

To a solution of (5S,8S,10aR)-5-amino-N-(8-(2-(2,6-dioxopiperidin-3-yl)-1-oxoisoindolin-4-yl)oct-7-yn-1-yl)-3-(5-methyl-1H-indazole-3-carbonyl)-6-oxodecahydropyrrolo[1,2-a][1,5]diazocine-8-carboxamide (45 mg, 61.2 μmol, 1.0 eq.) and (difluoro(7-((perfluorophenoxy)carbonyl)naphthalen-2-yl)methyl)phosphonic acid (28.6 mg, 61.2 μmol, 1.0 eq.) in DMF (2 mL) was added DIEA (23.6 mg, 183 μmol, 3.0 eq.) at room temperature. The resulting mixture was stirred at room temperature for 1 hr. After completion, the reaction mixture was purified by prep-HPLC to give ((7-(((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphonic acid (Compound 9, 21.0 mg, 20.6 μmol, 34%) as a white solid.

¹H NMR (400 MHz, DMSO-d6) δ 13.60-13.28 (m, 1H), 11.11-10.91 (m, 1H), 9.04-8.75 (m, 1H), 8.72-8.58 (m, 1H), 8.26-8.16 (m, 1H), 8.13-7.99 (m, 3H), 7.97-7.55 (m, 5H), 7.54-7.38 (m, 2H), 7.29-7.17 (m, 1H), 5.26-5.01 (m, 2H), 4.74-4.62 (m, 1H), 4.46-4.25 (m, 4H), 4.17-4.03 (m, 1H), 3.95-3.53 (m, 3H), 3.13-2.82 (m, 3H), 2.48-2.39 (m, 5H), 2.22-1.97 (m, 4H), 1.95-1.68 (m, 3H), 1.67-1.16 (m, 9H).

LCMS (ESI): m/z=1019.3 [M+H]⁺.

### STEP J: S,S′-(((((7-(((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) dibutanethioate

To a solution of (5S,8S,10aR)-5-amino-N-(8-(2-(2,6-dioxopiperidin-3-yl)-1-oxoisoindolin-4-yl)oct-7-yn-1-yl)-3-(5-methyl-1H-indazole-3-carbonyl)-6-oxodecahydropyrrolo[1,2-a][1,5]diazocine-8-carboxamide (45 mg, 61.2 μmol, 1.0 eq.) and perfluorophenyl 7-((bis(2-(butyrylthio)ethoxy)phosphoryl)difluoromethyl)-2-naphthoate (44.5 mg, 61.2 μmol, 1.0 eq.) in DMF (2 mL) was added DIEA (23.6 mg, 183 μmol, 3.0 eq.) at room temperature. The resulting mixture was stirred at room temperature for 1 hr. After completion, the reaction mixture was purified by prep-HPLC to give S,S′-(((((7-(((5S,8S,10aR)-8-((8-(2-(2,6-dioxopiperidin-3-yl)-1-oxoisoindolin-4-yl)oct-7-yn-1-yl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) dibutanethioate (Compound 10, 10.0 mg, 7.81 μmol, 13%) as a white solid.

¹H NMR (400 MHz, CDCl₃) δ 10.36-9.91 (m, 1H), 8.71-8.32 (m, 1H), 8.29-8.13 (m, 1H), 8.11-7.62 (m, 6H), 7.60-7.38 (m, 2H), 7.24-7.15 (m, 1H), 6.79-6.38 (m, 1H), 5.79-5.48 (m, 1H), 5.46-3.70 (m, 12H), 3.22-3.03 (m, 4H), 2.98-2.72 (m, 2H), 2.69-2.55 (m, 1H), 2.55-2.48 (m, 4H), 2.47-2.43 (m, 3H), 2.43-1.69 (m, 1H), 1.69-1.58 (m, 4H), 1.58-0.98 (m, 9H), 0.96-0.86 (m, 6H).

LCMS (ESI): m/z=1279.3 [M+H]⁺.

### Preparation of ((7-(((5S,8S,10aR)-8-((8-(2-(2,6-dioxopiperidin-3-yl)-1-oxoisoindolin-4-yl)oct-7-yn-1-yl)methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphonic acid (Compound 11) and S,S′-(((((7-(((5S,8S,10aR)-8-((S-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) dibutanethioate (Compound 12)

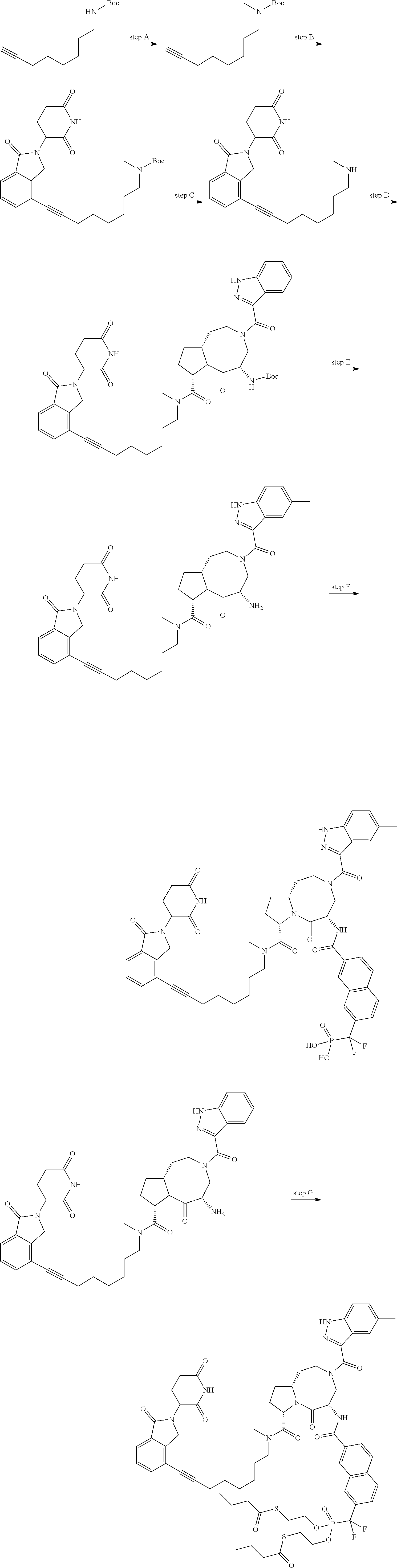

### STET A: tert-butyl methyl(oct-7-yn-1-yl)carbamate

To a solution of tert-butyl oct-7-yn-1-ylcarbamate (1.2 g, 5.33 mmol, 1.0 eq.) in dry DMF (20 mL) was added NaH (188 mg, 5.86 mmol, 1.1 eq) slowly at 0° C. under nitrogen. The mixture was stirred for additional 30 min at 0° C., then MeI (1.51 g, 10.7 mmol, 2.0 eq.) was added dropwise. After addition, the reaction mixture was allowed to warm to room temperature and stirred for 14 hrs. After completion, the reaction mixture was quenched by adding H₂O (20 mL), then extracted with EtOAc (150 mL×3). The organic layers were combined and washed with brine (150 mL), dried over anhydrous Na₂SO₄, and concentrated under reduced pressure. The residue was purified by flash column chromatography on silica gel to afford tert-butyl methyl(oct-7-yn-1-yl)carbamate (1.1 g, 4.60 mmol, 86%) as a yellow oil. LC-MS (ESI) m/z=240 [M+H]⁺.

### STEP B: tert-butyl (8-(2-(2,6-dioxopiperidin-3-yl)-1-oxoisoindolin)-4-yl)oct-7-yn-1-yl)(methyl)carbamate

A solution of 3-(4-bromo-1-oxoisoindolin-2-yl)piperidine-2,6-dione (650 mg, 2.01 mmol, 1.0 eq.), tert-butyl methyl(oct-7-yn-1-yl)carbamate (576 mg, 2.41 mmol, 1.2 eq.), Cs₂CO₃ (1.96 g, 6.03 mmol, 3.0 eq.) in DMF (15 mL) was stirred at room temperature under N₂, then Pd(PPh₃)₂Cl₂ (140 mg, 0.20 mmol, 0.1 eq.) and CuI (76 mg, 0.40 mmol, 0.2 eq.) were added, and the resulting mixture was stirred at 80° C. for 12 hrs. After completion, the reaction mixture was diluted with H₂O (40 mL) and extracted with EtOAc (30 mL×3). The organic layers were combined and washed with brine (10 mL), dried over anhydrous Na₂SO₄ and concentrated under reduced pressure. The residue was purified by flash column chromatography on silica gel to afford tert-butyl (8-(2-(2,6-dioxopiperidin-3-yl)-1-oxoisoindolin-4-yl)oct-7-yn-1-yl)(methyl)carbamate (330 mg, 0.69 mmol, 34%) as a white solid. LCMS (ESI): m/z=482 [M+H]⁺.

### STEP C: 3-(4-(8-(methylamino)oct-1-in-1-yl)-1-oxoisoindolin-2-yl)piperidine-2,6-dione

To a solution of tert-butyl (8-(2-(2,6-dioxopiperidin-3-yl)-1-oxoisoindolin-4-yl)oct-7-yn-1-yl)(methyl)carbamate (330 mg, 0.69 mmol, 1.0 eq.) in DCM (3 mL) was added TFA (1 mL), and the resulting mixture was stirred at room temperature for 2 hrs. After completion, the reaction mixture was cooled down in an ice bath, then neutralized carefully with NaHCO₃ (aq.) until the pH was adjusted to pH=8-9. The resulting mixture was extracted with DCM (10 mL×3), and the combined organic layers were washed with brine (10 mL×2), dried over with anhydrous Na₂SO₄, then concentrated under reduced pressure to give crude 3-(4-(8-(methylamino)oct-1-yn-1-yl)-1-oxoisoindolin-2-yl)piperidine-2,6-dione (260 mg, quant.) as a white solid, which was used in next step directly without further purification. LCMS (ESI): m/z=382 [M+H]⁺.

### STEP D: tert-butyl ((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamate

To a solution of 3-(4-(8-(methylamino)oct-1-yn-1-yl)-1-oxoisoindolin-2-yl)piperdine-2,6-dione (260 mg, 0.68 mmol, 1.2 eq.) and (5S,8S,10aR)-5-((tert-butoxycarbonyl)amino)-3-(5-methyl-1H-indazole-3-carbonyl)-6-oxodecahydropyrrolo[1,2-a][1,5]diazocine-8-carboxylic acid (210 mg, 0.56 mmol, 1.0 eq.) in DMF (10 mL) were added DIEA (361 mg, 2.80 mmol, 5.0 eq.), EDCI (108 mg, 0.56 mmol, 1.0 eq.), and HOBt (76 mg, 0.56 mmol, 1.0 eq.), and the resulting mixture was stirred at room temperature for 2 hrs. After completion, the reaction was diluted with H₂O (20 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamate (80 mg, 94.3 μmol, 17%) as a white solid. LCMS (ESI): m/z=849 [M+H]⁺.

### STEP E: (5S,8S,10aR)-5-amino-N-(8-(2-(2,6-dioxopiperidin-3-yl)-1-oxoisoindolin-4-yl)(oct-7-yn-1-yl)-N-methyl-3-(5-methyl-1H-indazole-3-carbonyl)-6-oxodecahydropyrrolo[1,2-a][1,5]diazocine-8-carboxamide

To a solution of tert-butyl ((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamate (80 mg, 94.3 μmol, 1.0 eq.) in DCM (3 mL) was added TFA (1 mL), and the resulting mixture was stirred at room temperature for 2 hrs. After completion, the reaction mixture was concentrated under reduced pressure to give crude (5S,8S,10aR)-5-amino-N-(8-(2-(2,6-dioxopiperidin-3-yl)-1-oxoisoindolin-4-yl)oct-7-yn-1-yl)-N-methyl-3-(5-methyl-1H-indazole-3-carbonyl)-6-oxodecahydropyrrolo[1,2-a][1,5]diazocine-8-carboxamide (70 mg, quant.) as a white solid, which was used in next step directly without further purification. LCMS (ESI): m/z=749 [M+H]⁺.

### STEP F: ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphonic acid

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methyl-1H-indazole-3-carbonyl)-6-oxodecahydropyrrolo[1,2-a][1,5]diazocine-8-carboxamide (42 mg, 56.1 μmol, 1.1 eq.) and (difluoro(7-((perfluorophenoxy)carbonyl)naphthalen-2-yl)methyl)phosphonic acid (24 mg, 51.0 μmol, 1.0 eq.) in DMF (2 mL) was added DIEA (33 mg, 0.25 mmol, 5.0 eq.) at room temperature. The resulting mixture was stirred at room temperature for an additional 2 hrs. After completion, the reaction mixture was concentrated under reduced pressure. The residue was purified by RP-prep HPLC to give ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphonic acid (Compound 11, 28 mg, 27.1 μmol, 53%) as a white solid.

¹H NMR (400 MHz, DMSO-d6) δ 13.57-13.28 (m, 1H), 11.04-10.91 (m, 1H), 8.97-8.69 (m, 1H), 8.69-8.57 (m, 1H), 8.24-8.12 (m, 1H), 8.11-7.81 (m, 4H), 7.78-7.37 (m, 5H), 7.27-7.19 (m, 1H), 5.24-5.01 (m, 2H), 4.82-4.76 (m, 1H), 4.45-4.14 (m, 5H), 3.90-3.80 (m, 1H), 3.66-3.56 (m, 2H), 3.37-3.29 (m, 2H), 3.22-3.12 (m, 1H), 3.03-2.77 (m, 4H), 2.63-2.54 (m, 1H), 2.47-2.40 (m, 4H), 2.31-1.87 (m, 4H), 1.84-1.63 (m, 3H), 1.59-1.48 (m, 2H), 1.46-1.20 (m, 6H).

LCMS (ESI): m/z=1031.2 [M−H]⁻.

### STEP G: S,S′-(((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(S-methyl-1H-indazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) dibutanethioate

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methyl-1H-indazole-3-carbonyl)-6-oxodecahydropyrrolo[1,2-a][1,5]diazocine-8-carboxamide (70 mg, 0.18 mmol, 1.1 eq.) and 7-((bis(2-(butyrylthio)ethoxy)phosphoryl)difluoromethyl)-2-naphthoic acid (92 mg, 0.16 mmol, 1.0 eq.) in DMF (2 mL) were added HATU (68 mg, 0.18 mmol, 1.1 eq.) and DIEA (103 mg, 0.80 mmol, 5.0 eq.) at room temperature. The resulting mixture was stirred at room temperature for an additional 2 hrs. After completion, the reaction mixture was concentrated under reduced pressure. The residue was purified by RP-prep HPLC to give S,S′-(((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) dibutanethioate (Compound 12, 13.7 mg, 10.6 μmol, 6%) as a white solid.

¹H NMR (400 MHz, CDCl₃) δ 10.61-9.53 (m, 1H), 9.49-8.77 (m, 1H), 8.54-8.32 (m, 1H), 8.30-8.19 (m, 1H), 8.16 (d, J=8.7 Hz, 1H), 8.02-7.64 (m, 6H), 7.57-7.46 (m, 1H), 7.45-7.27 (n, 2H), 7.18 (s, 1H), 5.86-5.45 (m, 1H), 5.42-4.10 (m, 1H), 3.68-3.39 (m, 2H), 3.21-3.05 (m, 6H), 3.01-2.91 (m, 1H), 2.85-2.62 (m, 2H), 2.53-2.48 (m, 4H), 2.47-2.43 (m, 3H), 2.37-1.80 (m, 1H), 1.68-1.27 (m, 13H), 0.97-0.86 (m, 6H).

LCMS (ESI) m/z=1293.3 [M+H]⁺

### Preparation of propyl (((7-((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)fluoromethyl)(phenoxy)phosphoryl)-L-alaninate (Compound 13) and ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)fluoromethyl)phosphonic acid (Compound 14)

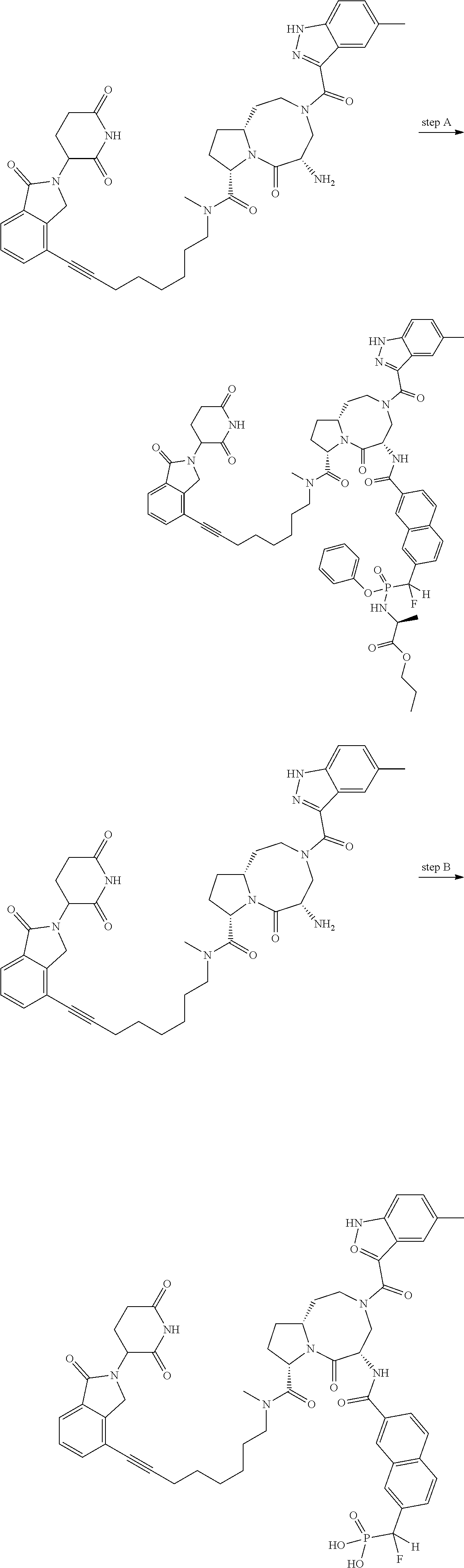

### STEP A: propyl (((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)fluoromethyl)(phenoxy)phosphoryl)-L-alaninate

To a solution of (5S,8S,10aR)-5-amino-N-(8-(2-(2,6-dioxopiperidin-3-1)-1-oxoisoindolin-4-yl)oct-7-yn-1-yl)-N-methyl-3-(5-methyl-1H-indazole-3-carbonyl)-6-oxodecahydropyrrolo[1,2-a][1,5]diazocine-8-carboxamide (48 mg, 64.2 μmol, 1.0 eq.) and 7-(fluoro((((S)-1-oxo-1-propoxypropan-2-yl)amino)(phenoxy)phosphoryl)methyl)-2-naphthoic acid (30 mg, 64.2 μmol, 1.0 eq.) in DMF (2 mL) were added HATU (29 mg, 77.0 μmol, 1.2 eq.) and DIEA (41 mg, 321 μmol, 5.0 eq.) at room temperature. The resulting mixture was stirred at room temperature for 2 hrs. After completion, the reaction mixture was concentrated under reduced pressure. The residue was purified by RP-prep HPLC to give propyl (((7-(((5S,8S,10aR)-8-((8-(2-(2,6-dioxopiperidin-3-%1)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)fluoromethyl)(phenoxy)phosphoryl)-L-alaninate (Compound 13, 10 mg, 8.3 μmol, 13%) as a white solid.

¹H NMR (400 MHz, CDCl₃) δ 8.36-8.26 (m, 1H), 8.16-7.60 (m, 8H), 7.59-7.27 (m, 4H), 7.26-6.97 (m, 6H), 6.16-5.92 (m, 1H), 5.84-5.60 (m, 1H), 5.31-5.09 (m, 1H), 4.87-4.76 (m, 1H), 4.58-3.94 (m, 8H), 3.71-3.19 (m, 4H), 3.15-3.05 (m, 2H), 2.97-2.68 (m, 3H), 2.52-2.38 (m, 5H), 2.28-2.11 (m, 4H), 1.98-1.75 (m, 4H), 1.64-1.37 (m, 9H), 1.37-1.24 (m, 4H), 0.93-0.81 (m, 3H).

LCMS (ESI): m/z=1204.4 [M+H]⁺

### STEP B: ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)fluoromethyl)phosphonic acid

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methyl-1H-indazole-3-carbonyl)-6-oxodecahydropyrrolo[1,2-a][1,5]diazocine-8-carboxamide (48 mg, 64.2 μmol, 1.0 eq.) and (fluoro(7-((perfluorophenoxy)carbonyl)naphthalen-2-yl)methyl)phosphonic acid (29 mg, 64.2 μmol, 1.0 eq.) in DMF (2 mL) was added DIEA (41 mg, 321 μmol, 5.0 eq.) at room temperature. The resulting mixture was stirred at room temperature for 2 hrs. After completion, the reaction mixture was concentrated under reduced pressure. The residue was purified by RP-prep HPLC to give ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methy 1-1H-indazole-3-carbonyl)-6-oxodecahydropyrrolo[1,2-a][1,5]diazocin-5-yl)carbamoyl)naphthalen-2-yl)fluoromethyl)phosphonic acid (Compound 14, 18.6 mg, 18.3 μmol, 29%) as a white solid.

¹H NMR (400 MHz, DMSO-d6) δ 13.64-13.18 (m, 1H), 10.98 (s, 1H), 9.00-8.30 (m, 2H), 8.16-7.78 (m, 5H), 7.75-7.38 (m, 5H), 7.31-7.15 (m, 1H), 6.05-5.80 (m, 1H), 5.29-4.98 (m, 2H), 4.87-4.67 (m, 2H), 4.60-4.09 (m, 5H), 3.90-3.82 (m, 1H), 3.62-3.57 (m, 2H), 3.51-3.44 (m, 3H), 3.21-3.17 (m, 1H), 3.05-2.98 (m, 2H), 2.95-2.75 (m, 3H), 2.68-2.58 (m, 1H), 2.42 (m, 3H), 2.22-1.93 (m, 3H), 1.85-1.61 (m, 3H), 1.59-1.50 (m, 2H), 1.47-1.33 (m, 3H), 1.30-1.04 (m, 2H).

LCMS (ESI): m/z=1013.5 [M−H]⁻.

### Preparation of ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)phosphonic acid (Compound 15) and propyl (((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)(phenoxy)phosphoryl)-L-alaninate (Compound 16)

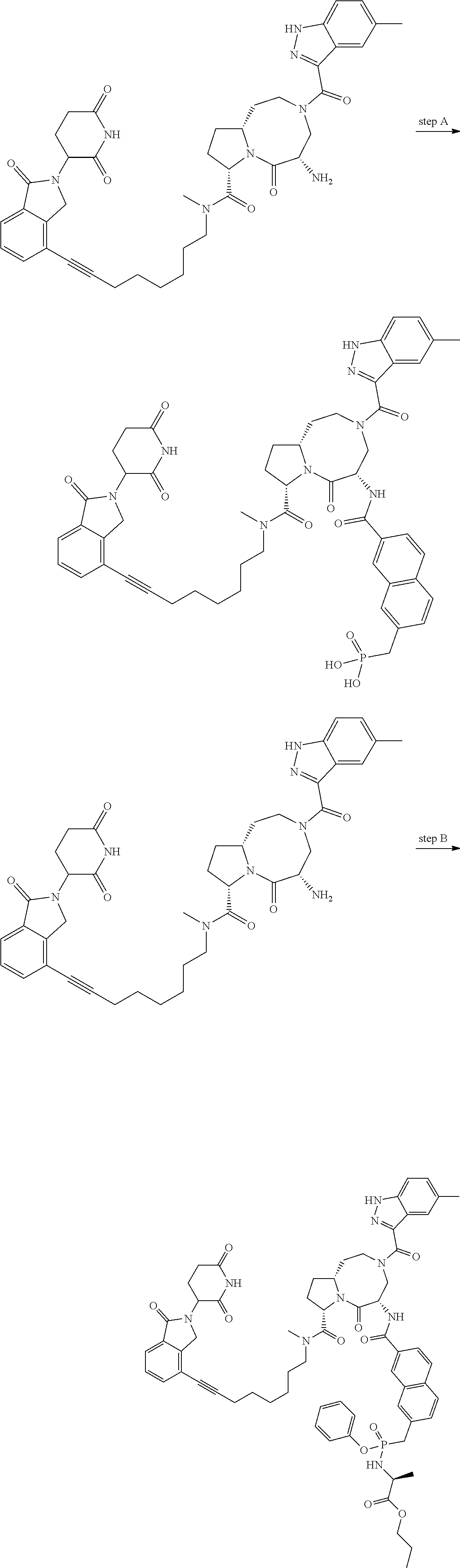

### STEP A: ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)phosphonic acid

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methyl-1H-indazole-3-carbonyl)-6-oxodecahydropyrrolo[1,2-a][1,5]diazocine-8-carboxamide (57.1 mg, 76.3 μmol, 1.1 eq.) and ((7-((perfluorophenoxy)carbonyl)naphthalen-2-yl)methyl)phosphonic acid (30 mg, 69.4 μmol, 1.0 eq.) in DMF (1 mL) was added DIEA (26.8 mg, 208 μmol, 3.0 eq.) at room temperature. The resulting mixture was stirred at room temperature for 1 hr. After completion, the reaction mixture was concentrated under reduced pressure. The residue was purified by RP-prep HPLC to give ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)phosphonic acid (Compound 15, 11.0 mg, 11.0 μmol, 15.9%) as a white solid.

¹H NMR (400 MHz, DMSO-d6) δ 13.66-13.15 (m, 1H), 10.98 (s, 1H), 8.89-8.62 (m, 1H), 8.52-8.37 (m, 1H), 8.01-7.36 (m, 10H), 7.27-7.21 (m, 1H), 5.35-4.94 (m, 4H), 4.92-4.72 (m, 3H), 4.63-4.12 (m, 5H), 3.92-3.46 (m, 3H), 3.26-2.75 (m, 7H), 2.64-2.53 (m, 1H), 2.48-2.45 (m, 1H), 2.44-2.41 (m, 3H), 2.35-2.24 (m, 1H), 2.17-1.95 (m, 3H), 1.87-1.64 (m, 3H), 1.61-1.51 (m, 3H), 1.49-1.39 (m, 3H), 1.35-1.26 (m, 2H). (TFA salt).

LCMS (ESI): m/z=997.3 [M+H]⁻.

### STEP B: propyl (((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl/)(phenoxy)phosphoryl)-L-alaninate

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methyl-1H-indazole-3-carbonyl)-6-oxodecahydropyrrolo[1,2-a][1,5]diazocine-8-carboxamide (40 mg, 53.5 μmol, 1.1 eq.) and 7-(((((S)-1-oxo-1-propoxypropan-2-yl)amino)(phenoxy)phosphoryl)methyl)-2-naphthoic acid (22 mg, 48.6 μmol, 1.0 eq.) in DMF (2 mL) were added HATU (20 mg, 53.5 μmol, 1.1 eq.) and DIEA (31.3 mg, 243 μmol, 5.0 eq.) at room temperature. The resulting mixture was stirred at room temperature for 2 hrs. After completion, the reaction mixture was concentrated under reduced pressure. The residue was purified by RP-prep HPLC to give propyl (((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methyl-1H-indazole-3-carbonyl)-6-oxodecahydropyrrolo[1,2-a][1,5]diazocin-5-yl)carbamoyl)naphthalen-2-yl)methyl)(phenoxy)phosphoryl)-L-alaninate (Compound 16, 10 mg, 8.44 μmol, 17%) as a white solid.

¹H NMR (400 MHz, CDCl₃) δ 8.33-8.22 (m, 1H), 8.12-7.31 (m, 12H), 7.24-7.01 (m, 6H), 5.87-5.56 (m, 1H), 5.21-5.07 (m, 1H), 4.84-4.74 (m, 1H), 4.61-4.34 (m, 3H), 4.31-4.14 (m, 1H), 4.05-3.81 (m, 4H), 3.68-3.22 (m, 7H), 3.10 (d, J=13.7 Hz, 2H), 3.02-2.90 (m, 1H), 2.80-2.67 (m, 1H), 2.49-2.43 (m, 3H), 2.38-2.18 (m, 4H), 2.16-1.77 (m, 12H), 1.67-1.60 (m, 2H), 1.40-1.31 (m, 2H), 1.18-1.06 (m, 3H), 0.86-0.80 (m, 3H).

LCMS (ESI): m/z=1186.7 [M+H]⁺

### Preparation of S-(2-((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)ethyl) 2,2-dimethylpropanethioate (Compound 17)

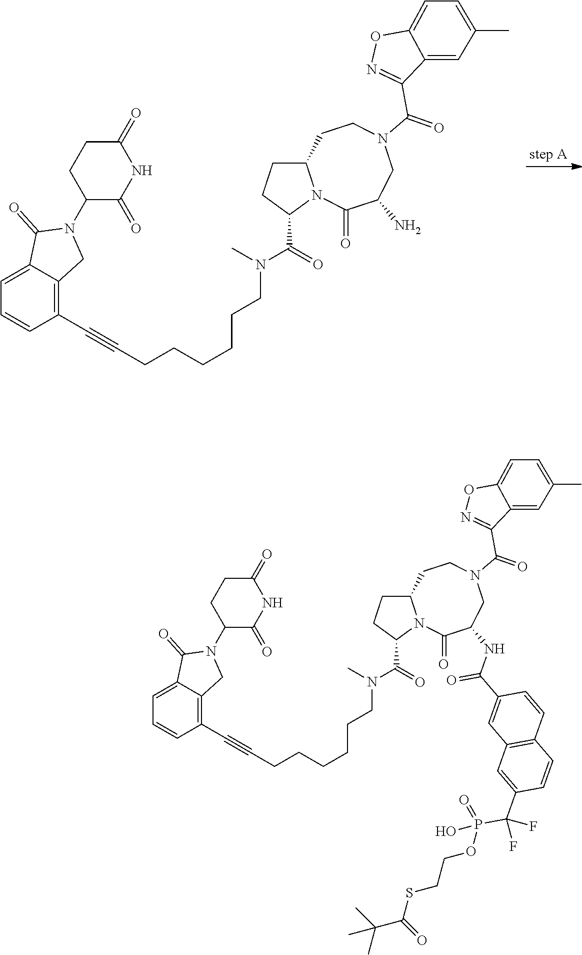 [SMILES: Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](N)C3)c2c1 ; Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(C(F)(F)P(=O)(O)OCCSC(=O)C(C)(C)C)cc5c4)C3)c2c1]

### STEP A: S-(2-((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)ethyl) 2,2-dimethylpropanethioate

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methylbenzo[d]isoxazole-3-carbonyl)-6-oxodecahydropyrrolo[1,2-a][1,5]diazocine-8-carboxamide (50 mg, 66.7 μmol, 1.0 eq.) and perfluorophenyl 7-(difluoro(hydroxy(2-(pivaloylthio)ethoxy)phosphoryl)methyl)-2-naphthoate (41 mg, 66.7 Nmol, 1.0 eq.) in DMF (2 mL) was added DIEA (26 mg, 200 μmol, 3 eq.) at room temperature. The reaction solution was stirred at room temperature for 1 hr. After completion, the reaction solution was purified by RP-prep-HPLC to afford S-(2-((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)ethyl) 2,2-dimethylpropanethioate (Compound 17, 17 mg, 14.4 μmol, 22%) as a white solid.

¹H NMR (400 MHz, DMSO-d6) δ 11.02-10.96 (m, 1H), 8.76-7.42 (m, 13H), 5.31-5.11 (m, 2H), 4.76-4.72 (m, 1H), 4.61-4.56 (m, 2H), 4.46-4.40 (m, 2H), 4.36-4.23 (m, 2H), 4.07-3.93 (m, 4H), 3.35-3.28 (m, 1H), 3.18-2.86 (m, 5H), 2.78-2.74 (m, 1H), 2.63-2.55 (m, 1H), 2.48-2.38 (m, 6H), 2.36-2.09 (m, 3H), 2.05-1.78 (m, 3H), 1.68-1.20 (m, 9H), 1.18-1.11 (m, 9H).

LCMS (ESI): m/z=1178.4 [M+H]⁺.

### Preparation of S,S′-(((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) bis(2,2-dimethylpropanethioate) (Compound 18)

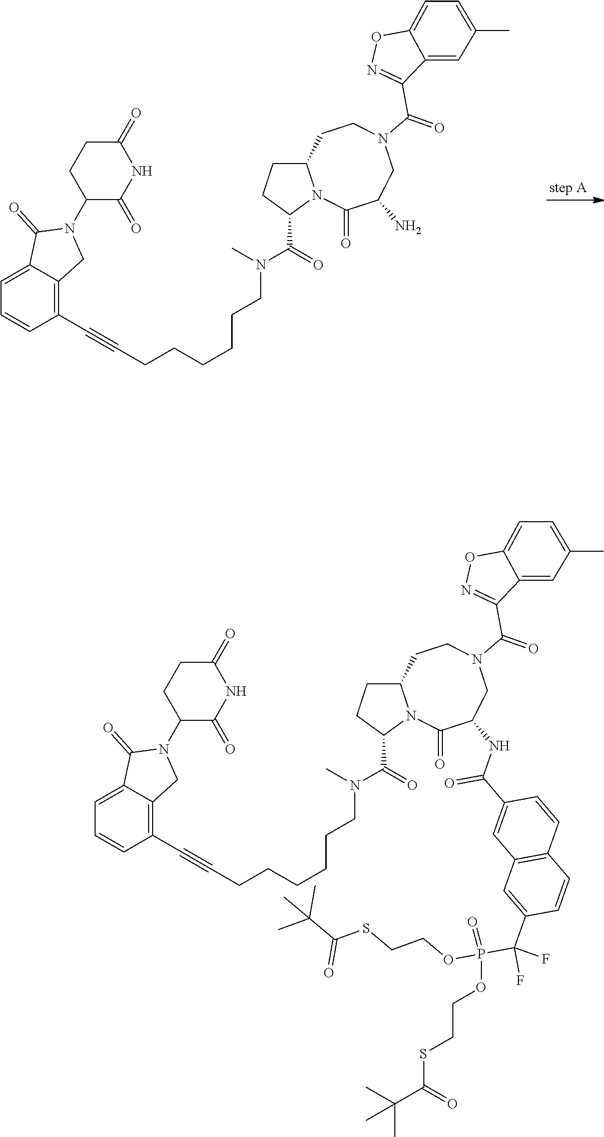 [SMILES: Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](N)C3)c2c1 ; Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(C(F)(F)P(=O)(OCCSC(=O)C(C)(C)C)OCCSC(=O)C(C)(C)C)cc5c4)C3)c2c1]

### STEP A: S,S′-(((((7-(((5S,8S,10aR)-8-((8-(2-(2,6-dioxopiperidin-3-yl)-1-oxoisoindolin-4-yl)oct-7-in-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) bis(2,2-dimethylpropanethioate)

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methylbenzo[d]isoxazole-3-carbonyl)-6-oxodecahydropyrrolo[1,2-a][1,5]diazocine-8-carboxamide (500 mg, 0.67 mmol, 1.0 eq.) and perfluorophenyl 7-((bis(2-(pivaloylthio)ethoxy)phosphoryl)difluoromethyl)-2-naphthoate (504 mg, 0.67 mmol, 1.0 eq.) in DMF (15 mL) was added DIEA (432 mg, 3.35 mmol, 5.0 eq.) at room temperature. The reaction solution was stirred at room temperature for 2 hrs. After completion, the reaction solution was concentrated under reduced pressure. The residue was purified by prep-HPLC to afford S,S′-(((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) bis(2,2-dimethylpropanethioate) (Compound 18, 415 mg, 0.31 mmol, 47%) as a white solid.

¹H NMR (400 MHz, CDCl₃) δ 10.31-8.70 (m, 1H), 8.61-8.32 (m, 1H), 8.30-7.57 (m, 8H), 7.56-7.36 (m, 4H), 5.64-5.26 (m, 1H), 5.26-5.03 (m, 1H), 4.89-4.72 (m, 1H), 4.71-4.33 (m, 3H), 4.33-4.08 (m, 6H), 4.03-3.68 (m, 1H), 3.41-3.30 (m, 1H), 3.21-3.03 (m, 6H), 2.96-2.68 (m, 3H), 2.49-2.40 (m, 4H), 2.39-1.70 (m, 12H), 1.66-1.25 (m, 7H), 1.24-1.14 (m, 18H).

LCMS (ESI): m/z=1322.6 [M+H]⁺.

### Preparation of ((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(methylene) diisopropyl bis(carbonate) (Compound 19) and ((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)methyl isopropyl carbonate (Compound 20)

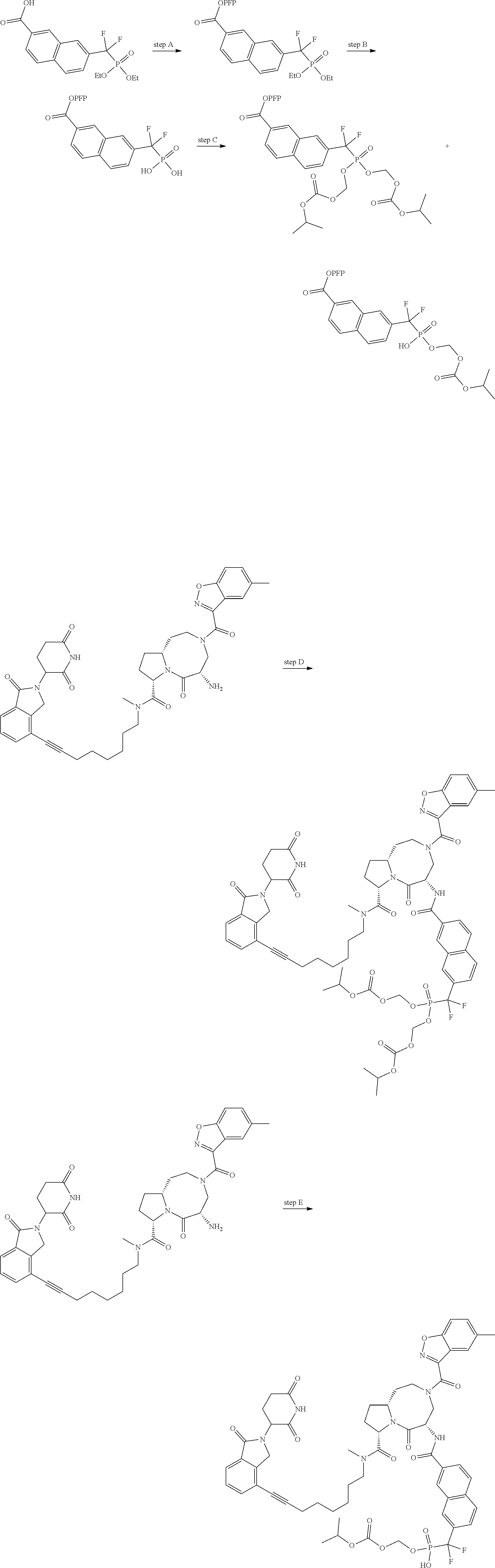

### STEP A: perfluorophenyl 7-((diethoxyphosphoryl)difluoromethyl)-2-naphthoate

Oxalyl chloride (7.53 g, 76.8 mmol, 5.0 eq.) was added dropwise to the solution of 7-((diethoxyphosphoryl)difluoromethyl)-2-naphthoic acid (5.5 g, 15.4 mmol, 1.0 eq.) in dry DCM (120 mL) and DMF (2 drops) at 0° C. The reaction mixture was warmed to room temperature, then stirred for an additional 2 hrs. The reaction was monitored by pipetting out a small amount of crude sample and quenching it with MeOH to ensure acid chloride had been formed completely. After completion, the excess oxalyl chloride and solvent were removed under reduced pressure. The residue was re-dissolved in anhydrous DCM (120 mL) then added to a mixture of 2,3,4,5,6-pentafluorophenol (2.98 g, 16.2 mmol, 1.05 eq.), triethylamine (7.77 g, 76.8 mmol, 5.0 eq.) in anhydrous DCM (100 mL) at 0° C. The reaction was allowed to warm to room temperature, and stirred for an additional 1 hr. The reaction progress was monitored by LCMS, and after completion, the reaction was concentrated under reduced pressure. The residue was purified by flash column chromatography on silica gel to afford perfluorophenyl 7-((diethoxyphosphoryl)difluoromethyl)-2-naphthoate (7.1 g, 13.5 mmol, 88%) as a white solid. LCMS (ESI): m/z=525 [M+H]⁺.

### STEP B: (difluoro(7-((perfluorophenoxy)carbonyl)naphthalen-2-yl)methyl)phosphonic acid

To a solution of perfluorophenyl 7-((diethoxyphosphoryl)difluoromethyl)-2-naphthoate (7.1 g, 13.5 mmol, 1.0 eq.) in DCM (100 mL) was added TMSBr (20 mL) at room temperature. The solution was stirred at room temperature overnight. After completion, the solvent was removed under reduced pressure and the residue was purified by flash column chromatography on silica gel to give (difluoro(7-((perfluorophenoxy)carbonyl)naphthalen-2-yl)methyl)phosphonic acid (5.8 g, 12.4 mmol, 92%). LCMS (ESI): m/z=469 [M+H]⁺.

### STEP C: perfluorophenyl 7-((bis(((isopropoxycarbonyl)oxy)methoxy)phosphoryl)difluoromethyl)-2-naphthoate

To a solution of (difluoro(7-((perfluorophenoxy)carbonyl)naphthalen-2-yl)methyl)phosphonic acid (2.1 g, 4.49 mmol, 1.0 eq.) in a mixture of deionized H₂O (20 mL) and THF (10 mL) was added Amberlite IR120® resin (Na⁺ form)(10 g). The resulting mixture was stirred at room temperature for 1 hr, then the excess of the resin was removed by filtration. AgNO₃ (3.05 g, 17.96 mmol, 4.0 eq.) in deionized H₂O (30 mL) was then added to the resulting solution. After addition, the resulting mixture was stirred at room temperature for an additional 1 hr. During this period, the silver salt formed as a white precipitate, which was collected via filtration. The filter cake was washed with cool H₂O (30 mL×3), and the silver salt was further dried under reduced pressure to get a dry powder. The isolated Ag salt was suspended in ACN (10 mL), and iodomethyl isopropyl carbonate (3.29 g, 13.5 mmol, 3.0 eq.) was added dropwise. After addition, the resulting mixture was stirred at room temperature for an additional 12 hrs. The reaction progress was monitored by LCMS, and after completion, the unreacted silver salt was recovered by filtration, and the filtrate was concentrated under reduced pressure. The resulting residue was purified by flash column chromatography on silica gel to give perfluorophenyl 7-((bis(((isopropoxycarbonyl)oxy)methoxy)phosphoryl)difluoromethyl)-2-naphthoate (1.4 g, 2.0 mmol, 45%) and perfluorophenyl 7-(difluoro(hydroxy(((isopropoxycarbonyl)oxy)methoxy)phosphoyl)methyl)-2-naphthoate (150 mg) as a white solid. LCMS (ESI) m/z=701 [M+H]⁺.

### STEP D: ((((7-(((5S,8S,10R)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(methylene) diisopropyl bis(carbonate)

To a solution of perfluorophenyl 7-((bis(((isopropoxycarbonyl)oxy)methoxy)phosphoryl)difluoromethyl)-2-naphthoate (660 mg, 0.94 mmol, 1.0 eq) and 3-(4-(8-(methylamino)oct-1-yn-1-yl)-1-oxoisoindolin-2-yl)piperidine-2,6-dione (359 mg, 0.94 mmol, 1.0 eq.) in DMF (15 mL) was added DIEA (606 mg, 4.70 mmol, 5.0 eq.) at room temperature. The reaction solution w % as stirred at room temperature for 1 hr, then concentrated under reduced pressure. The residue was purified by prep-HPLC to afford ((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(methylene) diisopropyl bis(carbonate) (Compound 19, 405 mg, 032 mmol, 34%) as a white solid.

¹H NMR (400 MHz, CDCl₃) δ 8.56-7.63 (m, 9H), 7.58-7.34 (m, 4H), 5.82-5.60 (m, 4H), 5.58-5.04 (m, 2H), 4.97-4.74 (m, 3H), 4.71-3.61 (m, 7H), 3.50-3.02 (m, 4H), 2.94-2.72 (m, 3H), 2.50-2.40 (m, 4H), 2.38-1.85 (m, 10H), 1.69-1.36 (m, 6H), 1.31-1.15 (m, 13H).

LCMS (ESI): m/z=1266.3 [M+H]⁺.

### STEP E: ((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)methyl isopropyl carbonate

To a solution of perfluorophenyl 7-(difluoro(hydroxy(((isopropoxycarbonyl)oxy)methoxy)phosphoryl)methyl)-2-naphthoate (30 mg, 51.3 μmol, 1.0 eq.) and 3-(4-(8-(methylamino)oct-1-in-1-yl)-1-oxoisoindolin-2-yl)piperidine-2,6-dione (19.5 mg, 51.3 μmol, 1.0 eq.) in DMF (5 mL) was added DIEA (33 mg, 0.26 mmol, 5.0 eq.) at room temperature. The reaction solution was stirred at room temperature for 1 hr, then concentrated under reduced pressure. The residue was purified by C18 column to afford ((((7-(((5S,8S,10aR)-8-4(8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)methyl isopropyl carbonate (Compound 20, 7.0 mg, 6.10 μmol, 12%) as a white solid.

¹H NMR (400 MHz, DMSO-d6) δ 11.11-10.93 (m, 1H), 8.82-8.47 (m, 1H), 8.24-8.10 (m, 1H), 8.08-7.85 (m, 4H), 7.82-7.40 (m, 7H), 5.48-5.37 (m, 2H), 5.33-5.05 (m, 2H), 4.80-4.68 (m, 2H), 4.51-4.37 (m, 2H), 4.34-4.23 (m, 1H), 4.08-3.91 (m, 3H), 3.90-3.78 (m, 1H), 3.34-3.08 (m, 2H), 3.01-2.97 (m, 1H), 2.95-2.84 (m, 1H), 2.79-2.73 (m, 1H), 2.52 (s, 3H), 2.46-2.38 (m, 7H), 2.06-1.94 (m, 2H), 1.85-1.70 (m, 2H), 1.58-1.46 (m, 3H), 1.45-1.29 (m, 4H), 1.25-1.19 (m, 8H). (TFA salt).

LCMS (ESI): m/z=1150.1 [M+H]⁺.

### Preparation of S,S′-(((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate) (Compound 21) and ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 22)

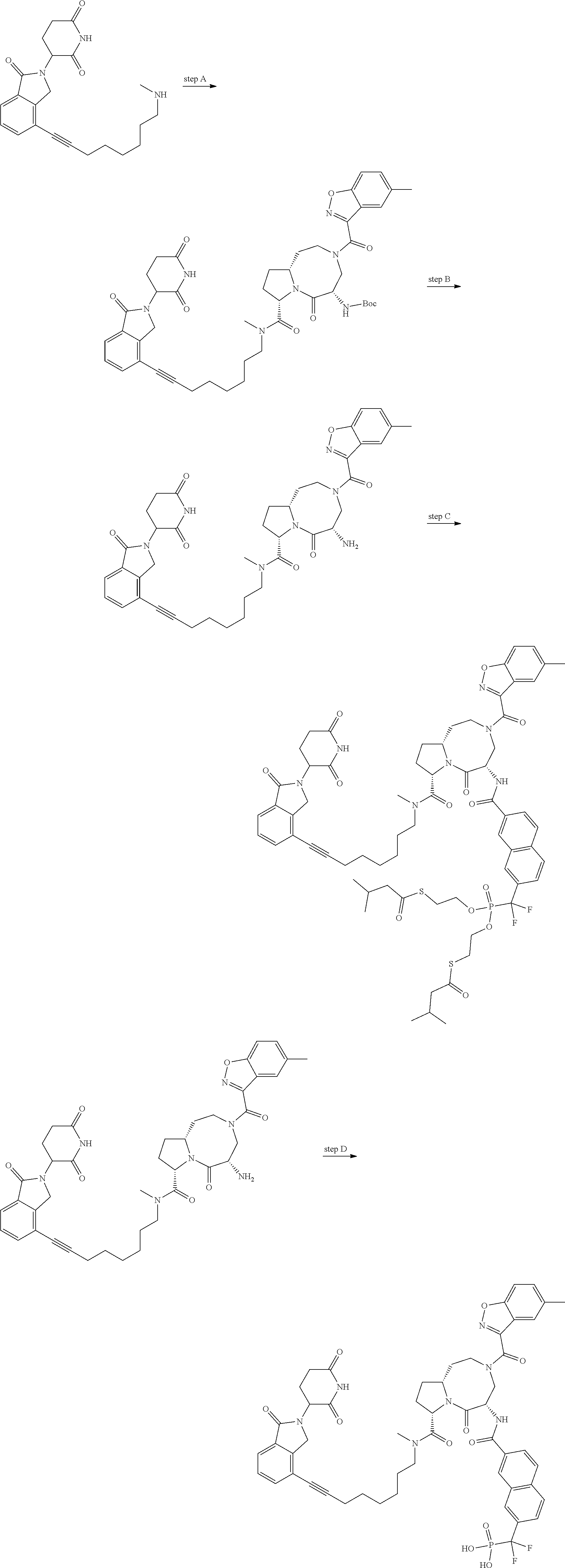

### STEP A: tert-butyl ((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl) carbamate

To a solution of 3-(4-(8-(methylamino) oct-1-yn-1-yl)-1-oxoisoindolin-2-yl)piperidine-2,6-dione (2 g, 5.25 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (2.55 g, 5.25 mmol, 1.0 eq.) in DMF (50 mL) were added DIEA (3.39 g, 26.3 mmol, 5.0 eq.) and HATU (2.19 g, 5.78 mmol, 1.1 eq.) at room temperature. The reaction solution was stirred at room temperature for 2 hrs. After completion, the reaction solution was concentrated under reduced pressure. The residue was purified by C18 column to afford tert-butyl ((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamate (1.67 g, 1.97 mmol, 37%) as a white solid. LCMS (ESI): m/z=850 [M+H]⁺.

### STEP B: (5S,8S,10aR)-5-amino-N-(8-(2-(2,6-d)oxopiperidin-3-yl)-1-oxoisoindolin-4-yl)oct-7-yn-1-yl)-N-methyl-3-(5-methylbenzo[d]isoxazole-3-carbonyl)-6-oxodecahydropyrrolo[1,2-a][1,5]diazocine-8-carboxamide

To a solution of tert-butyl ((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamate (700 mg, 0.82 mmol, 1.0 eq.) in DCM (10 mL) was added TFA (3 mL), and the resulting mixture was stirred at room temperature for 2 hrs. After completion, the reaction mixture was concentrated under reduced pressure to give crude (5S,8S,10aR)-5-amino-N-(8-(2-(2,6-dioxopiperidin-3-yl)-1-oxoisoindolin-4-yl)oct-7-yn-1-yl)-N-methyl-3-(5-methylbenzo[d]isoxazole-3-carbonyl)-6-oxodecahydropyrrolo[1,2-a][1,5]diazocine-8-carboxamide (617 mg, quant.) (TFA salt) as a white solid, which was used in next step directly without further purification. LCMS (ESI): m/z=750 [M+H]⁺.

### STEP C: S,S′-(((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate)

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methylbenzo[d]isoxazole-3-carbonyl)-6-oxodecahydropyrrolo[1,2-a][1,5]diazocine-8-carboxamide (530 mg, 706 μmol, 1.0 eq.) and perfluorophenyl 7-((bis(2-((3-methylbutanoyl)thio)ethoxy)phosphoryl)difluoromethyl)-2-naphthoate (560 mg, 741 μmol, 1.05 eq.) in DMF (9 mL) were added DIEA (272 mg, 2.11 mmol, 3.0 eq.) and DMAP (8 mg, 70.6 μmol, 0.1 eq.) at room temperature. The reaction solution was stirred at room temperature for 1 hr. After completion, the reaction solution was diluted with water (30 mL) and extracted with EtOAc (50 mL×3). The combined organic layers were washed with brine (30 mL×2), dried over dried over with anhydrous Na₂SO₄, then concentrated under reduced pressure. The residue was purified by flash column chromatography on silica gel to afford S,S′-(((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphoryl)bis(oxy))bis(ethane-2,1-diyl)) bis(3-methylbutanethioate) (Compound 21, 507 mg, 383 μmol, 54%) as a yellow solid.

¹H NMR (400 MHz, DMSO-d6) δ 11.02-10.96 (m, 1H), 8.74-8.61 (m, 1H), 8.33-8.00 (m, 4H), 7.96-7.88 (m, 1H), 7.79-7.39 (m, 7H), 5.36-5.06 (m, 2H), 4.82-4.67 (m, 1H), 4.53-3.76 (m, 1H), 3.25-2.62 (m, 10H), 2.62-2.55 (m, 1H), 2.47-2.38 (m, 9H), 2.34-1.09 (m, 18H), 0.89-0.81 (m, 12H).

LCMS (ESI): m/z=1322.5 [M+H]⁺.

### STEP D: ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methylbenzo[d]isoxazole-3-carbonyl)-6-oxodecahydropyrrolo[1,2-a][1,5]diazocine-8-carboxamide (617 mg, 822 μmol, 1.0 eq.) and (difluoro(7-((perfluorophenoxy)carbonyl)naphthalen-2-yl)methyl)phosphonic acid (384 mg, 822 μmol, 1.0 eq.) in DMF (15 mL) were added DIEA (317 mg, 2.46 mmol, 3.0 eq.) and DMAP (10.0 mg, 82.2 μmol, 0.1 eq.) at room temperature. The reaction solution was stirred at room temperature for 2 hrs. After completion, the reaction solution was concentrated under reduced pressure. The residue was purified by RP-pre-HPLC to afford ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 22, 162 mg, 157 μmol, 19%) as a white solid.

¹H NMR (400 MHz, DMSO-d6) δ 11.07-10.92 (m, 1H), 8.97-8.54 (m, 2H), 8.27-8.13 (m, 1H), 8.12-7.88 (m, 4H), 7.76-7.64 (m, 3H), 7.63-7.42 (m, 3H), 5.31-5.21 (m, 1H), 5.17-5.10 (m, 1H), 4.78-4.71 (m, 1H), 4.48-4.40 (m, 2H), 4.35-4.26 (m, 2H), 4.05-3.97 (m, 2H), 3.90-3.82 (m, 1H), 3.39-3.23 (m, 2H), 3.18-3.05 (m, 1H), 3.04-2.84 (m, 3H), 2.78-2.53 (m, 3H), 2.48-2.36 (m, 6H), 2.33-2.24 (m, 1H), 2.21-2.10 (m, 1H), 2.04-1.98 (m, 1H), 1.91 (m, 1H), 1.83-1.70 (m, 2H), 1.67-1.59 (m, 1H), 1.56-1.49 (m, 2H), 1.41-1.30 (m, 3H), 1.19-1.09 (m, 1H). (TFA salt).

LCMS (ESI): m/z=1034.1 [M+H]⁺.

### Preparation of S-(2-((((7-(((5S,8S,10aR)-8-((8-(2-(2,6-dioxopiperidin-3-yl)-1-oxoisoindolin-4-yl)oct-7-yn-1-yl)methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)ethyl) 3-methylbutanethioate (Compound 23)

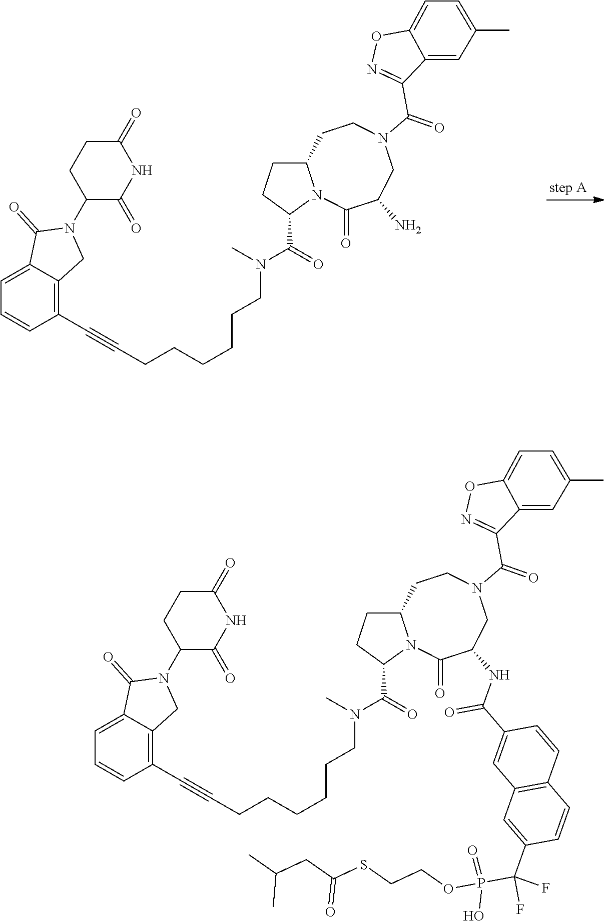 [SMILES: Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](N)C3)c2c1 ; Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(C(F)(F)P(=O)(O)OCCSC(=O)CC(C)C)cc5c4)C3)c2c1]

### STEP A: S-(2-((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)ethyl) 3-methylbutanethioate

To a solution of (5S,8S,10aR)-5-amino-N-(8-(2-(2,6-dioxopiperidin-3-yl)-1-oxoisoindolin-4-yl)oct-7-yn-1-yl)-N-methyl-3-(5-methylbenzo[d]isoxazole-3-carbonyl)-6-oxodecahydropyrrolo[1,2-a][1,5]diazocine-8-carboxamide (51.3 mg, 68.5 μmol, 1.0 eq) and perfluorophenyl 7-(difluoro(hydroxy(2-((3-methylbutanoyl)thio)ethoxy)phosphoryl)methyl)-2-naphthoate (42 mg, 68.5 μmol, 1.0 eq.) in DMF (2 mL) was added DIEA (26.4 mg, 205 μmol, 3 eq.) at room temperature. The reaction solution w % as stirred at room temperature for 1 hr. After completion, the reaction solution was purified by RP-pre-HPLC to afford S-(2-((((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)(hydroxy)phosphoryl)oxy)ethyl) 3-methylbutanethioate (Compound 23, 15.0 mg, 12.7 μmol, 19%) as a white solid.

¹H NMR (400 MHz, CDCl₃) δ 8.38-7.30 (m, 12H), 7.26-7.23 (m, 1H), 5.54-5.24 (m, 1H), 5.18-4.77 (m, 2H), 4.52-4.18 (m, 5H), 3.73-3.39 (m, 5H), 3.22-2.99 (m, 5H), 2.85-2.64 (m, 3H), 2.53-1.74 (m, 18H), 1.46-1.28 (m, 6H), 0.96-0.86 (m, 6H).

LCMS (ESI): m/z=1178.5 [M+H]⁺.

### Preparation of ((4-((E)-3-(((5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)amino)-3-oxoprop-1-en-1-yl)phenyl)difluoromethyl)phosphonic acid (Compound 24) and S,S′-(((((4-((E)-3-(((5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)amino)-3-oxoprop-1-en-1-yl)phenyl)difluoromethyl)phosphoryl)bis(oxy))bis(ethane-2,1-diyl)) dibutanethioate (Compound 25)

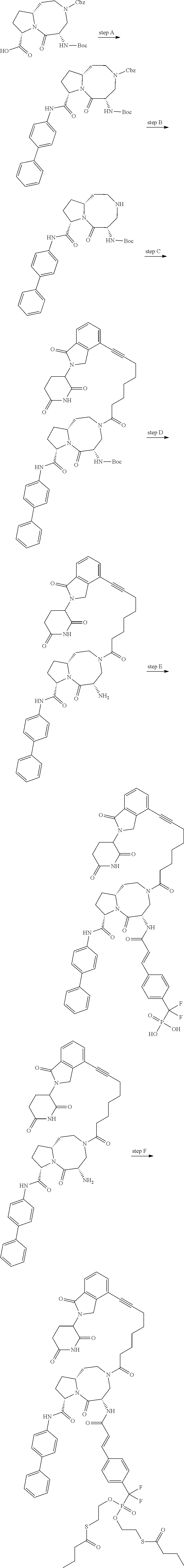

### STEP A: benzyl (5S,8S,10aR)-8-([1,1′-biphenyl]-4-ylcarbamoyl)-5-((tert-butoxycarbonyl)amino)-6-oxooctahydropyrrolo[1,2-a][1,5]diazocine-3(4H)-carboxylate

To a solution of (5S,8S,10aR)-3-((benzyloxy)carbonyl)-5-((tert-butoxycarbonyl)amino)-6-oxodecahydropyrrolo[1,2-a][1,5]diazocine-8-carboxylic acid (800 mg, 1.73 mmol, 1.0 eq.) and [1,1′-biphenyl]-4-amine (292 mg, 1.73 mmol, 1.0 eq.) in DMF (10 mL) were added DIEA (670 mg, 5.19 mmol, 3.0 eq.) and CMPI (443 mg, 1.73 mmol, 1.0 eq.), and the resulting mixture was stirred at room temperature for 2 hrs. After completion, the reaction was diluted with H₂O (20 mL) and extracted with EtOAc (10 mL×3). The organic layers were combined and washed with brine (10 mL), dried over anhydrous Na₂SO₄ and concentrated under reduced pressure. The residue was purified b) flash column chromatography on silica gel to afford benzyl (5S,8S,10aR)-8-([1,1′-biphenyl]-4-ylcarbamoyl)-5-((tert-butoxycarbonyl)amino)-6-oxooctahydropyrrolo[1,2-a][1,5]diazocine-3(4H)-carboxylate (800 mg, 1.31 mmol, 76%) as a white solid. LCMS (ESI): m/z=613 [M+H]⁺.

### STEP B: tert-butyl ((5S,8S,10aR)-8-([1,1′-biphenyl]-4-ylcarbamoyl)-6-oxodecahydropyrrolo[1,2-a][1,5]diazocin-5-yl)carbamate

To a solution of benzyl (5S,8S,10aR)-8-([1,1′-biphenyl]-4-ylcarbamoyl)-5-((tert-butoxycarbonyl)amino)-6-oxooctahydropyrrolo[1,2-a][1,5]diazocine-3(4H)-carboxylate (800 mg, 1.31 mmol, 1.0 eq.) in MeOH (80 mL) was added Pd/C (400 mg) under nitrogen. The suspension was degassed under vacuum and purged with H₂ several times. The resulting mixture was stirred at room temperature for 14 hrs. After completion, the suspension was filtered through a pad of Celite®, the filter cake was washed with MeOH (20 mL). The combined filtrates were concentrated to dryness to give tert-butyl ((5S,8S,10aR)-8-([1,1′-biphenyl]-4-ylcarbamoyl)-6-oxodecahydropyrrolo[1,2-a][1,5]diazocin-5-yl)carbamate (550 mg, quant.) as a white solid. LCMS (ESI): m/z=479 [M+H]⁺.

### STEP C: tert-butyl ((5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamate

To a solution of tert-butyl ((5S,8S,10aR)-8-([1,1′-biphenyl]-4-ylcarbamoyl)-6-oxodecahydropyrrolo[1,2-a][1,5]diazocin-5-yl)carbamate (120 mg, 0.25 mmol, 1.1 eq.) and 8-(2-(2,6-dioxopiperidin-3-yl)-1-oxoisoindolin-4-yl)oct-7-ynoic acid (90 mg, 0.23 mmol, 1.0 eq.) in DMF (2 mL) were added HATU (95 mg, 0.25 mmol, 1.1 eq.) and DIEA (89 mg, 0.69 mmol, 3.0 eq.) at room temperature. The resulting mixture was stirred at room temperature for 2 hrs. After completion, the reaction mixture was concentrated under reduced pressure. The residue was purified by C18-column to give tert-butyl ((5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamate (170 mg, 0.20 mmol, 88%) as a white solid. LCMS (ESI): m/z=843 [M+H]⁺.

### STEP D: (5S,8S,10aR)—N-([1,1′-biphenyl]-4-yl)-5-amino-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carboxamide

To a solution of tert-butyl ((5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamate (60 mg, 71.3 μmol, 1.0 eq.) in DCM (3 mL) was added TFA (1 mL), and the resulting mixture was stirred at room temperature for 2 hrs. After completion, the reaction mixture was concentrated under reduced pressure to give crude (5S,8S,10aR)—N-([1,1′-biphenyl]-4-yl)-5-amino-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carboxamide (50 mg, quant.) as a white solid, which was used in next step directly without further purification. LCMS (ESI): m/z=743 [M+H]⁺.

### STEP E: ((4-((E)-3-(((5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)amino)-3-oxoprop-1-en-1-yl)phenyl)difluoromethyl)phosphonic acid

To a solution of (5S,8S,10aR)—N-([1,1′-biphenyl]-4-yl)-5-amino-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carboxamide (50 mg, 67.4 μmol, 1.0 eq.) and (E)-(difluoro(4-(3-oxo-3-(perfluorophenoxy)prop-1-en-1-yl)phenyl)methyl)phosphonic acid (30 mg, 67.4 μmol, 1.0 eq.) in DMF (2 mL) was added DIEA (44 mg, 0.34 mmol, 5.0 eq.) at room temperature. The resulting mixture was stirred at room temperature for additional 2 hrs. After completion, the reaction mixture was concentrated under reduced pressure. The residue was purified by RP-prep HPLC to give ((4-((E)-3-1(5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)amino)-3-oxoprop-1-en-1-yl)phenyl)difluoromethyl)phosphonic acid (Compound 24, 18.1 mg, 18.0 μmol, 27%) as a white solid.

¹H NMR (400 MHz, DMSO-d6) δ 10.99 (s, 1H), 10.32-10.15 (m, 1H), 8.54-8.20 (m, 1H), 7.79-7.23 (m, 17H), 7.03-6.83 (m, 1H), 5.18-5.07 (m, 1H), 4.96-4.85 (m, 1H), 4.53-4.42 (m, 2H), 4.35-4.24 (m, 2H), 4.01-3.94 (m, 2H), 3.80-3.68 (m, 3H), 3.43-3.20 (m, 2H), 2.94-2.85 (m, 1H), 2.38-1.10 (m, 16H).

LCMS (ESI): m/z=1003.4 [M+H]⁺.

### STEP F: S,S′-(((((4-((E)-3-(((5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)amino)-3-oxoprop-1-en-1-yl)phenyl)difluoromethyl)phosphoryl)bis(oxy))bis(ethane-2,1-diyl)) dibutanethioate

To a solution of (5S,8S,10aR)—N-([1,1′-biphenyl]-4-yl)-5-amino-3-(8-(2-(2,6-dioxopiperidin-3-yl)-1-oxoisoindolin-4-yl)oct-7-ynoyl)-6-oxodecahydropyrrolo[1,2-a][1,5]diazocine-8-carboxamide (50 mg, 67.4 μmol, 1.0 eq.) and perfluorophenyl (E)-3-(4-((bis(2-(butyrylthio)ethoxy)phosphoryl)difluoromethyl)phenyl)acrylate (48 mg, 67.4 μmol, 1.0 eq.) in DMF (2 mL) was added DIEA (44 mg, 0.34 mmol, 5.0 eq.) at room temperature. The resulting mixture was stirred at room temperature for 2 hrs. After completion, the reaction mixture was concentrated under reduced pressure. The residue was purified by RP-prep HPLC to give S,S′-(((((4-((E)-3-(((5S,8S,10aR)-8-([1,1′-biphenyl]-4-ylcarbamoyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)amino)-3-oxoprop-1-en-1-yl)phenyl)difluoromethyl)phosphoryl)bis(oxy))bis(ethane-2,1-diyl)) dibutanethioate (Compound 25, 18.8 mg, 14.9 μmol, 22%) as a white solid.

¹H NMR (400 MHz, CDCl₃) δ 8.85-8.50 (m, 1H), 7.82-7.74 (m, 1H), 7.70-7.45 (m, 1H), 7.45-7.28 (m, 6H), 6.84-6.53 (m, 1H), 5.36-5.25 (m, 1H), 5.03-4.69 (m, 1H), 4.65-4.11 (m, 8H), 3.99-3.59 (m, 2H), 3.48-3.04 (m, 6H), 2.94-2.72 (m, 2H), 2.58-2.01 (m, 15H), 1.74-1.61 (m, 6H), 1.55-1.25 (m, 5H), 0.99-0.91 (m, 6H).

LCMS (ESI): m/z=1263.4 [M+H]⁺.

### Preparation of propyl (((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)fluoromethyl)(phenoxy)phosphoryl)-L-alaninate (Compound 26)

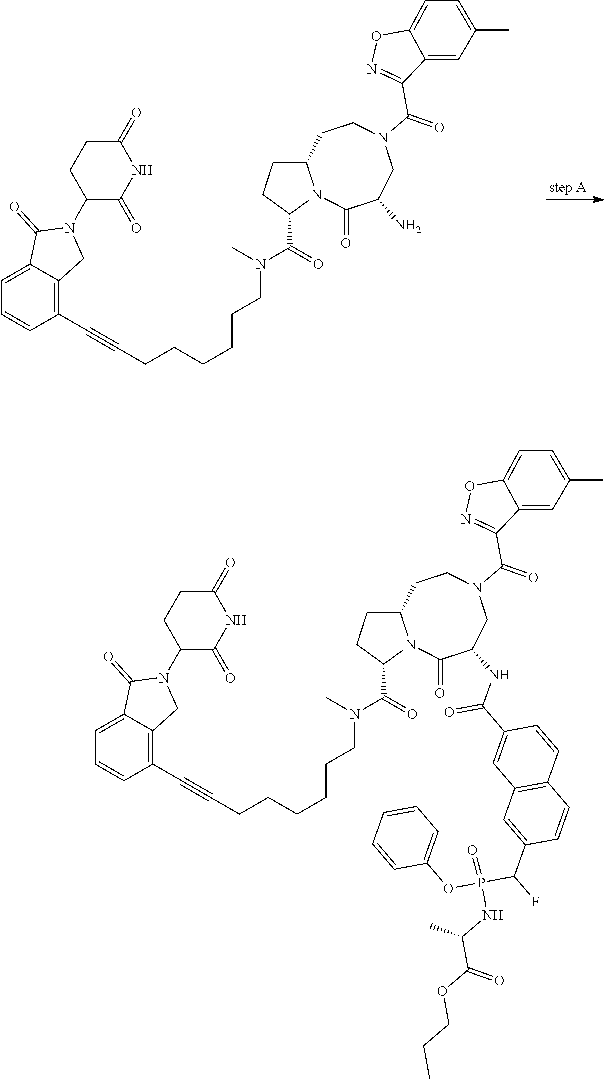 [SMILES: Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](N)C3)c2c1 ; CCCOC(=O)[C@H](C)NP(=O)(Oc1ccccc1)C(F)c1ccc2ccc(C(=O)N[C@H]3CN(C(=O)c4noc5ccc(C)cc45)CC[C@H]4CC[C@@H](C(=O)N(C)CCCCCCC#Cc5cccc6c5CN(C5CCC(=O)NC5=O)C6=O)N4C3=O)cc2c1]

### STEP A: propyl (((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]oxazole-3-carbonyl)-6-oxodecahydropyrrolo[1,2-a][1,5]diazocin-5-yl)carbamoyl)naphthalen-2-yl)fluoromethyl)(phenoxy)phosphoryl)-1-alaninate

To a solution of 7-(fluoro((((S)-1-oxo-1-propoxypropan-2-yl)amino)(phenoxy)phosphoryl)methyl)-2-naphthoic acid (317 mg, 0.67 mmol, 1.0 eq.), (5S,8S,10aR)-5-amino-N-(8-(2-(2,6-dioxopiperidin-3-yl)-1-oxoisoindolin-4-yl)oct-7-yn-1-yl)-N-methyl-3-(5-methylbenzo[d]isoxazole-3-carbonyl)-6-oxodecahydropyrrolo[1,2-a][1,5]diazocine-8-carboxamide (500 mg, 0.67 mmol, 1.0 eq.) and DIEA (432 mg, 3.35 mmol, 5.0 eq.) in DMF (15 mL) was added HATU (306 mg, 0.80 mmol, 1.2 eq.) at room temperature. The reaction solution was stirred at room temperature for 1 hr. After completion, the reaction solution was concentrated under reduced pressure. The residue was purified by prep-HPLC to afford propyl (((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)fluoromethyl)(phenoxy)phosphoryl)-L-alaninate (Compound 26, 420 mg, 035 mmol, 52%) as a white solid.

¹H NMR (400 MHz, DMSO-d6) δ 11.11-10.88 (m, 1H), 9.09-8.64 (m, 1H), 8.64-8.42 (m, 1H), 8.25-7.86 (m, 5H), 7.82-7.40 (m, 6H), 7.39-7.07 (m, 51H), 6.41-6.10 (m, 2H), 5.35-4.69 (m, 3H), 4.53-4.24 (m, 3H), 4.22-3.59 (m, 7H), 3.36-3.22 (m, 2H), 3.16-2.86 (m, 3H), 2.79-2.71 (m, 1H), 2.64-2.53 (m, 1H), 2.48-1.22 (m, 22H), 1.19-1.05 (m, 3H), 1.03-0.90 (m, 1H), 0.87-0.69 (m, 3H).

LCMS (ESI): m/z=1205.5 [M+H]⁺.

### Preparation of (difluoro(7-(((5S,8S,10aR)-8-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid (Compound 27)

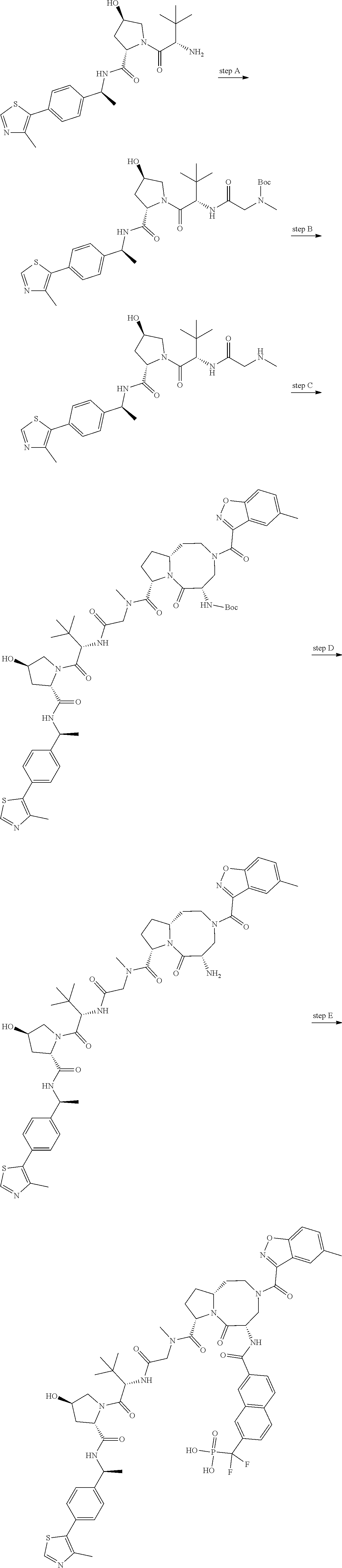

### STEP A: tert-butyl (2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamate

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (120 mg, 0.27 mmol, 1.0 eq.) and N-(tert-butoxycarbonyl)-N-methylglycine (51 mg, 0.27 mmol, 1.0 eq.) in DMA (3 mL) were added HATU (113 mg, 0.30 mmol, 1.1 eq.) and DIEA (104 mg, 0.81 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl (2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamate (110 mg, 0.18 mmol, 67%) as a white solid. LCMS (ESI): m/z=616 [M+H]⁺.

### STEP B: (2S,4R)-1-((S)-3,3-dimethyl-2-(2-(methylamino)acetamido)butanoyl)-4-hydroxy-N—((S)-1-(4-(4-methylthiazol-5-yl)phenyl)ethyl)pyrrolidine-2-carboxamide

To a solution of tert-butyl (2-(((S)-14(2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamate (110 mg, 0.18 mmol, 1.0 eq.) in DCM (3 mL) was added TFA (1 mL), and the resulting mixture was stirred at room temperature for 2 hrs. After completion, the reaction mixture was concentrated under reduced pressure to give crude (2S,4R)-1-((S)-3,3-dimethyl-2-(2-(methylamino)acetamido)butanoyl)-4-hydroxy-N—((S)-1-(4-(4-methylthiazol-5-yl)phenyl)ethyl)pyrrolidine-2-carboxamide (90 mg, quant.) (TFA salt) as a white solid, which was used in next step directly without further purification. LCMS (ESI): m/z=516 [M+H]⁺.

### STEP C: tert-butyl ((5S,8S,10aR)-8-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamoyl)-3-(5-methylbenzo[d]isoxazole-3-carbonyl)-6-oxodecahydropyrrolo[1,2-a][1,5]diazocin-5-yl)carbamate

To a solution of (2S,4R)-1-((S)-3,3-dimethyl-2-(2-(methylamino)acetamido)butanoyl)-4-hydroxy-N—((S)-1(4-(4-methylthiazol-5-yl)phenyl)ethyl)pyrrolidine-2-carboxamide (90 mg, 0.17 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (83 mg, 0.17 mmol, 1.0 eq.) in DMA (3 mL) were added HATU (71 mg, 0.19 mmol, 1.1 eq) and DIEA (66 mg, 0.51 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamoyl)-3-(5-methylbenzo[d]isoxazole-3-carbonyl)-6-oxodecahydropyrrolo[1,2-a][1,5]diazocin-5-yl)carbamate (90 mg, 91.6 μmol, 54%) as a white solid. LCMS (ESI): m/z=984 [M+H]⁺.

### STEP D: (5S,8S,10aR)-5-amino-N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methyl-3-(5-methylbenzo[d]isoxazole-3-carbonyl)-6-oxodecahydropyrrolo[1,2-a][1,5]diazocine-8-carboxamide

To a solution of tert-butyl ((5S,8S,10aR)-8-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamoyl)-3-(5-methylbenzo[d]isoxazole-3-carbonyl)-6-oxodecahydropyrrolo[1,2-a][1,5]diazocin-5-yl)carbamate (90 mg, 91.6 μmol, 1.0 eq.) in DCM (3 mL) was added TFA (1 mL), and the resulting mixture was stirred at room temperature for 2 hrs. After completion, the reaction mixture w as concentrated under reduced pressure to give crude (5S,8S,10aR)-5-amino-N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methyl-3-(5-methylbenzo[d]isoxazole-3-carbonyl)-6-oxodecahydropyrrolo[1,2-a][1,5]diazocine-8-carboxamide (80 mg, quant) (TFA salt) as a white solid, which was used in next step directly without further purification LCMS (ESI): m/z=884 [M+H]⁺.

### STEP E: (difluoro(7-(((5S,8S,10aR)-8-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid

To a solution of (5S,8S,10aR)-5-amino-N-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)-N-methyl-3-(5-methylbenzo[d]isoxazole-3-carbonyl)-6-oxodecahydropyrrolo[1,2-a][1,5]diazocine-8-carboxamide (80 mg, 90.6 μmol, 1.0 eq.) and (difluoro(7-((perfluorophenoxy)carbonyl)naphthalen-2-yl)methyl)phosphonic acid (42 mg, 90.6 μmol, 1.0 eq.) in DMA (2 mL) was added DIEA (58 mg, 0.45 mmol, 5.0 eq.) at room temperature. The resulting mixture was stirred at room temperature for additional 2 hrs. After completion, the reaction mixture was purified by RP-prep HPLC to give (difluoro(7-(((5S,8S,10aR-8-((2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid (Compound 27, 36 mg, 30.8 μmol, 34%) as a white solid.

¹H NMR (400 MHz, DMSO-d6) δ 9.03-8.96 (m, 1H), 8.80-8.28 (m, 3H), 8.26-7.65 (m, 8H), 7.56-7.50 (m, 1H), 7.48-7.34 (m, 4H), 5.29-5.18 (m, 1H), 4.94-4.84 (m, 2H), 4.54-4.47 (m, 4H), 4.30-4.28 (m, 1H), 4.16-3.80 (m, 6H), 3.77-3.51 (m, 3H), 3.03-3.00 (m, 1H), 2.82-2.79 (m, 1H), 2.47-2.41 (m, 6H), 2.37-1.74 (m, 8H), 1.44-1.28 (m, 3H), 0.99-0.86 (m, 9H). (TFA salt).

LCMS (ESI): m/z=1169.4 [M+H]⁺.

### Preparation or ((7-(((5S,8S,10aR)-8-((4-(2-(2,6-dioxopiperidin-3-yl)-1-oxoisoindolin-4-yl)but-3-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 28)

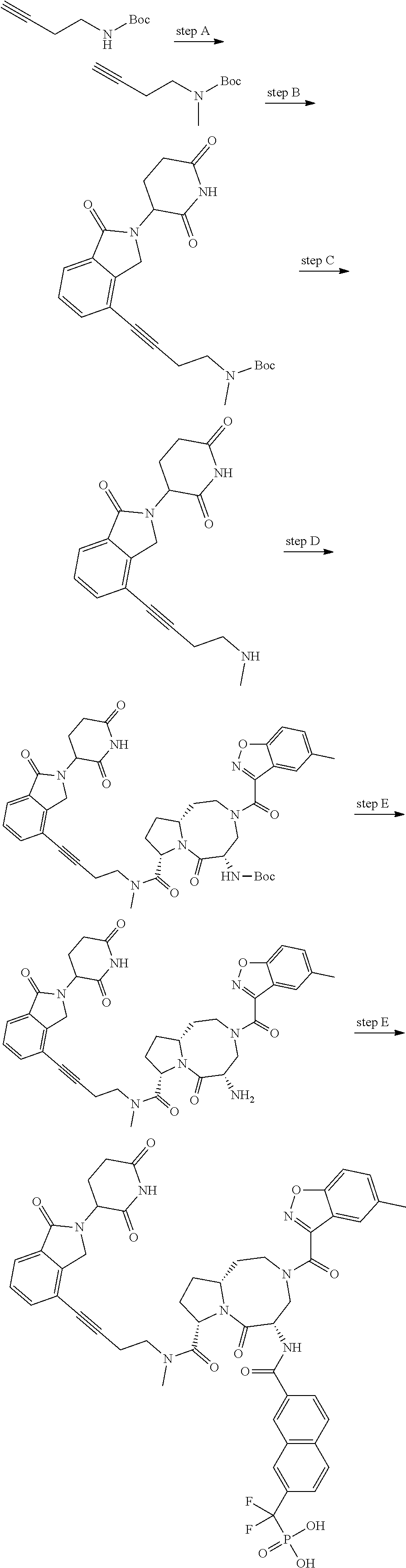

### STEP A: tert-butyl but-3-yn-1-yl(methyl)carbamate

To a solution of tert-butyl but-3-yn-1-ylcarbamate (200 mg, 1.18 mmol, 1.0 eq.) in dry DMF (5 mL) was added NaH (52 mg, 1.30 mmol, 1.1 eq) slowly at 0° C. under nitrogen. The mixture was stirred for additional 30 min at 0° C. then Mel (335 mg, 2.36 mmol, 2.0 eq.) was added drop-wisely. After addition complete addition of Mel, the reaction mixture was allowed to warm to room temperature and stirred for 3 hrs. The reaction mixture was quenched by adding H₂O (5 mL), then extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl but-3-yn-1-yl(methyl)carbamate (180 mg, 0.98 mmol, 83%) as a yellow oil. LC-MS (ESI) m/z=184 [M+H]⁺.

### STEP B: tert-butyl (4-(2-(2,6-dioxopiperidin-3-yl)-1-oxoisoindolin-4-yl)but-3-yn-1-yl)(methyl)carbamate

To a solution of 3-(4-bromo-1-oxoisoindolin-2-yl)piperidine-2,6-dione (263 mg, 0.82 mmol, 1.0 eq.), tert-butyl but-3-yn-1-yl(methyl)carbamate (180 mg, 0.98 mmol, 1.2 eq.), Cs₂CO₃ (800 mg, 2.46 mmol, 3.0 eq.) in DMF (10 mL) were added Pd(PPh₃)₂Cl₂ (41 mg, 0.08 mmol, 0.1 eq.) and CuI (31 mg, 0.16 mmol, 0.2 eq.), and the resulting mixture was stirred at 80° C. for additional 12 hrs. After completion, the reaction mixture was diluted with H₂O (10 mL) and extracted with EtOAc (10 mL×3). The organic layers were combined and washed with brine (10 mL), dried over anhydrous Na₂SO₄ and concentrated under reduced pressure. The residue was purified by flash column chromatography on silica gel to afford tert-butyl (4-(2-(2,6-dioxopiperidin-3-yl)-1-oxoisoindolin-4-yl)but-3-yn-1-yl)(methyl)carbamate (240 mg, 0.56 mmol, 69%) as a white solid. LCMS (ESI) m/z=426 [M+H]⁺.

### STEP C: 3-(4-(4-(methylamino)but-1-yn-1-yl)-1-oxoisoindolin-2-yl)piperidine-2,6-dione

To a solution of tert-butyl (4-(2-(2,6-dioxopiperidin-3-yl)-1-oxoisoindolin-4-yl)but-3-yn-1-yl)(methyl)carbamate (150 mg, 0.35 mmol, 1.0 eq.) in DCM (3 mL) was added TFA (1 mL), and the resulting mixture was stirred at room temperature for 2 hrs. After completion, the reaction mixture was concentrated under reduced pressure to give crude 3-(4-(4-(methylamino)but-1-yn-1-yl)-1-oxoisoindolin-2-yl)piperidine-2,6-dione (110 mg, quant.) as a white solid, which was used in next step directly without further purification. LCMS (ESI): m/z=326 [M+H]⁺.

### STEP D: tert-butyl ((5S,8S,10aR)-8-((4-(2-(2,6-dioxopiperidin-3-yl)-1-oxoisoindolin-4-yl)butyl-3-yn-1-yl)(methyl)carbamoyl)-3-(5-methylbenzo[d]isoxazole-3-carbonyl)-6-oxodecahydropyrrolo[1,2-a][1,5]diazocin-5-yl)carbamate

To a solution of 3-(4-(4-(methylamino)but-1-yn-1-yl)-1-oxoisoindolin-2-yl)piperidine-2,6-dione (110 mg 0.33 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (165 mg, 0.33 mmol, 1.0 eq.) in DMA (5 mL) were added DIEA (213 mg, 1.65 mmol, 5.0 eq.) and HATU (138 mg, 0.36 mmol, 1.1 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-((4-(2-(2,6-dioxopiperidin-3-yl)-1-oxoisoindolin-4-yl)but-3-yn-1-yl)(methyl)carbamoyl)-3-(5-methylbenzo[d]isoxazole-3-carbonyl)-6-oxodecahydropyrrolo[1,2-a][1,5]diazocin-5-yl)carbamate (65 mg, 82.0 μmol, 25%) as a white solid. LCMS (ESI): m/z=794 [M+H]⁺.

### STEP E: (5S,8S,10aR)-5-amino-N-(4-(2-(2,6-dioxopiperidin-3-yl)-1-oxoisoindolin-4-yl)but-3-yn-1-yl)-N-methyl-3-(5-methylbenzo[d]isoxazole-3-carbonyl)-6-oxodecahydropyrrolo[1,2-a][1,5]diazocine-8-carboxamide

To a solution of tert-butyl ((5S,8S,10aR)-8-((4-(2-(2,6-dioxopiperidin-3-yl)-1-oxoisoindolin-4-yl)but-3-yn-1-yl)(methyl)carbamoyl)-3-(5-methylbenzo[d]isoxazole-3-carbonyl)-6-oxodecahydropyrrolo[1,2-a][1,5]diazocin-5-yl)carbamate (65 mg, 82.0 μmol, 1.0 eq.) in DCM (3 mL) was added TFA (I mL), and the resulting mixture was stirred at room temperature for 2 hrs. After completion, the reaction mixture was concentrated under reduced pressure to give crude (5S,8S,10aR)-5-amino-N-(4-(2-(2,6-dioxopiperidin-3-yl)-1-oxoisoindolin-4-yl)but-3-yn-1-yl)-N-methyl-3-(5-methylbenzo[d]isoxazole-3-carbonyl)-6-oxodecahydropyrrolo[1,2-a][1,5]diazocine-8-carboxamide (50 mg, quant.) (TFA salt) as a white solid, which was used in next step directly without further purification. LCMS (ESI): m/z=694 [M+H]⁺.

### STEP F: ((7-(((5S,8S,10aR)-8-((4-(2-(2,6-dioxopiperidin-3-yl)-1-oxoisoindolin-4-yl)but-3-in-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of (5S,8S,10aR)-5-amino-N-(4-(2-(2,6-dioxopiperidin-3-yl)-1-oxoisoindolin-4-yl)but-3-yn-1-yl)-N-methyl-3-(5-methylbenzo[d]isoxazole-3-carbonyl)-6-oxodecahydropyrrolo[1,2-a][1,5]diazocine-8-carboxamide (50 mg, 72.2 μmol, 1.1 eq.) and (difluoro(7-((perfluorophenoxy)carbonyl)naphthalen-2-yl)methyl)phosphonic acid (31 mg, 65.6 μmol, 1.0 eq.) in DMA (2 mL) was added DIEA (42.3 mg, 328 μmol, 5.0 eq.) at room temperature. The resulting mixture was stirred at room temperature for 2 hrs. After completion, the reaction mixture was concentrated under reduced pressure. The residue was purified by RP-prep HPLC to give ((7-(((5S,8S,10aR)-8-((4-(2-(2,6-dioxopiperidin-3-yl)-1-oxoisoindolin-4-yl)but-3-yn-1-yl)(methyl)carbamoyl)-3-(5-methylbenzo[d]isoxazole-3-carbonyl)-6-oxodecahydropyrrolo[1,2-a][0.5]diazocin-5-yl)carbamoyl)naphthalen-2-yl)difluoromethyl)phosphonic acid (Compound 28, 26 mg, 26.6 μmol, 41%) as a white solid.

¹H NMR (400 MHz, DMSO-d6) δ 11.04-10.93 (m, 1H), 9.01-8.69 (m, 1H), 8.69-8.55 (m, 1H), 8.24-8.13 (m, 1H), 8.13-7.93 (m, 4H), 7.78-7.39 (m, 6H), 5.34-5.08 (m, 2H), 4.82-4.73 (m, 1H), 4.50-4.39 (m, 2H), 4.36-4.25 (m, 2H), 4.01-3.96 (m, 2H), 3.87-3.80 (m, 2H), 3.68-3.58 (m, 2H), 3.35-3.22 (m, 1H), 3.17-3.04 (m, 2H), 2.90-2.82 (m, 2H), 2.63-2.57 (m, 1H), 2.53-2.52 (m, 2H), 2.46-2.41 (m, 3H), 2.29-1.47 (m, 6H).

LCMS (ESI): m/z=978.2 [M+H]⁺.

### Preparation of (difluoro(7-(((5S,8S,10aR)-8-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid (Compound 29)

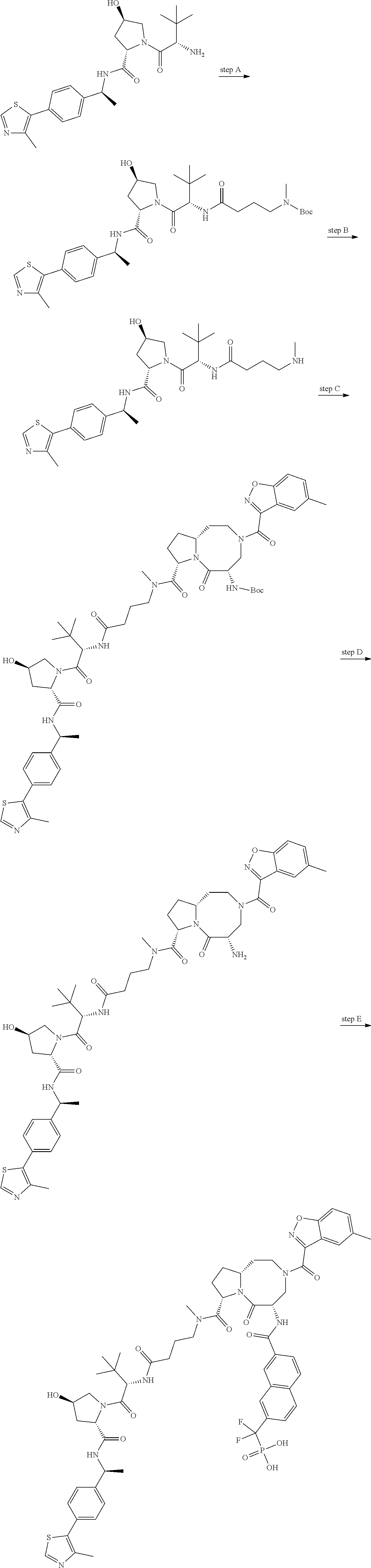

### STEP A: tert-butyl (4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamate

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (150 mg, 0.34 mmol, 1.0 eq.) and 4-((tert-butoxycarbonyl)(methyl)amino)butanoic acid (74 mg, 0.34 mmol, 1.0 eq.) in DMA (3 mL) were added HATU (142 mg, 0.37 mmol, 1.1 eq.) and DIEA (132 mg, 1.02 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl (4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamate (140 mg, 0.22 mmol, 65%) as a white solid. LCMS (ESI): m/z=644 [M+H]⁺.

### STEP B: (2S,4R)-1-((S)-3,3-dimethyl-2-(4-(methylamino)butanamido)butanoyl)-4-hydroxy-N—((S)-1-(4-(4-methylthiazol-5-yl)phenyl)ethyl)pyrrolidine-2-carboxamide

To a solution of tert-butyl (4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamate (140 mg, 0.22 mmol, 1.0 eq.) in DCM (3 mL) was added TFA (1 mL), and the resulting mixture was stirred at room temperature for 2 hrs. After completion, the reaction mixture was concentrated under reduced pressure to give crude (2S,4R)-1-((S)-3,3-dimethyl-2-(4-(methylamino)butanamido)butanoyl)-4-hydroxy-N—((S)-1-(4-(4-methylthiazol-5-yl)phenyl)ethyl)pyrrolidine-2-carboxamide (110 mg, quant.) (TFA salt) as a white solid, which was used in next step directly without further purification. LCMS (ESI): m/z=544 [M+H]⁺.

### STEP C: tert-butyl ((5S,8S,10aR)-8-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamoyl)-3-(5-methylbenzo[d]isoxazole-3-carbonyl)-6-oxodecahydropyrrolo[1,2-a][1,5d]diazocin-5-yl)carbamate

To a solution of (2S,4R)-1-((S)-3,3-dimethyl-2-(4-(methylamino)butanamido)butanoyl)-4-hydroxy-N—((S)-1-(4-(4-methylthiazol-5-yl)phenyl)ethyl)pyrrolidine-2-carboxamide (110 mg, 0.20 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (98 mg, 0.20 mmol, 1.0 eq.) in DMA (3 mL) were added HATU (84 mg, 0.22 mmol, 1.1 eq.) and DIEA (77 mg, 0.6 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamoyl)-3-(5-methylbenzo[d]isoxazole-3-carbonyl)-6-oxodecahydropyrrolo[1,2-a][1,5]diazocin-5-yl)carbamate (120 mg, 0.12 mmol, 59%) as a white solid. LCMS (ESI): m/z 1012 [M+H]⁺.

### STEP D: (5S,8S,10aR)-5-amino-N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)-N-methyl-3-(5-methylbenzo[d]isoxazole-3-carbonyl)-6-oxodecahydropyrrolo[1,2-a][1,5]diazocine-8-carboxamide

To a solution of tert-butyl ((5S,8S,10aR)-8-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamoyl)-3-(5-methylbenzo[d]isoxazole-3-carbonyl)-6-oxodecahydropyrrolo[1,2-a][1,5]diazocin-5-yl)carbamate (90 mg, 89.0 μmol, 1.0 eq.) in DCM (3 mL) was added TFA (1 mL), and the resulting mixture was stirred at room temperature for 2 hrs. After completion, the reaction mixture was concentrated under reduced pressure to give crude (5S,8S,10aR)-5-amino-N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)-N-methyl-3-(5-methylbenzo[d]isoxazole-3-carbonyl)-6-oxodecahydropyrrolo[1,2-a][1,5]diazocine-8-carboxamide (78 mg, quant) (TFA salt) as a white solid, which was used in next step directly without further purification. LCMS (ESI): m/z=912 [M+H]⁺.

### STEP E: (difluoro(7-(((5S,8S,10aR)-8-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid

To a solution of (5S,8S,10aR)-5-amino-N-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)-N-methyl-3-(5-methylbenzo[d]isoxazole-3-carbonyl)-6-oxodecahydropyrrolo[1,2-a][1,5]diazocine-8-carboxamide (78 mg, 85.6 μmol, 1.0 eq.) and (difluoro(7-((perfluorophenoxy)carbonyl)naphthalen-2-yl)methyl)phosphonic acid (40 mg, 85.6 μmol, 1.0 eq.) in DMA (2 mL) was added DIEA (55 mg, 0.43 mmol, 5.0 eq.) at room temperature. The resulting mixture was stirred at room temperature for additional 2 hrs. After completion, the reaction mixture was purified by RP-prep HPLC to give (difluoro(7-(((5S,8S,10aR)-8-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid (Compound 29, 27 mg, 22.6 μmol, 26%) as a white solid.

¹H NMR (400 MHz, DMSO-d6) δ 9.03-8.97 (m, 1H), 8.94-8.53 (m, 2H), 8.41-7.64 (m, 9H), 7.58-7.50 (m, 1H), 7.48-7.29 (m, 4H), 5.31-5.23 (m, 1H), 4.92-4.88 (m, 2H), 4.78-4.76 (m, 1H), 4.53-4.23 (m, 5H), 4.17-3.78 (m, 4H), 3.68-3.48 (m, 2H), 3.37-3.12 (m, 2H), 3.02-3.00 (m, 1H), 2.79-2.75 (M, 1H), 2.48-2.42 (m, 6H), 2.34-1.98 (m, 6H), 1.89-1.42 (m, 6H), 1.40-1.22 (m, 3H), 1.02-0.76 (m, 9H). (TFA salt).

LCMS (ESI): m/z=1197.0 [M+H]⁺.

### Preparation of ((7-(((5S,8S,10aR)-8-((4-(4-(2,6-dioxopiperidin-3-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 30)

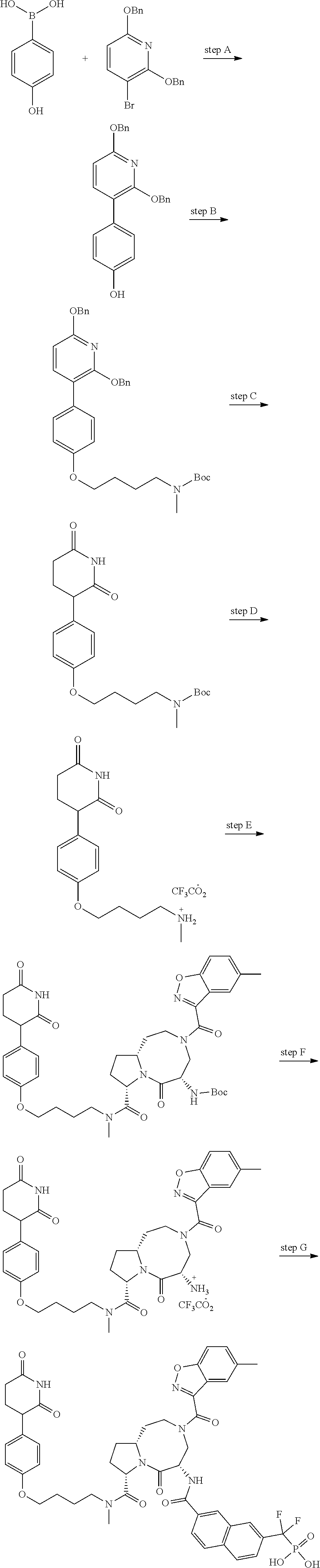

### STEP A: 4-(2,6-bis(benzyloxy)pyridin-3-yl)phenol

To solution of (4-hydroxyphenyl)boronic acid (206 mg, 1.50 mmol, 1.1 eq.), 2,6-bis(benzyloxy)-3-bromopyridine (500 mg, 1.36 mmol, 1.0 eq.), and K₂CO₃ (621 mg, 4.50 mmol, 3.0 eq.) in dioxane/H₂O (15 mL/3 mL), was added Pd(dppf)Cl₂ (99 mg, 0.14 mmol, 0.1 eq), and the resulting mixture was stirred at 100° C. for 12 hrs under N₂. After completion, the reaction mixture was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford 4-(2,6-bis(benzyloxy)pyridin-3-yl)phenol (230 mg, 0.60 mmol, 44%) as a white solid. LCMS (ESI): m/z=384 [M+H]⁺.

### STEP B: tert-butyl (4-(4-(2,6-bis(benzyloxy)pyridin-3-yl)phenoxy)butyl)(methyl)carbamate

To a solution of 4-(2,6-bis(benzyloxy)pyridin-3-yl)phenol (600 mg, 1.57 mmol, 1.0 eq.) and K₂CO₃ (649 mg, 4.70 mmol, 3.0 eq.) in DMF (10 mL) was added 4-((tert-butoxycarbonyl)(methyl)amino)butyl 4-methylbenzenesulfonate (841 mg, 2.36 mmol, 1.5 eq.), and the resulting mixture was stirred at 50° C. for 1 hr. After completion, the reaction mixture was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl (4-(4-(2,6-bis(benzyloxy)pyridin-3-yl)phenoxy)butyl)(methyl)carbamate (400 mg, 0.70 mmol, 45%) as a white solid. LCMS (ESI): m/z=569 [M+H]⁺.

### STEP C: tert-butyl (4-(4-(2,6-dioxopiperidin-3-yl)phenoxy)butyl)(methyl)carbamate

To a solution of tert-butyl (4-(4-(2,6-bis(benzyloxy)pyridin-3-yl)phenoxy)butyl)(methyl)carbamate (400 mg, 0.70 mmol, 1 eq.) in MeOH (50 mL) was added Pd/C (200 mg) under nitrogen. The suspension was degassed under vacuum and purged with H₂ several times. The resulting mixture was stirred at room temperature for 14 hrs. After completion, the suspension was filtered through a pad of Celite®, the filter cake was washed with MeOH (20 mL). The combined filtrates were concentrated to dryness to give tert-butyl (4-(4-(2,6-dioxopiperidin-3-yl)phenoxy)butyl)(methyl)carbamate (200 mg, 0.51 mmol, 73%) as a white solid. LCMS (ESI): m/z=391 [M+H]⁺.

### STEP D: 3-(4-(4-(methylamino)butoxy)phenyl)piperidine-2,6-dione

To a solution of tert-butyl (4-(4-(2,6-dioxopiperidin-3-yl)phenoxy)butyl)(methyl)carbamate (200 mg, 0.51 mmol, 1.0 eq.) in DCM (3 mL) was added TFA (1 mL), and the resulting mixture was stirred at room temperature for 2 hrs. After completion, the reaction mixture was concentrated under reduced pressure to give crude 344-(4-(methylamino)butoxy)phenyl)piperidine-2,6-dione (200 mg, quant.) (TFA salt) as a white solid, which was used in next step directly without further purification. LCMS (ESI): m/z=291 [M+H]⁺.

### STEP E: tert-butyl ((5S,8S,10aR)-8-((4-(4-(2,6-dioxopiperidin-3-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamate

To a solution of 3-(4-(4-(methylamino)butoxy)phenyl)piperidine-2,6-dione (200 mg, 0.69 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (335 mg, 0.69 mmol, 1.0 eq.) in DMF (3 mL) were added HATU (288 mg, 0.76 mmol, 1.1 eq.) and DIEA (294 mg, 2.28 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-((4-(4-(2,6-dioxopiperidin-3-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamate (150 mg, 97.8 μmol, 14%) as a white solid. LCMS (ESI): m/z=759 [M+H]⁺.

### STEP F: (5S,8S 10aR)-5-amino-N-(4-(4-(2,6-dioxopiperidin-3-yl)phenoxy)butyl)-N-methyl-3-(S-methylbenzo[d]isoxazole-3-carbonyl)-6-oxodecahydropyrrolo[1,2-a][1,5]diazocine-8-carboxamide

To a solution of tert-butyl ((5S,8S,10aR)-8-((4-(4-(2,6-dioxopiperidin-3-yl)phenoxy)butyl(methyl)carbamoyl)-3-(5-methylbenzo[d]isoxazole-3-carbonyl)-6-oxodecahydropyrrolo[1,2-a][1,5]diazocin-5-yl)carbamate (150 mg, 97.8 μmol, 1.0 eq.) in DCM (3 mL) was added TFA (1 mL), and the resulting mixture was stirred at room temperature for 2 hrs. After completion, the reaction mixture was concentrated under reduced pressure to give crude (5S,8S,10aR)-5-amino-N-(4-(4-(2,6-dioxopiperidin-3-yl)phenoxy)butyl)-N-methyl-3-(5-methylbenzo[d]isoxazole-3-carbonyl)-6-oxodecahydropyrrolo[1,2-a][1,5]diazocine-8-carboxamide (150 mg, quant.) (TFA salt) as a white solid, which was used in next step directly without further purification. LCMS (ESI): m/z=658 [M+H]⁺.

### STEP G: ((7-(((S,8S,10aR)-8-((4-(4-(2,6-dioxopiperidin-3-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of (5S,8S,10aR)-5-amino-N-(4-(4-(2,6-dioxopiperidin-3-yl)phenoxy)butyl)-N-methyl-3-(5-methylbenzo[d]isoxazole-3-carbonyl)-6-oxodecahydropyrrolo[1,2-a][1,5]diazocine-8-carboxamide (150 mg, 0.10 mmol, 1.0 eq.) and (difluoro(7-((perfluorophenoxy)carbonyl)naphthalen-2-yl)methyl)phosphonic acid (49 mg, 0.10 mmol, 1.0 eq.) in DMF (2 mL) was added DIEA (65 mg, 0.5 mmol, 5.0 eq.) at room temperature. The resulting mixture was stirred at room temperature for additional 2 hrs. After completion, the reaction mixture was purified by RP-prep HPLC to give ((7-(((5S,8S,10aR)-8-((4-(4-(2,6-dioxopiperidin-3-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 30, 27 mg, 28.7 μmol, 29%) as a white solid.

¹H NMR (400 MHz, DMSO-d6) δ 10.77 (d, J=5.2 Hz, 1H), 9.00-8.68 (m, 1H), 8.59 (s, 1H), 8.28-7.83 (m, 5H), 7.80-7.60 (m, 2H), 7.58-7.42 ((m, 1H), 7.14-7.04 (m, 1H), 7.02-6.91 (m, 1H), 6.90-6.71 (m, 2H), 5.33-5.16 (m, 1H), 4.82-4.72 (m, 1H), 4.49-4.43 (m, 1H), 3.98-3.66 (m, 7H), 3.43-3.16 (m, 2H), 3.05-2.74 (m, 3H), 2.69-2.58 (m, 1H), 2.55-2.52 (m, 1H), 2.47-2.40 (m, 3H), 2.35-1.37 (m, 12H).

LCMS (ESI): m/z=943.2 [M+H]⁺.

### Preparation of ((7-(((5S,8S,10aR)-8-((4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 31)

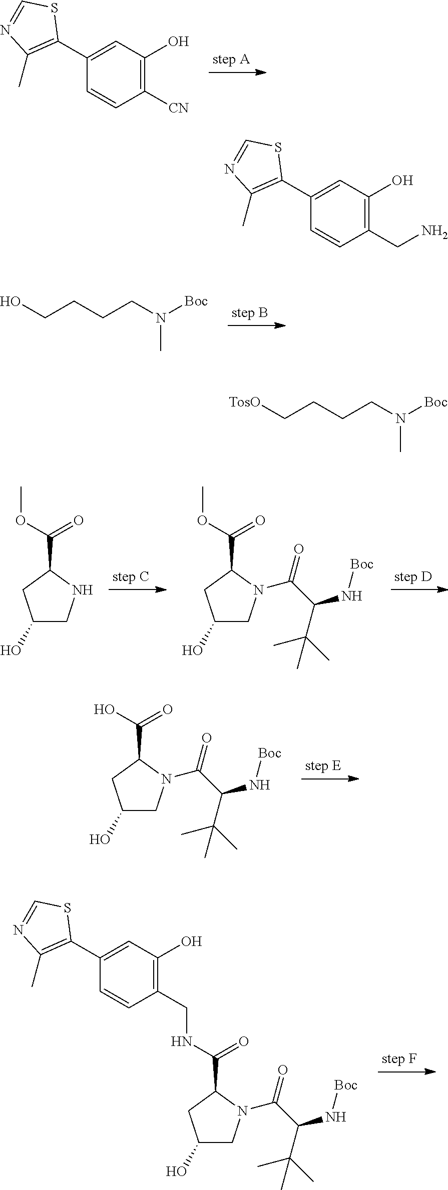

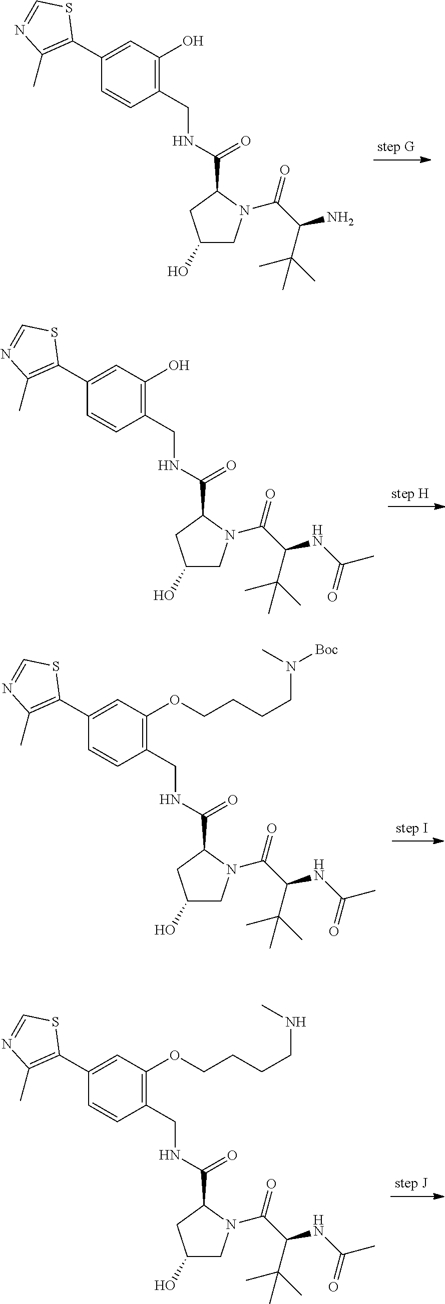

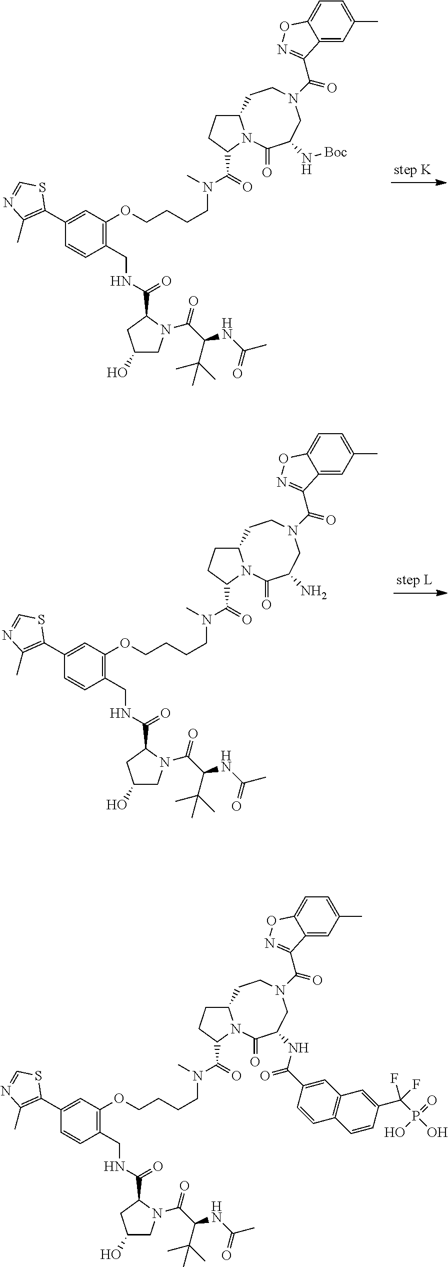

### STEP A: 2-(aminomethyl)-5-(4-methylthiazol-5-yl)phenol

To a solution of 2-hydroxy-4-(4-methylthiazol-5-yl)benzonitrile (800 mg, 3.70 mmol, 1.0 eq.) in dry THF (5 mL) was added LiAlH₄ (422 mg, 11.1 mmol, 3.0 eq) slowly at 0° C. under nitrogen. After addition, the reaction mixture was allowed to warm to room temperature and stirred for 3 hrs. After completion, the reaction mixture was quenched by adding 15% NaOH (4 mL) and filtered, the filtrate was then extracted with EtOAc (10 mL×3). The organic layers were combined and washed with brine (10 mL), dried over anhydrous Na₂SO₄, and concentrated under reduced pressure. The residue was purified by flash column chromatography on silica gel to afford 2-(aminomethyl)-5-(4-methylthiazol-5-yl)phenol (310 mg, 1.40 mmol, 38%) as a yellow oil. LC-MS (ESI) m/z=221 [M+H]⁺.

### STEP B: 4-((tert-butoxycarbonyl)(methyl)amino)butyl 4-methylbenzenesulfonate

To a solution of tert-butyl (4-hydroxybutyl)(methyl)carbamate (800 mg, 3.94 mmol, 1.0 eq.) and TEA (1.19 g, 11.8 mmol, 3.0 eq.) in dry DCM (5 mL) was added TsCl (399 mg, 2.10 mmol, 1.5 eq.) slowly at 0° C. under nitrogen. After addition, the reaction mixture was allowed to warm to room temperature and stirred for 3 hrs. After completion, the reaction mixture was quenched by adding H₂O (4 mL), then extracted with DCM (10 mL×3). The organic layers were combined and washed with brine (10 mL), dried over anhydrous Na₂SO₄, and concentrated under reduced pressure to afford 4-((tert-butoxycarbonyl)(methyl)amino)butyl 4-methylbenzenesulfonate (1.4 g. quant.) as a yellow oil. LC-MS (ESI) m/z=358 [M+H]⁺.

### STEP C: methyl (2S,4R)-1-((S)-2-((tert-butoxycarbonyl)amino)-3,3-dimethylbutanoyl)-4-hydroxypyrrolidine-2-carboxylate

To a solution of methyl (2S,4R)-4-hydroxypyrrolidine-2-carboxylate hydrochloride (1.8 g, 9.94 mmol, 1.0 eq.) and (S)-2-((tert-butoxycarbonyl)amino)-3,3-dimethylbutanoic acid (2.30 g, 9.94 mmol, 1.0 eq) in DMF (100 mL) were added HATU (4.15 g, 10.9 mmol, 1.1 eq) and DIEA (3.85 g, 29.8 mmol, 3.0 eq.), and the resulting mixture was stirred at room temperature for 2 hrs. After completion, the reaction was diluted with H₂O (100 mL) and extracted with EtOAc (100 mL×3). The organic layers were combined and washed with brine (100 mL), dried over anhydrous Na₂SO₄, and concentrated under reduced pressure. The residue was purified by flash column chromatography on silica gel to afford methyl (2S,4R)-1-((S)-2-((tert-butoxycarbonyl)amino)-3,3-dimethylbutanoyl)-4-hydroxypyrrolidine-2-carboxylate (3 g, 8.38 mmol, 84%) as a white solid LCMS (ESI): m/z=359 [M+H]⁺.

### STEP D: (2S,4R)-1-((S)-2-((tert-butoxycarbonyl)amino)-3,3-dimethylbutanoyl)-4-hydroxypyrrolidine-2-carboxylic acid

To a solution of methyl (2S,4R)-1-((S)-2-((tert-butoxycarbonyl)amino)-3,3-dimethylbutanoyl)-4-hydroxypyrrolidine-2-carboxylate (3 g, 8.38 mmol, 1.0 eq.) in THF/H₂O (20 mL/7 mL) was added LiOH (1.05 g, 25.1 mmol, 1.5 eq.), and the resulting mixture was stirred at room temperature for 2 hrs. After completion, the reaction mixture was cooled down in an ice bath, then neutralized carefully with HCl (aq., 1N) until the pH was adjusted to pH=2-3. The resulting mixture was extracted with EtOAc (30 mL×3), and the combined organic layers were washed with brine (10 mL×2), dried over with anhydrous Na₂SO₄, then concentrated under reduced pressure to give crude (2S,4R)-1-((S)-2-((tert-butoxycarbonyl)amino)-3,3-dimethylbutanoyl)-4-hydroxypyrrolidine-2-carboxylic acid (2.5 g, quant.) as a white solid, which was used in next step directly without further purification. LCMS (ESI): m/z=345 [M+H]⁺.

### STEP E: tert-butyl ((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)carbamate

To a solution of (2S,4R)-1-((S)-2-((tert-butoxycarbonyl)amino)-3,3-dimethylbutanoyl)-4-hydroxypyrrolidine-2-carboxylic acid (310 mg, 0.90 mmol, 1.0 eq.) and 2-(aminomethyl)-5-(4-methylthiazol-5-yl)phenol (198 mg, 0.90 mmol, 1.0 eq.) in DMF (mL) were added HATU (376 mg, 0.99 mmol, 1.1 eq.) and DIEA (348 mg, 2.70 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)carbamate (360 mg, 0.65 mmol, 72%) as a white solid. LCMS (ESI): m/z=547 [M+H]⁺.

### STEP F: (2S,4R)-1-((S)-2-amino-3,3-dimethylbutanoyl-4-hydroxy-N-(2-hydroxy-4-(4-methylthiazol-5-yl)benzyl)pyrrolidine-2-carboxamide

To a solution of tert-butyl ((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl)carbamate (360 mg, 0.65 mmol, 1.0 eq.) in DCM (3 mL) was added TFA (1 mL), and the resulting mixture was stirred at room temperature for 2 hrs. After completion, the reaction mixture was concentrated under reduced pressure to give crude (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide (300 mg, quant.) (TFA salt) as a white solid, which was used in next step directly without further purification. LCMS (ESI): m/z=447 [M+H]⁺.

### STEP G: (2S,4R)-1-((S)-2-acetamido-3,3-dimethylbutanoyl)-4-hydroxy-N-(2-hydroxy-4-(4-methylthiazol-5-yl)benzyl)pyrrolidine-2-carboxamide

To a solution of (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide (300 mg, 0.67 mmol, 1.0 eq.) and NaHCO₃ (169 mg, 2.01 mmol, 3.0 eq.) in THF/H₂O (5 mL/1 mL) was added Ac₂O (75 mg, 0.74 mmol, 1.1 eq.), and the resulting mixture was stirred at room temperature for 2 hrs. After completion, the reaction mixture was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford (2S,4R)-1-((S)-2-acetamido-3,3-dimethylbutanoyl)-4-hydroxy-N-(2-hydroxy-4-(4-methylthiazol-5-yl)benzyl)pyrrolidine-2-carboxamide (200 mg, quant.) as an oil. LCMS (ESI): m/z=489 [M+H]⁺.

### STEP H: tert-butyl (4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamate

To a solution of (2S,4R)-1-((S)-2-acetamido-3,3-dimethylbutanoyl)-4-hydroxy-N-(2-hydroxy-4-(4-methylthiazol-5-yl)benzyl)pyrrolidine-2-carboxamide (200 mg, 0.41 mmol, 1.0 eq.) and K₂CO; (170 mg, 1.23 mmol, 3.0 eq.) in DMF (5 mL) was added 4-((tert-butoxycarbonyl)(methyl)amino)butyl 4-methylbenzenesulfonate (220 mg, 0.62 mmol, 1.5 eq.), and the resulting mixture was stirred at 90° C. for 1 hr. After completion, the reaction mixture was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl (4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamate (103 mg, 0.21 mmol, 51%) as an oil. LCMS (ESI): m/z=674 [M+H]⁺.

### STEP I: (2S,4R)-1-((S)-2-acetamido-3,3-dimethylbutanoyl)-4-hydroxy-N-(2-(4-(methylamino)butoxy)-4-(4-methylthiazol-5-yl)benzyl)pyrrolidine-2-carboxamide

To a solution of tert-butyl (4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamate (60 mg, 89.2 μmol, 1.0 eq.) in DCM (3 mL) was added TFA (1 mL), and the resulting mixture was stirred at room temperature for 2 hrs. After completion, the reaction mixture was concentrated under reduced pressure to give crude (2S,4R)-1-((S)-2-acetamido-3,3-dimethylbutanoyl)-4-hydroxy-N-(2-(4-(methylamino)butoxy)-4-(4-methylthiazol-5-yl)benzyl)pyrrolidine-2-carboxamide (60 mg, quant.) (TFA salt) as a white solid, which was used in next step directly without further purification. LCMS (ESI): m/z=574 [M+H]⁺.

### STEP J: tert-butyl ((5S,8S,10aR)-8-((4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamate

To a solution of (2S,4R)-1-((S)-2-acetamido-3,3-dimethylbutanoyl)-4-hydroxy-N-(2-(4-(methylamino)butoxy)-4-(4-methylthiazol-5-yl)benzyl)pyrrolidine-2-carboxamide (60 mg, 0.10 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (49 mg, 0.10 mmol, 1.0 eq.) in DMF (3 mL) were added HATU (42 mg, 0.11 mmol, 1.1 eq.) and DIEA (43 mg, 0.33 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-((4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamate (57 mg, 55 μmol, 55%) as a white solid. LCMS (ESI): m/z=1043 [M+H]⁺.

### STEP K: (5S,8S,10aR)—N-(4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)-5-amino-N-methy-3-(5-methylbenzo[d]isoxazole-3-carbonyl)-6-oxodecahydropyrrolo[1,2-a][1,5]diazocine-8-carboxamide

To a solution of tert-butyl ((5S,8S,10aR)-8-((4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamate (57 mg, 55 μmol, 1.0 eq.) in DCM (3 mL) was added TFA (1 mL), and the resulting mixture was stirred at room temperature for 2 hrs. After completion, the reaction mixture was concentrated under reduced pressure to give crude (5S,8S,10aR)—N-(4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)-5-amino-N-methyl-3-(5-methylbenzo[d]isoxazole-3-carbonyl)-6-oxodecahydropyrrolo[1,2-a][1,5]diazocine-8-carboxamide (45 mg, quant.) (TFA salt) as a white solid, which was used in next step directly without further purification. LCMS (ESI): m/z=943 [M+H]⁺.

### STEP L: ((7-(((5S,8S,10aR)-8-((4-(2-(((2S 4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of (5S,8S,10aR)—N-(4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)-5-amino-N-methyl-3-(5-methylbenzo[d]isoxazole-3-carbonyl)-1-oxodecahydropyrrolo[1,2-a][1,5]diazocine-8-carboxamide (45 mg, 47.8 μmol, 1.0 eq.) and (difluoro(7-((perfluorophenoxy)carbonyl)naphthalen-2-yl)methyl)phosphonic acid (22 mg, 47.8 μmol, 1.0 eq.) in DMF (2 mL) was added DIEA (31 mg, 239 μmol, 5.0 eq.) at room temperature. The resulting mixture was stirred at room temperature for additional 2 hrs. After completion, the reaction mixture was purified by RP-prep HPLC to give ((7-(((5S,8S,10aR)-8-((4-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)butyl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 31, 30 mg, 24.5 μmol, 51%) as a white solid.

¹H NMR (400 MHz, CDCl₃) δ 9.00-8.94 (m, 1H), 8.83-8.69 (m, 1H), 8.58 (s, 1H), 8.51-8.35 (m, 1H), 8.24-7.89 (m, 6H), 7.78-7.59 (m, 2H), 7.57-7.30 (m, 2H), 7.00-6.83 (m, 2H), 5.30-5.19 (m, 1H), 4.82-4.71 (m, 2H), 4.53-4.45 (m, 3H), 4.33-4.26 (m, 2H), 4.16-4.07 (m, 2H), 4.03-3.97 (m, 2H), 3.89-3.82 (m, 1H), 3.64 (s, 2H), 3.52-3.15 (m, 3H), 3.04-2.75 (m, 311), 2.47-2.38 (m, 6H), 2.34-1.92 (m, 511), 1.90-1.86 (m, 3H), 1.85-1.41 (m, 711), 1.02-0.72 (m, 9H). (TFA salt).

LCMS (ESI): m/z=1226.8 [M+H]⁺.

### Preparation of (difluoro(7-(((5S,8S,10aR)-8-((2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid (Compound 32)

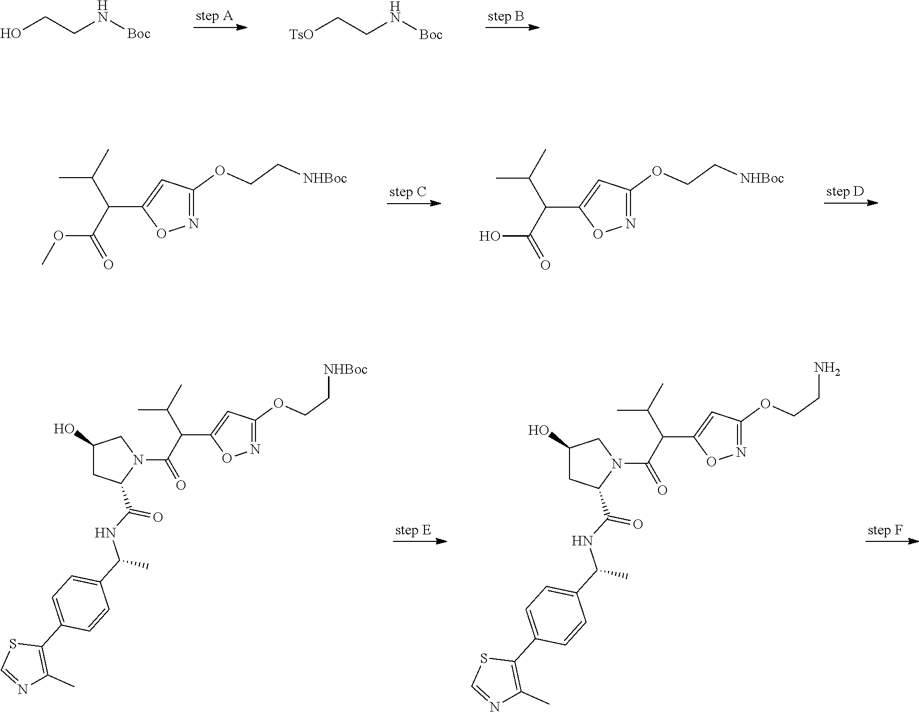 [SMILES: CC(C)(C)OC(=O)NCCO ; Cc1ccc(S(=O)(=O)OCCNC(=O)OC(C)(C)C)cc1 ; COC(=O)C(c1cc(OCCNC(=O)OC(C)(C)C)no1)C(C)C ; CC(C)C(C(=O)O)c1cc(OCCNC(=O)OC(C)(C)C)no1 ; Cc1ncsc1-c1ccc([C@@H](C)NC(=O)[C@@H]2C[C@@H](O)CN2C(=O)C(c2cc(OCCNC(=O)OC(C)(C)C)no2)C(C)C)cc1 ; Cc1ncsc1-c1ccc([C@@H](C)NC(=O)[C@@H]2C[C@@H](O)CN2C(=O)C(c2cc(OCCN)no2)C(C)C)cc1]

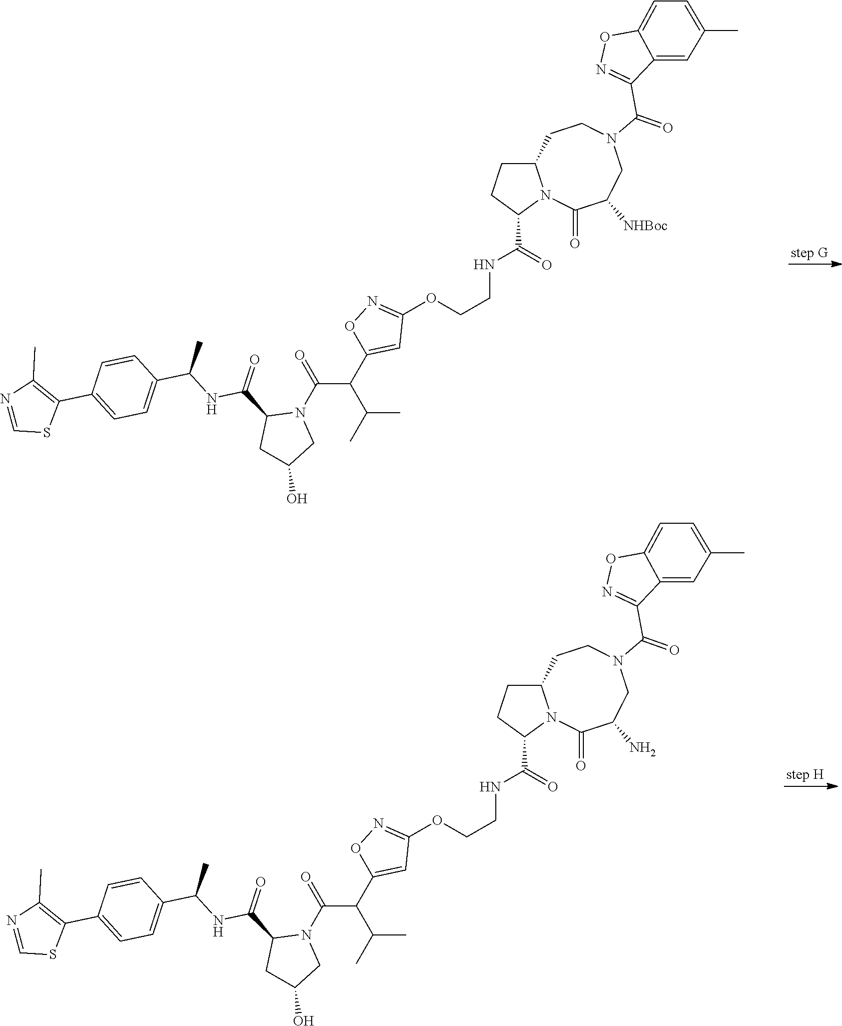 [SMILES: Cc1ccc2onc(C(=O)N3CC[C@H]4CC[C@@H](C(=O)NCCOc5cc(C(C(=O)N6C[C@H](O)C[C@H]6C(=O)N[C@H](C)c6ccc(-c7scnc7C)cc6)C(C)C)on5)N4C(=O)[C@@H](NC(=O)OC(C)(C)C)C3)c2c1 ; Cc1ccc2onc(C(=O)N3CC[C@H]4CC[C@@H](C(=O)NCCOc5cc(C(C(=O)N6C[C@H](O)C[C@H]6C(=O)N[C@H](C)c6ccc(-c7scnc7C)cc6)C(C)C)on5)N4C(=O)[C@@H](N)C3)c2c1]

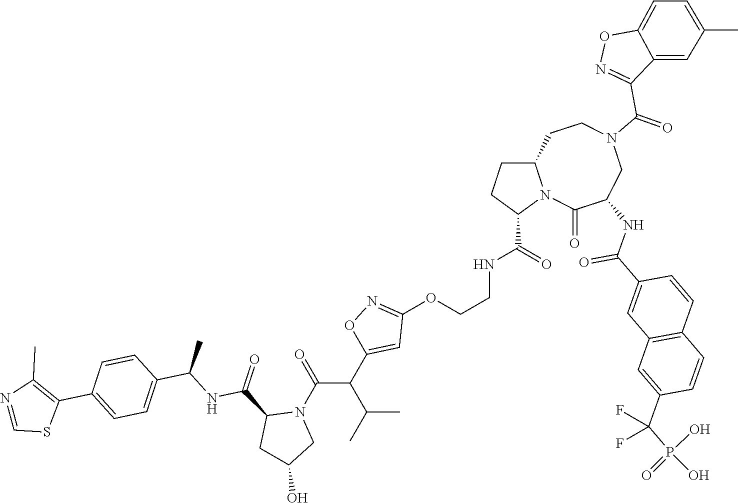 [SMILES: Cc1ccc2onc(C(=O)N3CC[C@H]4CC[C@@H](C(=O)NCCOc5cc(C(C(=O)N6C[C@H](O)C[C@H]6C(=O)N[C@H](C)c6ccc(-c7scnc7C)cc6)C(C)C)on5)N4C(=O)[C@@H](NC(=O)c4ccc5ccc(C(F)(F)P(=O)(O)O)cc5c4)C3)c2c1]

### STEP A: 2-((tert-butoxycarbonyl)amino)ethyl 4-methylbenzenesulfonate

To solution of tert-butyl (2-hydroxyethyl)carbamate (500 mg, 3.11 mmol, 1.0 eq.), TEA (941 mg, 9.32 mmol, 3.0 eq.) and DMAP (38 mg, 0.31 mmol, 0.1 eq.) in DCM (11 mL) was added TsCl (891 mg, 4.67 mmol, 1.5 eq.) at 0° C., and the resulting mixture was warmed up to room temperature and stirred for for 3 hrs under N₂. After completion, the reaction mixture was quenched by adding H₂O (10 mL), then extracted with EtOAc (15 mL×3). The organic layers were combined and washed with brine (15 mL), dried over anhydrous Na₂SO₄, and concentrated under reduced pressure. The residue was purified by flash column chromatography on silica gel to afford 2-((tert-butoxycarbonyl)amino)ethyl 4-methylbenzenesulfonate (500 mg, 1.59 mmol, 51%) as a yellow oil. LC-MS (ESI) m/z=316 [M+H]⁺

### STEP B: methyl 2-(3-(2-((tert-butoxycarbonyl)amino)ethoxy)isoxazol-5-yl)-3-methylbutanoate

To a solution of methyl 2-(3-hydroxyisoxazol-5-yl)-3-methylbutanoate (198 mg, 0.63 mmol, 1.0 eq.) and K₂CO₃ (261 mg, 1.89 mmol, 3.0 eq.) in DMF (10 mL) was added 2-((tert-butoxycarbonyl)amino)ethyl 4-methylbenzenesulfonate (400 mg, 1.26 mmol, 2.0 eq.) at room temperature. The resulting mixture was stirred at room temperature for 2 hrs. After completion, the reaction mixture was purified by C18 column chromatography to give methyl 2-(3-(2-(tert-butoxycarbonyl)amino)ethoxy)isoxazol-5-yl)-3-methylbutanoate (200 mg, 0.58 mmol, 93%) as a white solid. LC-MS (ESI) m/z=343 [M+H]⁺

### STEP C: 2-(3-(2-((tert-butoxycarbonyl)amino)ethoxy)isoxazol-5-yl)-3-methylbutanoic acid

To a stirring solution of methyl 2-(3-(2-((tert-butoxycarbonyl)amino)ethoxy)isoxazol-5-yl)-3-methylbutanoate (200 mg, 0.58 mmol, 1.0 eq) in MeOH/H₂O (5 mL/0.5 mL) was added LiOH H₂O (75 mg, 1.74 mmol, 3.0 eq.) at room temperature. The reaction mixture was stirred at room temperature for 4 hrs. The resulting solution was acidified to pH-~5 with 1 N HCl aqueous solution and concentrated under reduced pressure. The residue was purified by C18 column chromatography to afford 2-(3-(2-((tert-butoxycarbonyl)amino)ethoxy)isoxazol-5-yl)-3-methylbutanoic acid (180 mg, 0.55 mmol, 95%) as a white solid. LCMS (ESI): m/z=329 [M+H]⁺.

### STEP D: tert-butyl (2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamate

To a solution of 2-(3-(2-((tert-butoxycarbonyl)amino)ethoxy)isoxazol-5-yl)-3-methylbutanoic acid (180 mg 0.55 mmol, 1.0 eq.) and (2S,4R)-4-hydroxy-N—((R)-1-(4-(4-methylthiazol-5-yl)phenyl)ethyl)pyrrolidine-2-carboxamide (182 mg, 0.55 mmol, 1.0 eq.) in DMF (3 mL) were added HATU (230 mg, 0.61 mmol, 1.1 eq.) and DIEA (213 mg, 1.65 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl (2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamate (250 mg, 0.39 mmol, 71%) as a white solid. LCMS (ESI): m/z=642 [M+H]⁺.

### STEP E: (2S,4R)-1-(2-(3-(2-aminoethoxy)isoxazol-5-yl)-3-methylbutanoyl)-4-hydroxy-N—((R)-1-(4-(4-methylthiazol-5-yl)phenyl)ethyl)pyrrolidine-2-carboxamide

To a solution of tert-buty 1 (2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamate (250 mg, 0.39 mmol, 1.0 eq.) in DCM (3 mL) was added TFA (1 mL), and the resulting mixture was stirred at room temperature for 2 hrs. After completion, the reaction mixture was concentrated under reduced pressure to give crude (2S,4R)-1-(2-(3-(2-aminoethoxy)isoxazol-5-yl)-3-methylbutanoyl)-4-hydroxy-N—((R)-1-(4-(4-methylthiazol-5-yl)phenyl)ethyl)pyrrolidine-2-carboxamide (200 mg, quant.) (TFA salt) as a white solid, which was used in next step directly without further purification. LCMS (ESI): m/z=542 [M+H]⁺.

### STEP F: tert-butyl ((5S,8S,10aR)-8-((2-((j-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamoyl)-3-(5-methylbenzo[d]isoxazole-3-carbonyl)-6-oxodecahydropyrrolo[1,2-a][1,5]diazocin-5-yl)carbamate

To a solution of (2S,4R)-1-(2-(3-(2-aminoethoxy)isoxazol-5-yl)-3-methylbutanoyl)-4-hydroxy-N—((R)-1-(4-(4-methylthiazol-5-yl)phenyl)ethyl)pyrrolidine-2-carboxamide (200 mg, 0.37 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (180 mg, 0.37 mmol, 1.0 eq.) in DMF (3 mL) were added HATU (155 mg, 0.41 mmol, 1.1 eq.) and DIEA (143 mg, 1.11 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-((2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamoyl)-3-(5-methylbenzo[d]isoxazole-3-carbonyl)-6-oxodecahydropyrrolo[1,2-a][1,5]diazocin-5-yl)carbamate (200 mg, 0.20 mmol, 54%) as a white solid. LCMS (ESI): m/z=1010 [M+H]⁺.

### STEP G: (5S,8S,10aR)-5-amino-N-(2-((S-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-3-(5-methylbenzo[d]isoxazole-3-carbonyl)-6-oxodecahydropyrrolo[1,2-a][1,5]diazocine-8-carboxamide

To a solution of tert-butyl ((5S,8S,10aR)-8-((2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamoyl)-3-(5-methylbenzo[d]isoxazole-3-carbonyl)-6-oxodecahydropyrrolo[1,2-a][1,5]diazocin-5-yl)carbamate (200 mg, 0.20 mmol, 1.0 eq.) in DCM (3 mL) was added TFA (1 mL), and the resulting mixture was stirred at room temperature for 2 hrs. After completion, the reaction mixture was concentrated under reduced pressure to give crude (5S,8S,10aR)-5-amino-N-(2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-3-(5-methylbenzo[d]isoxazole-3-carbonyl)-6-oxodecahydropyrrolo[1,2-a][1,5]diazocine-8-carboxamide (45 mg, quant.) (TFA salt) as a white solid, which was used in next step directly without further purification. LCMS (ESI): m/z=910 [M+H]⁺.

### STEP H: (difluoro(7-(((5S,8S,10aR)-8-((2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid

To a solution of (5S,8S,10aR)-5-amino-N-(2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-3-(5-methylbenzo[d]isoxazole-3-carbonyl)-6-oxodecahydropyrrolo[1,2-a][1,5]diazocine-8-carboxamide (45 mg, 49.5 μmol, 1.0 eq.) and (difluoro(7-((perfluorophenoxy)carbonyl)naphthalen-2-yl)methyl)phosphonic acid (23.2 mg, 49.5 μmol, 1.0 eq.) in DMF (2 mL) was added DIEA (32 mg, 248 μmol, 5.0 eq.) at room temperature. The resulting mixture was stirred at room temperature for additional 2 hrs. After completion, the reaction mixture was purified by RP-prep HPLC to give (difluoro(7-(((5S,8S,10aR)-8-((2-((5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid (Compound 32, 36.7 mg, 30.8 μmol, 62%) as a white solid.

¹H NMR (400 MHz, DMSO-d6) δ 9.05-8.88 (m, 1H), 8.86-8.64 (m, 1H), 8.58 (s, 1H), 8.46-8.30 (m, 1H), 8.28-8.13 (m, 2H), 8.11-7.90 (m, 4H), 7.79-7.68 (m, 1H), 7.67-7.59 (m, 1H), 7.57-7.33 (m, 5H), 6.18-5.78 (m, 1H), 5.39-5.07 (m, 1H), 5.02-4.73 (m, 1H), 4.53-4.24 (m, 5H), 4.16-4.04 (m, 3H), 4.00-3.89 (m, 4H), 3.50-3.18 (m, 4H), 2.47-2.34 (m, 6H), 2.33-1.64 (m, 9H), 1.49-1.27 (m, 3H), 1.07-0.68 (m, 5H), 0.51-0.28 (m, 1H).

LCMS (ESI): m/z=1194.5 [M+H]⁺.

### Preparation of (difluoro(7-(((5S,8S,10aR)-8-(4-(5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid (Compound 33)

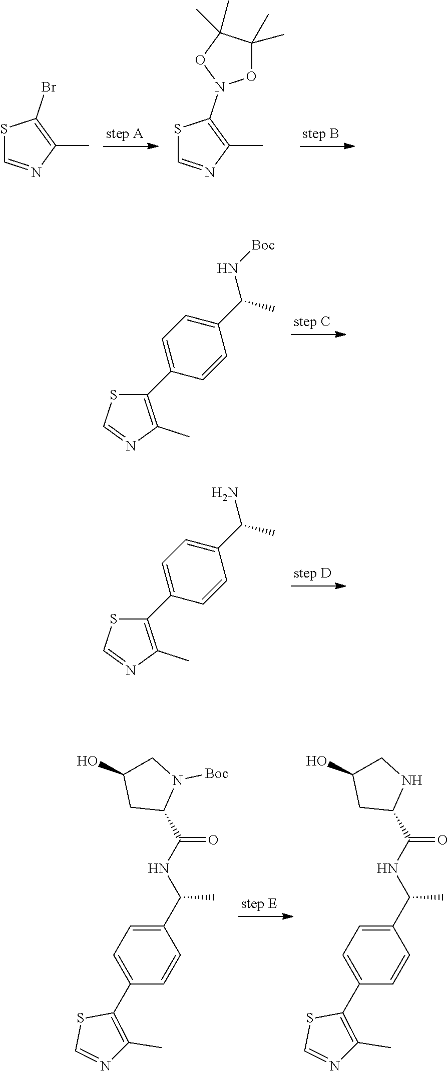

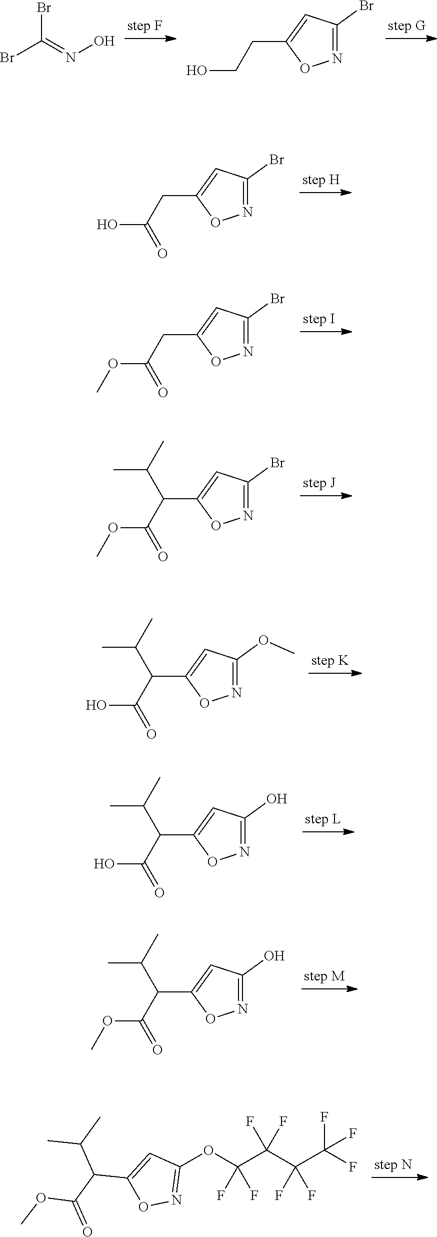

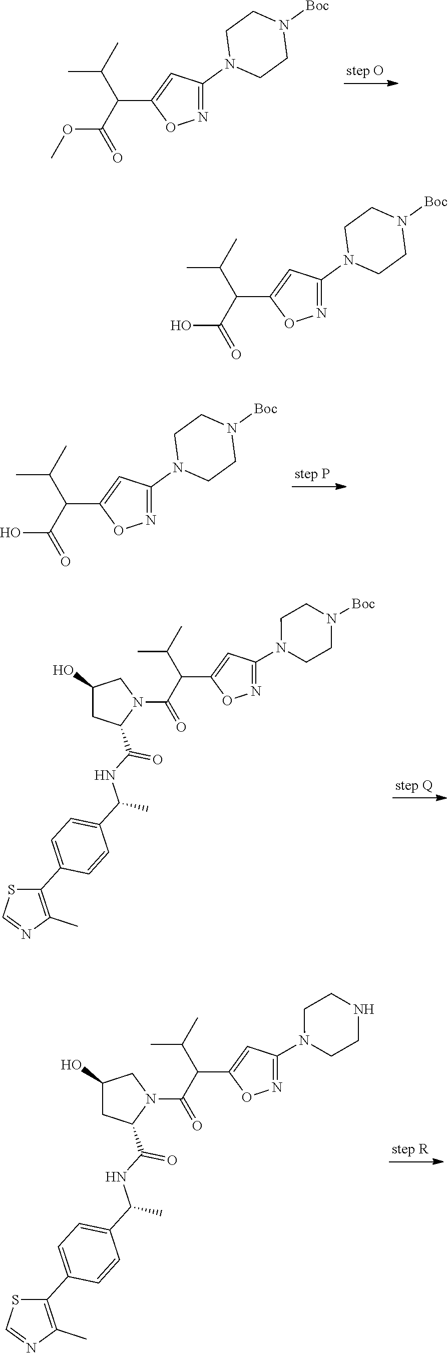 [SMILES: COC(=O)C(c1cc(N2CCN(C(=O)OC(C)(C)C)CC2)no1)C(C)C ; CC(C)C(C(=O)O)c1cc(N2CCN(C(=O)OC(C)(C)C)CC2)no1 ; CC(C)C(C(=O)O)c1cc(N2CCN(C(=O)OC(C)(C)C)CC2)no1 ; Cc1ncsc1-c1ccc([C@@H](C)NC(=O)[C@@H]2C[C@@H](O)CN2C(=O)C(c2cc(N3CCN(C(=O)OC(C)(C)C)CC3)no2)C(C)C)cc1 ; Cc1ncsc1-c1ccc([C@@H](C)NC(=O)[C@@H]2C[C@@H](O)CN2C(=O)C(c2cc(N3CCNCC3)no2)C(C)C)cc1]

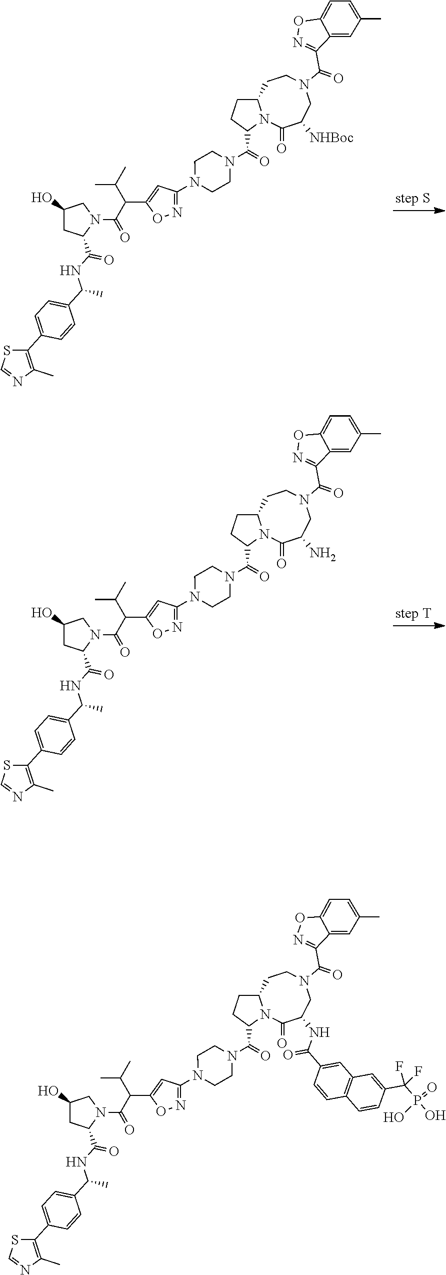

### STEP A: 4-methyl-5-(4,4,5,5-tetramethyl-1,3,2-dioxaborolan-2-yl)thiazole

To solution of 5-bromo-4-methylthiazole (2 g, 11.3 mmol, 1.0 eq.), 4,4,4′,4′,5,5,5′,5′-octamethyl-2,2′-bi(1,3,2-dioxaborolane) (3.44 g, 13.6 mmol, 1.2 eq.), and KOAc (3.29 g, 33.9 mmol, 3.0 eq.) in dioxane (110 mL), was added Pd(dppf)Cl₂ (820 mg, 1.13 mmol, 0.1 eq), and the resulting mixture was stirred at 90° C. for 3 hrs under N₂. After completion, the reaction mixture was filtered and the filtrate was concentrated under reduced pressure to afford crude 4-methyl-5-(4,4,5,5-tetramethyl-1,3,2-dioxaborolan-2-yl)thiazole (2.5 g, quant.) as an oil, which was used in next step directly without further purification. LCMS (ESI): m/z=226 [M+H]⁺.

### STEP B: tert-butyl (R)-(1-(4-(4-methylthiazol-5-yl)phenyl)ethyl)carbamate

To solution of tert-butyl (R)-(1-(4-bromophenyl)ethyl)carbamate (2.2 g, 7.4 mmol, 1.0 eq.), 4-methyl-5-(4,4,5,5-tetramethyl-1,3,2-dioxaborolan-2-yl)thiazole (2.5 g, 11.1 mmol, 1.5 eq.) and K₂CO₃ (3.06 g, 22.2 mmol, 3.0 eq.) in dioxane/H₂O (70 mL/14 mL), was added Pd(dppf)Cl₂ (537 mg, 0.74 mmol, 0.1 eq.), and the resulting mixture was stirred at 90° C. for 12 hrs under N₂. After completion, the reaction mixture was concentrated under reduced pressure. The residue was purified by flash column chromatography on silica gel to afford tert-butyl (R)-(1-(4-(4-methylthiazol-5-yl)phenyl)ethyl)carbamate (1.5 g, 4.72 mmol, 64%) as a white solid. LCMS (ESI): m/z=319 [M+H]⁺.

### STEP C: (R)-1-(4-(4-methylthiazol-5-yl)phenyl)ethan-1-amine

To a solution of tert-butyl (R)-1-(4-(4-methylthiazol-5-yl)phenyl)ethyl)carbamate (1.5 g, 4.72 mmol, 1.0 eq.) in DCM (10 mL) was added TFA (3 mL), and the resulting mixture was stirred at room temperature for 2 hrs. After completion, the reaction mixture was concentrated under reduced pressure to give crude (R)-1-(4-(4-methylthiazol-5-yl)phenyl)ethan-1-amine (800 mg, quant.) (TFA salt) as a white solid, which was used in next step directly without further purification. LCMS (ESI): m/z=219 [M+H]⁺.

### STEP D: tert-butyl (2S,4R)-4-hydroxy-2-(((R)-1-(4-(4-methylthiazol-5-yl)phenyl)ethyl)carbamoyl)pyrrolidine-1-carboxylate

To a solution of (R)-1-(4-(4-methylthiazol-5-yl)phenyl)ethan-1-amine (800 mg, 3.65 mmol, 1.0 eq.) and (2S,4R)-1-(tert-butoxycarbonyl)-4-hydroxypyrrolidine-2-carboxylic acid (843 mg, 3.65 mmol, 1.0 eq.) in DMF (40 mL) were added HOBt (493 mg, 3.65 mmol, 1.0 eq.), EDCI (701 mg, 3.65 mmol, 1.0 eq.), and DIEA (1.41 g, 11.0 mmol, 3.0 eq.), and the resulting mixture was stirred at room temperature for 2 hrs. After completion, the reaction was diluted with H₂O (40 mL) and extracted with EtOAc (50 mL×3). The organic layers were combined and washed with brine (40 mL), dried over anhydrous Na₂SO₄, and concentrated under reduced pressure. The residue was purified b) flash column chromatography on silica gel to afford tert-butyl (2S,4R)-4-hydroxy-2-(((R)-1-(4-(4-methylthiazol-5-yl)phenyl)ethyl)carbamoyl)pyrrolidine-1-carboxylate (1 g, 2.32 mmol, 64%) as a white solid. LCMS (ESI): m/z=432 [M+H]⁺.

### STEP E: (2S,4R)-4-hydroxy-N—((R)-1-(4-(4-methylthiazol-5-yl)phenyl)ethyl)pyrrolidine-2-carboxamide

To a solution of tert-butyl (2S,4R)-4-hydroxy-2-(((R)-1-(4-(4-methylthiazol-5-yl)phenyl)ethyl)carbamoyl)pyrrolidine-1-carboxylate (1 g, 2.32 mmol, 1.0 eq.) in DCM (3 mL) was added TFA (1 mL), and the resulting mixture was stirred at room temperature for 2 hrs. After completion, the reaction mixture was concentrated under reduced pressure to give crude (2S,4R)-4-hydroxy-N—((R)-1(4-(4-methylthiazol-5-yl)phenyl)ethyl)pyrrolidine-2-carboxamide (80) mg, quant.) (TFA salt) as a white solid, which was used in next step directly without further purification. LCMS (ESI): m/z=332 [M+H]⁺.

### STEP F: 2-(3-bromoisoxazol-5-yl)ethan-1-ol

To a stirring solution of but-3-yn-1-ol (14 g, 200 mmol, 4.0 eq.) and KHCO₃ (15 g, 150 mmol, 3.0 eq.) in EtOAc/water (100/10 mL) was added a solution of hydroxycarbonimidic dibromide (10 g, 49.3 mmol, 1.0 eq.) in EtOAc (25 mL) dropwise at room temperature. The resulting solution was stirred at room temperature for 16 hrs and then diluted with water. The organic laver was separated and the aqueous laver was extracted with EtOAc. The combined organic layers were washed with brine (150 mL), dried over anhydrous Na₂SO₄ and concentrated under reduced pressure. The residue was purified by flash chromatography on silica gel column to afford 2-(3-bromoisoxazol-5-yl)ethan-1-ol (8.0 g, 41.9 mmol, 85%) as a yellow oil. LCMS (ESI): m/z=192 [M+H]⁻.

### STEP G: 2-(3-bromoisoxazol-5-yl)acetic acid

To a stirring solution of 2-(3-bromoisoxazol-5-yl)ethan-1-ol (8.0 g, 41.9 mmol, 1.0 eq.) in acetone (95 mL) was added Jones reagent (40 mL) dropwise at 0° C. The resulting solution was stirred at 25° C. for 12 hrs. The mixture was diluted with water and extracted with EtOAc. The combined organic layers were washed with brine, dried over anhydrous Na₂SO₄ and concentrated under reduced pressure to afford 2-(3-bromoisoxazol-5-yl)acetic acid (7.5 g, 36.6 mmol, 87%) as a yellow oil. LCMS (ESI): m/z=206 [M+H]⁺.

### STEP H: methyl 2-(3-bromoisoxazol-5-yl)acetate

A solution of 2-(3-bromoisoxazol-5-yl)acetic acid (7.5 g, 36.6 mmol, 1.0 eq.) and concentrated H₂SO₄ (0.6 mL, 10.9 mmol, 0.3 eq.) in methanol (150 mL) was stirred at 70° C. for 2 hrs. The resulting solution was concentrated under reduced pressure. The residue was diluted with water and extracted with ethyl acetate. The combined organic layers were washed with brine, dried over anhydrous sodium sulfate and concentrated under reduced pressure. The residue was purified by flash chromatography on silica gel column to afford methyl 2-(3-bromoisoxazol-5-yl)acetate (7.0 g, 32.0 mmol, 87%) as a yellow oil. LCMS (ESI): m/z=220 [M+H]⁺.

### STEP I: methyl 2-(3-bromoisoxazol-5-yl)-3-methylbutanoate

To a stirring solution of methyl 2-(3-bromoisoxazol-5-yl)acetate (6.8 g, 31.1 mmol, 1.0 eq) and t-BuOK (5.22 g, 46.7 mmol, 1.5 eq.) in THF (70 mL) was added 2-iodopropane (6.9 g, 40.4 mmol, 1.3 eq.) dropwise at 0° C. The reaction mixture was stirred at room temperature for 16 hrs and then quenched with ice-water. The resulting solution was extracted with ethyl acetate. The combined organic layers were washed with brine, dried over anhydrous sodium sulfate and concentrated under reduced pressure. The residue was purified by flash chromatography on silica gel to afford (4.1 g, 15.7 mmol, 51%) as a yellow oil.

LCMS (ESI): m/z=262 [M+H]⁺.

### STEP J: 2-(3-methoxyisoxazole-5-yl)-3-methylbutanoic acid

To a stirring solution of methyl 2-(3-bromoisoxazol-5-yl)-3-methylbutanoate (2.8 g, 10.7 mmol, 1.0 eq.) in methanol (30 mL) was added KOH (5.99 g, 107 mmol, 10.0 eq.) at room temperature. The reaction mixture was stirred at reflux for 4 hrs, and then allowed to cool to room temperature. The resulting solution was acidified to pH~5 with 1 N HCl aqueous solution and concentrated under reduced pressure. The residue was purified by C18 column chromatography to afford tert-butyl 4-(3-amino-4-nitrophenyl)piperazine-1-carboxylate (1.4 g, 7 mmol, 65%) as colorless thick oil. LCMS (ESI): m/z=200 [M+H]⁺.

### STEP K: 2-(3-hydroxyisoxazol-5-yl)-3-methylbutanoic acid

A solution of tert-butyl 4-(3-amino-4-nitrophenyl)piperazine-1-carboxylate (1.4 g, 7.0 mmol, 1.0 eq.) in HOAc (5 mL) and HBr (5 mL, 40% in water) was stirred at 60° C. for 16 hrs. The resulting mixture was concentrated under reduced pressure. The residue was purified by C18 column chromatography to afford 2-(3-hydroxyisoxazol-5-yl)-3-methylbutanoic acid (1.3 g, 7.0 mmol, 98%) as a yellow solid. LCMS (ESI): m/z=186 [M+H]⁺.

### STEP L: methyl 2-(3-hydroxyisoxazol-5-yl)-3-methylbutanoate

A solution of 2-(3-hydroxyisoxazol-5-yl)-3-methylbutanoic acid (1.7 g, 9.19 mmol, 1.0 eq.) and SOCl₂ (2.8 mL, 41.4 mmol, 4.5 eq.) in methanol (6 mL) was stirred at room temperature for 3 hrs. The solvent was removed under reduced pressure. The residue was diluted with water and extracted with ethyl acetate. The combined organic lavers were washed with brine, dried over anhydrous sodium sulfate and concentrated under reduced pressure. The residue was purified by flash chromatography on silica gel to afford methyl 2-(3-hydroxyisoxazol-5-yl)-3-methylbutanoate (1.8 g, 9.05 mmol, 98%) as a yellow oil. LCMS (ESI): m/z=200 [M+H]⁺.

### STEP M: methyl 3-methyl-2-(3-(((perfluorobutyl)sulfonyl)oxy)isoxazol-5-yl)butanoate

To a solution of methyl 2-(3-hydroxyisoxazol-5-yl)-3-methylbutanoate (800 mg, 4.02 mmol, 1.0 eq.) and K₂CO₃ (1.66 g, 12.1 mmol, 3.0 eq.) in ACN (20 mL) was added 1,1,2,2,3,3,4,4,4-nonafluorobutane-1-sulfonyl fluoride (1.82 g, 6.03 mmol, 1.5 eq.). The resulting mixture was stirred at room temperature for 12 hrs. After completion, the reaction mixture was concentrated under reduced pressure. The residue was purified by flash column chromatography on silica gel to afford methyl 3-methyl-2-(3-(((perfluorobutyl)sulfonyl)oxy)isoxazol-5-yl)butanoate (1.3 g, 2.70 mmol, 67%) as a white solid. LCMS (ESI): m/z=482 [M+H]⁺.

### STEP N: tert-butyl 4-(5-(1-methoxy-3-methyl-1-oxobutan-2-yl)isoxazol-3-yl)piperazine-1-carboxylate

A solution of methyl 3-methyl-2-(3-(((perfluorobutyl)sulfonyl)oxy)isoxazol-5-yl)butanoate (1.0 g, 2.08 mmol, 1.0 eq.) and tert-butyl piperazine-1-carboxylate (1.55 g, 8.32 mmol, 4.0 eq.) in DMF (25 mL) was stirred at 100° C. for 3 hrs. After completion, the reaction mixture was diluted with H₂O (20 mL) and extracted with EtOAc (20 mL×3). The organic layers were combined and washed with brine (20 mL), dried over anhydrous Na₂SO₄, and concentrated under reduced pressure. The residue was purified by flash column chromatography on silica gel to afford tert-butyl 4-(5-(1-methoxy-3-methyl-1-oxobutan-2-yl)isoxazol-3-yl)piperazine-1-carboxylate (400 mg, 1.09 mmol, 52%) as a white solid. LCMS (ESI): m/z=368 [M+H]⁺.

### STEP O: 2-(3-(4-(tert-butoxycarbonyl)piperazin-1-yl)isoxazol-5-yl)-3-methylbutanoic acid

To a solution of tert-butyl 4-(5-(1-methoxy-3-methyl-1-oxobutan-2-yl)isoxazol-3-yl)piperazine-1-carboxylate (100 mg, 0.27 mmol, 1.0 eq.) in MeOH/THF/H₂O (1/1/0.1 mL) was added LiOH (23 mg, 0.54 mmol, 2.0 eq.). The resulting mixture was stirred at room temperature for 2 hrs. After completion, the reaction mixture was acidified to pH~5 with 1 N HCl aqueous solution and concentrated under reduced pressure. The residue was purified by C18 column chromatography to afford 2-(3-(4-(tert-butoxycarbonyl)piperazin-1-yl)isoxazol-5-yl)-3-methylbutanoic acid (57 mg, 0.16 mmol, 60%) as colorless thick oil. LCMS (ESI): m/z=354 [M+H]⁺.

### STEP P: tert-butyl 4-(5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carboxylate

To a solution of 2-(3-(4-(tert-butoxycarbonyl)piperazin-1-yl)isoxazol-5-yl)-3-methylbutanoic acid (57 mg, 0.16 mmol, 1.0 eq.) and (2S,4R)-4-hydroxy-N—((R)-1-(4-(4-methylthiazol-5-yl)phenyl)ethyl)pyrrolidine-2-carboxamide (53 mg, 0.16 mmol, 1.0 eq.) in DMF (3 mL) were added HATU (67 mg, 0.18 mmol, 1.1 eq.) and DIEA (62 mg, 0.48 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl 4-(5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carboxylate (60 mg, 90.1 μmol, 56%) as a white solid. LCMS (ESI): m/z=667 [M+H]⁺.

### STEP Q: (2S,4R)-4-hydroxy-1-(3-methyl-2-(3-(piperazin-1-yl)isoxazol-5-yl)butanoyl)-N—((R)-1-(4-(4-methylthiazol-5-yl)phenyl)ethyl)pyrrolidine-2-carboxamide

To a solution of tert-butyl 4-(5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carboxylate (60 mg, 90.1 μmol, 1.0 eq.) in DCM (3 mL) was added TFA (1 mL), and the resulting mixture was stirred at room temperature for 2 hrs. After completion, the reaction mixture was concentrated under reduced pressure to give crude (2S,4R)-4-hydroxy-1-(3-methyl-2-(3-(piperazin-1-yl)isoxazol-5-yl)butanoyl)-N—((R)-1-(4-(4-methylthiazol-5-yl)phenyl)ethyl)pyrrolidine-2-carboxamide (54 mg, quant.) (TFA salt) as a white solid, which was used in next step directly without further purification. LCMS (ESI): m/z=567 [M+H]⁺.

### STEP R: tert-butyl ((5S,8S,10aR)-8-(4-(5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carbonyl)-3-(5-methylbenzo[d]isoxazole-3-carbonyl)-6-oxodecahydropyrrolo[1,2-a][1,5]diazocin-5-yl)carbamate

To a solution of (2S,4R)-4-hydroxy-1-(3-methyl-2-(3-(piperazin-1-yl)isoxazol-5-yl)butanoyl)-N—((R)-1-(4-(4-methylthiazol-5-yl)phenyl)ethyl)pyrrolidine-2-carboxamide (54 mg, 96 μmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (47 mg, 96 μmol, 1.0 eq.) in DMF (3 mL) were added HATU (40 mg, 106 μmol, 1.1 eq.) and DIEA (41 mg, 0.32 mmol, 3.0 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-(4-(5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carbonyl)-3-(5-methylbenzo[d]isoxazole-3-carbonyl)-6-oxodecahydropyrrolo[1,2-a][1,5]diazocin-5-yl)carbamate (50 mg, 48 μmol. %) as a white solid LCMS (ESI): m/z=1035 [M+H]⁺.

### STEP S: (2S,4R)-1-(2-(3-(4-((5S,8S,10aR)-5-amino-3-(S-methylbenzo[d]isoxazole-3-carbonyl)-6-oxodecahydropyrrolo[1,2-a][1,5]diazocine-8-carbonyl)piperazin-1-yl)isoxazol-5-yl)-3-methylbutanoyl)-4-hydroxy-N—((R)-1-(4-(4-methylthiazol-5-yl)phenyl)ethyl)pyrrolidine-2-carboxamide

To a solution of tert-butyl ((5S,8S,10aR)-8-(4-(5-(1-((2S,4R)-4-hydroxyl-2-(((R)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carbonyl)-3-(5-methylbenzo[d]isoxazole-3-carbonyl)-1-oxodecahydropyrrolo[1,2-a][1,5]diazocin-5-yl)carbamate (50 mg, 48 μmol, 1.0 eq.) in DCM (3 mL) was added TFA (1 mL), and the resulting mixture was stirred at room temperature for 2 hrs. After completion, the reaction mixture was concentrated under reduced pressure to give crude (2S,4R)-1-(2-(3-(4-((5S,8S,10aR)-5-amino-3-(5-methylbenzo[d]isoxazole-3-carbonyl)-6-oxodecahydropyrrolo[1,2-a][1,5]diazocine-8-carbonyl)piperazin-1-yl)isoxazol-5-yl)-3-methylbutanoyl)-4-hydroxy-N—((R)-1-(4-(4-methylthiazol-5-yl)phenyl)ethyl)pyrrolidine-2-carboxamide (48 mg, quant.) (TFA salt) as a white solid, which was used in next step directly without further purification. LCMS (ESI): m/z=935 [M+H]⁺.

### STEP T: (difluoro(7-(((5S,8S,10aR)-8-(4-(5-(1-((2S,4R)-4-hydroxy-2-(((R)-1-(4-(4-methylthiazol-5-yl)phenyl)ethyl)carbamoyl)-pyrrolidin-1-yl)-3-methyl-1-oxobutan-2-yl)isoxazol-3-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid

To a solution of (2S,4R)-1-(2-(3-(4-((5S,8S,10aR)-5-amino-3-(5-methylbenzo[d]isoxazole-3-carbonyl)-6-oxodecahydropyrrolo[1,2-a][1,5]diazocine-8-carbonyl)piperazin-1-yl)isoxazol-5-yl)-3-methylbutanoyl)-4-hydroxy-N—((R)-1-(4-(4-methylthiazol-5-yl)phenyl)ethyl)pyrrolidine-2-carboxamide (48 mg, 51.0 μmol, 1.0 eq.) and (difluoro(7-((perfluorophenoxy)carbonyl)naphthalen-2-yl)methyl)phosphonic acid (22 mg, 51.0 μmol, 1.0 eq.) in DMF (2 mL) was added DIEA (33 mg, 255 μmol, 5.0 eq.) at room temperature. The resulting mixture was stirred at room temperature for additional 2 hrs. After completion, the reaction mixture was purified by RP-prep HPLC to give (difluoro(7-(((5S,8S,10aR)-8-(4-(5-(1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)methyl)phosphonic acid (Compound 33, 25.5 mg, 20.9 μmol, 50%) as a white solid.

¹H NMR (400 MHz, DMSO-d6) δ 9.02-8.76 (m, 2H), 8.72-8.55 (m, 1H), 8.51-8.27 (m, 1H), 8.26-7.80 (m, 5H), 7.78-7.62 (m, 2H), 7.57-7.24 (m, 5H), 6.22-6.04 (m, 1H), 5.36-5.14 (m, 2H), 4.95-4.89 (m, 1H), 4.82-4.77 (m, 1H), 4.51-4.47 (m, 1H), 4.39-4.34 (m, 1H), 4.29-4.23 (m, 1H), 4.09-3.98 (m, 2H), 3.88-3.80 (m, 1H), 3.71-3.20 (m, 8H), 3.16-2.76 (m, 4H), 2.48-2.32 (m, 6H), 2.31-2.03 (m, 4H), 2.02-1.70 (m, 4H), 1.70-1.50 (m, 1H), 1.46-1.24 (m, 3H), 1.01-0.33 (m, 6H). (TFA salt).

LCMS (ESI): m/z=1217.1 [M−H]⁻.

### Preparation of ((7-(((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 34)

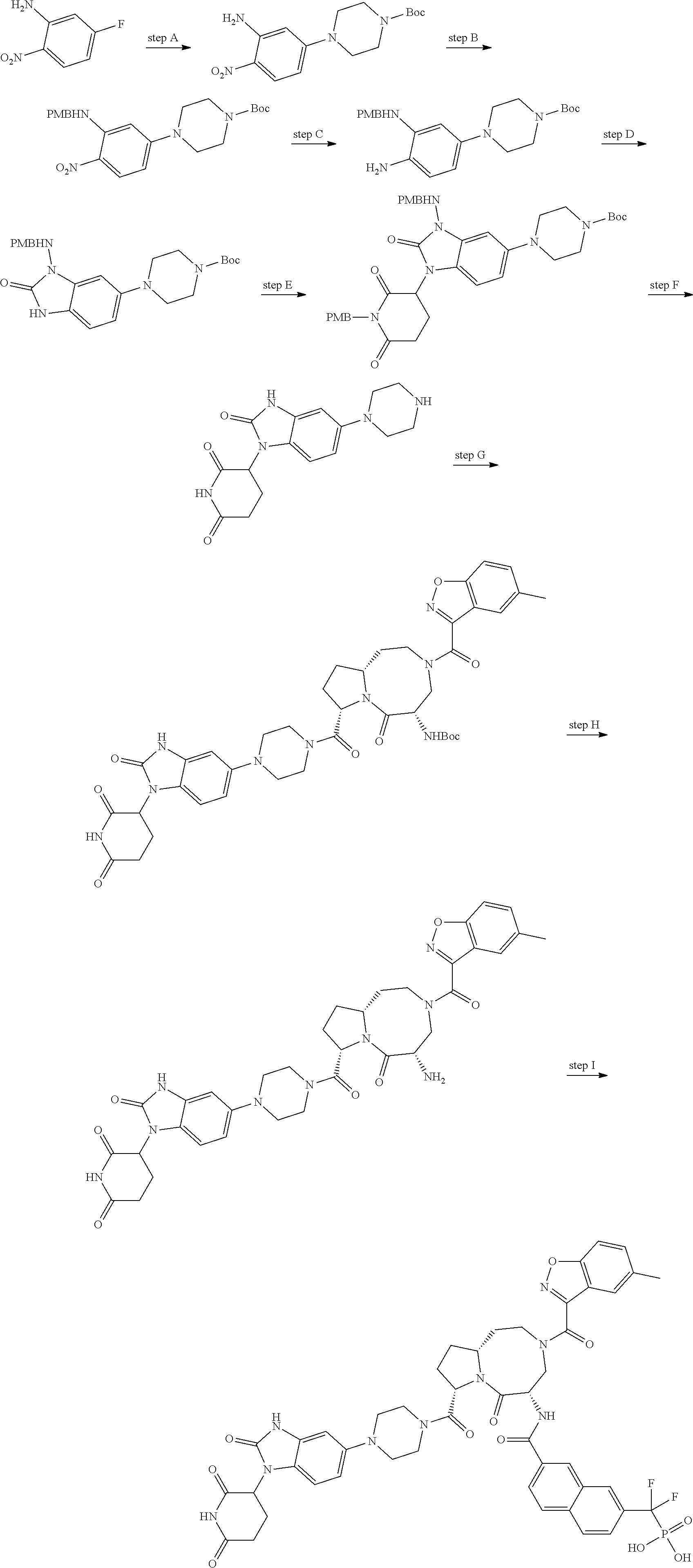

### STEP A: tert-butyl 4-(3-amino-4-nitrophenyl)piperazine-1-carboxylate

To a solution of 5-fluoro-2-nitroaniline (2.0 g, 12.8 mmol, 1.0 eq.) and tert-butyl piperazine-1-carboxylate (2.38 g, 12.8 mmol, 1.0 eq.) in DMF (100 mL) was added K₂CO₃ (5.30 g, 38.4 mmol, 3.0 eq.). The resulting mixture was stirred at 80° C. for 12 hrs. After completion, the reaction mixture was diluted with H₂O (100 mL) and extracted with EtOAc (100 mL×3). The organic layers were combined and washed with brine (100 mL), dried over anhydrous Na₂SO₄, and concentrated under reduced pressure. The residue was purified by flash column chromatography on silica gel to afford tert-butyl 4-(3-amino-4-nitrophenyl)piperazine-1-carboxylate (3 g, 9.32 mmol, 73%) as a white solid. LCMS (ESI): m/z=323 [M+H]⁺.

### STEP B: tert-butyl 4-(3-((4-methoxybenzyl)amino)-4-nitrophenyl)piperazine-1-carboxylate

To a solution of tert-butyl 4-(3-amino-4-nitrophenyl)piperazine-1-carboxylate (3 g, 9.32 mmol, 1.0 eq.) in DMF (50 mL) was added NaH (536 mg, 14.0 mmol, 1.5 eq) at 0° C. The mixture was stirred for additional 30 min at 0° C., then PMBCl (1.60 g, 10.3 mmol, 1.1 eq.) was added drop-wisely. After complete addition of PBMCl, the reaction mixture was allowed to warm to room temperature and stirred for 14 hrs. The reaction mixture was quenched by adding H₂O (20 mL), then extracted with EtOAc (150 mL×3). The organic layers were combined and washed with brine (150 mL), dried over anhydrous Na₂SO₄, and concentrated under reduced pressure. The residue was purified by flash column chromatography on silica gel to afford tert-butyl 4-(3-((4-methoxybenzyl)amino)-4-nitrophenyl)piperazine-1-carboxylate (3 g, 6.79 mmol, 73%) as a white solid. LC-MS (ESI) m/z=443 [M+H]⁺

### STEP C: tert-butyl 4-(4-amino-3-((4-methoxybenzyl)amino)phenyl)piperazine-1-carboxylate

To a solution of tert-butyl 4-(3-((4-methoxybenzyl)amino)-4-nitrophenyl)piperazine-1-carboxylate (3 g, 6.79 mmol, 1.0 eq) and HOAc (2.04 g, 34.0 mmol, 5.0 eq.) in THF/EtOH (50 mL/50 mL) was added Zn (2.18 g, 34.0 mmol, 5.0 eq.). The resulting mixture was stirred at 70° C. for 3 hrs. After completion, the reaction mixture filtered. The filtrate was concentrated under reduced pressure. The residue was diluted with H₂O (100 mL) and extracted with EtOAc (100 mL×3). The organic layers were combined and washed with brine (100 mL), dried over anhydrous Na₂SO₄, and concentrated under reduced pressure. The residue was purified by flash column chromatography on silica gel to afford tert-butyl 4-(4-amino-3-((4-methoxybenzyl)amino)phenyl)piperazine-1-carboxylate (2.5 g, 6.07 mmol, 89%) as a white solid. LCMS (ESI): m/z=413 [M+H]⁺.

### STEP D: tert-butyl 4-(3-((4-methoxybenzyl)amino)-2-oxo-2,3-dihydro-1H-benzo[d]imidazol-5-yl)piperazine-1-carboxylate

To a solution of tert-butyl 4-(4-amino-3-((4-methoxybenzyl)amino)phenyl)piperazine-1-carboxylate (2.5 g, 6.07 mmol, 1.0 eq.) in THF (60 mL) was added CDI (2.95 g, 18.2 mmol, 3.0 eq.). The resulting mixture was stirred at 50° C. for 12 hrs. After completion, the reaction mixture was concentrated under reduced pressure. The residue was purified by flash column chromatography on silica gel to afford tert-butyl 4-(3-((4-methoxy benzyl)amino)-2-oxo-2,3-dihydro-1H-benzo[d]imidazol-5-yl)piperazine-1-carboxylate (2.5 g, 5.52 mmol, 91%) as a white solid. LCMS (ESI): m/z=454 [M+H]⁺.

### STEP E: tert-butyl 4-(1-(1-(4-methoxybenzyl)-2,6-dioxopiperidin-3-yl)-3-((4-methoxybenzyl)amino)-2-oxo-2,3-dihydro-1H-benzo[d]imidazol-5-yl)piperazine-1-carboxylate

To a solution of tert-butyl 4-(3-((4-methoxybenzyl)amino)-2-oxo-2,3-dihydro-1H-benzo[d]imidazol-5-yl)piperazine-1-carboxylate (200 mg, 0.44 mmol, 1.0 eq.) in dry THF (5 mL) was added LDA (0.24 mL, 0.48 mmol, 1.1 eq.) slowly at −78° C. under nitrogen. The mixture was stirred for additional 30 min at −78° C., then 1-(4-methoxybenzyl)-2,6-dioxopiperidin-3-yl trifluoromethanesulfonate (184 mg, 0.48 mmol, 1.1 eq.) in THF (2 mL) was added drop-wisely. After complete addition, the reaction mixture was allowed to warm to room temperature and stirred for 14 hrs. The reaction mixture was quenched by adding H₂O (10 mL), then extracted with EtOAc (10 mL×3). The organic layers were combined and washed with brine (15 mL), dried over anhydrous Na₂SO₄, and concentrated under reduced pressure. The residue was purified by flash column chromatography on silica gel to afford tert-butyl 4-(1-(1-(4-methoxy benzyl)-2,6-dioxopiperidin-3-yl)-3-((4-methoxybenzyl)amino)-2-oxo-2,3-dihydro-1H-benzo[d]imidazol-5-yl)piperazine-1-carboxylate (180 mg, 0.26 mmol, 60%) as a yellow solid. LC-MS (ESI) m/z=685 [M+H]⁺

### STEP F: 3-(2-oxo-5-(piperazin-1-yl)-2,3-dihydro-1H-benzo[d]imidazol-1-yl)piperidine-2,6-dione

To a solution of tert-butyl 4-(1-(1-(4-methoxybenzyl)-2,6-dioxopiperidin-3-yl)-3-((4-methoxybenzyl)amino)-2-oxo-2,3-dihydro-1H-benzo[d]imidazol-5-yl)piperazine-1-carboxylate (180 mg, 0.26 mmol, 1.0 eq.) in toluene (5 mL) was added MsOH (1 mL). The resulting mixture was stirred at 100° C. for 12 hrs. After completion, the reaction mixture was concentrated under reduced pressure. The residue was purified by flash column chromatography on silica gel to afford 3-(2-oxo-5-(piperazin-1-yl)-2,3-dihydro-1H-benzo[d]imidazol-1-yl)piperidine-2,6-dione (40 mg, 0.12 mmol, 47%) as a white solid. LCMS (ESI): m/z=330 [M+H]⁺.

### STEP G: tert-butyl ((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamate

To a solution of 3-(2-oxo-5-(piperazin-1-yl)-2,3-dihydro-1H-benzo[d]imidazol-1-yl)piperidine-2,6-dione (40 mg, 0.12 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (59 mg, 0.12 mmol, 1.0 eq.) in DMF (3 mL) were added DIEA (46 mg, 0.36 mmol, 3.0 eq.) and HATU (55 mg, 0.14 mmol. 1.2 eq.), and the resulting mixture was stirred at room temperature for 12 hrs. After completion, the reaction mixture was purified by C18 column to afford tert-butyl ((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamate (40 mg, 50.2 μmol, 42%) as a yellow solid. LCMS (ESI): m/z=798 [M+H]⁺.

### STEP H: 3-(5-(4-((5S,8S,10aR)-5-amino-3-(5-methylbenzo[d]isoxazole-3-carbonyl)-6-oxodecahydropyrrolo[1,2-a][1,5]diazocine-8-carbonyl)piperazin-1-yl)-2-oxo-2,3-dihydro-1H-benzo[d]imidazol-1-yl)piperidine-2,6-dione

To a solution of tert-butyl ((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamate (40 mg, 50.2 μmol, 1.0 eq.) in DCM (1 mL) was added TFA (0.3 mL), and the resulting mixture was stirred at room temperature for 1 hr. After completion, the reaction mixture was concentrated under reduced pressure to give crude 3-(5-(4-((5S,8S,10aR)-5-amino-3-(5-methylbenzo[d]isoxazole-3-carbonyl)-6-oxodecahydropyrrolo[1,2-a][1,5]diazocine-8-carbonyl)piperazin-1-yl)-2-oxo-2,3-dihydro-1H-benzo[d]imidazol-1-yl)piperidine-2,6-dione (35 mg quant.) (TFA salt) as a colorless oil, which was used in next step directly without further purification. LCMS (ESI): m/z=698 [M+H]⁺.

### STEP I: ((7-(((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of 3-(5-(4-((5S,8S,10aR)-5-amino-3-(5-methylbenzo[d]isoxazole-3-carbonyl)-6-oxodecahydropyrrolo[1,2-a][1,5]diazocine-8-carbonyl)piperazin-1-yl)-2-oxo-2,3-dihydro-1H-benzo[d]imidazol-1-yl)piperidine-2,6-dione (35 mg, 50.2 μmol, 1.0 eq.) and (difluoro(7-((perfluorophenoxy)carbonyl)naphthalen-2-yl)methyl)phosphonic acid (23 mg, 50.2 μmol, 1.0 eq.) in DMF (1 mL) was added DIEA (32 mg, 251 μmol, 5.0 eq.) at room temperature. The resulting mixture was stirred at room temperature for 2 hrs. After completion, the reaction mixture was purified by prep-HPLC to give ((7-(((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 34, 26 mg, 26.5 μmol, 53%) as a white solid.

¹H NMR (400 MHz, DMSO-d6) δ 11.04 (s, 1H), 10.83 (s, 1H), 8.97-8.55 (m, 2H), 8.25-8.14 (m, 1H), 8.11-7.95 (m, 4H), 7.76-7.68 (m, 2H), 7.58-7.51 (m, 1H), 6.93-6.61 (m, 3H), 5.31-5.20 (m, 2H), 4.93-4.86 (m, 1H), 4.52-4.47 (m, 1H), 4.08-3.99 (m, 3H), 3.74-3.68 (m, 2H), 3.52-3.45 (m, 1H), 3.05-2.84 (m, 4H), 2.69-2.55 (m, 2H), 2.48-2.45 (m, 3H), 2.38-1.44 (m, 10H). (TFA salt).

LCMS (ESI): m/z=982.2 [M+H]⁺.

### Preparation of ((7-(((5S,8S,10aR)-8-((2-(2,6-dioxopiperidin-3-yl)-1-oxoisoindolin-4-yl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 35)

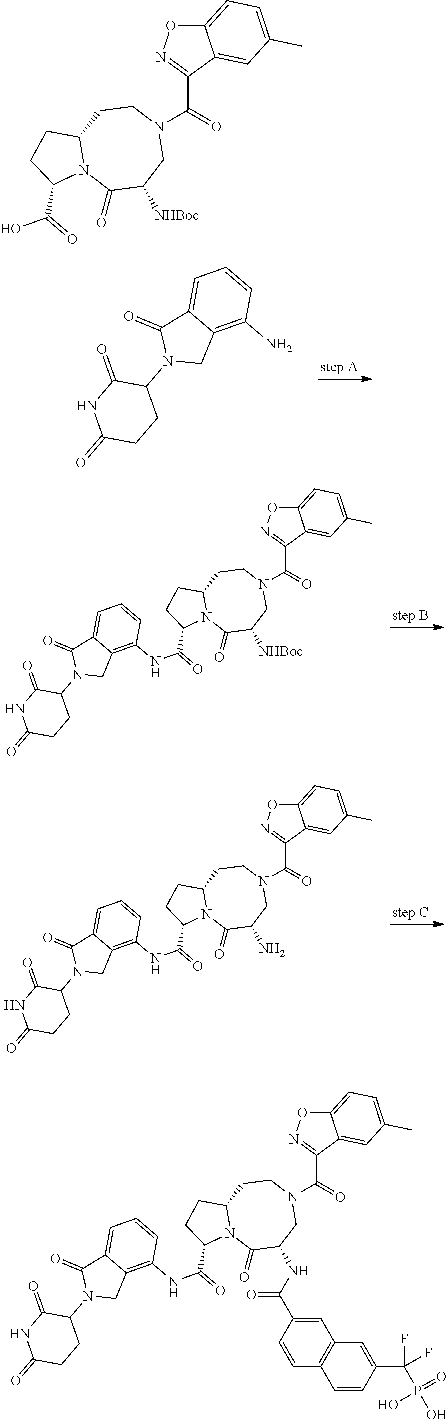

### STEP A: tert-butyl ((5S,8S,10aR)-8-((2-(2,6-dioxopiperidin-3-yl)-1-oxoisoindolin-4-yl)carbamoyl)-3-(5-methylbenzo[d]isoxazole-3-carbonyl)-6-oxodecahydropyrrolo[1,2-a][1,5]diazocin-5-yl)carbamate

To a solution of 3-(4-amino-1-oxoisoindolin-2-yl)piperidine-2,6-dione (107 mg, 0.41 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (200 mg, 0.41 mmol, 1.0 eq.) in DMF (10 mL) were added CMPI (125 mg, 0.49 mmol, 1.2 eq.), DABCO (138 mg, 1.23 mmol, 3.0 eq.) and DMAP (72 mg, 0.59 mmol, 1.2 eq.), and the resulting mixture was stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((5S,8S,10aR)-8-((2-(2,6-dioxopiperidin-3-yl)-1-oxoisoindolin-4-yl)carbamoyl)-3-(5-methylbenzo[d]isoxazole-3-carbonyl)-6-oxodecahydropyrrolo[1,2-a][1,5]diazocin-5-yl)carbamate (220 mg, 0.30 mmol, 74%) as a white solid. LCMS (ESI): m/z=728 [M+H]⁺.

### STEP B: (5S,8S,10aR)-5-amino-N-(2-(2,6-dioxopiperidin-3-yl)-1-oxoisoindolin-4-yl)-3-(5-methylbenzo[d]isoxazole-3-carbonyl)-6-oxodecahydropyrrolo[1,2-a][1,5]diazocine-8-carboxamide

To a solution of tert-butyl ((5S,8S,10aR)-8-((2-(2,6-dioxopiperidin-3-yl)-1-oxoisoindolin-4-yl)carbamoyl)-3-(5-methylbenzo[d]isoxazole-3-carbonyl)-6-oxodecahydropyrrolo[1,2-a][1,5]diazocin-5-yl)carbamate (220 mg, 0.30 mmol, 1.0 eq.) in DCM (3 mL) was added TFA (1 mL), and the resulting mixture was stirred at room temperature for 2 hrs. After completion, the reaction mixture was concentrated under reduced pressure to give crude (5S,8S,10aR)-5-amino-N-(2-(2,6-dioxopiperidin-3-yl)-1-oxoisoindolin-4-yl)-3-(5-methylbenzo[d]isoxazole-3-carbonyl)-6-oxodecahydropyrrolo[1,2-a][1,5]diazocine-8-carboxamide (220 mg, quant.) (TFA salt) as a white solid, which was used in next step directly without further purification. LCMS (ESI): m/z=628 [M+H]⁺.

### STEP C: ((7-(((5S,8S,10aR)-8-((2-(2,6-dioxopiperidin-3-yl)-1-oxoisoindolin-4-yl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of (5S,8S,10aR)-5-amino-N-(2-(2,6-dioxopiperidin-3-yl)-1-oxoisoindolin-4-yl)-3-(5-methylbenzo[d]isoxazole-3-carbonyl)-6-oxodecahydropyrrolo[1,2-a][1,5]diazocine-8-carboxamide (90 mg, 0.14 mmol, 1.0 eq.) and (difluoro(7-((perfluorophenoxy)carbonyl)naphthalen-2-yl)methyl)phosphonic acid (66 mg, 0.14 μmol, 1.0 eq.) in DMF (2 mL) was added DIEA (90 mg, 0.70 mmol, 5.0 eq.) at room temperature. The resulting mixture was stirred at room temperature for 2 hrs. After completion, the reaction mixture was concentrated under reduced pressure. The residue was purified by RP-prep HPLC to give ((7-(((5S,8S,10aR)-8-((2-(2,6-dioxopiperidin-3-yl)-1-oxoisoindolin-4-yl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 35, 44 mg, 48.3 μmol, 35%) as a white solid.

¹H NMR (400 MHz, DMSO-d6) δ 11.08-10.92 (m, 1H), 10.06-9.82 (m, 1H), 9.10-8.78 (m, 1H), 8.75-8.52 (m, 1H), 8.26-8.13 (m, 1H), 8.12-7.95 (m, 3H), 7.89-7.64 (m, 4H), 7.52-7.43 (m, 2H), 7.34-7.19 (m, 1H), 5.37-5.26 (m, 1H), 5.16-5.08 (m, 1H), 4.61-4.57 (m, 1H), 4.46-4.41 (m, 1H), 4.31 (s, 2H), 4.04-3.81 (m, 4H), 2.96-2.83 (m, 1H), 2.65-2.54 (m, 1H), 2.43-2.11 (m, 7H), 2.09-1.79 (m, 4H).

LCMS (ESI): n z=912.2 [M+H]⁺.

### Preparation of ((7-(((5S,8S,10aR)-8-(4-(1-(2,6-dioxopiperidin-3-yl)-2-oxo-2,3-dihydro-1H-benzo[d]imidazol-4-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 36)

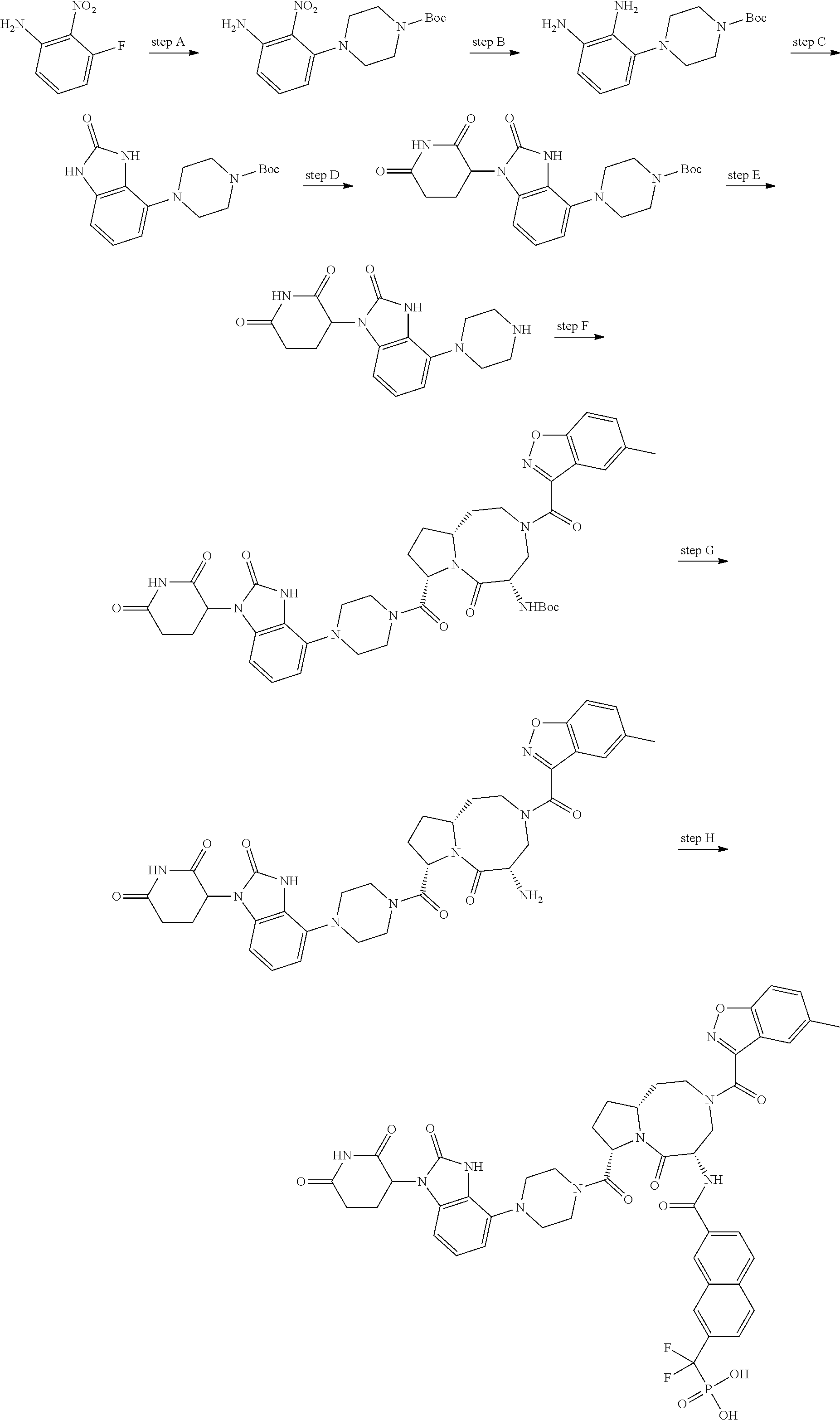

### STEP A: tert-butyl 4-(3-amino-2-nitrophenyl)piperazine-1-carboxylate

To a solution of 3-fluoro-2-nitroaniline (2.0 g, 12.8 mmol, 1.0 eq.) and tert-butyl piperazine-1-carboxylate (2.38 g, 12.8 mmol, 1.0 eq.) in DMF (100 mL) was added K₂CO₃ (5.30 g, 38.4 mmol, 3.0 eq.). The resulting mixture was stirred at 110° C. for 12 hrs. After completion, the reaction mixture was diluted with H₂O (100 mL) and extracted with EtOAc (100 mL×3). The organic layers were combined and washed with brine (100 mL), dried over anhydrous Na₂SO₄ and concentrated under reduced pressure. The residue was purified by flash column chromatography on silica gel to afford tert-butyl 4-(3-amino-2-nitrophenyl)piperazine-1-carboxylate (3.6 g, 11.2 mmol, 88%) as a white solid LCMS (ESI) m/z=323 [M+H]⁺.

### STEP B: tert-butyl 4-(2,3-diaminophenyl)piperazine-1-carboxylate

To a solution of tert-butyl 4-(3-amino-2-nitrophenyl)piperazine-1-carboxylate (3.6 g, 11.2 mmol, 1.0 eq.) in MeOH (50 mL) was added Pd/C (1.8 g) under nitrogen. The suspension was degassed under vacuum and purged with H₂ several times. The resulting mixture was stirred at room temperature for 14 hrs. After completion, the suspension was filtered through a pad of Celite®, the filter cake was washed with MeOH (20 mL). The combined filtrates were concentrated to dryness to give tert-butyl 4-(2,3-diaminophenyl)piperazine-1-carboxylate (3.3 g. quant.) as a white solid. LCMS (ESI): m/z=293 [M+H]⁺.

### STEP C: tert-butyl 4-(2-oxo-2,3-dihydro-1H-benzo[d]imidazol-4-yl)piperazine-1-carboxylate

To a solution of tert-butyl 4-(2,3-diaminophenyl)piperazine-1-carboxylate (3.3 g, 11.3 mmol, 1.0 eq.) in THF (80 mL) was added CDI (5.49 g, 33.9 mmol, 3.0 eq.). The resulting mixture was stirred at 50° C. for 12 hrs. After completion, the reaction mixture was concentrated under reduced pressure. The residue was purified by flash column chromatography on silica gel to afford tert-butyl 4-(2-oxo-2,3-dihydro-1H-benzo[d]imidazol-4-yl)piperazine-1-carboxylate (3.0 g, 9.43 mmol, 83%) as a white solid. LCMS (ESI): m/z=319 [M+H]⁺.

### STEP D: tert-butyl 4-(1-(2,6-dioxopiperidin-3-yl)-2-oxo-2,3-dihydro-1H-benzo[d]imidazol-4-yl)piperazine-1-carboxylate

To a solution of tert-butyl 4-(2-oxo-2,3-dihydro-1H-benzo[d]imidazol-4-yl)piperazine-1-carboxylate (I g, 3.14 mmol, 1.0 eq.) in dry DMF (10 mL) was added NaH (151 mg, 3.77 mmol, 1.2 eq.) in portions at 0° C. under nitrogen. The mixture was stirred for additional 30 min at 0° C., then 3-bromopiperidine-2,6-dione (720 mg, 3.77 mmol, 1.2 eq.) in DMF (5 mL) was added slowly, after addition, the reaction mixture was allowed to warm to room temperature and stirred for 14 hrs. After completion, the reaction mixture was quenched by adding H₂O (10 mL), then extracted with EtOAc (10 mL×3). The organic layers were combined and washed with brine (15 mL), dried over anhydrous Na₂SO₄ and concentrated under reduced pressure. The residue was purified by flash column chromatography on silica gel to afford tert-butyl 4-(1-(2,6-dioxopiperidin-3-yl)-2-oxo-2,3-dihydro-1H-benzo[d]imidazol-4-yl)piperazine-1-carboxylate (100 mg, 0.23 mmol, 7%) as a yellow solid. LC-MS (ESI) m/z=430 [M+H]⁺

### STEP E: 3-(2-oxo-4-(piperazin-1-yl)-2,3-dihydro-1H-benzo[d]imidazol-1-yl)piperidine-2,6-dione

To a solution of tert-butyl 4-(1-(2,6-dioxopiperidin-3-yl)-2-oxo-2,3-dihydro-1H-benzo[d]imidazol-4-yl)piperazine-1-carboxylate (100 mg, 0.23 mmol, 1.0 eq.) in DCM (3 mL) was added TFA (1 mL), and the resulting mixture was stirred at room temperature for 2 hrs. After completion, the reaction mixture was concentrated under reduced pressure to give crude 3-(2-oxo-4-(piperazin-1-yl)-2,3-dihydro-1H-benzo[d]imidazol-1-yl)piperidine-2,6-dione (100 mg, quant.) (TFA salt) as a white solid, which was used in next step directly without further purification. LCMS (ESI): m/z=330 [M+H]⁺.

### STEP F: tert-butyl ((5S,8S,10aR)-8-(4-(1-(2,6-dioxopiperidin-3-yl)-2-oxo-2,3-dihydro-1H-benzo[d]imidazol-4-yl)piperazine-1-carbonyl)-3-(5-methylbenzo[d]isoxazole-3-carbonyl)-6-oxodecahydropyrrolo[1,2-a][1,5]diazocin-5-yl)carbamate

To a solution of 3-(2-oxo-4-(piperazin-1-yl)-2,3-dihydro-1H-benzo[d]imidazol-1-yl)piperidine-2,6-dione (100 mg, 0.30 mmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (146 mg, 0.30 mmol, 1.0 eq.) in DMF (3 mL) were added HATU (125 mg, 0.33 mmol, 1.1 eq.) and TEA (91 mg, 0.90 mmol, 3.0 eq.), and the resulting mixture w % as stirred at room temperature for 2 hrs. After completion, the reaction was diluted with H₂O (10 mL) and extracted with EtOAc (10 mL×3). The organic layers were combined and washed with brine (10 mL), dried over anhydrous Na₂SO₄ and concentrated under reduced pressure. The residue was purified by flash column chromatography on silica gel to afford tert-butyl ((5S,8S,10aR)-8-(4-(1-(2,6-dioxopiperidin-3-yl)-2-oxo-2,3-dihydro-1H-benzo[d]imidazol-4-yl)piperazine-1-carbonyl)-3-(5-methylbenzo[d]isoxazole-3-carbonyl)-6-oxodecahydropyrrolo[1,2-a][1,5]diazocin-5-yl)carbamate (80 mg, 0.10 mmol, 33%) as a white solid. LCMS (ESI): m/z=798 [M+H]⁺.

### STEP G: 3-(4-(4-((5S,8S,10aR)-5-amino-3-(5-methylbenzo[d]isoxazole-3-carbonyl)-6-oxodecahydropyrrolo[1,2-a][1,5]diazocine-8-carbonyl)piperazin-1-yl)-2-oxo-2,3-dihydro-1H-benzo[d]imidazol-1-yl)piperidine-2,6-dione

To a solution of tert-butyl ((5S,8S,10aR)-8-(4-(1-(2,6-dioxopiperidin-3-yl)-2-oxo-2,3-dihydro-1H-benzo[d]imidazol-4-yl)piperazine-1-carbonyl)-3-(5-methylbenzo[d]isoxazole-3-carbonyl)-6-oxodecahydropyrrolo[1,2-a][1,5]diazocin-5-yl)carbamate (80 mg, 0.10 mmol, 1.0 eq.) in DCM (3 mL) was added TFA (1 mL), and the resulting mixture was stirred at room temperature for 2 hrs. After completion, the reaction mixture was concentrated under reduced pressure to give crude 3-(4-(4-((5S,8S,10aR)-5-amino-3-(5-methylbenzo[d]isoxazole-3-carbonyl)-6-oxodecahydropyrrolo[1,2-a][1,5]diazocine-8-carbonyl)piperazin-1-yl)-2-oxo-2,3-dihydro-1H-benzo[d]imidazol-1-yl)piperidine-2,6-dione (80 mg, quant.) (TFA salt) as a white solid, which was used in next step directly without further purification. LCMS (ESI): m/z=698 [M+H]⁺.

### STEP H: ((7-(((5S,8S,10aR)-8-(4-(1-(2,6-dioxopiperidin-3-yl)-2-oxo-2,3-dihydro-1H-benzo[d]imidazol-4-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of 3-(4-(4-((5S,8S,10aR)-5-amino-3-(5-methylbenzo[d]isoxazole-3-carbonyl)-6-oxodecahydropyrrolo[1,2-a][1,5]diazocine-8-carbonyl)piperazin-1-yl)-2-oxo-2,3-dihydro-1H-benzo[d]imidazol-1-yl)piperidine-2,6-dione (80 mg, 0.11 mmol, 1.0 eq.) and (difluoro(7-((perfluorophenoxy)carbonyl)naphthalen-2-yl)methyl)phosphonic acid (51 mg, 0.11 mmol, 1.0 eq.) in DMF (2 mL) was added TEA (43 mg, 0.33 mmol, 5.0 eq.) at room temperature. The resulting mixture was stirred at room temperature for 2 hrs. After completion, the reaction mixture was purified by RP-prep HPLC to give ((7-(((5S,8S,10aR)-8-(4-(1-(2,6-dioxopiperidin-3-yl)-2-oxo-2,3-dihydro-1H-benzo[d]imidazol-4-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 36, 31.2 mg, 31.8 μmol, 29%) as a white solid.

1H NMR (400 MHz, DMSO-d6) δ 11.29-10.81 (m, 2H), 9.14-8.49 (m, 2H), 8.31-7.84 (m, 5H), 7.82-7.41 (m, 3H), 7.26-6.70 (m, 3H), 6.63-6.17 (m, 1H), 5.43-5.29 (m, 2H), 4.95-4.76 (m, 1H), 4.57-3.45 (m, 8H), 3.10-2.57 (m, 7H), 2.44 (d, J=13.5 Hz, 3H), 2.36-1.52 (m, 7H).

LCMS (ESI): m/z=982.2 [M+H]⁻.

### Preparation of ((7-(((5S,8S,10aR)-8-(4-(3-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 37)

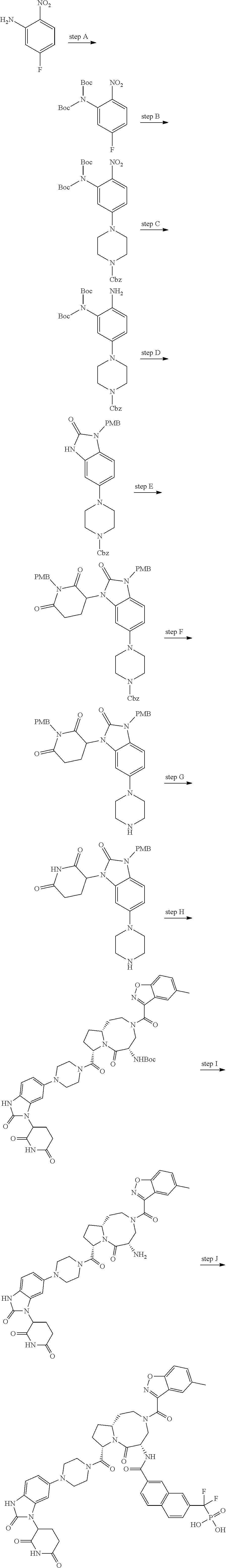

### STEP A: 2-methylpropan-2-yl [(5-fluoro-2-nitrophenyl){[(2-methylprop-2-yl)oxy]carbonyl}amino]methanoate

To a solution of 5-fluoro-2-nitroaniline (3.2 g, 20.5 mmol, 1.0 eq.) and TEA (6.21 g, 61.5 mmol, 3.0 eq.) in DCM (200 mL) were added Boc₂O (9.4 g, 43.1 mmol, 2.1 eq.) and DMAP (252 mg, 2.05 mmol, 0.1 eq.). The resulting mixture was stirred at room temperature for 5 hrs. After completion, the reaction mixture was concentrated under reduced pressure. The residue was purified by flash column chromatography on silica gel to afford 2-methylpropan-2-yl [(5-fluoro-2-nitrophenyl){[(2-methylprop-2-yl)oxy]carbonyl}amino]methanoate (6 g, 16.9 mmol, 82%) as a white solid. LCMS (ESI): m/z=357 [M+H]⁺.

### STEP B: 2-methylpropan-2-yl [(5-{4-[(benzyloxy)carbonyl]piperazin-1-yl}-2-nitrophenyl)({[2-methylprop-2-yl)oxy]carbonyl}amino]methanoate

To a solution of 2-methylpropan-2-yl [(5-fluoro-2-nitrophenyl){[(2-methylprop-2-yl)oxy]carbonyl}amino]methanoate (4 g, 11.2 mmol, 1.0 eq.) and benzyl piperazine-1-carboxylate (2.46 g, 11.2 mmol, 1.0 eq.) in DMF (100 mL) was added K₂CO₃ (4.64 g, 33.6 mmol, 3.0 eq.). The resulting mixture was stirred at 80° C. for 12 hrs. After completion, the reaction mixture was diluted with H₂O (100 mL) and extracted with EtOAc (100 mL×3). The organic layers were combined and washed with brine (100 mL), dried over anhydrous Na₂SO₄ and concentrated under reduced pressure. The residue was purified by flash column chromatography on silica gel to afford 2-methylpropan-2-yl [(5-{4-[(benzyloxy)carbonyl]piperazin-1-yl}-2-nitrophenyl) {[(2-methylprop-2-yl)oxy]carbonyl}amino]methanoate (4.5 g, 8.09 mmol, 72%) as a white solid. LCMS (ESI): m/z=557 [M+H]⁺.

### STEP C: 2-methylpropan-2-yl [(2-amino-5-{4-[(benzyloxy)carbonyl]piperazin-1-yl}phenyl){[(2-methylprop-2-yl)oxy]carbonyl}amino]methanoate

To a solution of 2-methylpropan-2-yl [(5-{4-[(benzyloxy)carbonyl]piperazin-1-yl}-2-nitrophenyl){[(2-methylprop-2-yl)oxy]carbonyl}amino]methanoate (1.5 g, 2.70 mmol, 1.0 eq.) and NH₄Cl (716 mg, 13.5 mmol, 5.0 eq.) in EtOH/H₂O (20 mL/4 mL) was added Zn (878 mg, 13.5 mmol, 5.0 eq.). The resulting mixture was stirred at room temperature for 3 hrs. After completion, the reaction mixture was filtered. The filtrate was concentrated under reduced pressure. The residue was diluted with H₂O (20 mL) and extracted with EtOAc (20 mL×3). The organic layers were combined and washed with brine (20 mL), dried over anhydrous Na₂SO₄ and concentrated under reduced pressure. The residue was purified by flash column chromatography on silica gel to afford tert-butyl 4-(4-amino-3-((4-methoxybenzyl)amino)phenyl)piperazine-1-carboxylate (1 g, 1.90 mmol, 70%) as a white solid LCMS (ESI): m/z=527 [M+H]⁺.

### STEP D: benzyl 4-(1-(4-methoxybenzyl)-2-oxo-2,3-dihydro-1H-benzo[d]imidazol-5-yl)piperazine-1-carboxylate

To a solution of tert-butyl 4-(4-amino-3-((4-methoxybenzyl)amino)phenyl)piperazine-1-carboxylate (1 g, 1.90 mmol, 1.0 eq.) in DMF (20 mL) was added NaH (114 mg, 2.85 mmol, 1.5 eq.) at 0° C. The mixture was stirred for an additional 30 min at 0° C. then PMBCl (326 mg, 2.09 mmol, 1.1 eq.) was added dropwise. After addition, the reaction mixture was allowed to warm to room temperature and stirred for 14 hrs. After completion, the reaction mixture was quenched by adding H₂O (20 mL), then extracted with EtOAc (15 mL×3). The organic layers were combined and washed with brine (15 mL), dried over anhydrous Na₂SO₄ and concentrated under reduced pressure. The residue was purified by flash column chromatography on silica gel to afford tert-butyl 4-(3-((4-methoxybenzyl)amino)-4-nitrophenyl)piperazine-1-carboxylate (350 mg, 0.74 mmol, 39%) as a white solid. LC-MS (ESI) m/z=473 [M+H]⁺

### STEP E: benzyl 4-(1-(4-methoxybenzyl)-3-(1-(4-methoxybenzyl)-2,6-dioxopiperidin-3-yl)-2-oxo-2,3-dihydro-1H-benzo[d]imidazol-5-yl)piperazine-1-carboxylate

To a solution of tert-butyl 4-(3-((4-methoxybenzyl)amino)-4-nitrophenyl)piperazine-1-carboxylate (350 mg, 0.74 mmol, 1.0 eq.) in dry THF (5 mL) was added LDA (0.41 mL, 0.81 mmol, 1.1 eq.) slowly at 0° C. under nitrogen. The mixture was stirred for additional 30 min at 0° C., then 1-(4-methoxybenzyl)-2,6-dioxopiperidin-3-yl trifluoromethanesulfonate (309 mg, 0.81 mmol, 1.1 eq.) in THF (2 mL) was added dropwise. After complete addition of the reagent, the reaction mixture was allowed to warm to room temperature and stirred for 14 hrs. The reaction mixture was quenched by adding H₂O (10 mL), then extracted with EtOAc (10 mL×3). The organic layers were combined and washed with brine (15 mL), dried over anhydrous Na₂SO₄ and concentrated under reduced pressure. The residue was purified by flash column chromatography on silica gel to afford benzyl 4-(1-(4-methoxy benzyl)-3-(1-(4-methoxybenzyl)-2,6-dioxopiperidin-3-yl)-2-oxo-2,3-dihydro-1H-benzo[d]imidazol-5-yl)piperazine-1-carboxylate (250 mg, 0.36 mmol, 49%) as a yellow solid. LC-MS (ESI) m/z=704 [M+H]⁺

### STEP F: 1-(4-methoxybenzyl)-3-(3-(4-methoxybenzyl)-2-oxo-6-(piperazin-1-yl)-2,3-dihydro-1H-benzo[d]imidazol-1-yl)piperidine-2,6-dione

To a solution of benzyl 4-(1-(4-methoxybenzyl)-3-(1-(4-methoxybenzyl)-2,6-dioxopiperidin-3-yl)-2-oxo-2,3-dihydro-1H-benzo[d]imidazol-5-yl)piperazine-1-carboxylate (250 mg, 0.36 mmol, 1.0 eq.) in THF (5 mL) was added Pd/C (125 mg) under nitrogen. The suspension was degassed under vacuum and purged with H₂ several times. The resulting mixture was stirred at room temperature for 14 hrs. After completion, the suspension was filtered through a pad of Celite®, the filter cake was washed with MeOH (10 mL). The combined filtrates were concentrated to dryness to give 1-(4-methoxybenzyl)-3-(3-(4-methoxybenzyl)-2-oxo-6-(piperazin-1-yl)-2,3-dihydro-1H-benzo[d]imidazol-1-yl)piperidine-2,6-dione (130 mg, 0.23 mmol, 64%) as a white solid. LCMS (ESI): m/z=570 (M+H)⁺.

### STEP G: 3-(2-oxo-6-(piperazin-1-yl)-2,3-dihydro-1H-benzo[d]imidazol-1-yl)piperidine-2,6-dione

A solution of 1-(4-methoxybenzyl)-3-(3-(4-methoxybenzyl)-2-oxo-6-(piperazin-1-yl)-2,3-dihydro-1H-benzo[d]imidazol-1-yl)piperidine-2,6-dione (130 mg, 0.23 mmol, 1.0 eq.) in MsOH (3 mL) was stirred at 100° C. for 12 hrs. After completion, the reaction mixture was concentrated under reduced pressure. The residue was purified by flash column chromatography on silica gel to afford 3-(2-oxo-6-(piperazin-1-yl)-2,3-dihydro-1H-benzo[d]imidazol-1-yl)piperidine-2,6-dione (15 mg, 45.6 μmol, 20%) as a white solid LCMS (ESI): m/z=330 [M+H]⁺.

### STEP H: tert-butyl ((5S,8S,10aR)-8-(4-(3-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamate

To a solution of 3-(2-oxo-6-(piperazin-1-yl)-2,3-dihydro-1H-benzo[d]imidazol-1-yl)piperidine-2,6-dione (15 mg, 45.6 μmol, 1.0 eq.) and (5S,8S,10aR)-5-((tert-butoxycarbonyl)amino)-3-(5-methylbenzo[d]isoxazole-3-carbonyl)-6-oxodecahydropyrrolo[1,2-a][1,5]diazocine-8-carboxylic acid (22 mg, 45.6 μmol, 1.0 eq.) in DMF (3 mL) were added HATU (19 mg, 50.2 μmol, 1.1 eq.) and DIEA (18 mg, 0.14 mmol, 3.0 eq.). The resulting mixture was stirred at room temperature for 2 hrs. After completion, the reaction mixture was diluted with 1120 (10 mL) and extracted with EtOAc (10 mL×3). The organic layers were combined and washed with brine (10 mL), dried over anhydrous Na₂SO₄ and concentrated under reduced pressure. The residue was purified by flash column chromatography on silica gel to afford tert-butyl ((5S,8S,10aR)-8-(4-(3-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamate (20 mg, 25.1 μmol, 55%) as a white solid. LCMS (ESI): m/z=798 [M+H]⁺.

### STEP I: ((7-(((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of tert-butyl ((5S,8S,10aR)-8-(4-(3-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamate (20 mg, 25.1 μmol, 1.0 eq.) in DCM (3 mL) was added TFA (1 mL), and the resulting mixture was stirred at room temperature for 2 hrs. After completion, the reaction mixture was concentrated under reduced pressure to give crude ((7-(((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (15 mg, quant.) (TFA salt) as a white solid, which was used in next step directly without further purification. LCMS (ESI): m/z=698 [M+H]⁺.

### STEP J: ((7-(((5S,8S,10aR)-8-(4-(3-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid

To a solution of ((7-(((5S,8S,10aR)-8-(4-(1-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (15 mg, 21.5 μmol, 1.0 eq.) and (difluoro(7-((perfluorophenoxy)carbonyl)naphthalen-2-yl)methyl)phosphonic acid (10 mg, 21.5 μmol, 1.0 eq.) in DMF (2 mL) was added DIEA (14 mg, 108 μmol, 5.0 eq.) at room temperature. The resulting mixture was stirred at room temperature for 2 hrs. After completion, the reaction mixture was purified by RP-prep HPLC to give ((7-(((5S,8S,10aR)-8-(4-(3-(2,6-dioxopiperidin-3-yl)-2-oxo-2,3-dihydro-1H-benzo[d]imidazol-5-yl)piperazine-1-carbonyl)-3-(5-methylbenzo[d]isoxazole-3-carbonyl)-6-oxodecahydropyrrolo[1,2-a][1,5]diazocin-5-yl)carbamoyl)naphthalen-2-yl)difluoromethyl)phosphonic acid (Compound 37, 9.5 mg, 9.7 μmol, 45%) as a white solid.

¹H NMR (400 MHz, DMSO-d6) δ 11.06-10.66 (m, 1H), 9.05-8.69 (m, 1H), 8.58 (s, 1H), 8.25-8.03 (m, 4H), 8.00-7.91 (m, 1H), 7.80-7.67 (m, 3H), 7.57-7.50 (m, 1H), 6.94-6.56 (m, 2H), 5.34-5.18 (m, 2H), 4.94-4.81 (m, 1H), 4.55-4.43 (m, 1H), 4.37-4.26 (m, 1H), 4.10-3.97 (m, 2H), 3.92-3.83 (m, 1H), 3.73-3.65 (m, 1H), 3.53-3.43 (m, 1H), 3.12-2.57 (m, 6H), 2.47-2.44 (m, 3H), 2.34-1.47 (m, 10H).

LCMS (ESI): m/z=982.2 [M+H]⁺.

### Preparation of S,S′-(((((2-(((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphoryl)bis(oxy))bis(ethane-2,1-diyl)) dibutanethioate (Compound 38) and ((2-(((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphonic acid (Compound 39)

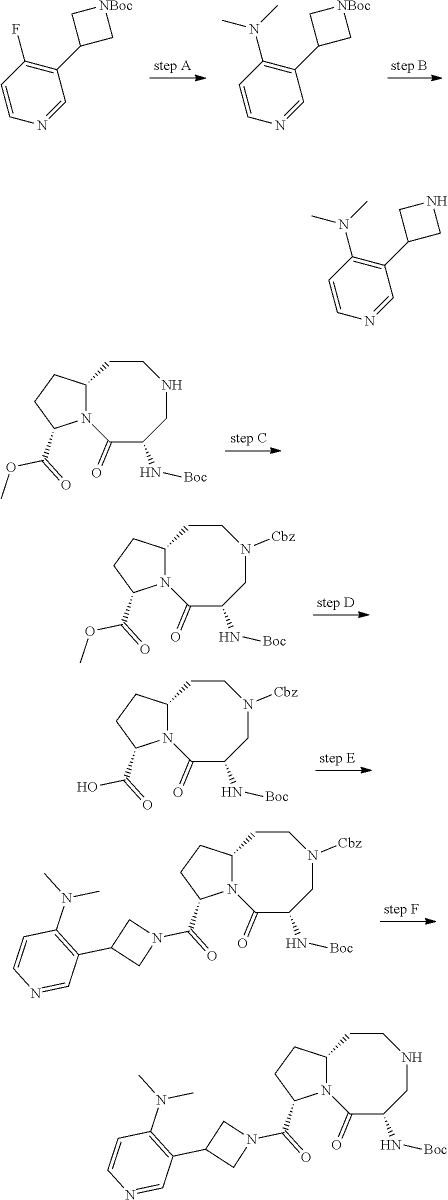

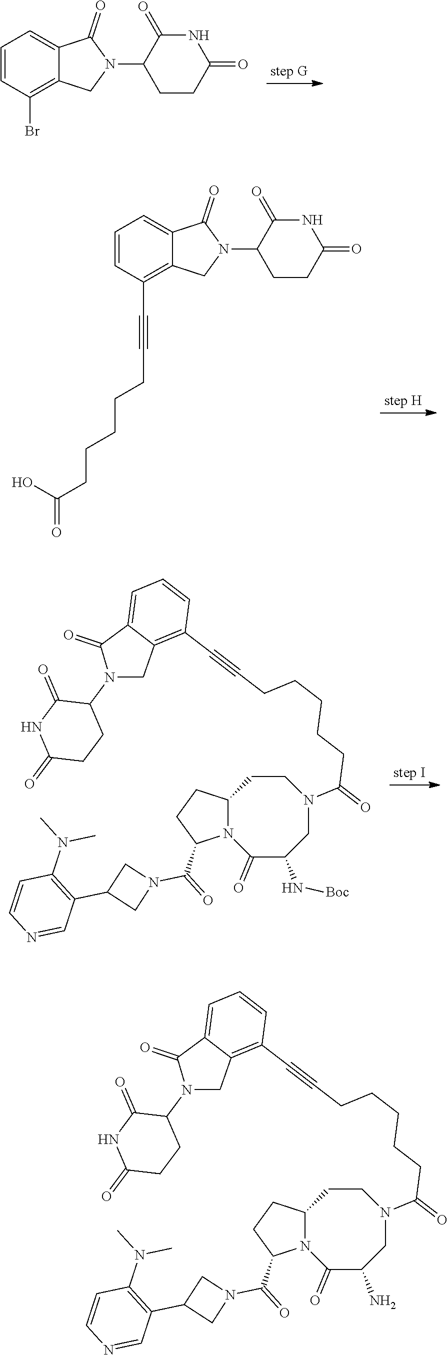 [SMILES: O=C1CCC(N2Cc3c(Br)cccc3C2=O)C(=O)N1 ; O=C(O)CCCCCC#Cc1cccc2c1CN(C1CCC(=O)NC1=O)C2=O ; CN(C)c1ccncc1C1CN(C(=O)[C@@H]2CC[C@@H]3CCN(C(=O)CCCCCC#Cc4cccc5c4CN(C4CCC(=O)NC4=O)C5=O)C[C@H](NC(=O)OC(C)(C)C)C(=O)N32)C1 ; CN(C)c1ccncc1C1CN(C(=O)[C@@H]2CC[C@@H]3CCN(C(=O)CCCCCC#Cc4cccc5c4CN(C4CCC(=O)NC4=O)C5=O)C[C@H](N)C(=O)N32)C1]

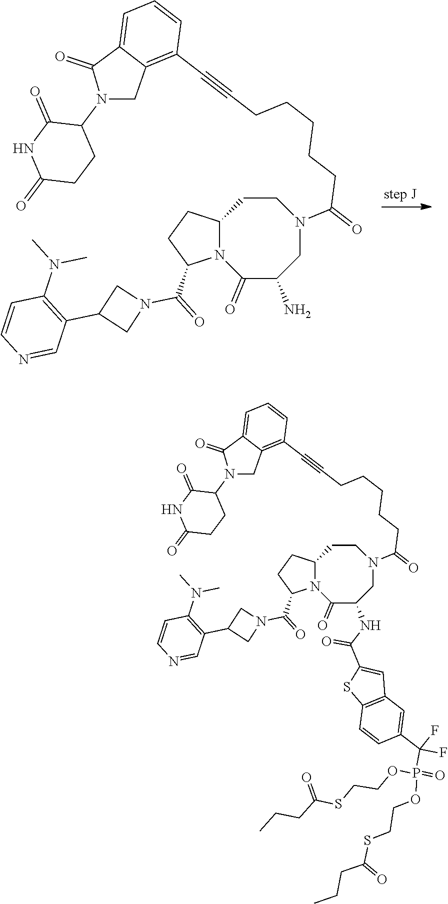 [SMILES: CN(C)c1ccncc1C1CN(C(=O)[C@@H]2CC[C@@H]3CCN(C(=O)CCCCCC#Cc4cccc5c4CN(C4CCC(=O)NC4=O)C5=O)C[C@H](N)C(=O)N32)C1 ; CCCC(=O)SCCOP(=O)(OCCSC(=O)CCC)C(F)(F)c1ccc2sc(C(=O)N[C@H]3CN(C(=O)CCCCCC#Cc4cccc5c4CN(C4CCC(=O)NC4=O)C5=O)CC[C@H]4CC[C@@H](C(=O)N5CC(c6cnccc6N(C)C)C5)N4C3=O)cc2c1]

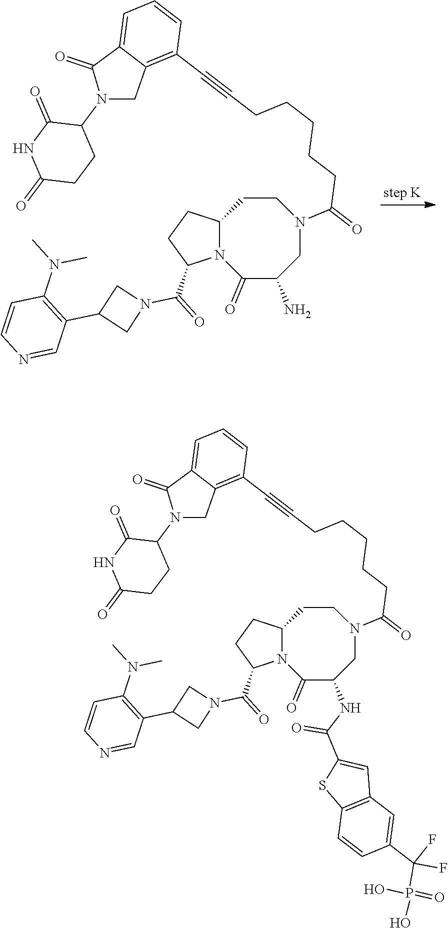 [SMILES: CN(C)c1ccncc1C1CN(C(=O)[C@@H]2CC[C@@H]3CCN(C(=O)CCCCCC#Cc4cccc5c4CN(C4CCC(=O)NC4=O)C5=O)C[C@H](N)C(=O)N32)C1 ; CN(C)c1ccncc1C1CN(C(=O)[C@@H]2CC[C@@H]3CCN(C(=O)CCCCCC#Cc4cccc5c4CN(C4CCC(=O)NC4=O)C5=O)C[C@H](NC(=O)c4cc5cc(C(F)(F)P(=O)(O)O)ccc5s4)C(=O)N32)C1]

### STEP A: tert-butyl 3-(4-(dimethylamino)pyridin-3-yl)azetidine-1-carboxylate

To a solution of tert-butyl 3-(4-fluoropyridin-3-yl)azetidine-1-carboxylate (500 mg, 1.98 mmol, 1.0 eq.) and dimethylamine (178 mg, 3.96 mmol, 2.0 eq.) in DMF (10 mL) was added DIEA (550 mg, 3.96 mmol, 2.0 eq.). The reaction mixture was stirred at 100° C. overnight in a sealed tube. After completion, the reaction mixture was diluted with H₂O (30 mL) and extracted with EtOAc (30 mL×2). The combined organic layer was washed with brine (10 mL), dried over anhydrous Na₂SO₄, and concentrated under reduced pressure. The residue was purified by C18 column chromatography to give tert-butyl 3-(4-(dimethylamino)pyridin-3-yl)azetidine-1-carboxylate (480 mg, 1.73 mmol, 87%) as a white solid. LCMS (ESI): m/z=278 [M+H]⁻.

### STEP B: 3-(azetidin-3-yl)-N,N-dimethylpyridin-4-amine

To a solution of tert-butyl 3-(4-(dimethylamino)pyridin-3-yl)azetidine-1-carboxylate (480 mg, 1.73 mmol, 1.0 eq.) in DCM (10 mL) was added TFA (5 mL) and the reaction mixture was stirred at room temperature for 2 hrs. After completion, the reaction mixture was concentrated under reduced pressure to get crude 3-(azetidin-3-yl)-N,N-dimethylpyridin-4-amine (450 mg, quant.) as a white solid, which was used in next step directly without further purification. LCMS (ESI): m/z=178 [M+H]⁻.

### STEP C: 3-benzyl 8-methyl (5S,8S,10aR)-5-((tert-butoxycarbonyl)amino)-6-oxooctahydropyrrolo[1,2-a][1,5]diazocine-3,8(4H)-dicarboxylate

To a solution of methyl (5S,8S,10aR)-5-((tert-butoxycarbonyl)amino)-6-oxodecahydropyrrolo[1,2-a][1,5]diazocine-8-carboxylate (500 mg, 1.47 mmol, 1.0 eq.) and Na₂CO₃ (310 mg, 2.93 mmol, 2.0 eq.) in THF (10 mL) and H₂O (5 mL) was added CbzCl (300 mg, 1.76 mmol, 1.2 eq.). The reaction mixture was stirred at room temperature for 4 hrs. After completion, the reaction mixture was quenched with H₂O (30 mL) and extracted with EtOAc (30 mL×2). The organic layers were combined and washed with brine (50 mL), dried over anhydrous Na₂SO₄, and concentrated under reduced pressure. The residue was purified by flash column chromatography on silica gel to afford 3-benzyl 8-methyl (5S,8S,10aR)-5-((tert-butoxycarbonyl)amino)-6-oxooctahydropyrrolo[1,2-a][1,5]diazocine-3,8(4H)-dicarboxylate (600 mg, 1.26 mmol, 86%) as a white solid. LCMS (ESI): m/z=476 [M+H]⁺.

### STEP D: (5S,8S,10aR)-3-((benzyloxy)carbonyl)-5-((tert-butoxycarbonyl)amino)-6-oxodecahydropyrrolo[1,2-a][1,5]diazocine-8-carboxylic acid

To a solution of methyl 3-benzyl 8-methyl (5S,8S,10aR)-5-((tert-butoxycarbonyl)amino)-6-oxooctahydropyrrolo[1,2-a][1,5]diazocine-3,8(4H)-dicarboxylate (600 mg, 1.26 mmol, 1.0 eq.) in MeOH (10 mL) and H₂O (5 ml) was added LiOH (103 mg, 2.52 mmol, 2.0 eq.). The reaction mixture was stirred at room temperature for 4 hrs. After completion, the reaction mixture was acidified with HCl (1N, aq.) until the pH was adjusted pH=3-4 and extracted with EtOAc (30 mL×2). The organic layers were combined and washed with brine (20 mL), dried over anhydrous Na₂SO₄, and concentrated under reduced pressure to give (5S,8S,10aR)-3-((benzyloxy)carbonyl)-5-((tert-butoxycarbonyl)amino)-6-oxodecahydropyrrolo[1,2-a][1,5]diazocine-8-carboxylic acid (550 mg, 1.19 mmol, 95%) as a white solid LCMS (ESI): m/z=462 [M+H]⁺.

### STEP E: benzyl (5S,8S,10aR)-5-((tert-butoxycarbonyl)amino)-8-(3-(4-(dimethylamino)pyridin-3-yl)azetidine-1-carbonyl)-6-oxooctahydropyrrolo[1,2-a][1,5]diazocine-3(4H)-carboxylate

To a mixture of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (550 mg, 1.19 mmol, 1.0 eq.) and HATU (498 mg, 1.31 mmol, 1.1 eq.) in DMF (5 mL) were added DIEA (307 mg, 2.39 mmol, 2.0 eq.) and 3-(azetidin-3-yl)-N,N-dimethylpyridin-4-amine (210 mg, 1.19 mmol, 1.0 eq.). The resulting mixture was stirred at room temperature for 30 min. After completion, the reaction mixture was purified directly by Biotage® C18 column chromatography to afford benzyl (5S,8S,10aR)-5-((tert-butoxycarbonyl)amino)-8-(3-(4-(dimethylamino)pyridin-3-yl)azetidine-1-carbonyl)-6-oxooctahydropyrrolo[1,2-a][1,5]diazocine-3(4H)-carboxylate (650 mg, 1.04 mmol, 88%) as a white solid. LCMS (ESI): m/z=621 [M+H]⁺.

### STEP F: tert-butyl ((5S,8S,10aR)-8-(3-(4-(dimethylamino)pyridin-3-yl)azetidine-1-carbonyl)-6-oxodecahydropyrrolo[1,2-a][1,5]diazocin-5-yl)carbamate

To a solution of benzyl (5S,8S,10aR)-5-((tert-butoxycarbonyl)amino)-8-(3-(4-(dimethylamino)pyridin-3-yl)azetidine-1-carbonyl)-6-oxooctahydropyrrolo[1,2-a][1,5]diazocine-3(4H)-carboxylate (650 mg, 1.04 mmol, 1.0 eq.) in MeOH (20 mL) was added Pd/C (100 mg) under nitrogen. The suspension was degassed under vacuum and purged with 12 several times. The resulting mixture was stirred at room temperature for 3 hrs. After completion, the suspension was filtered through a pad of Celite®, the filter cake was washed with MeOH (20 mL). The combined filtrates were concentrated to dryness to give tert-butyl ((5S,8S,10aR)-8-(3-(4-(dimethylamino)pyridin-3-yl)azetidine-1-carbonyl)-6-oxodecahydropyrrolo[1,2-a][1,5]diazocin-5-yl)carbamate (420 mg, 864 μmol, 83%) as a white solid. LCMS (ESI): m/z=487 [M+H]⁺.

### STEP G: 8-(2-(2,6-dioxopiperidin-3-yl)-1-oxoisoindolin-4-yl)oct-7-ynoic acid

To a solution of 3-(4-bromo-1-oxoisoindolin-2-yl)piperidine-2,6-dione (500 mg, 1.55 mmol, 1.0 eq.), oct-7-ynoic acid (239 mg, 1.7 mmol, 1.1 eq.) and TEA (310 mg, 3.1 mmol, 2.0 eq.) in DMF (10 mL) were added Pd(dppf)Cl₂ (113 mg, 0.16 mmol, 0.1 eq.) and CuI (294 mg, 1.55 mmol, 10 eq.). The reaction mixture was stirred at 80° C. overnight. After completion, the reaction mixture was diluted with H₂O (30 mL) and extracted with EtOAc (30 mL×2). The organic layers were combined and washed with brine (20 mL), dried over anhydrous Na₂SO₄, and concentrated under reduced pressure. The residue was purified by flash column chromatography on silica gel to afford 8-(2-(2,6-dioxopiperidin-3-yl)-1-oxoisoindolin-4-yl)oct-7-ynoic acid (400 mg, 1.05 mmol, 68%) as a white solid. LCMS (ESI): m/z=383 [M+H]⁺.

### STEP H: tert-butyl ((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamate

To a mixture of 8-(2-(2,6-dioxopiperidin-3-yl)-1-oxoisoindolin-4-yl)oct-7-ynoic acid (157 mg, 0.41 mmol, 1.0 eq.) and HATU (171 mg, 0.45 mmol, 1.1 eq.) in DMF (5 mL) were added DIEA (106 mg, 0.82 mmol, 2.0 eq.) and tert-butyl ((5S,8S,10aR)-8-(3-(4-(dimethylamino)pyridin-3-yl)azetidine-1-carbonyl)-6-oxodecahydropyrrolo[1,2-a][1,5]diazocin-5-yl)carbamate (200 mg, 0.41 mmol, 1.0 eq.). The resulting mixture was stirred at 25° C. for 2 hrs. After completion, the reaction mixture was purified directly by Biotage® C18 column chromatography to afford tert-butyl ((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamate (250 mg, 0.29 mmol, 72%) as a white solid. LCMS (ESI): m/z=851 [M+H]⁺.

### STEP I: 3-(4-(8-((5S,8S,10aR)-5-amino-8-(3-(4-(dimethylamino)pyridin-3-yl)azetidine-1-carbonyl)-6-oxooctahydropyrrolo[1,2-a][1,5]diazocin-3(4H)-yl)-8-oxooct-1-yn-1-yl)-1-oxoisoindolin-2-yl)piperidine-2,6-dione

To a solution of tert-butyl ((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamate (250 mg, 0.29 mmol, 1.0 eq.) in DCM (10 mL) was added TFA (5 mL) and the reaction mixture was stirred at room temperature for 2 hrs. After completion, the reaction mixture was concentrated under reduced pressure to get crude 3-(4-(8-((5S,8S,10aR)-5-amino-8-(3-(4-(dimethylamino)pyridin-3-yl)azetidine-1-carbonyl)-6-oxooctahydropyrrolo[1,2-a][1,5]diazocin-3(4H)-yl)-8-oxooct-1-yn-1-yl)-1-oxoisoindolin-2-yl)piperidine-2,6-dione (230 mg, quant.) as a white solid, which was used in next step directly without further purification. LCMS (ESI): m/z=751 [M+H]⁺.

### STEP J: S,S′-(((((2-(((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphoryl)bis(oxy))bis(ethane-2,1-diyl)) dibutanethioate

To a solution of 3-(4-(8-((5S,8S,10aR)-5-amino-8-(3-(4-(dimethylamino)pyridin-3-yl)azetidine-1-carbonyl)-6-oxooctahydropyrrolo[1,2-a][1,5]diazocin-3(4H)-yl)-8-oxooct-1-yn-1-yl)-1-oxoisoindolin-2-yl)piperidine-2,6-dione (40 mg, 53 μmol, 1.0 eq.) and perfluorophenyl 5-((bis(2-(butyrylthio)ethoxy)phosphoryl)difluoromethyl)benzo[b]thiophene-2-carboxylate (39 mg, 53.2 μmol, 1.0 eq.) in DMF (5 mL) was added DIEA (54 mg, 415 μmol, 5.0 eq.). The reaction mixture was stirred at room temperature for 8 hrs. After completion, the residue was purified by prep-HPLC to give S,S′-(((((2-(((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphoryl)bis(oxy))bis(ethane-2,1-diyl)) dibutanethioate (Compound 38, 14.6 mg, 11.2 μmol, 21%) as white solid.

¹H NMR (400 MHz, DMSO-d6) δ 10.98 (s, 1H), 9.36-8.65 (m, 1H), 8.45-8.28 (m, 2H), 8.6-8.172 (m, 3H), 7.79-7.34 (m, 4H), 6.92-6.79 (m, 1H), 5.18-5.06 (m, 1H), 5.05-4.58 (m, 2H), 4.56-3.97 (m, 1H), 3.98-3.35 (m, 6H), 3.14-3.12 (m, 4H), 2.97-2.82 (m, 1H), 2.72-2.65 (m, 6H), 2.57-2.51 (m, 8H), 2.23-2.17 (m, 1H), 2.12-1.91 (m, 3H), 1.91-1.69 (m, 4H), 1.67-1.43 (m, 10H), 0.88-0.83 (m, 6H).

LCMS (ESI): m/z=1302.2 [M+H]⁺.

### STEP K: ((2-(((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphonic acid

To a solution of 3-(4-(8-((5S,8S,10aR)-5-amino-8-(3-(4-(dimethylamino)pyridin-3-yl)azetidine-1-carbonyl)-6-oxooctahydropyrrolo[1,2-a][1,5]diazocin-3(4H)-yl)-8-oxooct-1-yn-1-yl)-1-oxoisoindolin-2-yl)piperidine-2,6-dione (20 mg, 27 μmol, 1.0 eq.) and (difluoro(2-((perfluorophenoxy)carbonyl)benzo[b]thiophen-5-yl)methyl)phosphonic acid (13 mg, 27 mol, 1.0 eq.) in DMF (5 mL) was added DIEA (17 mg, 133 μmol, 5.0 eq) and the reaction mixture was stirred at room temperature for 4 hrs. After completion, the residue was purified by prep-HPLC to give ((2-(((5S,8S,10aR)-8-(3-(4-(dimethylamino)pyridin-3-yl)azetidine-1-carbonyl)-3-(8-(2-(2,6-dioxopiperidin-3-yl)-1-oxoisoindolin-4-yl)oct-7-ynoyl)-6-oxodecahydropyrrolo[1,2-a][1,5]diazocin-5-yl)carbamoyl)benzo[b]thiophen-5-yl)difluoromethyl)phosphonic acid (Compound 39, 15 mg, 14 μmol, 54%) as white solid.

¹H NMR (400 MHz, DMSO-d6) δ 11.00 (s, 1H), 8.34-8.22 (m, 2H), 8.18-8.11 (m, 1H), 8.08-7.99 (m, 2H), 7.99-7.84 (m, 1H), 7.74-7.44 (m, 5H), 5.19-5.08 (m, 1H), 5.04-4.81 (m, 1H), 4.69-4.53 (m, 1H), 4.51-4.37 (m, 2H), 4.36-4.18 (m, 5H), 4.15-4.04 (m, 1H), 3.96-3.79 (m, 2H), 3.76-3.55 (m, 3H), 3.14-3.08 (m, 1H), 2.93-2.87 (m, 6H), 2.28-2.12 (m, 1H), 2.07-1.93 (m, 2H), 1.67-1.53 (m, 3H), 1.52-1.37 (m, 2H), 1.32-1.20 (m, 9H).

LCMS (ESI): m/z=1041.3 [M+H]⁺.

### ((7-(((5S,8S,10aR)-8-((8-(2-(2,6-dioxopiperidin-3-yl)-1-oxoisoindolin-4-yl)oct-7-yn-1-yl)(methyl)carbamoyl)-3-(5-methylbenzo[d]isoxazole-3-carbonyl)-6-oxodecahydropyrrolo[1,2-a][1,5]diazocin-5-yl)carbamoyl)naphthalen-2-yl)fluoromethyl)phosphonic acid (Compound 40)

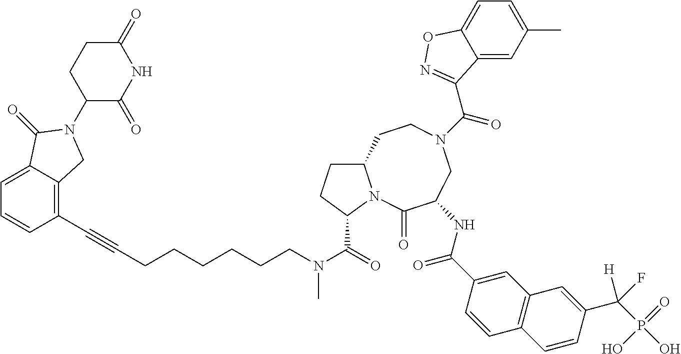 [SMILES: Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(C(F)P(=O)(O)O)cc5c4)C3)c2c1]

¹H NMR (400 MHz, DMSO) δ 11.07-10.89 (m, 1H), 8.94-8.65 (m, 1H), 8.53 (d, J=51.8 Hz, 1H), 8.11-7.84 (m, 5H), 7.78-7.24 (m, 7H), 6.01-5.82 (m, 1H), 5.35-5.18 (m, 1H), 5.17-5.09 (m, 1H), 4.79-4.70 (m, 1H), 4.51-4.38 (m, 2H), 4.37-4.17 (m, 2H), 4.04-3.96 (m, 2H), 3.36-3.25 (m, 2H), 3.19-3.09 (m, 1H), 3.02-2.83 (m, 3H), 2.79-2.56 (m, 3H), 2.46-2.42 (m, 3H), 2.34-2.09 (m, 3H), 2.04-1.88 (m, 2H), 1.85-1.70 (m, 2H), 1.67-1.58 (m, 1H), 1.56-1.48 (m, 2H), 1.43-1.30 (m, 3H), 1.28-1.22 (m, 2H), 1.20-1.11 (m, 1H). (TFA salt). ¹⁹F NMR (376 MHz, DMSO) δ −196.86-−197.34 (m). ³¹P NMR (162 MHz, DMSO) δ 10.87 (d, J=79.9 Hz). LCMS: Rt=1.128 min, 1016.1 (M+H)⁺

### Biochemical and Cellular Assays

### STAT3 Fluorescence Polarization (FP) Assay

An FP assay was developed to determine IC₅₀ values for test substances. Recombinant STAT3 protein (STAT3(G127-1722)) at 25 nM was combined with a fluorescently labeled, phosphotyrosine peptide probe (5-FAM-GpYLPQTV-NH₂) at 2 nM in FP buffer (10 mM HEPES pH 7.4, 50 mM NaCl, 1 mM EDTA, 0.05% Tween 20.2 mM DTT). 50 μL of STAT3-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 2. For STAT3 FP assay. A=≤100 nM; B=>100-1000 nM; C=>1-10 μM; and D=>10 μM.

### STAT6 Fluorescence Polarization (FP) Assay

An FP assay was developed to determine IC₅₀ values for test substances. Recombinant STAT6 protein (STAT6(W123-T658)) at 250 nM was combined with a fluorescently labeled, phosphotyrosine peptide probe (5-FAM-ApYKPFQDLI-NH₂) at 2 nM in FP buffer (10 mM HEPES pH 7.4, 50 mM NaCl, 1 mM EDTA, 0.05% Tween 20, 2 mM DTT), 50 μL of STAT6-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 2. For STAT6 FP assay. A=≤300 nM; B=>300-3000 nM; C=>3-30 μM; and D=>30 μM.

TABLE 2

| Cmp# | STAT3 | STAT6 |
|---|---|---|
| 1 | C | B |
| 3 | B | B |
| 5 | A | C |
| 6 | C | A |
| 7 | C | A |
| 9 | C | A |
| 11 | C | A |
| 14 | D | B |
| 15 | D | B |
| 22 | D | A |
| 27 | C | A |
| 28 | C | A |
| 29 | C | A |
| 30 | C | A |
| 31 | C | |
| 32 | C | B |
| 33 | C | A |
| 34 | C | A |
| 35 | D | A |
| 36 | C | A |
| 37 | D | A |
| 39 | A | C |
| 40 | D | A |

### MSD-pSTAT-PBMC Assay

### Materials

Cryopreserved Peripheral Blood Mononuclear Cells (PBMC) are from AllCells. Recombinant Human IL-4 and IL-6 are from Peprotech. Mouse monoclonal anti-STAT3 antibody, rabbit monoclonal anti-pY705-STAT3 antibody, rabbit monoclonal Anti-pY641-STAT6 antibody, and lysis buffer are from Cell Signaling Technology (CST). Mouse monoclonal anti-STAT6 antibody is from BioLegend. Assay plates, blocker, and anti-rabbit secondary antibody are from Meso Scale Discovery (MSD).

### Assay Method:

Cryopreserved PBMCs were thawed out and allowed to recover overnight in IMDM+10% heat-inactivated FBS prior to plating 50,000 (STAT3) or 25,000 (STAT6) cells per well in 96-well U-bottom tissue culture plates. Cells were treated with compound for 3 hrs. then stimulated with 10 ng/mL IL-6 (STAT3) or 1 ng/mL IL-4 (STAT6) for 10 min. Cells were then spun down and washed with ice-cold PBS prior to lysing the cell pellet with 1× lysis buffer (CST) with 1×HALT protease and phosphatase inhibitor cocktail (Thermo). Lysates were transferred to and incubated overnight at 4° C. with shaking in QuickPlex 96-well high bind assay plates (MSD) pre-coated overnight with 30 μL per well of 0.6 μg/mL mouse monoclonal anti-STAT3 antibody (STAT3) or 2 μg/mL mouse monoclonal anti-STAT6 antibody (STAT6) in 1×PBS, and pre-blocked for 1 hour with 3% Blocker-A (MSD). Captured protein in the assay plates were then washed and probed with 25 μL per well of 0.25 μg/ml rabbit monoclonal anti-pY705-STAT3 antibody (STAT3) or 0.18 μg/ml rabbit monoclonal Anti-pY641-STAT6 antibody (STAT6) in 1% Blocker-A for 1 hour at room temperature with shaking, then washed and probed with 25 ul per well of 1 ug/ml Sulfo-TAG Labeled Goat Anti-Rabbit Antibody (MSD) in 1% Blocker-A for 1 hour at room temperature with shaking. Assay plates were then washed and 150 μL of 1× Read Buffer-T (MSD) was added to each well prior to reading on an SQ120 MSD Plate Reader. Assay signal from each sample was subtracted by the signal from unstimulated control wells and normalized to DMSO control wells. IC₅₀ values were calculated using Graph Pad Prism Dose-Response Nonlinear Regression with variable slope. Results are shown in Table 3. For both pSTAT3 and pSTAT6 A=≤100 nM; B=>100-1000 nM; C=>1000-3000 nM; and D=>3 μM.

TABLE 3

| Cmp# | pSTAT3 | pSTAT6 |
|---|---|---|
| 1 | D | D |
| 2 | C | C |
| 3 | D | D |
| 4 | C | D |
| 5 | D | D |
| 6 | D | C |
| 7 | D | D |
| 8 | D | C |
| 9 | D | C |
| 10 | D | A |
| 11 | | B |
| 12 | D | A |
| 13 | D | D |
| 14 | | D |
| 16 | | D |
| 15 | | D |
| 17 | | D |
| 18 | | A |
| 20 | | C |
| 21 | D | A |
| 23 | | D |
| 19 | | A |
| 22 | | B |
| 26 | | D |
| 27 | | D |
| 28 | | D |
| 29 | | D |
| 30 | | D |
| 31 | | D |
| 32 | | D |
| 33 | | D |
| 34 | | D |
| 35 | | D |
| 36 | | D |
| 37 | | D |
| 38 | A | |
| 39 | B | |
| 40 | | D |

### MSD-tSTAT-PBMC Assay

### Materials

Cryopreserved Peripheral Blood Mononuclear Cells (PBMC) for STAT3 assay are from AllCells and for STAT6 assay are from STEMCELL Technologies. Mouse monoclonal anti-STAT6 antibody is from BioLegend. The rabbit monoclonal anti-STAT6 antibody and cell lysis buffer are from Cell Signaling Technologies. Total STAT3 assay kit is from Meso Scale Discovery (MSD). Assay plates. Blocker A. Blocker D-R. Blocker D-M, and anti-rabbit secondary antibody for the total STAT6 assay is from Meso Scale Discovery (MSD).

### Assay Method:

Cryopreserved PBMCs were thawed out and allowed to recover overnight in IMDM+10% heat-inactivated FBS and 1× Glutamax prior to plating 50,000 (STAT3) or 100.000 (STAT6) cells per well in a 96-well U-bottom tissue culture plate. Cells were treated with 10 uM compound for 20 hours, then spun down and washed with ice-cold PBS prior to lysing the cell pellet with 1× lysis buffer (CST) supplemented with 1×HALT protease and phosphatase inhibitor cocktail (Thermo). Cells were then frozen at −80 C. thawed, and incubated with shaking at 4 C for 1 hour to allow cellular lysis. For the total STAT3 (tSTAT3) assay, assay ready plates (MSD) pre-blocked for 1 hour with 3% Blocker A (MSD) were used. Then, cell lysates were transferred to and incubated overnight at 4 C with shaking in QuickPlex 96-well high bind assay plates (MSD). For the total STAT6 (tSTAT6) assay, assay plates were pre-coated overnight with 30 ul per well of 2 ug/mL mouse monoclonal anti-STAT6 antibody (BioLegend) in 1×PBS, and pre-blocked for 1 hour with 3% Blocker A (MSD). Then, cell lysates were transferred to and incubated for 72 h at 4 C with shaking in QuickPlex 96-well high bind assay plates (MSD). Following protein capture on the assay plates, plates were washed and probed with appropriate antibody. For the STAT3 assay, 25 ul SULFO-tag conjugated anti-total STAT3 antibody in 1% Blocker A/0.1% Blocker D-M/0.1% Blocker D-R (MSD) was added and plates were incubated for 1 hr with shaking at room temperature. For the STAT6 assay, 25 ul per well of 0.25 ug/mL rabbit monoclonal anti-STAT6 antibody (CST) in 1% Blocker A was added and plates were incubated for 1 hour at room temperature with shaking, then washed and probed with 25 ul per well of 1 ug/mL. Sulfo-TAG labeled Goat anti-Rabbit Antibody (MSD) in 1% Blocker A for 1 hour at room temperature with shaking. Assay plates were then washed and 150 ul of 1× Read Buffer-T (MSD) was added to each well prior to reading on an SQ120 MSD Plate Reader. Assay signal from each sample was subtracted by the signal from control wells and normalized to DMSO control wells to calculate percent control and percent degradation. Each condition was run in duplicate across two separate assays and values were averaged. Results are shown in Table 4. For both tSTAT3 and tSTAT6 A=85% degradation; B=>70%-85% degradation; C=50%-70% degradation; and D=<50% degradation.

TABLE 4

| Cmp# | tSTAT3 | tSTAT6 |
|---|---|---|
| 1 | | D |
| 2 | | D |
| 3 | | D |
| 4 | | D |
| 6 | | A |
| 7 | | C |
| 8 | | C |
| 9 | | A |
| 10 | | A |
| 11 | | A |
| 12 | | A |
| 13 | | A |
| 14 | | A |
| 16 | | D |
| 15 | | D |
| 17 | | C |
| 18 | | C |
| 20 | | A |
| 21 | | A |
| 23 | | B |
| 19 | | B |
| 22 | | A |
| 26 | | A |
| 27 | | D |
| 28 | | D |
| 29 | | D |
| 30 | | A |
| 31 | | C |
| 32 | | D |
| 33 | | D |
| 34 | | D |
| 35 | | D |
| 36 | | D |
| 37 | | D |
| 5 | A | |
| 38 | A | |
| 39 | A | |
| 40 | | D |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the structural formula I: 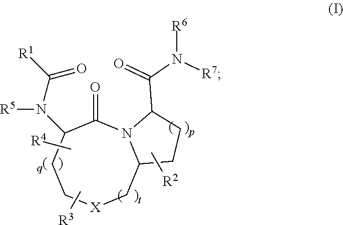
or a pharmaceutically acceptable salt thereof, wherein:
q is 0 or 1 and t is 0, 1, or 2, provided that at least one of q or t is 1;
p is 1 or 2;
X is selected from S, SO₂, —S(═O)═NH, —NR⁸, and —NR⁹;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}], wherein said aryl may be further optionally substituted with 1 or 2 groups independently selected from cyano, (C₁-C₄)alkoxy, and halo; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], —[P(O)[NH(AA)C(O)OR^{T}][NH(AA)C(O)OR^{T}], or —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —P(O)OR^{1b}OR^{2b}, —[P(O)[NHR^{Ty}][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alky]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl, —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
R⁵ and R⁶ are each independently selected from hydrogen, phenyl, and (C₁-C₄)alkyl;
R⁷ is selected from E, —R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q};
R⁸ is selected from hydrogen, (C₁-C₄)alkyl, halo(C₁-C₄)alkyl, (C₃-C₆)cycloalkyl, 5- to 7-membered heterocyclyl, —(C₁-C₄)[5- to 7-membered heterocyclyl], 5- to 10-membered heteroaryl, —(C₁-C₄)[5- to 10-membered heteroaryl], phenyl, —(C₁-C₄)alkylphenyl, —C(O)R^{Ha}, —C(O)OR^{Ha}, —C(O)NR^{Ha}R^{Hb}, —C(O)OR^{Ha}, —SOR^{Ha}R^{Hb} and —SO₂R^{Ha}, wherein said (C₃-C₆)cycloalkyl, 5- to 7-membered heterocyclyl, 5- to 10-membered heteroaryl, phenyl, the phenyl on (C₁-C₄)alkylphenyl, the 5- to 7-membered heterocyclyl on —(C₁-C₄)[5- to 7-membered heterocyclyl], and the 5- to 6-membered heteroaryl on —(C₁-C₄)[5- to 6-membered heteroaryl] are each optionally substituted with, as valency permits, 1 to 3 groups selected from R^{U};
R⁹ is selected from-R^{10E}E and —C(O)R^{10E};
R¹⁰, R^{10A}, R^{10B}, R^{10C}, and R^{10D} are each independently a chemical spacer unit;
R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with-OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with-OR^{10C}E, —NHR^{10C}-E, —[N(C₁-C₄)alkyl]R^{10C}E, or R^{10C}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
R^{Ha} is selected from (C₁-C₁₀)alkyl, (C₂-C₁₀)alkenyl, (C₂-C₁₀)alkynyl, phenyl, 5- to 10-membered monocyclic or bicyclic heteroaryl, and 4- to 10-membered monocyclic or bicyclic heterocyclyl, wherein said (C₁-C₁₀)alkyl, (C₂-C₁₀)alkenyl, (C₂-C₁₀)alkynyl are each optionally substituted with, as valency permits, 1 to 2 groups selected from R^{O} and wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, cyano, and 4- to 6-membered heterocyclyl optionally substituted with C₁-C₄alkyl;
R^{O} is selected from halo, (C₁-C₄)alkoxy, OH, phenyl, NH₂, —NH(C₁-C₁₀)alkyl, —N[(C₁-C₁₀)alkyl], (C₃₋₆cycloalkyl), 4- to 10-membered monocyclic or fused bicyclic heterocyclyl and 5- to 10-membered monocyclic or bicyclic heteroaryl, wherein said 5- to 10-membered monocyclic or bicyclic heteroaryl and said 4- to 10-membered monocyclic or bicyclic heterocyclyl are each optionally substituted with, as valency permits, 1 to 3 groups selected from halo, (C₁-C₄)alkyl, halo(C₁-C₄)alkyl, (C₁-C₄)alkoxy, halo(C₁-C₄)alkoxy, oxo, and cyano;
R^{Hb} is hydrogen or (C₁-C₄)alkyl;
AA is the residue of an alpha or beta natural or non-natural amino acid;
R^{T} and R^{Ty} are each independently selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} and R^{U} are each independently selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{e}R^{d}, —NR^{a}R^{b}, —S(O)R^{e}R^{f}, —S(O)₂R^{f}, —S(O)—NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{c}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{e}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, and R^{X} are each independently selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{e}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alknyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo,
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

2. The compound of claim 1, or a pharmaceutically acceptable salt thereof, wherein
X is NR⁹;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{e}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alknyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo.

3. The compound of claim 1, or a pharmaceutically acceptable salt thereof, wherein
X is selected from S, SO₂, —S(═O)═NH, —NR⁸;
R⁷ is selected from E, —R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo.

4. The compound of claim 1, or a pharmaceutically acceptable salt thereof, wherein
X is selected from S, SO₂, —S(═O)═NH, —NR⁸;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{c}R^{d}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo.

5. The compound of claim 1, or a pharmaceutically acceptable salt thereof, wherein
X is selected from S, SO₂, —S(═O)═NH, —NR,
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo.

6. The compound of claim 1, or a pharmaceutically acceptable salt thereof, wherein
X is selected from S, SO₂, —S(═O)═NH, —NR⁸;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}; and
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alknyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alknyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo.

7. The compound of any one of claims 1 to 6, wherein the compound is of the structural formula II: 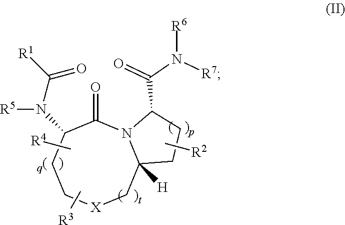
or a pharmaceutically acceptable salt thereof.

8. The compound of any one of claims 1 to 7, or a pharmaceutically acceptable salt thereof, wherein p is 1.

9. The compound of any one of claims 1 to 8, or a pharmaceutically acceptable salt thereof, wherein R² is hydrogen.

10. The compound of any one of claims 1 to 9, or a pharmaceutically acceptable salt thereof, wherein R³ is hydrogen.

11. The compound of any one of claims 1 to 10, or a pharmaceutically acceptable salt thereof, wherein R⁴ is hydrogen.

12. The compound of any one of claims 1 to 11, or a pharmaceutically acceptable salt thereof, wherein q is 1 and t is 1.

13. The compound of any one of claims 1 to 12, or a pharmaceutically acceptable salt thereof, wherein q is 0 and t is 2.

14. The compound of any one of claims 1 to 13, wherein the compound is of the structural formula III, IV, V, VI, VII, VIII, IIIa, VIa, or VIIIa: 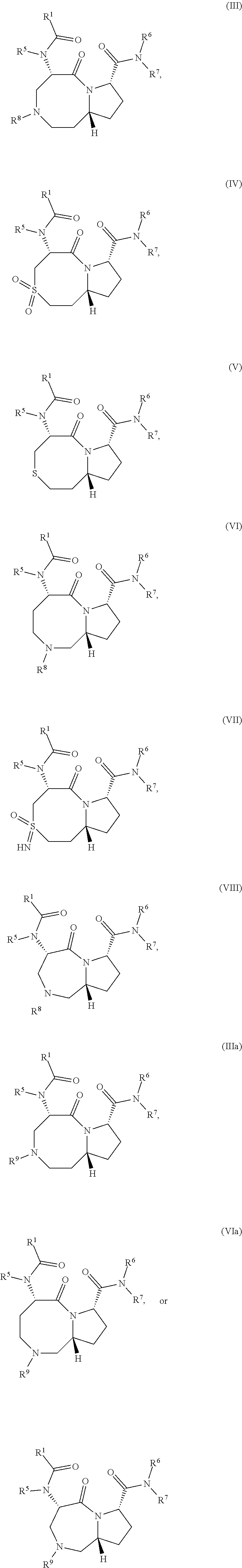
(VIIIa); or a pharmaceutically acceptable salt thereof.

15. The compound of any one of claims 1 to 14, wherein the compound is of the structural formula

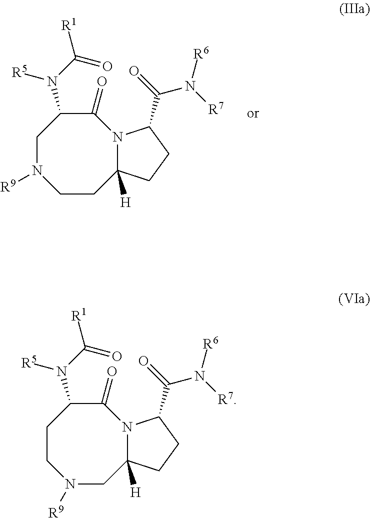

16. The compound of any one of claims 1 to 15, or a pharmaceutically acceptable salt thereof, wherein R⁵ is hydrogen.

17. The compound of any one of claims 1 to 16, or a pharmaceutically acceptable salt thereof, wherein R¹ is selected from 8- to 10-membered fused bicyclic heteroaryl, aryl, and (C₂-C₄)alkenyl(aryl), wherein said 8- to 10-membered fused bicyclic heteroaryl, aryl, and aryl portion of (C₂-C₄)alkenyl(aryl) are each substituted with —CR^{1a}R^{2a}P(O)OR^{1b}OR^{2b}.

18. The compound of any one of claims 1 to 17, or a pharmaceutically acceptable salt thereof, wherein, R¹ is selected from (C₂-C₄)alkenyl(phenyl) benzothiophenyl, indolyl, naphthalenyl, thienopyridinyl, benzothiazoyl, quinolinyl, isoquinolinyl, 4,5,6,7-tetrahydropyrrolopyridinyl, 5,6,7,8-tetrahydroimidazopyrazinyl, each of which are substituted with —CR^{1a}R^{2a}P(O)OR^{1b}OR^{2b}.

19. The compound of any one of claims 1 to 18, or a pharmaceutically acceptable salt thereof, wherein, R¹ is selected from

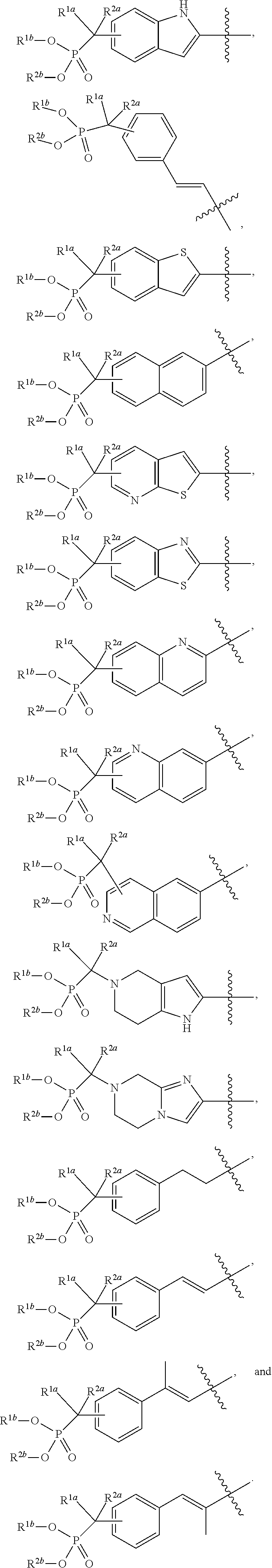

20. The compound of any one of claims 1 to 19, or a pharmaceutically acceptable salt thereof, wherein, R¹ is

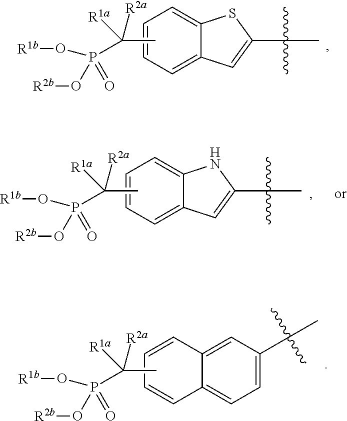

21. The compound of any one of claims 1 to 20, or a pharmaceutically acceptable salt thereof, wherein R^{1a} is hydrogen and R^{2a} is fluoro or R^{1a} is fluoro and R^{2a} is fluoro.

22. The compound of any one of claims 1 to 21, or a pharmaceutically acceptable salt thereof, wherein R^{1a} and R^{2a} are fluoro.

23. The compound of any one of claims 1 to 22, or a pharmaceutically acceptable salt thereof, wherein R^{1b} and R^{2b} are each independently selected from hydrogen, (C₁-C₄)alkyl, —[(C₁-C₄)alkyl]-OC(O)—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-OC(O)NR^{2c}R^{2d}], [(C₁-C₄)alkyl]-OC(O)O-[5- to 7-membered heterocyclyl], and phenyl, wherein the 5- to 7-membered heterocyclyl of [(C₁-C₄)alkyl]-OC(O)O-[5- to 7-membered heterocyclyl] is optionally substituted with C(O)OR^{2c}, wherein any of the (C₁—C)₄alkyl groups are optionally substituted with 1 or 2 (C₁-C₄)alkyl.

24. The compound of any one of claims 1 to 23, or a pharmaceutically acceptable salt thereof, wherein R^{1b} and R^{2b} are each —[(C₁-C₄)alkyl]-OC(O)—[(C₁-C₄)alkyl].

25. The compound of any one of claims 1 to 23, or a pharmaceutically acceptable salt thereof, wherein R^{1b} and R^{2b} are hydrogen.

26. The compound of any one of claims 1 to 20 and 23, or a pharmaceutically acceptable salt thereof, wherein —CR^{1a}R^{2a}P(O)OR^{1b}OR^{2b} is selected from

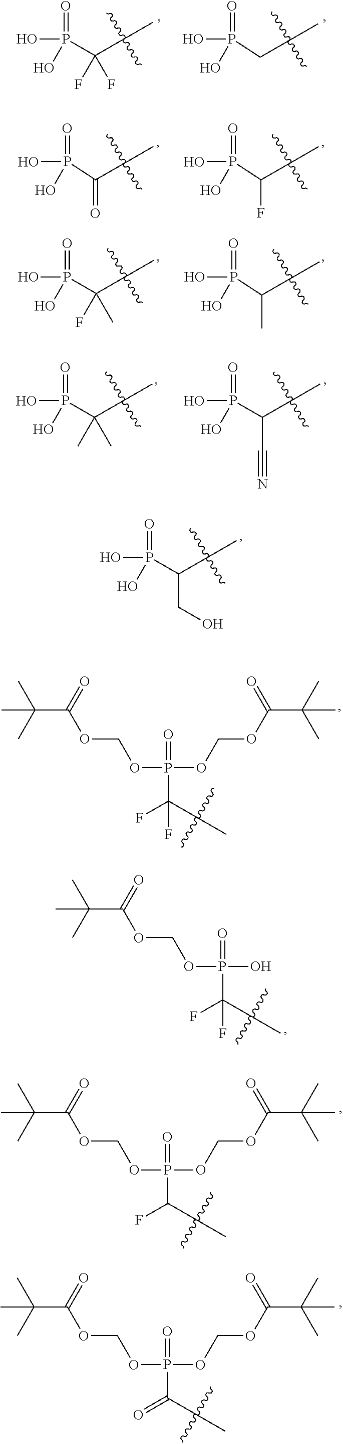

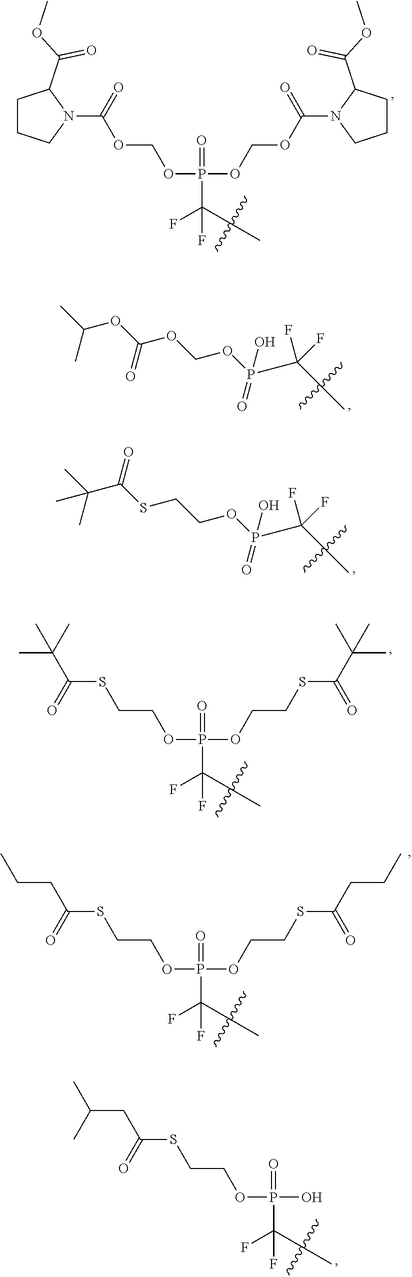

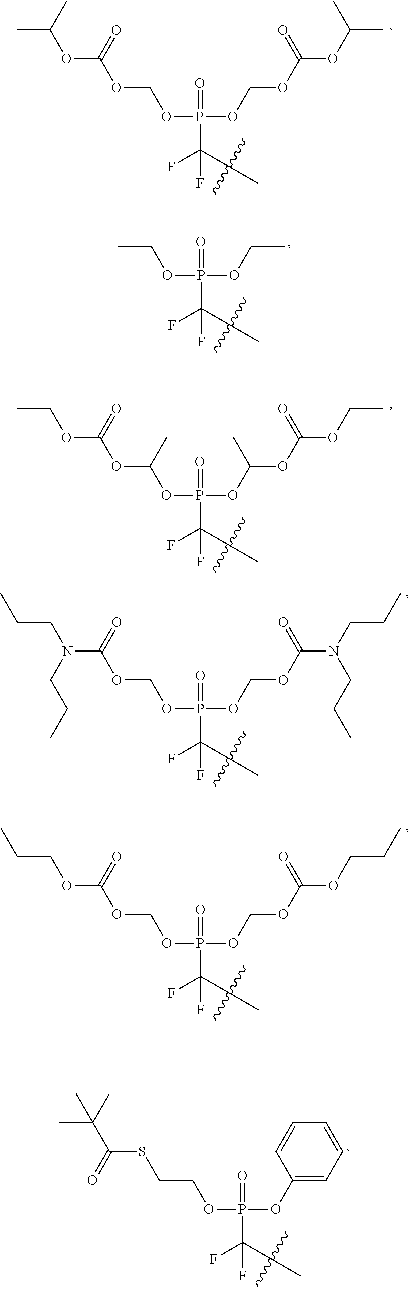

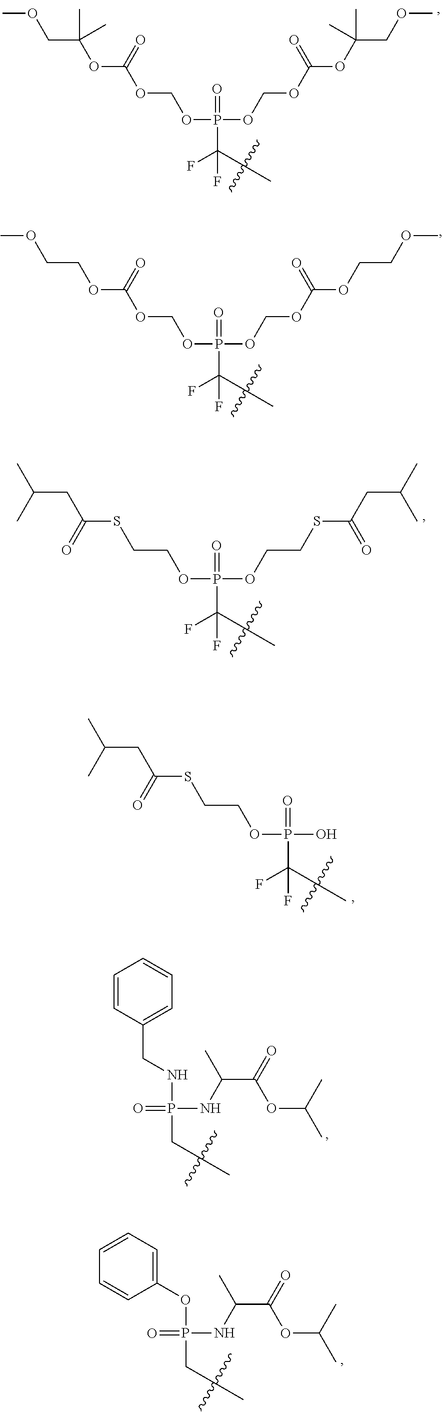

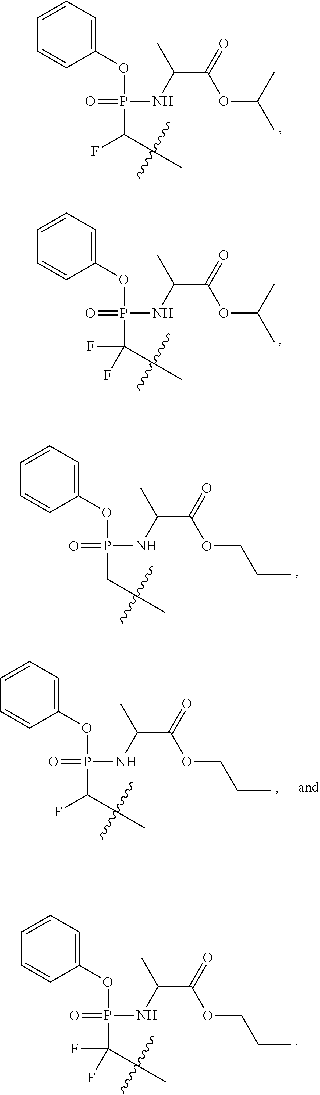 [SMILES: *C(F)P(=O)(NC(C)C(=O)OC(C)C)Oc1ccccc1 ; *C(F)(F)P(=O)(NC(C)C(=O)OC(C)C)Oc1ccccc1 ; *CP(=O)(NC(C)C(=O)OCCC)Oc1ccccc1 ; *C(F)P(=O)(NC(C)C(=O)OCCC)Oc1ccccc1 ; *C(F)(F)P(=O)(NC(C)C(=O)OCCC)Oc1ccccc1]

27. The compound of any one of claims 1 to 20, 21, and 26, or a pharmaceutically acceptable salt thereof, wherein —CR^{1a}R^{2a}P(O)OR^{1b}OR^{2b} is selected from

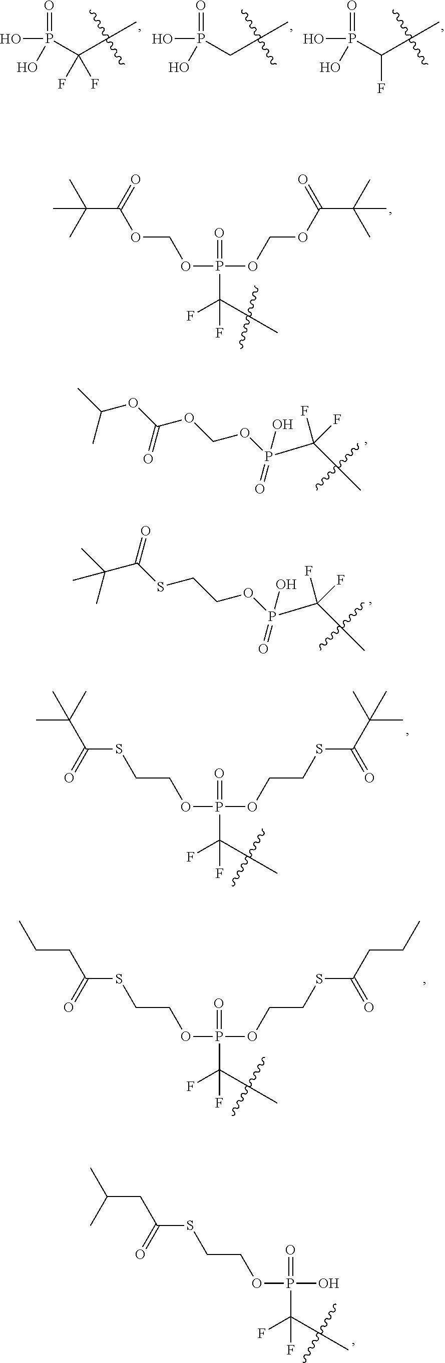

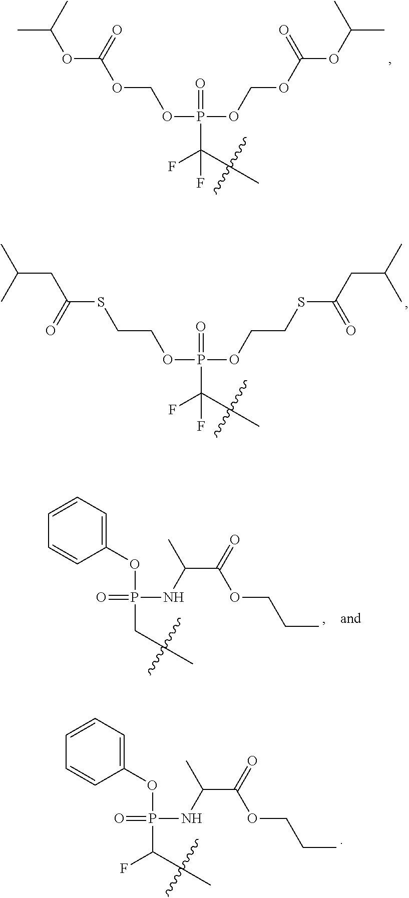

28. The compound of any one of claims 1 to 27, or a pharmaceutically acceptable salt thereof, wherein R⁶ is selected from hydrogen and (C₁-C₄)alkyl.

29. The compound of any one of claims 1 to 28, or a pharmaceutically acceptable salt thereof, wherein R⁷ is selected from E, —R^{10A}E, and phenyl.

30. The compound of any one of claims 1, 4, and 7 to 28, or a pharmaceutically acceptable salt thereof, wherein R⁷ is phenyl substituted with R¹¹.

31. The compound of any one of claims 1, 4, and 7 to 28, or a pharmaceutically acceptable salt thereof, wherein R¹¹ is —NHC(O)R^{10B}E.

32. The compound of any one of claims 1 to 31, or a pharmaceutically acceptable salt thereof, wherein R⁶ and R⁷ together with the nitrogen atom to which they are attached form a 4- to 6-membered monocyclic heterocyclyl optionally substituted with, as valency permits, R¹² and/or 1 to 3 groups selected from R^{Q}.

33. The compound of any one of claims 1 to 32, or a pharmaceutically acceptable salt thereof, wherein R⁶ and R⁷ together with the nitrogen atom to which they are attached form azetidinyl, pyrrolidyl, or piperidinyl, each optionally substituted with, as valency permits, R¹² and/or 1 to 3 groups selected from R^{Q}.

34. The compound of any one of claims 1 to 27, 32, and 33, or a pharmaceutically acceptable salt thereof, wherein R^{Q} is cyano, phenyl, or 5- to 7-membered monocyclic heteroaryl optionally substituted with, as valency permits, 1 to 3 groups selected from R^{F}.

35. The compound of any one of claims 1 to 27 and 32 to 34, or a pharmaceutically acceptable salt thereof, wherein R^{Q} is cyano, phenyl, or pyridinyl optionally substituted with, as valency permits, 1 to 3 groups selected from R^{F}.

36. The compound of any one of claims 1 to 27 and 32 to 35, or a pharmaceutically acceptable salt thereof, wherein R^{F} is —NR^{a}R^{b}.

37. The compound of any one of claims 1 to 27 and 33, or a pharmaceutically acceptable salt thereof, wherein R¹² is E.

38. The compound of any one of claims 1 to 37, or a pharmaceutically acceptable salt thereof, wherein R⁸ is —C(O)R^{Ha}.

39. The compound of any one of claims 1 to 38, or a pharmaceutically acceptable salt thereof, wherein R^{Ha} is 8- to 10-membered bicyclic heteroaryl optionally substituted with C₁-C₄alkyl.

40. The compound of any one of claims 1 to 39, or a pharmaceutically acceptable salt thereof, wherein R^{Ha} is indazolyl or benzoisoxazolyl, each optionally substituted with C₁-C₄alkyl.

41. The compound of any one of claims 1, 2, and 7 to 37, or a pharmaceutically acceptable salt thereof, wherein R⁹ is selected from-R¹⁰E and —C(O)R¹⁰E.

42. The compound of any one of claims 1 to 41, wherein R¹⁰, R^{10A}, R^{10B}, R^{10C}, and R^{10D} are each independently selected from (C₁-C₁₀)alkylene and (C₂-C₁₀)alkynelene each optionally substituted with one or more groups selected from halo and oxo, and wherein said (C₁-C₆)alkylene and (C₂-C₁₀)alkynelene may also be optionally interrupted by one or more heteroatoms selected from S, N, and O, and/or optionally interrupted by one or more rings selected from 5- to 7-membered heteroaryl, (C₃-C₆)cycloakyl, phenyl, and 4- to 7-membered heterocyclyl.

43. The compound of any one of claims 1 to 42, wherein R¹⁰, R^{10A}, R^{10B}, R^{10C}, and R^{10D} are each independently selected from (C₁-C₁₀)alkylene and (C₂-C₁₀)alkynelene.

44. The compound of any one of claims 1 to 43, wherein R¹⁰, R^{10A}, R^{10B}, R^{10C}, and R^{10D} are each independently selected from (C₁-C₁₀)alkylene and (C₂-C₁₀)alkynelene, 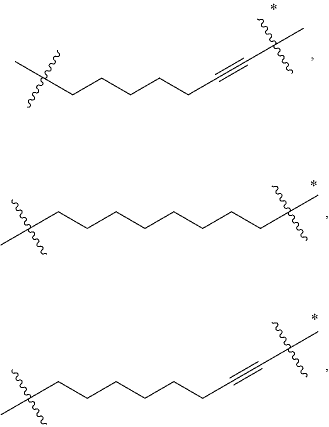 [SMILES: *C#CCCCCC* ; *CCCCCCCC* ; *C#CCCCCCC*]
CH₂, and (CH₂)₂, (CH₂)₃, (CH₂)₄.

45. The compound of any one of claims 1 to 44, or pharmaceutically acceptable salt thereof, wherein E is selected from a CRBN, VHL, IAP, or MDM2 based E3 binder.

46. The compound of any one of claims 1 to 45, or pharmaceutically acceptable salt thereof, wherein E is selected from 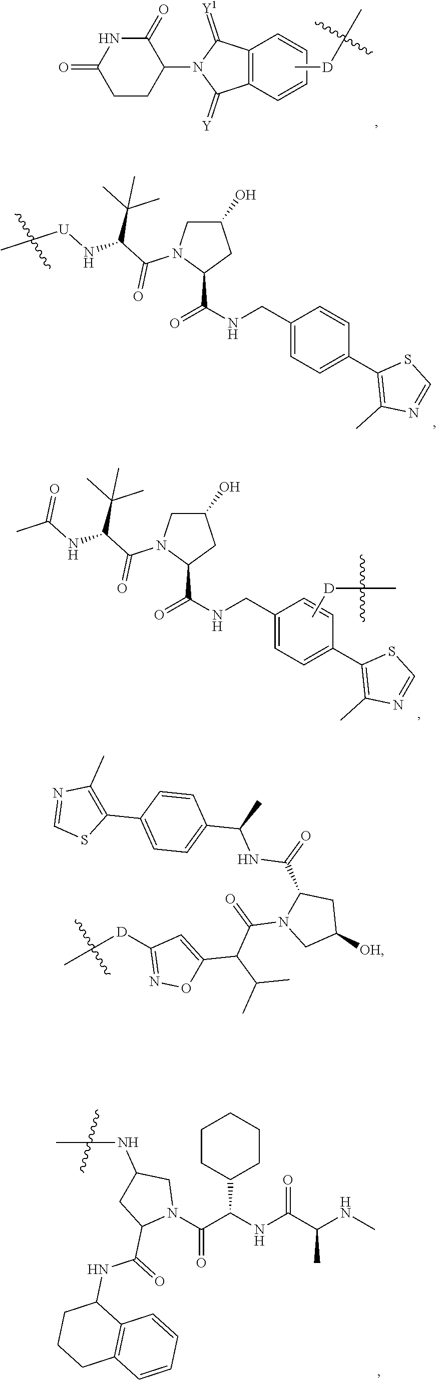 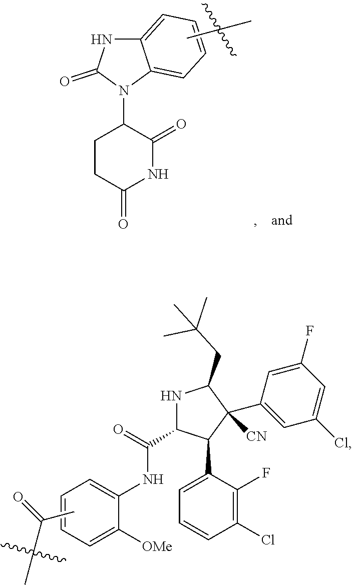
wherein:
Y and Y¹ are each independently is O or CH₂;
D is O, NH, or a bond;
U is absent or C(O); and
R is halo or cyano.

47. The compound of any one of claims 1 to 46, or pharmaceutically acceptable salt thereof, wherein E is selected from

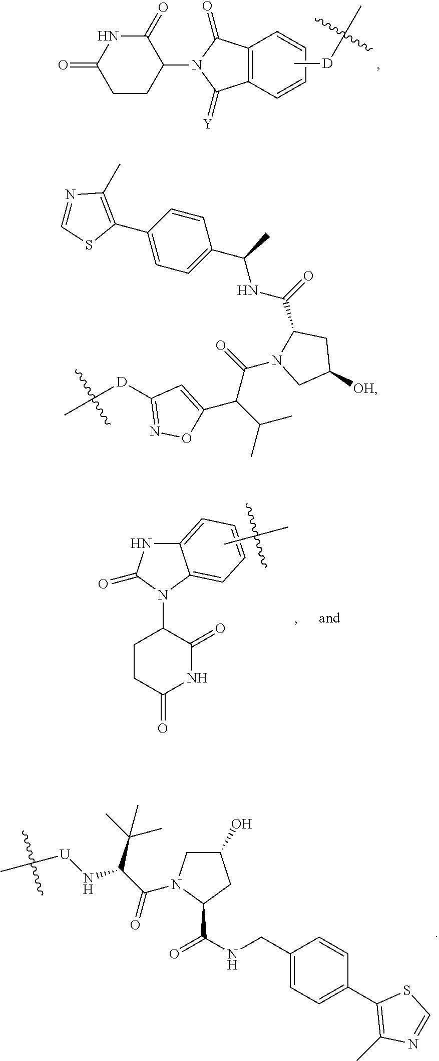

48. The compound of claim 1, or pharmaceutically acceptable salt thereof, wherein the compound is selected from

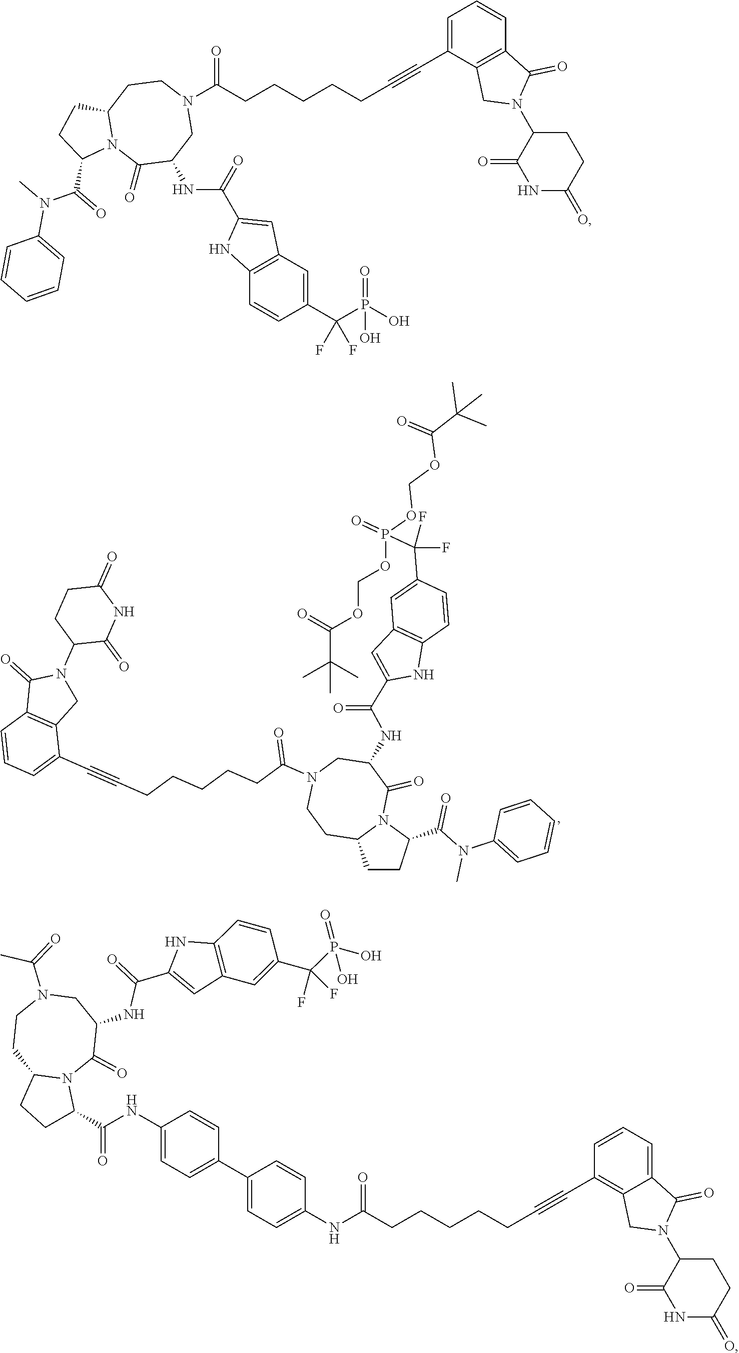 [SMILES: CN(C(=O)[C@@H]1CC[C@@H]2CCN(C(=O)CCCCCC#Cc3cccc4c3CN(C3CCC(=O)NC3=O)C4=O)C[C@H](NC(=O)c3cc4cc(C(F)(F)P(=O)(O)O)ccc4[nH]3)C(=O)N21)c1ccccc1 ; CN(C(=O)[C@@H]1CC[C@@H]2CCN(C(=O)CCCCCC#Cc3cccc4c3CN(C3CCC(=O)NC3=O)C4=O)C[C@H](NC(=O)c3cc4cc(C(F)(F)P(=O)(OCOC(=O)C(C)(C)C)OCOC(=O)C(C)(C)C)ccc4[nH]3)C(=O)N21)c1ccccc1 ; CC(=O)N1CC[C@H]2CC[C@@H](C(=O)Nc3ccc(-c4ccc(NC(=O)CCCCCC#Cc5cccc6c5CN(C5CCC(=O)NC5=O)C6=O)cc4)cc3)N2C(=O)[C@@H](NC(=O)c2cc3cc(C(F)(F)P(=O)(O)O)ccc3[nH]2)C1]

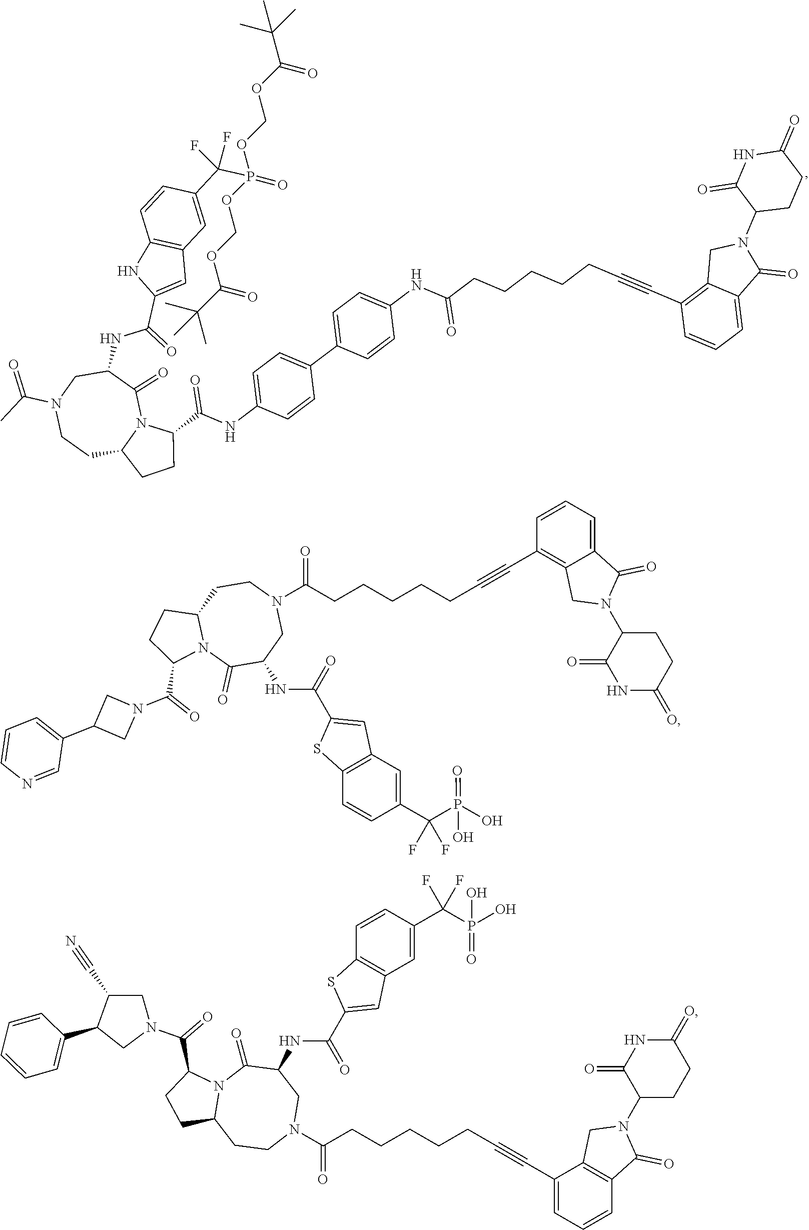

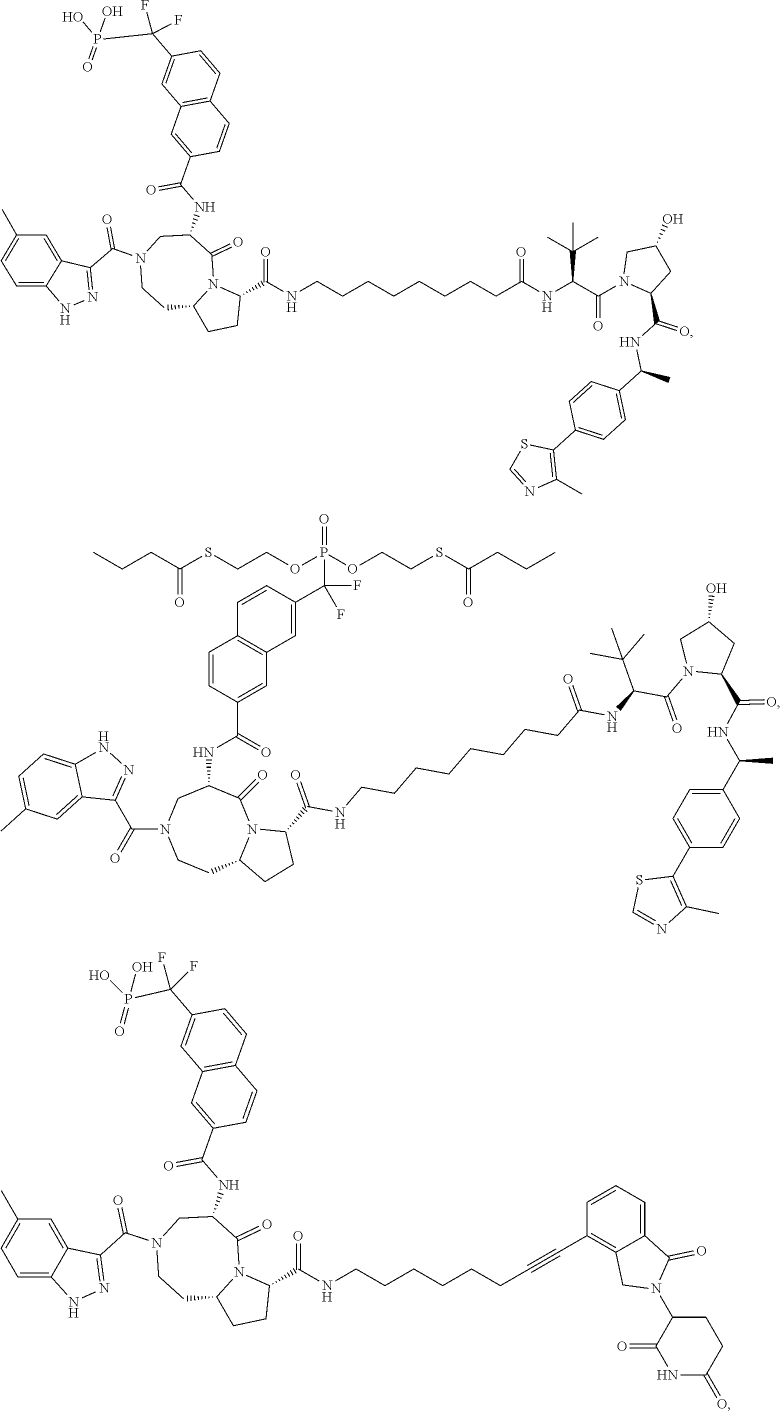

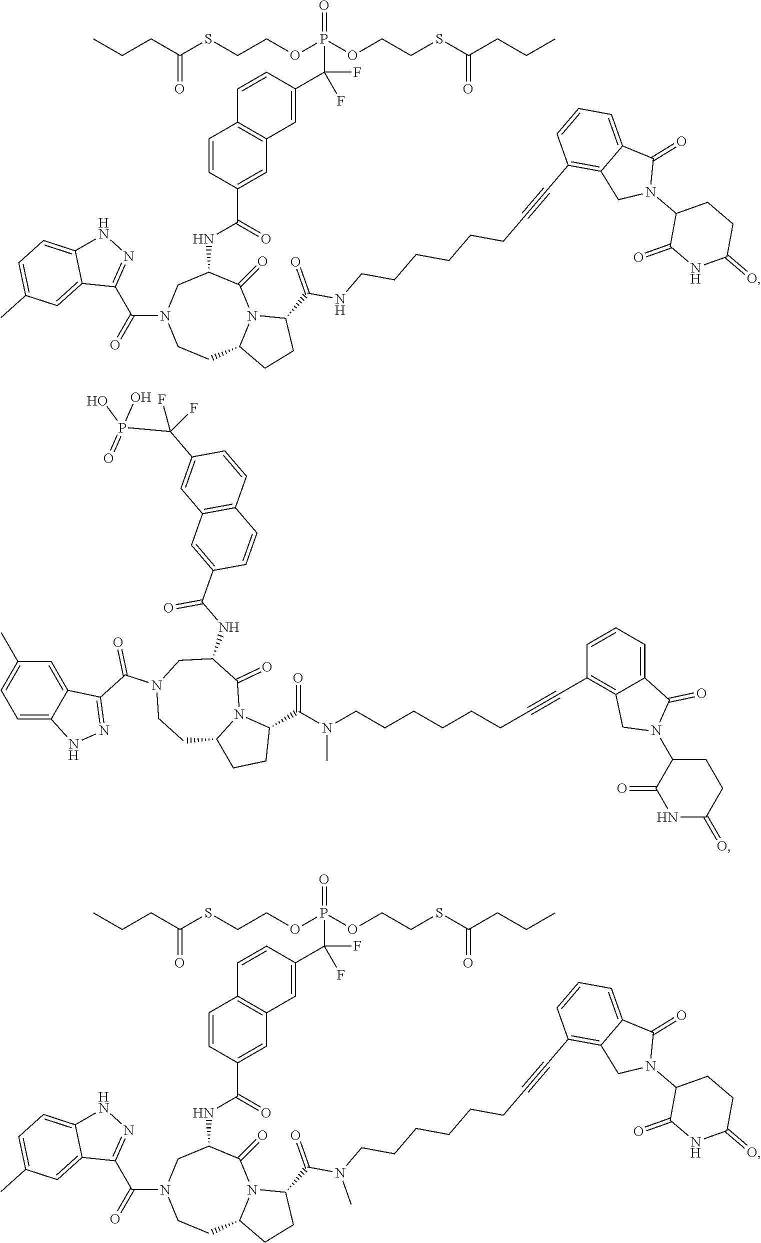 [SMILES: CCCC(=O)SCCOP(=O)(OCCSC(=O)CCC)C(F)(F)c1ccc2ccc(C(=O)N[C@H]3CN(C(=O)c4n[nH]c5ccc(C)cc45)CC[C@H]4CC[C@@H](C(=O)NCCCCCCC#Cc5cccc6c5CN(C5CCC(=O)NC5=O)C6=O)N4C3=O)cc2c1 ; Cc1ccc2[nH]nc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(C(F)(F)P(=O)(O)O)cc5c4)C3)c2c1 ; CCCC(=O)SCCOP(=O)(OCCSC(=O)CCC)C(F)(F)c1ccc2ccc(C(=O)N[C@H]3CN(C(=O)c4n[nH]c5ccc(C)cc45)CC[C@H]4CC[C@@H](C(=O)N(C)CCCCCCC#Cc5cccc6c5CN(C5CCC(=O)NC5=O)C6=O)N4C3=O)cc2c1]

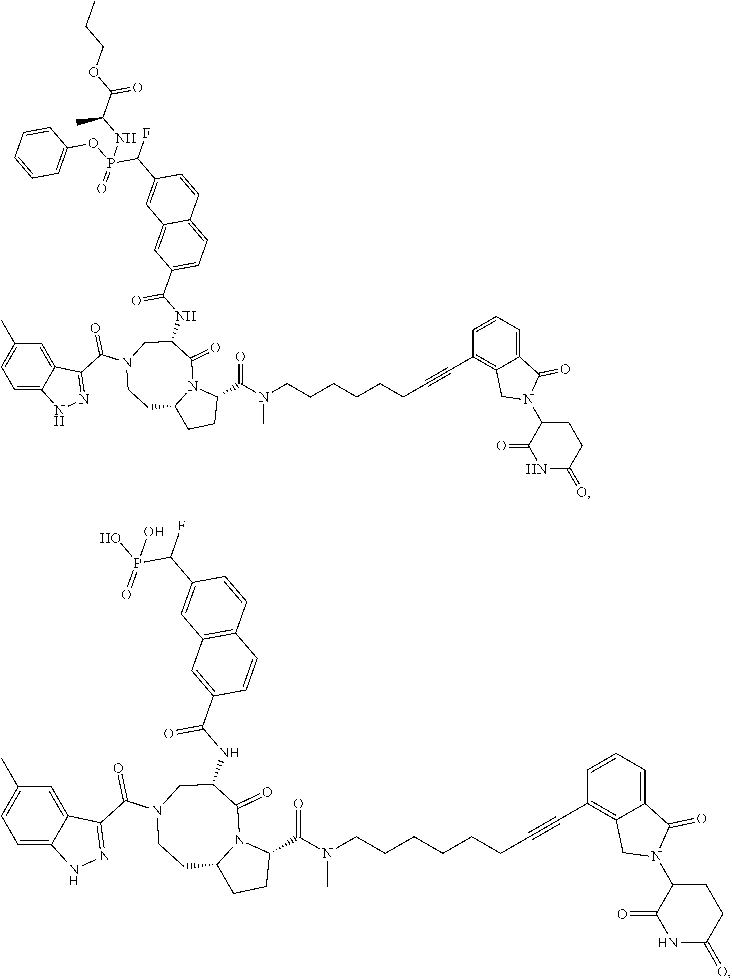 [SMILES: CCCOC(=O)[C@H](C)NP(=O)(Oc1ccccc1)C(F)c1ccc2ccc(C(=O)N[C@H]3CN(C(=O)c4n[nH]c5ccc(C)cc45)CC[C@H]4CC[C@@H](C(=O)N(C)CCCCCCC#Cc5cccc6c5CN(C5CCC(=O)NC5=O)C6=O)N4C3=O)cc2c1 ; Cc1ccc2[nH]nc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(C(F)P(=O)(O)O)cc5c4)C3)c2c1]

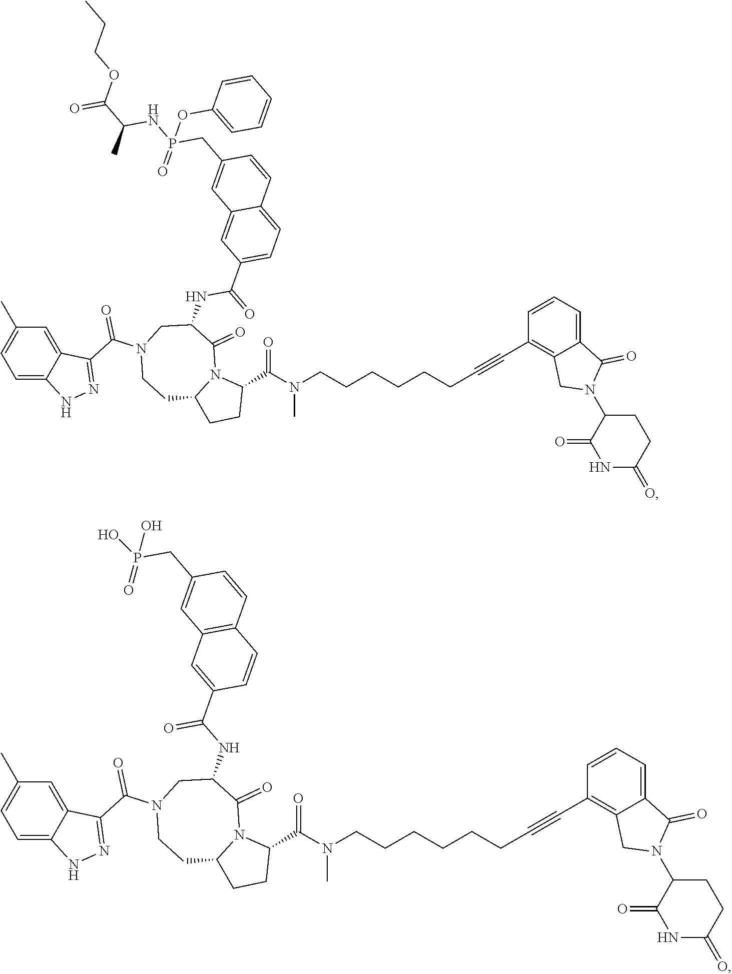 [SMILES: CCCOC(=O)[C@H](C)NP(=O)(Cc1ccc2ccc(C(=O)N[C@H]3CN(C(=O)c4n[nH]c5ccc(C)cc45)CC[C@H]4CC[C@@H](C(=O)N(C)CCCCCCC#Cc5cccc6c5CN(C5CCC(=O)NC5=O)C6=O)N4C3=O)cc2c1)Oc1ccccc1 ; Cc1ccc2[nH]nc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(CP(=O)(O)O)cc5c4)C3)c2c1]

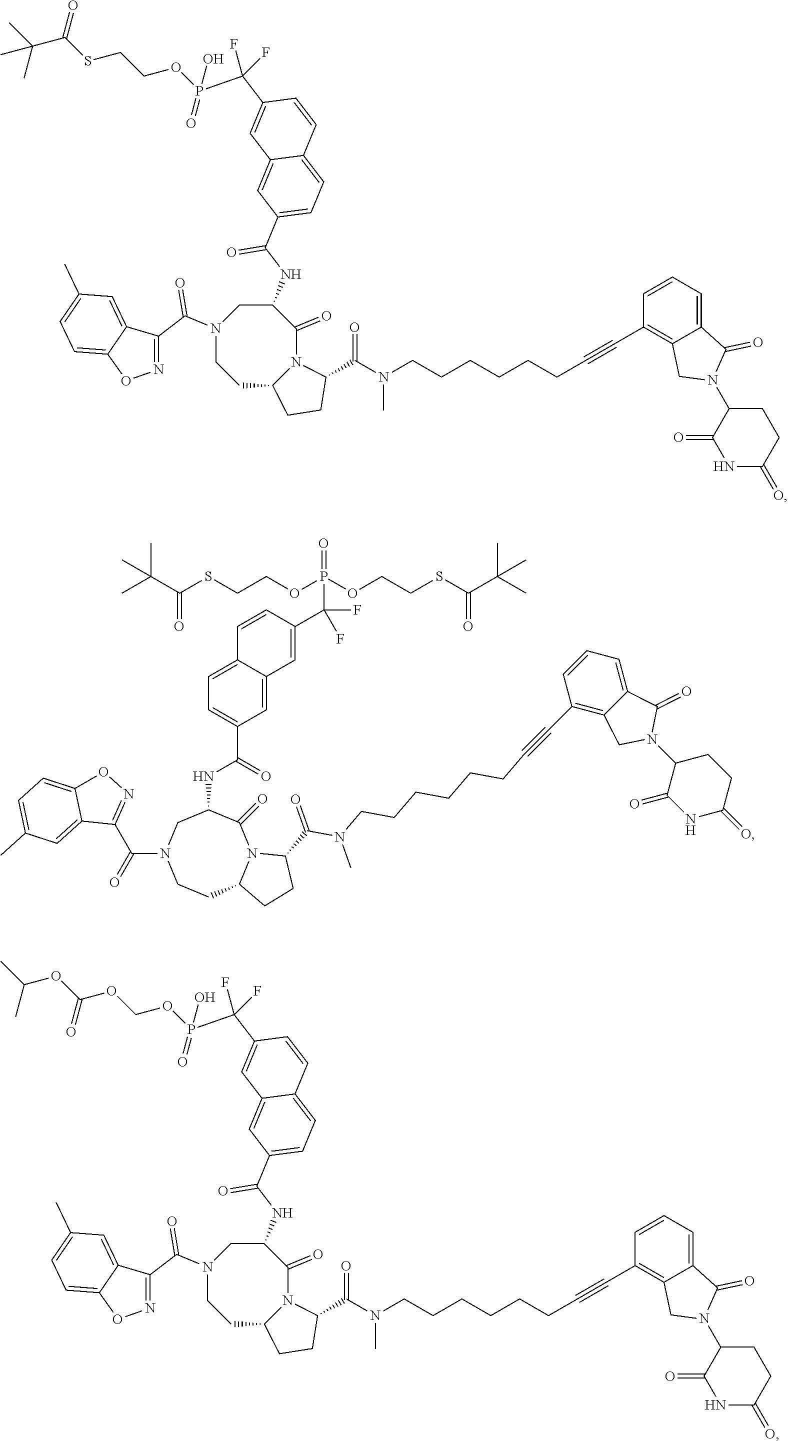

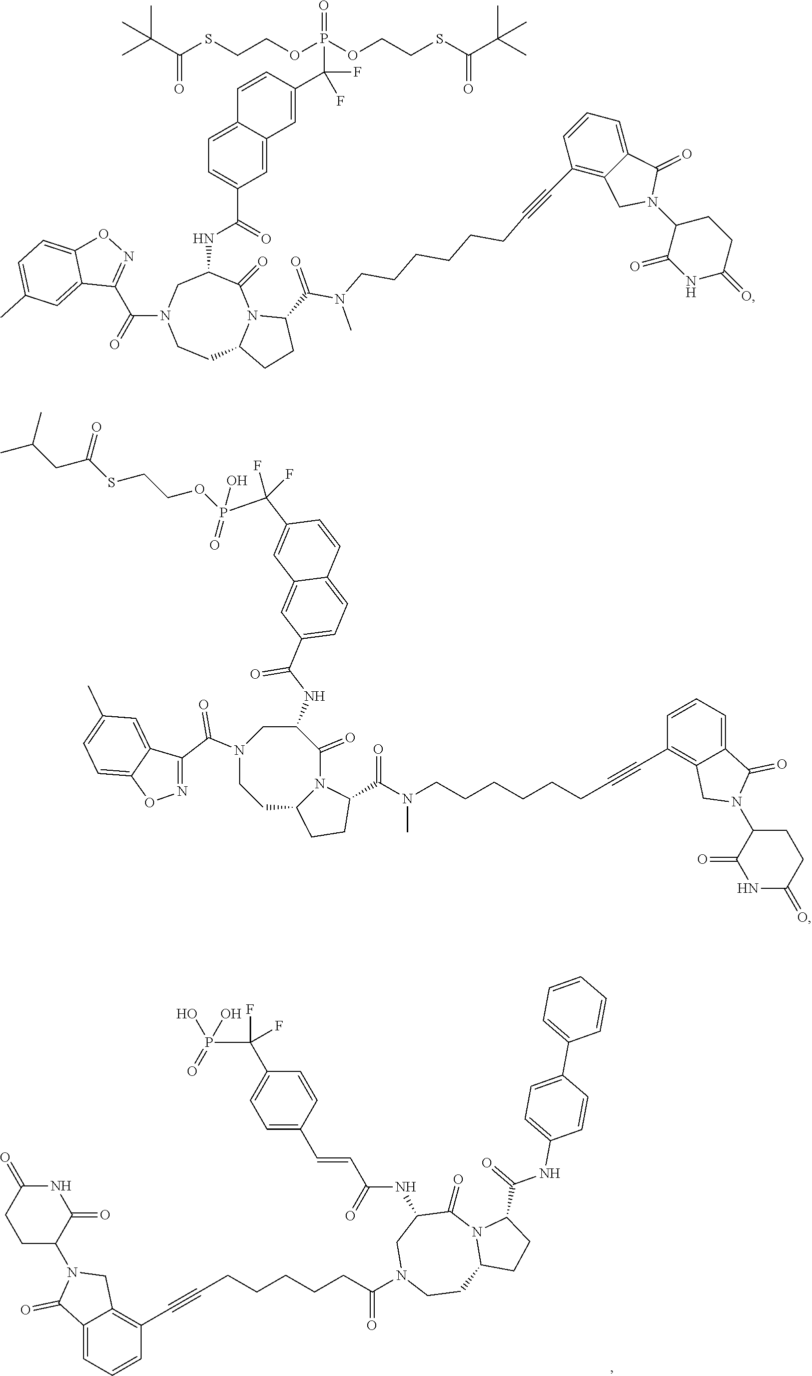 [SMILES: Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(C(F)(F)P(=O)(OCCSC(=O)C(C)(C)C)OCCSC(=O)C(C)(C)C)cc5c4)C3)c2c1 ; Cc1ccc2onc(C(=O)N3CC[C@H]4CC[C@@H](C(=O)N(C)CCCCCCC#Cc5cccc6c5CN(C5CCC(=O)NC5=O)C6=O)N4C(=O)[C@@H](NC(=O)c4ccc5ccc(C(F)(F)P(=O)(O)OCCSC(=O)CC(C)C)cc5c4)C3)c2c1 ; O=C(/C=C/c1ccc(C(F)(F)P(=O)(O)O)cc1)N[C@H]1CN(C(=O)CCCCCC#Cc2cccc3c2CN(C2CCC(=O)NC2=O)C3=O)CC[C@H]2CC[C@@H](C(=O)Nc3ccc(-c4ccccc4)cc3)N2C1=O]

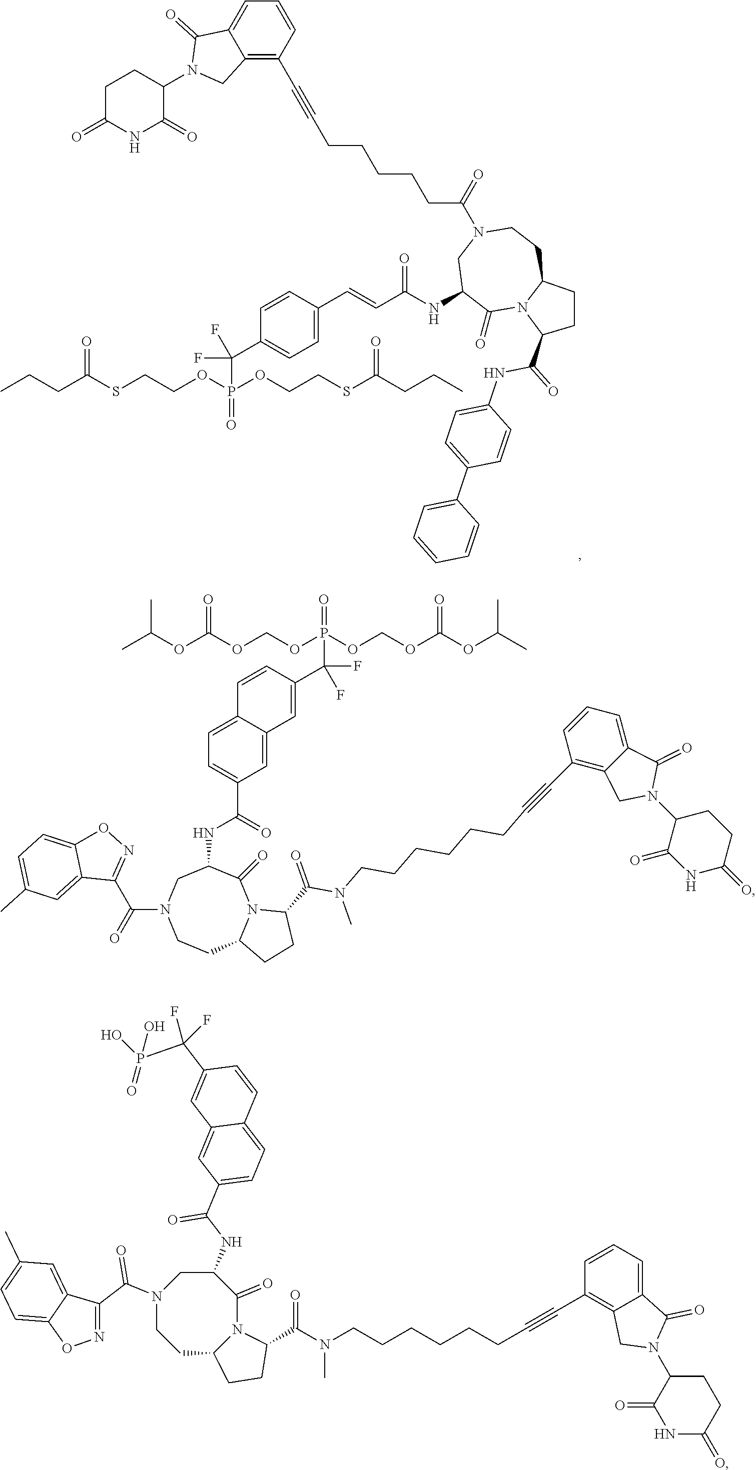

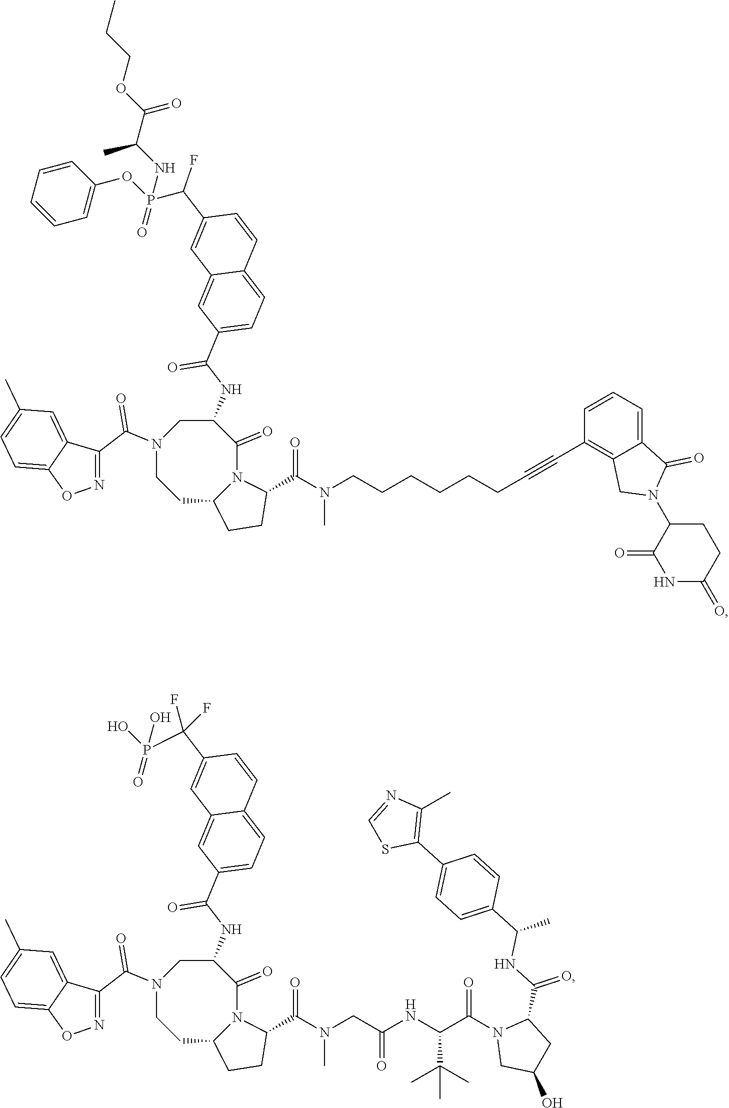 [SMILES: CCCOC(=O)[C@H](C)NP(=O)(Oc1ccccc1)C(F)c1ccc2ccc(C(=O)N[C@H]3CN(C(=O)c4noc5ccc(C)cc45)CC[C@H]4CC[C@@H](C(=O)N(C)CCCCCCC#Cc5cccc6c5CN(C5CCC(=O)NC5=O)C6=O)N4C3=O)cc2c1 ; Cc1ccc2onc(C(=O)N3CC[C@H]4CC[C@@H](C(=O)N(C)CC(=O)N[C@H](C(=O)N5C[C@H](O)C[C@H]5C(=O)N[C@@H](C)c5ccc(-c6scnc6C)cc5)C(C)(C)C)N4C(=O)[C@@H](NC(=O)c4ccc5ccc(C(F)(F)P(=O)(O)O)cc5c4)C3)c2c1]

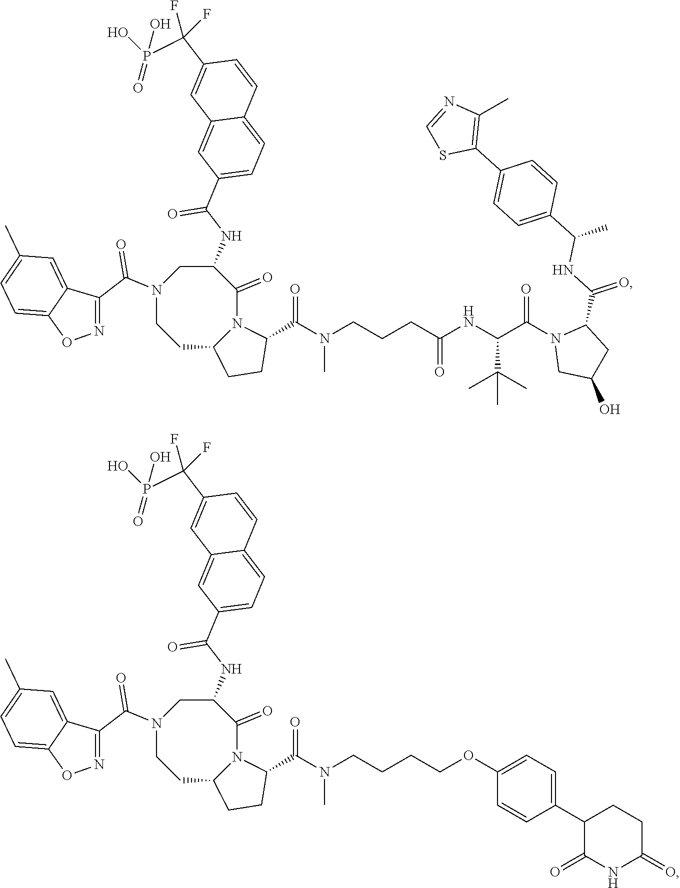 [SMILES: Cc1ccc2onc(C(=O)N3CC[C@H]4CC[C@@H](C(=O)N(C)CCCC(=O)N[C@H](C(=O)N5C[C@H](O)C[C@H]5C(=O)N[C@@H](C)c5ccc(-c6scnc6C)cc5)C(C)(C)C)N4C(=O)[C@@H](NC(=O)c4ccc5ccc(C(F)(F)P(=O)(O)O)cc5c4)C3)c2c1 ; Cc1ccc2onc(C(=O)N3CC[C@H]4CC[C@@H](C(=O)N(C)CCCCOc5ccc(C6CCC(=O)NC6=O)cc5)N4C(=O)[C@@H](NC(=O)c4ccc5ccc(C(F)(F)P(=O)(O)O)cc5c4)C3)c2c1]

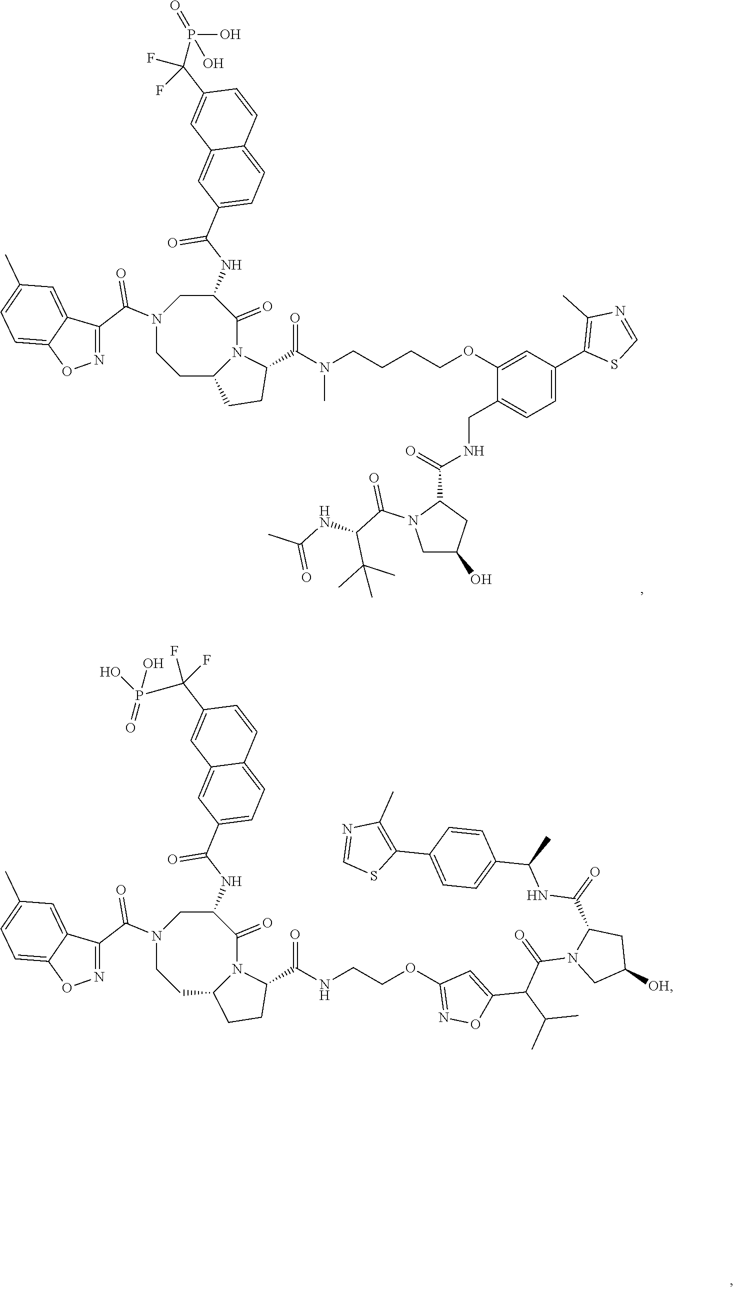

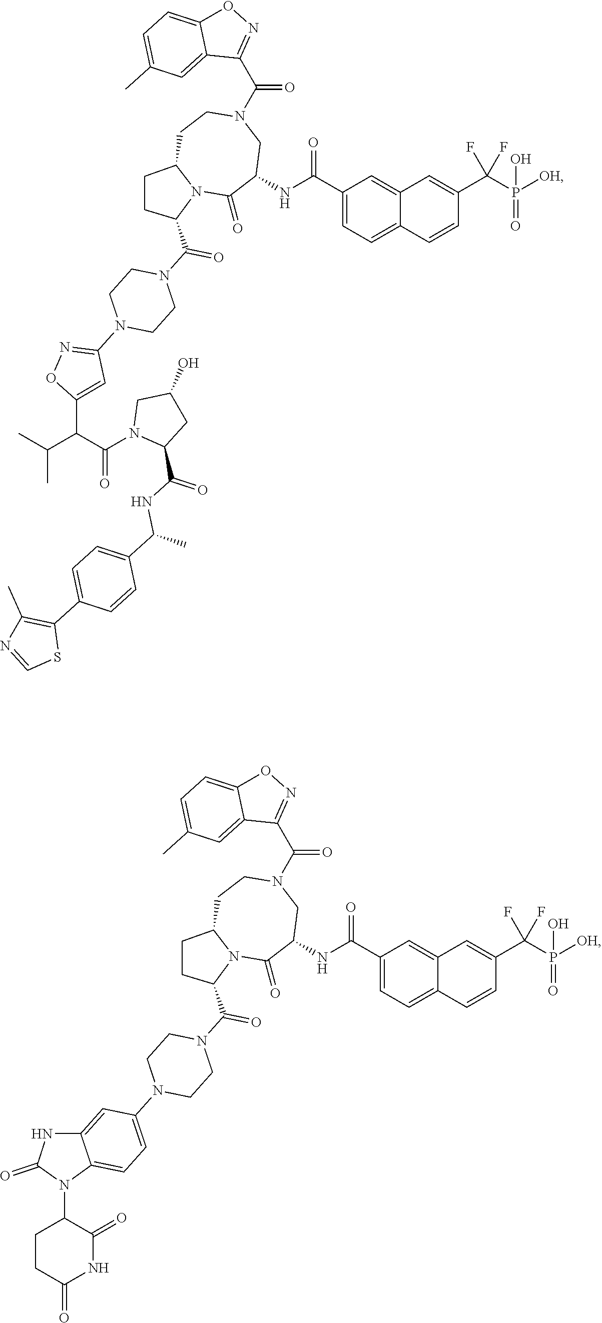 [SMILES: Cc1ccc2onc(C(=O)N3CC[C@H]4CC[C@@H](C(=O)N5CCN(c6cc(C(C(=O)N7C[C@H](O)C[C@H]7C(=O)N[C@H](C)c7ccc(-c8scnc8C)cc7)C(C)C)on6)CC5)N4C(=O)[C@@H](NC(=O)c4ccc5ccc(C(F)(F)P(=O)(O)O)cc5c4)C3)c2c1 ; Cc1ccc2onc(C(=O)N3CC[C@H]4CC[C@@H](C(=O)N5CCN(c6ccc7c(c6)[nH]c(=O)n7C6CCC(=O)NC6=O)CC5)N4C(=O)[C@@H](NC(=O)c4ccc5ccc(C(F)(F)P(=O)(O)O)cc5c4)C3)c2c1]

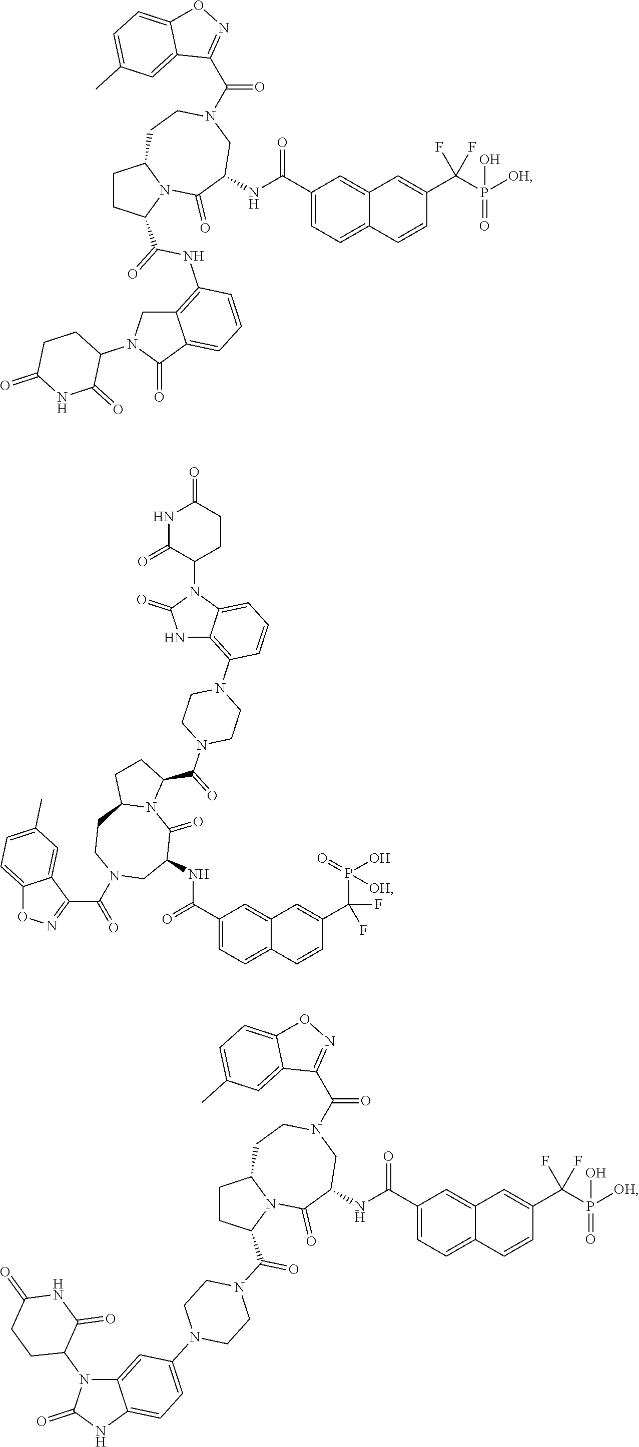 [SMILES: Cc1ccc2onc(C(=O)N3CC[C@H]4CC[C@@H](C(=O)Nc5cccc6c5CN(C5CCC(=O)NC5=O)C6=O)N4C(=O)[C@@H](NC(=O)c4ccc5ccc(C(F)(F)P(=O)(O)O)cc5c4)C3)c2c1 ; Cc1ccc2onc(C(=O)N3CC[C@H]4CC[C@@H](C(=O)N5CCN(c6cccc7c6[nH]c(=O)n7C6CCC(=O)NC6=O)CC5)N4C(=O)[C@@H](NC(=O)c4ccc5ccc(C(F)(F)P(=O)(O)O)cc5c4)C3)c2c1 ; Cc1ccc2onc(C(=O)N3CC[C@H]4CC[C@@H](C(=O)N5CCN(c6ccc7[nH]c(=O)n(C8CCC(=O)NC8=O)c7c6)CC5)N4C(=O)[C@@H](NC(=O)c4ccc5ccc(C(F)(F)P(=O)(O)O)cc5c4)C3)c2c1]

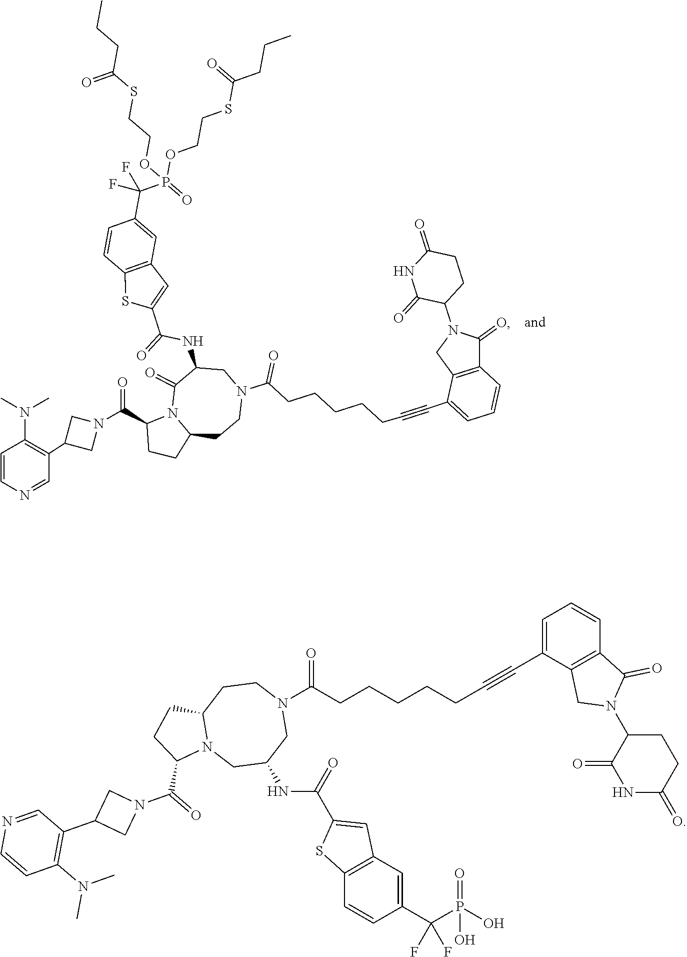 [SMILES: CCCC(=O)SCCOP(=O)(OCCSC(=O)CCC)C(F)(F)c1ccc2sc(C(=O)N[C@H]3CN(C(=O)CCCCCC#Cc4cccc5c4CN(C4CCC(=O)NC4=O)C5=O)CC[C@H]4CC[C@@H](C(=O)N5CC(c6cnccc6N(C)C)C5)N4C3=O)cc2c1 ; CN(C)c1ccncc1C1CN(C(=O)[C@@H]2CC[C@@H]3CCN(C(=O)CCCCCC#Cc4cccc5c4CN(C4CCC(=O)NC4=O)C5=O)C[C@H](NC(=O)c4cc5cc(C(F)(F)P(=O)(O)O)ccc5s4)CN32)C1]

49. A pharmaceutically acceptable composition comprising the compound of any one of claims 1 to 48, or a pharmaceutically acceptable salt thereof; and a pharmaceutically acceptable carrier.

50. A method of treating a condition responsive to the modulation of STAT3 or STAT6 in a subject comprising administering to the subject a therapeutically effective amount of the compound of any one of claims 1 to 48 or a pharmaceutically acceptable salt thereof, or the pharmaceutically acceptable composition of claim 49.
